(19) United States
(12) Patent Application Publication
(10) Pub. No.: US 2026/0226156 A1
(43) Pub. Date: Aug. 6, 2026

(54) IMMUNE EFFECTOR CELLS STABLY AND TRANSIENTLY EXPRESSING NUCLEIC ACIDS

(71) Applicant: BIONTECH CELL & GENE THERAPIES GMBH, Mainz (DE)

(72) Inventors: Ivana GRABUNDZIJA, Mainz (DE); Ilija BILIC, Mainz (DE); Razieh MONJEZI, Mainz (DE); Arne BILLMEIER, Mainz (DE); Benjamin RENGSTL, Mainz (DE); Ugur SAHIN, Mainz (DE); Andrew KAISER, Mainz (DE)

(21) Appl. No.: 19/154,106

(22) PCT Filed: Feb. 7, 2024
(86) PCT No.: PCT/EP2024/053043

§ 371 Date: Aug. 6, 2025

(30) Foreign Application Priority Data
Feb. 7, 2023 (WO) PCT/EP2023/052981

(51) Int. Cl. C07K 16/28; A61K 40/11; A61K 40/31; A61K 40/42; A61P 35/00; C12N 5/0783; C12N 9/12; C12N 15/88

(52) U.S. Cl. CPC C07K 16/28; A61K 40/11; A61K 40/31; A61K 40/4202; A61P 35/00; C12N 5/0638; C12N 9/1241; C12N 15/88; C12N 2510/00

(57) ABSTRACT

The present invention concerns immune effector cells comprising a first nucleic acid molecule comprising a first nucleotide sequence encoding a first cell-surface expressed antigen receptor and a second nucleic acid molecule comprising a second nucleotide sequence encoding an immune effector cell-activator molecule, cellular and pharmaceutical compositions comprising such immune effector cells, and methods of producing such immune effector cells. Furthermore, the invention concerns particles comprising a first nucleic acid molecule comprising a first nucleotide sequence encoding a first cell-surface expressed antigen receptor and a second nucleic acid molecule comprising a second nucleotide sequence encoding an immune effector cell-activator molecule and complexes comprising these particles. Further, the invention concerns methods of treating a subject having a disease, disorder or condition associated with expression or elevated expression of an antigen with the immune effector cells, particles or complexes.

### TECHNICAL FIELD

The present invention concerns immune effector cells comprising a first nucleic acid molecule comprising a first nucleotide sequence encoding a first cell-surface expressed antigen receptor and a second nucleic acid molecule comprising a second nucleotide sequence encoding an immune effector cell-activator molecule, cellular and pharmaceutical compositions comprising such immune effector cells, and methods of producing such immune effector cells. Furthermore, the invention concerns particles comprising a first nucleic acid molecule comprising a first nucleotide sequence encoding a first cell-surface expressed antigen receptor and a second nucleic acid molecule comprising a second nucleotide sequence encoding an immune effector cell-activator molecule and complexes comprising these particles. Further, the invention concerns methods of treating a subject having a disease, disorder or condition associated with expression or elevated expression of an antigen with the immune effector cells, particles or complexes.

### BACKGROUND

With hundreds of ongoing clinical trials to test the efficacy of engineered T cells as cancer therapies following on the successes of CD19 CAR-T cells for the treatment of hematological malignancies, the number of eligible patients for therapeutic modalities based on T cells is expected to increase massively. However, current T cell manufacturing technologies are ill-equipped to meet an increasing demand for CAR-T cells and related engineered T-cell immunotherapies, especially as current manufacturing approaches depend on time- and cost-intensive production protocols (Rafiq et al., Nat. Rev. Clin. Oncol. 2020 March; 17(3):147-167).

Standard production protocols for engineered T cells are largely performed manually or in a semi-automated manner, limiting throughput and increasing product variability and costs. The method generally involves collection of the patient's leukapheresis material and transport to a production facility, T-cell stimulation and transduction (typically by viral vectors encoding for the antigen receptor), expansion and cryopreservation of the T cell product under Good Manufacturing Practices (GMP) conditions, and then transport back to the original patient. Therefore, increases in the speed, scalability, and cost effectiveness—while maintaining or improving the biosafety and therapeutic potency of the product—are major priorities for optimizing CAR-T cell manufacturing (Blache et al., Nat. Commun., 2022 Sep. 5; 13(1):5225).

Currently approved CAR-T cell products typically depend on using γ-retroviruses or lentiviruses with high transduction efficiency for introduction of the CAR nucleic acid sequences into the patient's T cells. Further developments of these viral transduction methods have led to gains in manufacturing time (Ghassemi et al., Nat. Biomed. Eng. 2022 February; 6(2):118-128). However, viral vectors have limited genetic cargo capacity, typically require a T cell activation step detrimental to their persistence and anti-tumor efficacy, involve high production costs under GMP conditions, and face a complex regulatory environment. Hence, the development of alternative genetic engineering modalities has been a focus point in efforts to resolve the current limitations of CAR-T cell manufacturing. In this regard, transposon-based systems have emerged as a potential solution given their advantages in the context of safety, reproducibility, and speed over traditional viral transduction (Irving et al., Hum. Gene Ther. 2021 October; 32(19-20):1044-1058).

However, so far used transposon-based protocols have the disadvantages that they are less efficient for integration or bear the risk of excising again the once integrated sequence in case the transposase is expressed for a longer time. The efficiency is even more reduced with resting cells, such as non-activated T cells.

Hence, there remains an urgent need to improve the production of sufficient amounts of immune effector cells in vivo and/or in vitro, preferably in a safe and efficient way. This need is particularly prevalent for immune effector cells which should stably express a receptor.

The present invention fulfills such needs. The present invention has the particular advantages that it provides immune effector cells with less manufacturing time, fewer quality tests and/or reduced costs. In particular, there is no need for a further activation step.

### SUMMARY

The present disclosure generally relates to immune effector cells comprising a first nucleic acid molecule comprising a first nucleotide sequence encoding a first cell-surface expressed antigen receptor and a second nucleic acid molecule comprising a second nucleotide sequence encoding an immune effector cell-activator molecule and to particles and complexes useful for producing such immune effector cells. The immune effector cells, particles and complexes can further be used in methods for treating subjects. The immune effector cells of the present disclosure are characterized in that (i) the second nucleotide sequence is not integrated into a genomic nucleic acid molecule of the immune effector cell and/or (ii) the activator molecule is transiently expressed. In case the second nucleic acid is not integrated into a genomic nucleic acid molecule, which would be equally propagated to the daughter cells during cell division, the activator molecule is only expressed for a limited time.

The transient expression makes it possible that the activator molecule be present for the time needed, but thereafter will be lost. Transient expression may be achieved in that the second nucleic acid is not integrated into a genomic nucleic acid molecule, but it can also achieved by other means, for example, by regulating the induction of expression from the second nucleic acid molecule.

One aspect provided by the present disclosure is an immune effector cell comprising a first nucleic acid molecule comprising a first nucleotide sequence encoding a first cell-surface expressed antigen receptor and a second nucleic acid molecule comprising a second nucleotide sequence encoding an immune effector cell-activator molecule, wherein (i) the second nucleotide sequence is not integrated into a genomic nucleic acid molecule of the immune effector cell and/or (ii) the activator molecule is transiently expressed.

Without being bound by theory, providing an immune effector cell with both a first antigen receptor and an activator molecule has the advantage that additional activation steps can be omitted from or be integrated into the production method. Furthermore, the activation, in particular activation with a second antigen receptor, may help in overall transfection efficiency, as it may help the first nucleotide sequence overcome the nuclear envelope and be in the nucleus for propagation, in particular by integration such as by a transposon-based system. In addition, no further cells are needed for expression of the activator molecule, which also increases regulatory acceptance. Because the second nucleic acid is not integrated and/or the activator molecule is transiently expressed, the activation signal is lost when it is no longer needed, which increases the safety of the employed protocol.

In an embodiment, the immune effector cell can be isolated. The immune effector cell can be present in vitro, for example, in a cell culture or frozen sample, or in vivo.

In an embodiment, the first nucleic acid molecule can be DNA or RNA. Preferably, the first nucleic acid molecule can be DNA. An important characteristic of the first nucleic acid molecule is that it can ensure for stable, long-term expression of the encoded first cell-surface expressed antigen receptor.

In an embodiment, the first nucleotide sequence can be integrated into a genomic nucleic acid molecule of the immune effector cell. Preferably the genomic nucleic acid molecule can be a chromosome, an episome, such as a non-viral episome.

In an embodiment, the first nucleotide sequence can be integrated into the genomic nucleic acid molecule, preferably a chromosome, via a DNA-based transposon system, a viral-based retrotransposon system, or a poly-A-based retrotransposon system. According to this embodiment, the first nucleotide sequence can be comprised within an appropriate transposable element.

In an embodiment, the immune effector cell can further comprise a third nucleic acid molecule comprising a third nucleotide sequence encoding a molecule having transposase activity, preferably a transposase.

In an embodiment, the third nucleic acid molecule can be DNA or RNA. Preferably, the third nucleic acid molecule can be RNA, more preferably mRNA.

In an embodiment, the molecule having transposase activity can be Sleeping Beauty, PiggyBac, Frog, Prince, Himarl, Passport, Minos, hAT, Tol1, Tol2, AciDs, PIF, Harbinger, Harbinger3-DR, Hsmar1, or a functionally equivalent variant thereof having transposase activity. Preferably, the molecule having transposase activity is Sleeping Beauty transposase SB100X.

In an embodiment, the third nucleic acid molecule is not integrated into a genomic nucleic acid molecule of the immune effector cell and/or the molecule encoded by the third nucleic acid molecule having transposase activity can be transiently expressed. A characteristic of the third nucleic acid molecule is that it is not suitable for stable, long-term expression of the molecule having transposase activity. Without being bound by theory, this helps prevent the excision of the integrated first nucleotide sequence.

In an embodiment, the first cell-surface expressed antigen receptor can be stably expressed.

In an embodiment, the activator molecule encoded by the second nucleotide sequence can allow for the activation, expansion, differentiation and/or proliferation of the immune effector cell. Preferably, the activator molecule can be a non-coding RNA or protein.

In an embodiment, the activator molecule can bind to the extracellular portion of the first cell-surfaced expressed antigen receptor. Preferably, the activator molecule can be an antigen targeted/bound by the first cell-surface expressed antigen receptor.

In an embodiment, the activator molecule can be a cytokine.

In an embodiment, the activator molecule can be a second cell-surface expressed antigen receptor, wherein the extracellular portions of the first and second cell-surfaced expressed antigen receptors do not bind to the same binding target.

In an embodiment, the immune effector cell can further comprise a fourth nucleic acid molecule comprising a fourth nucleotide sequence encoding the binding target of the first cell-surface expressed antigen receptor and/or the immune effector cell can further comprise a fifth nucleic acid molecule comprising a fifth nucleotide sequence encoding the binding target of the second cell-surfaced expressed antigen receptor. In this embodiment, the fourth nucleic acid molecule or the fifth nucleic acid molecule can both comprise the fourth nucleotide sequence encoding the binding target of the first cell-surfaced expressed antigen receptor and the fifth nucleotide sequence encoding the binding target of the second cell-surfaced expressed antigen receptor.

In an embodiment, the fourth and/or fifth nucleic acid molecule can be DNA or RNA. Preferably, the fourth and/or fifth nucleic acid molecule can be RNA, more preferably mRNA.

In an embodiment, the first and/or the second cell-surfaced expressed antigen receptor can be a chimeric antigen receptor (CAR) or a T cell receptor (TCR), such as an artificial T cell receptor.

In an embodiment, the binding target of the second cell-surfaced expressed antigen receptor can be expressed on or from cells different from cells expressing the binding target of the first cell-surfaced expressed antigen receptor.

In an embodiment, the binding target of the first cell-surfaced expressed antigen receptor can be a tumor-associated antigen or an antigen of an infectious agent, or an epitope thereof.

In an embodiment, the binding target of the second cell-surfaced expressed antigen receptor can be a cell-surface-expressed protein or a soluble protein, or an epitope thereof. The cell-surface expressed protein can be a glycoprotein or a cell-surface expressed cytokine. Preferably, the cell-surface expressed protein can be a cluster of differentiation (CD) protein and the soluble protein can be preferably a soluble cytokine.

In an embodiment, the binding target of the second cell-surfaced expressed antigen receptor can be a cell-surface protein expressed on a blood cell, which blood cell is preferably another immune effector cell. Preferably, the blood cell can be a T cell, a NK cell, a dendritic cell, a macrophage, or a B cell.

In an embodiment, the cell-surface protein can be CD19 or CLDN18.2.

In an embodiment, the second nucleic acid molecule can be DNA or RNA. Preferably, the second nucleic acid molecule can be RNA, more preferably mRNA. Preferably, the RNA or mRNA comprises a ribonucleobase other than A, C, G and U. The ribonucleobase can be pseudouridine, preferably 1-methyl-pseudouridine. A characteristic of the second nucleic acid molecule is that it is not suitable for stable, long-term expression of the activator molecule.

In an embodiment, the RNA can comprise a 5′ cap structure. Preferably, the 5′ cap structure is a natural occurring cap or a cap analog. The 5′ cap structure can be one of the following: cap0, cap1, cap2, cap3, cap4, ARCA (Anti-Reverse Cap Analogs), modified ARCA, inosine, N1-methyl-guanosine, 2′-fluoro-guanosine, 7-deaza-guanosine, 8-oxo-guanosine, 2-amino-guanosine, LNA-guanosine and 2-azido-guanosine. Preferably, the 5′ cap structure can be cap0, which is m7G(5′)ppp(5′) or cap1, which is m7G(5′)ppp(5′)(N₁^{2′-OMe}). In this embodiment, the N₁ can be chosen from A, C, G or U. The cap1 can further comprise a second nucleotide N₂, which is a cap proximal A, G, C or U at position +2 and is represented as m7G(5′)ppp(5′)(N₁^{2′-OMe})pN₂. In an embodiment, the structure of the 5′ terminus with is m7G(5′)ppp(5′)(A^{2′-OMe})pGpApApU.

In an embodiment, the immune effector cell can be a T cell, a B cell, a dendritic cell, or a NK cell. Preferably, the immune effector cell is a CD8+ and/or CD4+ T cell, more preferably a cytotoxic T cell.

In an embodiment, the second nucleic acid may not be inherited by the progeny cells of the immune effector cell in the same manner as chromosomes are inherited, and/or the second nucleic acid can be diluted out compared to the total number of cells in each generation of progeny cells of the immune effector cell, and/or after one round of cell division, the amount of the second nucleic acid molecule may be less in each daughter cell compared to the amount in the parental cell.

A further embodiment may be an immune effector cell comprising (i) a DNA molecule comprising a transposable element, which element comprises a nucleotide sequence encoding a first T cell receptor or chimeric antigen receptor which binds to a tumor-associated antigen; (ii) an mRNA molecule encoding a second T cell receptor or chimeric antigen receptor which binds to a target different from the tumor-associated antigen; and (iii) an mRNA molecule encoding for a transposase.

A further embodiment may be an immune effector cell comprising (i) a nucleotide sequence encoding a first T cell receptor or a chimeric antigen receptor which binds to a tumor-associated antigen, which nucleotide sequence is integrated into the genome of the immune effector cell or is comprised within an episome present in the immune effector cell; and (ii) a nucleic acid molecule encoding a second T cell receptor or a chimeric antigen receptor which binds to a target different from the tumor-associated antigen, which nucleic acid molecule is not integrated into a genomic nucleic acid molecule of the immune effector cell.

A further embodiment may be an immune effector cell comprising (i) a DNA sequence encoding a first chimeric antigen receptor which binds to a tumor-associated antigen, which DNA sequence is integrated into the genome of the immune effector cell; and (ii) an mRNA molecule encoding a second chimeric antigen receptor which binds to a target different from the tumor-associated antigen.

In an embodiment, the immune effector cell can be a CD8+ cytotoxic T cell.

In an embodiment, the immune effector cell can be activated by the binding of the second T cell receptor (TCR) or chimeric antigen receptor (CAR) binding to its target.

In an embodiment, the immune effector cell may not comprise a DNA nucleotide sequence encoding for the activator molecule.

In an embodiment, the immune effector cell may have reduced cell-surface expression of the endogenous T cell receptor to a level that prevents graft-versus-host activity of the immune effector cell when administered to a subject different from the subject from whom the immune effector cell was derived. Preferably, the immune effector cell does not express its endogenous T cell receptor on its cell-surface.

In an embodiment, the immune effector cell may have reduced cell-surface expression of the endogenous HLA complex to a level that prevents host-versus-graft activity in a subject to whom the immune effector cell has been administered, wherein the subject administered the immune effector cell is different from the subject from whom the immune effector cell was derived. Preferably, the immune effector cell does not express its endogenous HLA complex on its cell-surface.

A further aspect is a cellular composition comprising the immune effector cell according to the present disclosure. The cellular composition can further comprise a cryopreservation agent.

A further aspect is a pharmaceutical composition comprising the immune effector cell according to the present disclosure or the cellular composition according to the present disclosure, and a pharmaceutically acceptable carrier.

In an embodiment, the immune effector cell according to the present disclosure, the cellular composition according to the present disclosure, or the pharmaceutical composition according to the present disclosure is for use in a method of treating a subject having a disease, disorder or condition associated with expression or elevated expression of the binding target of the first cell-surface expressed antigen receptor, wherein the method comprises administering the immune effector cell, the cellular composition or the pharmaceutical composition to the subject. Preferably, the disease, disorder or condition can be cancer, preferably the cancer is a solid cancer.

In an embodiment, the immune effector cell, the cellular composition or the pharmaceutical composition for use according to the present disclosure, the disease, disorder or condition can be an infection, preferably the infection is a viral infection.

In an embodiment, the immune effector cell, the cellular composition or the pharmaceutical composition for use according to present disclosure, the immune effector cell is autologous or heterologous to the subject being administered the immune effector cell, the cellular composition or the pharmaceutical composition.

All embodiments applying to the immune effector cells equally apply to the particles, complexes and methods disclosed herein.

A further aspect is a particle comprising (i) a first nucleic acid molecule comprising a first nucleotide sequence encoding a first cell-surfaced expressed antigen receptor, which first nucleotide sequence is comprised within a transposable element; and (ii) a second nucleic acid molecule comprising a second nucleotide sequence encoding an immune effector cell-activator molecule, wherein the second nucleotide sequence is not comprised within a transposable element, preferably wherein the particle further comprises a third nucleic acid molecule comprising a third nucleotide sequence encoding a molecule having transposase activity, preferably transposase, wherein the third nucleotide sequence is not comprised within a transposable element. Preferably, the first nucleic acid molecule or episome is a DNA minicircle or a linear DNA molecule.

In an embodiment, the particle can comprise (i) a DNA episome comprising a first nucleotide sequence encoding a first cell-surfaced expressed antigen receptor, preferably a non-viral episome; and (ii) a second nucleic acid molecule comprising a second nucleotide sequence encoding an immune effector cell-activator molecule, wherein the second nucleic acid molecule, when present in a cell provides for transient expression of the activator molecule. Preferably, the first nucleic acid molecule can be DNA or RNA.

In an embodiment of a particle according to the present disclosure, the transposable element in the first nucleic acid can be derived from a DNA-based transposon system, a viral-based transposon system, or a poly-A-based retrotransposon system.

In an embodiment of a particle according to the present disclosure, the third nucleic acid molecule can be DNA or RNA. Preferably, the third nucleic acid molecule is RNA, more preferably mRNA.

In an embodiment of a particle according to the present disclosure, the molecule having transposase activity can be Sleeping Beauty, PiggyBac, Frog, Prince, Himarl, Passport, Minos, hAT, Tol1, Tol2, AciDs, PIF, Harbinger, Harbinger3-DR, Hsmar1, or a functionally equivalent variant thereof having transposase activity. Preferably, the molecule having transposase activity is Sleeping Beauty transposase SB100X.

In an embodiment of a particle according to the present disclosure, the second nucleic acid molecule can be DNA or RNA. Preferably the second nucleic acid molecule is RNA, more preferably mRNA.

In an embodiment of a particle according to the present disclosure, the first cell-surfaced expressed antigen receptor can bind to a tumor-associated antigen or an antigen of an infectious agent, or epitope thereof.

In an embodiment of a particle according to the present disclosure, the first cell-surfaced expressed antigen receptor can be a chimeric antigen receptor (CAR) or T cell receptor (TCR).

In an embodiment of a particle according to the present disclosure, the activator molecule allows for the activation, expansion, differentiation and/or proliferation of the immune effector cell. The activator molecule can be a non-coding RNA or protein.

In an embodiment of a particle according to the present disclosure, the activator molecule can bind to the extracellular portion of the first cell-surfaced expressed antigen receptor. Preferably, the activator molecule is an antigen targeted by the first cell-surface expressed antigen receptor.

In an embodiment of a particle according to the present disclosure, the activator molecule can be a cytokine.

In an embodiment of a particle according to the present disclosure, the activator molecule can be a second cell-surface expressed antigen receptor, wherein the extracellular portions of the first and second cell-surfaced expressed antigen receptors do not bind to the same binding target. Preferably, the second cell-surface expressed antigen receptor is a chimeric antigen receptor (CAR) or a T cell receptor (TCR).

In an embodiment, the particle can further comprise a fourth nucleic acid molecule comprising a fourth nucleotide sequence encoding the binding target of the first cell-surface expressed antigen receptor, wherein the fourth nucleotide sequence is not comprised within a transposable element, or the particle can further comprise a fifth nucleic acid molecule comprising a fifth nucleotide sequence encoding the binding target of the second cell-surface expressed antigen receptor, wherein the fifth nucleotide sequence is not comprised within a transposable element. Preferably, the fourth nucleic acid molecule or the fifth nucleic acid molecule comprise both the fourth nucleotide sequence encoding the binding target of the first cell-surface expressed antigen receptor and the fifth nucleotide sequence encoding the binding target of the second cell-surface expressed antigen receptor.

In an embodiment of a particle according to the present disclosure, the fourth and/or fifth nucleic acid molecule can be DNA or RNA. Preferably, the fourth and/or fifth nucleic acid molecule is RNA, more preferably mRNA.

In an embodiment, the particle can comprise the first, second and third nucleic acid molecule specified herein. Preferably, the particle further comprises the fourth and/or fifth nucleic acid molecule specified herein.

In an embodiment, the particle can comprise the first, second, third and fourth nucleic acid molecule specified herein. Preferably, the particle further comprises the fifth nucleic acid molecule specified herein.

In an embodiment, the particle can comprise a polyalkyleneimine or a lipid. Preferably, the particle comprises a lipid, preferably comprising a lipid with a cationic headgroup and/or a pH responsive lipid and/or a PEGylated-lipid.

In an embodiment, the particle can be a lipid particle, polymer particle, or a mixture thereof.

In an embodiment, the particle can be a nanoparticle.

In an embodiment, the particle can be a lipid nanoparticle (LNP), a lipoplex, (LPX), a polyplex (PLX), or a lipopolyplex (LPLX) particle.

In an embodiment, the particle can further comprise at least one phosphatidylserine.

In an embodiment, the particles can be nanoparticles, in which:
(i) the number of positive charges in the nanoparticles does not exceed the number of negative charges in the nanoparticles and/or
(ii) the nanoparticles have a neutral or net negative charge and/or
(iii) the zeta potential of the nanoparticles is 0 or less. Optionally or also, the charge ratio of positive charges to negative charges in the nanoparticles can be 1.4:1 or less.

In an embodiment, the particle can comprise polyalkyleneimine, preferably wherein (a) the molar ratio of the number of nitrogen atoms (N) in the polyalkyleneimine to the number of phosphor atoms (P) in the first, second, and optionally third nucleic acid molecules (N:P ratio) can be 2.0 to 15.0, preferably 6.0 to 12.0; or (b) the molar ratio of the number of the number of nitrogen atoms (N) in the polyalkyleneimine to the number of phosphor atoms (P) in the first, second, and optionally third nucleic acid molecules (N:P ratio) is at least about 48, optionally about 48 to 300, about 60 to 200, or about 80 to 150, or preferably wherein the ionic strength of the composition is 50 mM or less, preferably wherein the concentration of monovalent cationic ions is 25 mM or less and the concentration of divalent cationic ions is 20 μM or less.

In an embodiment, the particle can be a polyplex particle.

In an embodiment, the particle can comprise a hydrophobic moiety having a binding moiety covalently attached thereto, preferably wherein the hydrophobic moiety having a binding moiety covalently attached thereto and the particle are non-covalently associated with each other. The hydrophobic moiety having a binding moiety covalently attached thereto can be an integral part of the particle. Preferably, the hydrophobic moiety having a binding moiety covalently attached thereto comprises a polymer.

In an embodiment, the hydrophobic moiety having a binding moiety covalently attached thereto can comprise a compound of Formula I

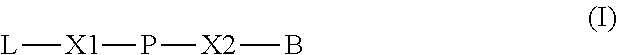

wherein
P comprises a polymer;
L comprises a hydrophobic moiety attached to a first end of the polymer;
B comprises a binding moiety attached to a second end of the polymer;
X1 is absent or a first linking moiety; and
X2 is absent or a second linking moiety, preferably X1 comprises a carbonyl group and/or preferably X2 comprises the reaction product of a maleimide group with a thiol or cysteine group of a compound comprising the binding moiety.

In an embodiment, the hydrophobic moiety can be or can be comprised in a lipid.

In an embodiment, the polymer can provide stealth property, extend circulation half-life and/or reduce non-specific protein binding or cell adhesion.

In an embodiment, the polymer can comprise polyethyleneglycol (PEG).

In an embodiment of a particle according to the present disclosure, the hydrophobic moiety having a binding moiety covalently attached thereto can comprise a compound of Formula II

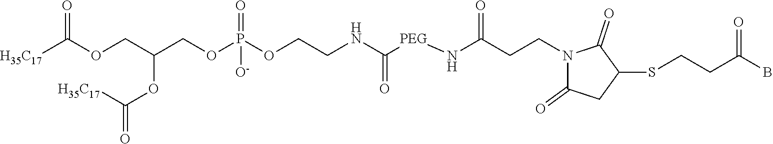

wherein B comprises the binding moiety, preferably B comprises a moiety comprising the structure —N-peptide-C(O)—NH₂.

In an embodiment, the binding moiety covalently attached to the hydrophobic moiety can comprise an antibody or an antibody derivative.

In an embodiment, the particle is complexed with the nucleic acid molecules and/or encapsulates the nucleic acid molecules.

A further aspect is a pharmaceutical composition comprising a particle described herein, and a pharmaceutically acceptable carrier.

In an embodiment, a particle or pharmaceutical composition according to the present disclosure can be for use in a method of treating a subject having a disease, disorder or condition associated with expression or elevated expression of the binding target of the first cell-surface expressed antigen receptor, wherein the method comprises administering the particle or the pharmaceutical composition to the subject, preferably the disease, disorder or condition is cancer, wherein the cancer is preferably a solid cancer.

In an embodiment, the particle or the pharmaceutical composition can be for use according to the present disclosure, wherein the disease, disorder or condition can be an infection, preferably wherein the infection is a viral infection.

A further aspect of the present disclosure is a complex comprising
(a) the particle according to the present disclosure, wherein the particle comprises a hydrophobic moiety having a binding moiety covalently attached thereto, and (b) a compound comprising (i) a moiety binding to the binding moiety covalently attached to the hydrophobic moiety and (ii) a moiety targeting a cell-surface antigen, preferably wherein the moiety binding to the binding moiety covalently attached to the hydrophobic moiety comprises an antibody or an antibody derivative, preferably wherein the binding moiety covalently attached to the hydrophobic moiety comprises a peptide comprising an ALFA-tag; and the moiety binding to the binding moiety covalently attached to the hydrophobic moiety comprises an antibody or an antibody derivative, preferably a nanobody, comprising a VHH domain comprising the CDR1 sequence VTISALNAMAMG, the CDR2 sequence AVSERGNAM, and the CDR3 sequence LEDRVDSFHDY. Preferably, (i) the moiety binding to the binding moiety covalently attached to the hydrophobic moiety and (ii) the moiety targeting a cell-surface antigen are linked to each other.

In an embodiment, the compound under (b) can comprise a peptide or polypeptide.

In an embodiment, the moiety targeting a cell-surface antigen can comprise an antibody or an antibody derivative.

In an embodiment, the cell-surface antigen can be characteristic for an immune effector cell. Preferably, the cell-surface antigen comprises CD4 and/or CD8 and/or CD3.

In an embodiment, the complex according to the present disclosure may be for use in the treatment of a subject having a disease, disorder or condition associated with expression or elevated expression of the binding target of the first cell-surface expressed antigen receptor.

A further aspect is a method of producing an immune effector cell expressing a first antigen receptor on the cell-surface, the method comprising contacting an immune effector cell with (i) a first nucleic acid molecule comprising a first nucleotide sequence encoding a first cell-surface expressed antigen receptor, and (ii) a second nucleic acid molecule comprising a second nucleotide sequence encoding an immune effector cell-activator molecule, wherein the second nucleotide sequence is not comprised within a transposable element, and wherein the first cell-surface expressed antigen receptor is stably expressed in the cell and the activator molecule is transiently expressed in the cell. Preferably, the first nucleic acid molecule is DNA or RNA.

In an embodiment, the method can further comprise integrating the first nucleotide sequence into a genomic nucleic acid molecule of the immune effector cell. Preferably, the first nucleotide sequence is comprised within a transposable element.

In an embodiment, the method can further comprise contacting the immune effector cell with a third nucleic acid molecule comprising a third nucleotide sequence encoding a molecule having transposase activity, preferably transposase, wherein the third nucleotide sequence is not comprised within a transposable element. Preferably, the third nucleic acid molecule is DNA or RNA. Preferably, the third nucleic acid molecule is RNA, more preferably mRNA. A characteristic of the third nucleic acid molecule is that it is not suitable for stable, long-term expression of the molecule having transposase activity.

In an embodiment, the method can further comprise contacting the immune effector cell with a fourth and/or fifth nucleic acid molecule comprising a fourth nucleotide sequence encoding the binding target of the first and/or second cell-surfaced expressed antigen receptor. Preferably, the fourth nucleic acid molecule or the fifth nucleic acid molecule comprise both the fourth nucleotide sequence encoding the binding target of the first cell-surfaced expressed antigen receptor and the fifth nucleotide sequence encoding the binding target of the second cell-surfaced expressed antigen receptor. The fourth and/or fifth nucleic acid molecule can be DNA or RNA. Preferably, the fourth and/or fifth nucleic acid molecule is RNA, more preferably mRNA.

In an embodiment, the first cell-surfaced expressed antigen receptor and/or the second cell-surfaced expressed antigen receptor can be a chimeric antigen receptor (CAR) or a T cell receptor (TCR).

In an embodiment of the methods of producing an immune effector cell, the binding target of the second cell-surfaced expressed antigen receptor is expressed on or from cells different from cells expressing the binding target of the first cell-surface expressed antigen receptor. The binding target of the first cell-surfaced expressed antigen receptor can be a tumor-associated antigen or an antigen of an infectious agent, or an epitope thereof.

In an embodiment of the methods of producing an immune effector cell, the second nucleic acid molecule is DNA or RNA. Preferably, the second nucleic acid molecule is RNA, more preferably mRNA.

In an embodiment of the methods of producing an immune effector cell, the RNA comprises a 5′ cap structure, preferably the 5′ cap structure is a natural occurring cap or a cap analog.

In an embodiment of the methods of producing an immune effector cell, the immune effector cell is a T cell, a B cell, a dendritic cell, or a NK cell. Preferably, the immune effector cell is a CD8+ and/or CD4+ T cell, preferably a cytotoxic T cell.

In an embodiment of the methods of producing an immune effector cell, the second nucleic acid is not inherited by the progeny cells of the immune effector cell in the same manner as chromosomes are inherited, and/or the second nucleic acid can be diluted out in each generation of progeny cells of the immune effector cell, and/or after one round of cell division, the amount of the second nucleic acid molecule may be less in each daughter cell compared to the amount in the parental cell.

In an embodiment, the method comprises contacting an immune effector cell with a particle or a complex according to the present disclosure. Preferably, the contacting occurs in vitro. The method can further comprise, after contacting the nucleic acid molecules to the immune effector cell, a step of contacting the immune effector cell with the binding target of the second cell-surface expressed antigen receptor or a cell expressing the binding target.

In an embodiment, the method can be a method of producing an immune effector cell expressing two antigen receptors on the cell-surface, the method comprising contacting, in vitro or ex vivo, an immune effector cell with (i) a DNA molecule comprising a first nucleotide sequence encoding a first cell-surface expressed antigen receptor, which first nucleotide sequence is comprised with a transposable element; (ii) an RNA molecule comprising a second nucleotide sequence encoding a second cell-surface expressed antigen receptor, which second nucleotide sequence is not comprised within a transposable element; and (iii) an RNA molecule comprising a third nucleotide sequence encoding a transposase, which third nucleotide sequence is not comprises within a transposable element, wherein the extracellular domains of the first and second cell-surface expressed antigen receptors bind to different targets, preferably wherein the binding target of the first cell-surface expressed antigen receptor is a tumor or tumor-associated antigen and the binding target of the second cell-surface expressed antigen receptor is expressed on the surface of a blood cell. Preferably, the method further comprises contacting the immune effector cell with the binding target of the second cell-surface expressed antigen receptor or with a cell expressing the binding target of the second cell-surface expressed antigen receptor.

In an embodiment, the method can be a method of producing an immune effector cell expressing two antigen receptors on the cell-surface, the method comprising contacting an immune effector cell with a particle, which particle comprises (i) a DNA molecule comprising a first nucleotide sequence encoding a first cell-surface expressed antigen receptor, which first nucleotide sequence is comprised with a transposable element; (ii) an mRNA molecule comprising a second nucleotide sequence encoding a second cell-surface expressed antigen receptor; and (iii) an mRNA molecule comprising a third nucleotide sequence encoding a transposase; wherein the extracellular domains of the first and second cell-surface expressed antigen receptors bind to different targets, preferably wherein the binding target of the first cell-surface expressed antigen receptor is a tumor or tumor-associated antigen and the binding target of the second cell-surface expressed antigen receptor is expressed on the surface of a blood cell. Preferably, the contacting is in vivo.

A further aspect of the present disclosure is a method of treating a subject having a disease, disorder or condition associated with expression or elevated expression of an antigen, the method comprising administering to the subject a first nucleic acid molecule comprising a first nucleotide sequence encoding a first cell-surface expressed antigen receptor and a second nucleic acid molecule comprising a second nucleotide sequence encoding an immune effector cell activator molecule, wherein the binding target of the first cell-surface expressed antigen receptor is the antigen that is associated with the disease, disorder or condition, wherein (i) the second nucleotide sequence does not integrate into a genomic nucleic acid molecule of the cells of the subject or is comprised within an episome present in the cells of the subject and/or (ii) the activator molecule is transiently expressed in the subject. Preferably, the nucleic acid molecules are in particle comprising a lipid.

In an embodiment, the method can be a method of treating a subject having a disease, disorder or condition associated with expression or elevated expression of an antigen, the method comprising administering to the subject a particle, which particle comprises (i) a DNA molecule comprising a first nucleotide sequence encoding a first cell-surface expressed antigen receptor, which first nucleotide sequence is comprised with a transposable element and wherein the binding target of the first cell-surface expressed antigen receptor is the antigen that is associated with the disease, disorder or condition; (ii) an mRNA molecule comprising a second nucleotide sequence encoding a second cell-surface expressed antigen receptor; and (iii) an mRNA molecule comprising a third nucleotide sequence encoding a transposase; wherein the extracellular domains of the first and second cell-surface expressed antigen receptors bind to different targets.

In an embodiment, the method can be a method of treating a subject having a disease, disorder or condition associated with expression or elevated expression of an antigen, the method comprising administering to the subject the immune effector cell according to the present disclosure, the cellular composition according to the present disclosure, or the pharmaceutical composition according to the present disclosure, wherein the binding target of the first cell-surface expressed antigen receptor is the antigen that is associated with the disease, disorder or condition.

In an embodiment, the method can be a method of treating a subject having a disease, disorder or condition associated with expression or elevated expression of an antigen, the method comprising administering to the subject a particle according to the present disclosure or the pharmaceutical composition according to the present disclosure, wherein the binding target of the first cell-surface expressed antigen receptor is the antigen that is associated with the disease, disorder or condition.

In an embodiment, the method can be a method of treating a subject having a disease, disorder or condition associated with expression or elevated expression of an antigen, the method comprising administering to the subject a complex according to the present disclosure, wherein the binding target of the first cell-surface expressed antigen receptor is the antigen that is associated with the disease, disorder or condition

In an embodiment of the methods of treating a subject, the antigen associated with a disease, disorder or condition can be a tumor-associated antigen. Preferably, the method is a method for treating or preventing cancer in a subject.

In an embodiment of the methods of treating a subject, the antigen associated with a disease, disorder or condition comprises an antigen of an infectious agent. Preferably, the infectious agent is a virus.

In an embodiment of the methods of treating a subject, the method is a method for treating or preventing an infection in a subject.

### DETAILED DESCRIPTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present invention will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to disclose and encompass embodiments which combine the explicitly described embodiments with any number of the disclosed and/or preferred elements. Furthermore, any permutations and combinations of all described elements in this application should be considered disclosed by this description unless the context indicates otherwise.

The term “about” means approximately or nearly, and in the context of a numerical value or range set forth herein preferably means+/−10% of the numerical value or range recited or claimed.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed.

No language in the specification should be construed as indicating any non-claimed element essential to the practice of the invention.

Unless expressly specified otherwise, the term “comprising” is used in the context of the present document to indicate that further members may optionally be present in addition to the members of the list introduced by “comprising”. It is, however, contemplated as a specific embodiment of the present invention that the term “comprising” encompasses the possibility of no further members being present, i.e., for the purpose of this embodiment “comprising” is to be understood as having the meaning of “consisting of”.

Indications of relative amounts of a component characterized by a generic term are meant to refer to the total amount of all specific variants or members covered by said generic term. If a certain component defined by a generic term is specified to be present in a certain relative amount, and if this component is further characterized to be a specific variant or member covered by the generic term, it is meant that no other variants or members covered by the generic term are additionally present such that the total relative amount of components covered by the generic term exceeds the specified relative amount; more preferably no other variants or members covered by the generic term are present at all.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present invention was not entitled to antedate such disclosure.

Terms such as “reduce” or “inhibit” as used herein means the ability to cause an overall decrease, preferably of 5% or greater, 10% or greater, 20% or greater, more preferably of 50% or greater, and most preferably 75% or greater, in the level. The term “inhibit” or similar phrases includes a complete or essentially complete inhibition, i.e., a reduction to zero or essentially to zero.

Terms such as “increase” or “enhance” preferably relate to an increase or enhancement by about at least 10%, preferably at least 20%, preferably at least 30%, more preferably at least 40%, more preferably at least 50%, even more preferably at least 80%, and most preferably at least 100%.

The term “net charge” refers to the charge on a whole object, such as a compound or particle.

An ion having an overall net positive charge is a cation, while an ion having an overall net negative charge is an anion. Thus, according to the invention, an anion is an ion with more electrons than protons, giving it a net negative charge; and a cation is an ion with fewer electrons than protons, giving it a net positive charge.

Terms as “charged”, “net charge”, “negatively charged” or “positively charged”, with reference to a given compound or particle, refer to the electric net charge of the given compound or particle when dissolved or suspended in water at pH 7.0.

The term “nucleic acid” according to the invention also comprises a chemical derivatization of a nucleic acid on a nucleotide base, on the sugar or on the phosphate, and nucleic acids containing non-natural nucleotides and nucleotide analogs. In some embodiments, the nucleic acid is a deoxyribonucleic acid (DNA) or a ribonucleic acid (RNA). In general, a nucleic acid molecule or a nucleic acid sequence refers to a nucleic acid which is preferably deoxyribonucleic acid (DNA) or ribonucleic acid (RNA). According to the invention, nucleic acids comprise genomic DNA, cDNA, mRNA, viral RNA, recombinantly prepared and chemically synthesized molecules. According to the invention, a nucleic acid may be in the form of a single-stranded or double-stranded and linear or covalently closed circular molecule.

According to the invention “nucleic acid sequence” refers to the sequence of nucleotides in a nucleic acid, e.g.; a ribonucleic acid (RNA) or a deoxyribonucleic acid (DNA). The term may refer to an entire nucleic acid molecule (such as to the single strand of an entire nucleic acid molecule) or to a part (e.g. a fragment) thereof.

According to the present invention, the term “RNA” or “RNA molecule” relates to a molecule which comprises ribonucleotide residues and which is preferably entirely or substantially composed of ribonucleotide residues. The term “ribonucleotide” relates to a nucleotide with a hydroxyl group at the 2′-position of a β-D-ribofuranosyl group. The term “RNA” comprises double-stranded RNA, single stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of an RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs, particularly analogs of naturally occurring RNAs.

According to the invention, RNA may be single-stranded or double-stranded. In some embodiments of the present invention, single-stranded RNA is preferred. The term “single-stranded RNA” generally refers to an RNA molecule to which no complementary nucleic acid molecule (typically no complementary RNA molecule) is associated. Single-stranded RNA may contain self-complementary sequences that allow parts of the RNA to fold back and to form secondary structure motifs including without limitation base pairs, stems, stem loops and bulges. Single-stranded RNA can exist as minus strand [(−) strand] or as plus strand [(+) strand]. The (+) strand is the strand that comprises or encodes genetic information. The genetic information may be for example a polynucleotide sequence encoding a protein. When the (+) strand RNA encodes a protein, the (+) strand may serve directly as template for translation (protein synthesis). The (−) strand is the complement of the (+) strand. In the case of double-stranded RNA, (+) strand and (−) strand are two separate RNA molecules, and both these RNA molecules associate with each other to form a double-stranded RNA (“duplex RNA”).

The term “stability” of RNA relates to the “half-life” of RNA. “Half-life” relates to the period of time which is needed to eliminate half of the activity, amount, or number of molecules. In the context of the present invention, the half-life of an RNA is indicative for the stability of said RNA. The half-life of RNA may influence the “duration of expression” of the RNA. It can be expected that RNA having a long half-life will be expressed for an extended time period.

The term “translation efficiency” relates to the amount of translation product provided by an RNA molecule within a particular period of time.

“Fragment”, with reference to a nucleic acid sequence, relates to a part of a nucleic acid sequence, i.e.; a sequence which represents the nucleic acid sequence shortened at the 5′- and/or 3′-end(s). Preferably, a fragment of a nucleic acid sequence comprises at least 80%, preferably at least 90%, 95%, 96%, 97%, 98%, or 99% of the nucleotide residues from said nucleic acid sequence. In the present invention those fragments of RNA molecules are preferred which retain RNA stability and/or translational efficiency.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises e.g. at least 1%, at least 2%, at least 3%, at least 4%, at least 5%, at least 10%, at least 20%, at least 30%, at least 40%, at least 50%, at least 60%, at least 70%, at least 80%, at least 90% of the amino acid residues from an amino acid sequence.

The term “variant” with respect to, for example, nucleic acid and amino acid sequences, according to the invention includes any variants, in particular mutants, viral strain variants, splice variants, conformations, isoforms, allelic variants, species variants and species homologs, in particular those which are naturally present. An allelic variant relates to an alteration in the normal sequence of a gene, the significance of which is often unclear. Complete gene sequencing often identifies numerous allelic variants for a given gene. With respect to nucleic acid molecules, the term “variant” includes degenerate nucleic acid sequences, wherein a degenerate nucleic acid according to the invention is a nucleic acid that differs from a reference nucleic acid in codon sequence due to the degeneracy of the genetic code. A species homolog is a nucleic acid or amino acid sequence with a different species of origin from that of a given nucleic acid or amino acid sequence. A virus homolog is a nucleic acid or amino acid sequence with a different virus of origin from that of a given nucleic acid or amino acid sequence.

Nucleic acid variants include single or multiple nucleotide deletions, additions, mutations, substitutions and/or insertions in comparison with the reference nucleic acid. Deletions include removal of one or more nucleotides from the reference nucleic acid. Addition variants comprise 5′- and/or 3′-terminal fusions of one or more nucleotides, such as 1, 2, 3, 5, 10, 20, 30, 50, or more nucleotides. In the case of substitutions, at least one nucleotide in the sequence is removed and at least one other nucleotide is inserted in its place (such as transversions and transitions). Mutations include abasic sites, crosslinked sites, and chemically altered or modified bases. Insertions include the addition of at least one nucleotide into the reference nucleic acid.

According to the invention, “nucleotide change” can refer to single or multiple nucleotide deletions, additions, mutations, substitutions and/or insertions in comparison with the reference nucleic acid. In some embodiments, a “nucleotide change” is selected from the group consisting of a deletion of a single nucleotide, the addition of a single nucleotide, the mutation of a single nucleotide, the substitution of a single nucleotide and/or the insertion of a single nucleotide, in comparison with the reference nucleic acid. According to the invention, a nucleic acid variant can comprise one or more nucleotide changes in comparison with the reference nucleic acid.

Variants of specific nucleic acid sequences preferably have at least one functional property of said specific sequences and preferably are functionally equivalent to said specific sequences, e.g., nucleic acid sequences exhibiting properties identical or similar to those of the specific nucleic acid sequences.

As described below, some embodiments of the present invention are characterized, inter alia, by nucleic acid sequences that are homologous to other nucleic acid sequences. These homologous sequences are variants of other nucleic acid sequences.

Preferably the degree of identity between a given nucleic acid sequence and a nucleic acid sequence which is a variant of said given nucleic acid sequence will be at least 70%, preferably at least 75%, preferably at least 80%, more preferably at least 85%, even more preferably at least 90% or most preferably at least 95%, 96%, 97%, 98% or 99%. The degree of identity is preferably given for a region of at least about 30, at least about 50, at least about 70, at least about 90, at least about 100, at least about 150, at least about 200, at least about 250, at least about 300, or at least about 400 nucleotides. In preferred embodiments, the degree of identity is given for the entire length of the reference nucleic acid sequence.

“Sequence similarity” indicates the percentage of amino acids that either are identical or that represent conservative amino acid substitutions. “Sequence identity” between two polypeptide or nucleic acid sequences indicates the percentage of amino acids or nucleotides that are identical between the sequences.

The term “% identical” is intended to refer, in particular, to a percentage of nucleotides which are identical in an optimal alignment between two sequences to be compared, with said percentage being purely statistical, and the differences between the two sequences may be randomly distributed over the entire length of the sequence and the sequence to be compared may comprise additions or deletions in comparison with the reference sequence, in order to obtain optimal alignment between two sequences. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2:482, with the aid of the local homology algorithm by Needleman and Wunsch, 1970, J. Mol. Biol. 48:443, and with the aid of the similarity search algorithm by Pearson and Lipman, 1988, Proc. Natl Acad. Sci. USA 85:2444 or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions in which the sequences to be compared correspond, dividing this number by the number of positions compared and multiplying this result by 100.

For example, the BLAST program “BLAST 2 sequences” which is available on the website http://www.ncbi.nlm.nih.gov/blast/bl2seq/wblast2.cgi may be used.

A nucleic acid is “capable of hybridizing” or “hybridizes” to another nucleic acid if the two sequences are complementary with one another. A nucleic acid is “complementary” to another nucleic acid if the two sequences are capable of forming a stable duplex with one another. According to the invention, hybridization is preferably carried out under conditions which allow specific hybridization between polynucleotides (stringent conditions). Stringent conditions are described, for example, in Molecular Cloning: A Laboratory Manual, J. Sambrook et al., Editors, 2nd Edition, Cold Spring Harbor Laboratory press, Cold Spring Harbor, New York, 1989 or Current Protocols in Molecular Biology, F. M. Ausubel et al., Editors, John Wiley & Sons, Inc., New York and refer, for example, to hybridization at 65° C. in hybridization buffer (3.5×SSC, 0.02% Ficoll, 0.02% polyvinylpyrrolidone, 0.02% bovine serum albumin, 2.5 mM NaH2PO4 (pH 7), 0.5% SDS, 2 mM EDTA). SSC is 0.15 M sodium chloride/0.15 M sodium citrate, pH 7. After hybridization, the membrane to which the DNA has been transferred is washed, for example, in 2×SSC at room temperature and then in 0.1-0.5×SSC/0.1×SDS at temperatures of up to 68° C.

A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or “fully complementary” means that all the contiguous residues of a nucleic acid sequence will hydrogen bond with the same number of contiguous residues in a second nucleic acid sequence. Preferably, the degree of complementarity according to the invention is at least 70%, preferably at least 75%, preferably at least 80%, more preferably at least 85%, even more preferably at least 90% or most preferably at least 95%, 96%, 97%, 98% or 99%. Most preferably, the degree of complementarity according to the invention is 100%.

The term “derivative” comprises any chemical derivatization of a nucleic acid on a nucleotide base, on the sugar or on the phosphate. The term “derivative” also comprises nucleic acids which contain nucleotides and nucleotide analogs not occurring naturally. Preferably, a derivatization of a nucleic acid increases its stability.

A “nucleic acid sequence which is derived from a nucleic acid sequence” refers to a nucleic acid which is a variant of the nucleic acid from which it is derived. Preferably, a sequence which is a variant with respect to a specific sequence, when it replaces the specific sequence in an RNA molecule retains RNA stability and/or translational efficiency.

“nt” is an abbreviation for nucleotide; or for nucleotides, preferably consecutive nucleotides in a nucleic acid molecule.

According to the invention, the term “codon” refers to a base triplet in a coding nucleic acid that specifies which amino acid will be added next during protein synthesis at the ribosome.

The terms “transcription” and “transcribing” relate to a process during which a nucleic acid molecule with a particular nucleic acid sequence (the “nucleic acid template”) is read by an RNA polymerase so that the RNA polymerase produces a single-stranded RNA molecule. During transcription, the genetic information in a nucleic acid template is transcribed. The nucleic acid template may be DNA; however, e.g.; in the case of transcription from an alphaviral nucleic acid template, the template is typically RNA. Subsequently, the transcribed RNA may be translated into protein. According to the present invention, the term “transcription” comprises “in vitro transcription”, wherein the term “in vitro transcription” relates to a process wherein RNA, in particular mRNA, is in vitro synthesized in a cell-free system. Preferably, cloning vectors are applied for the generation of transcripts. These cloning vectors are generally designated as transcription vectors and are according to the present invention encompassed by the term “vector”. The cloning vectors are preferably plasmids. According to the present invention, RNA preferably is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

The single-stranded nucleic acid molecule produced during transcription typically has a nucleic acid sequence that is the complementary sequence of the template.

According to the invention, the terms “template” or “nucleic acid template” or “template nucleic acid” generally refer to a nucleic acid sequence that may be replicated or transcribed.

“Nucleic acid sequence transcribed from a nucleic acid sequence” and similar terms refer to a nucleic acid sequence, where appropriate as part of a complete RNA molecule, which is a transcription product of a template nucleic acid sequence. Typically, the transcribed nucleic acid sequence is a single-stranded RNA molecule.

“3′ end of a nucleic acid” refers according to the invention to that end which has a free hydroxy group. In a diagrammatic representation of double-stranded nucleic acids, in particular DNA, the 3′ end is always on the right-hand side. “5′ end of a nucleic acid” refers according to the invention to that end which has a free phosphate group. In a diagrammatic representation of double-strand nucleic acids, in particular DNA, the 5′ end is always on the left-hand side.

| | | |
|---|---|---|
| 5' end | 5′--P-NNNNNNN-OH-3′ | 3′ end |
| | 3′-HO-NNNNNNN-P--5′ | |

“Upstream” describes the relative positioning of a first element of a nucleic acid molecule with respect to a second element of that nucleic acid molecule, wherein both elements are comprised in the same nucleic acid molecule, and wherein the first element is located nearer to the 5′ end of the nucleic acid molecule than the second element of that nucleic acid molecule. The second element is then said to be “downstream” of the first element of that nucleic acid molecule. An element that is located “upstream” of a second element can be synonymously referred to as being located “5′” of that second element. For a double-stranded nucleic acid molecule, indications like “upstream” and “downstream” are given with respect to the (+) strand.

According to the invention, “functional linkage” or “functionally linked” relates to a connection within a functional relationship. A nucleic acid is “functionally linked” if it is functionally related to another nucleic acid sequence. For example, a promoter is functionally linked to a coding sequence if it influences transcription of said coding sequence. Functionally linked nucleic acids are typically adjacent to one another, where appropriate separated by further nucleic acid sequences, and, in particular embodiments, are transcribed by RNA polymerase to give a single RNA molecule (common transcript).

In particular embodiments, a nucleic acid is functionally linked according to the invention to expression control sequences which may be homologous or heterologous with respect to the nucleic acid.

The term “expression control sequence” comprises according to the invention promoters, ribosome-binding sequences and other control elements which control transcription of a gene or translation of the derived RNA. In particular embodiments of the invention, the expression control sequences can be regulated. The precise structure of expression control sequences may vary depending on the species or cell type but usually includes 5′-untranscribed and 5′- and 3′-untranslated sequences involved in initiating transcription and translation, respectively. More specifically, 5′-untranscribed expression control sequences include a promoter region which encompasses a promoter sequence for transcription control of the functionally linked gene. Expression control sequences may also include enhancer sequences or upstream activator sequences. An expression control sequence of a DNA molecule usually includes 5′-untranscribed and 5′- and 3′-untranslated sequences such as TATA box, capping sequence, CAAT sequence and the like. An expression control sequence of alphaviral RNA may include a subgenomic promoter and/or one or more conserved sequence element(s). A specific expression control sequence according to the present invention is a subgenomic promoter of an alphavirus, as described herein.

The nucleic acid sequences specified herein, in particular transcribable and coding nucleic acid sequences, may be combined with any expression control sequences, in particular promoters, which may be homologous or heterologous to said nucleic acid sequences, with the term “homologous” referring to the fact that a nucleic acid sequence is also functionally linked naturally to the expression control sequence, and the term “heterologous” referring to the fact that a nucleic acid sequence is not naturally functionally linked to the expression control sequence.

A transcribable nucleic acid sequence, in particular a nucleic acid sequence coding for a peptide or protein, and an expression control sequence are “functionally” linked to one another, if they are covalently linked to one another in such a way that transcription or expression of the transcribable and in particular coding nucleic acid sequence is under the control or under the influence of the expression control sequence. If the nucleic acid sequence is to be translated into a functional peptide or protein, induction of an expression control sequence functionally linked to the coding sequence results in transcription of said coding sequence, without causing a frame shift in the coding sequence or the coding sequence being unable to be translated into the desired peptide or protein.

The term “promoter” or “promoter region” refers to a nucleic acid sequence which controls synthesis of a transcript, e.g. a transcript comprising a coding sequence, by providing a recognition and binding site for RNA polymerase. The promoter region may include further recognition or binding sites for further factors involved in regulating transcription of said gene. A promoter may control transcription of a prokaryotic or eukaryotic gene. A promoter may be “inducible” and initiate transcription in response to an inducer, or may be “constitutive” if transcription is not controlled by an inducer. An inducible promoter is expressed only to a very small extent or not at all, if an inducer is absent. In the presence of the inducer, the gene is “switched on” or the level of transcription is increased. This is usually mediated by binding of a specific transcription factor. A specific promoter according to the present invention is a subgenomic promoter, e.g., of an alphavirus, as described herein. Other specific promoters are genomic plus-strand or negative-strand promoters, e.g., of an alphavirus.

The term “core promoter” refers to a nucleic acid sequence that is comprised by the promoter. The core promoter is typically the minimal portion of the promoter required to properly initiate transcription. The core promoter typically includes the transcription start site and a binding site for RNA polymerase.

A “polymerase” generally refers to a molecular entity capable of catalyzing the synthesis of a polymeric molecule from monomeric building blocks. An “RNA polymerase” is a molecular entity capable of catalyzing the synthesis of an RNA molecule from ribonucleotide building blocks. A “DNA polymerase” is a molecular entity capable of catalyzing the synthesis of a DNA molecule from deoxy ribonucleotide building blocks. For the case of DNA polymerases and RNA polymerases, the molecular entity is typically a protein or an assembly or complex of multiple proteins. Typically, a DNA polymerase synthesizes a DNA molecule based on a template nucleic acid, which is typically a DNA molecule. Typically, an RNA polymerase synthesizes an RNA molecule based on a template nucleic acid, which is either a DNA molecule (in that case the RNA polymerase is a DNA-dependent RNA polymerase, DdRP), or is an RNA molecule (in that case the RNA polymerase is an RNA-dependent RNA polymerase, RdRP).

An “RNA-dependent RNA polymerase” or “RdRP”, is an enzyme that catalyzes the transcription of RNA from an RNA template. In the case of alphaviral RNA-dependent RNA polymerase, sequential synthesis of (−) strand complement of genomic RNA and of (+) strand genomic RNA leads to RNA replication. RNA-dependent RNA polymerase is thus synonymously referred to as “RNA replicase” or simply “replicase”. In nature, RNA-dependent RNA polymerases are typically encoded by all RNA viruses except retroviruses. Typical representatives of viruses encoding an RNA-dependent RNA polymerase are alphaviruses.

According to the present invention, “RNA replication” generally refers to an RNA molecule synthesized based on the nucleotide sequence of a given RNA molecule (template RNA molecule). The RNA molecule that is synthesized may be, e.g., identical or complementary to the template RNA molecule. In general, RNA replication may occur via synthesis of a DNA intermediate, or may occur directly by RNA-dependent RNA replication mediated by an RNA-dependent RNA polymerase (RdRP). In the case of alphaviruses, RNA replication does not occur via a DNA intermediate, but is mediated by a RNA-dependent RNA polymerase (RdRP): a template RNA strand (first RNA strand)—or a part thereof—serves as template for the synthesis of a second RNA strand that is complementary to the first RNA strand or to a part thereof. The second RNA strand—or a part thereof—may in turn optionally serve as a template for synthesis of a third RNA strand that is complementary to the second RNA strand or to a part thereof. Thereby, the third RNA strand is identical to the first RNA strand or to a part thereof. Thus, RNA-dependent RNA polymerase is capable of directly synthesizing a complementary RNA strand of a template, and of indirectly synthesizing an identical RNA strand (via a complementary intermediate strand).

According to the invention, the term “template RNA” refers to RNA that can be transcribed or replicated by an RNA-dependent RNA polymerase.

According to the invention, the term “gene” refers to a particular nucleic acid sequence which is responsible for producing one or more cellular products and/or for achieving one or more intercellular or intracellular functions. More specifically, said term relates to a nucleic acid section (typically DNA; but RNA in the case of RNA viruses) which comprises a nucleic acid coding for a specific protein or a functional or structural RNA molecule.

An “isolated molecule” as used herein, is intended to refer to a molecule which is substantially free of other molecules such as other cellular material. The term “isolated nucleic acid” means according to the invention that the nucleic acid has been (i) amplified in vitro, for example by polymerase chain reaction (PCR), (ii) recombinantly produced by cloning, (iii) purified, for example by cleavage and gel-electrophoretic fractionation, or (iv) synthesized, for example by chemical synthesis. An isolated nucleic acid is a nucleic acid available to manipulation by recombinant techniques.

The term “vector” is used here in its most general meaning and comprises any intermediate vehicles for a nucleic acid which, for example, enable said nucleic acid to be introduced into prokaryotic and/or eukaryotic host cells and, where appropriate, to be integrated into a genome. Such vectors are preferably replicated and/or expressed in the cell. Vectors comprise plasmids, phagemids, virus genomes, and fractions thereof.

The term “recombinant” in the context of the present invention means “made through genetic engineering”. Preferably, a “recombinant object” such as a recombinant cell in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

According to the invention, the term “expression” is used in its most general meaning and comprises production of RNA and/or protein. It also comprises partial expression of nucleic acids. Furthermore, expression may be transient or stable. With respect to RNA, the term “expression” or “translation” relates to the process in the ribosomes of a cell by which a strand of coding RNA (e.g. messenger RNA) directs the assembly of a sequence of amino acids to make a peptide or protein.

According to the invention, the term “mRNA” means “messenger-RNA” and relates to a transcript which is typically generated by using a DNA template and encodes a peptide or protein. Typically, mRNA comprises a 5′-UTR, a protein coding region, a 3′-UTR, and a poly(A) sequence. mRNA may be generated by in vitro transcription from a DNA template. The in vitro transcription methodology is known to the skilled person. For example, there is a variety of in vitro transcription kits commercially available. According to the invention, mRNA may be modified by stabilizing modifications and capping.

According to the invention, the terms “poly(A) sequence” or “poly(A) tail” refer to an uninterrupted or interrupted sequence of adenylate residues which is typically located at the 3′ end of an RNA molecule. An uninterrupted sequence is characterized by consecutive adenylate residues. In nature, an uninterrupted poly(A) sequence is typical. While a poly(A) sequence is normally not encoded in eukaryotic DNA, but is attached during eukaryotic transcription in the cell nucleus to the free 3′ end of the RNA by a template-independent RNA polymerase after transcription, the present invention encompasses poly(A) sequences encoded by DNA.

According to the invention, the term “primary structure”, with reference to a nucleic acid molecule, refers to the linear sequence of nucleotide monomers.

According to the invention, the term “secondary structure”, with reference to a nucleic acid molecule, refers to a two-dimensional representation of a nucleic acid molecule that reflects base pairings; e.g.; in the case of a single-stranded RNA molecule particularly intramolecular base pairings. Although each RNA molecule has only a single polynucleotide chain, the molecule is typically characterized by regions of (intramolecular) base pairs. According to the invention, the term “secondary structure” comprises structural motifs including without limitation base pairs, stems, stem loops, bulges, loops such as interior loops and multi-branch loops. The secondary structure of a nucleic acid molecule can be represented by a two-dimensional drawing (planar graph), showing base pairings (for further details on secondary structure of RNA molecules, see Auber et al., 2006; J. Graph Algorithms Appl. 10:329-351). As described herein, the secondary structure of certain RNA molecules is relevant in the context of the present invention.

According to the invention, secondary structure of a nucleic acid molecule, particularly of a single-stranded RNA molecule, is determined by prediction using the web server for RNA secondary structure prediction (http://ma.urmc.rochester.edu/RNAstructureWeb/Servers/Predict1/Predict1.html). Preferably, according to the invention, “secondary structure”, with reference to a nucleic acid molecule, specifically refers to the secondary structure determined by said prediction. The prediction may also be performed or confirmed using MFOLD structure prediction (http://unafold.ma.albany.edu/?q=mfold).

According to the invention, a “base pair” is a structural motif of a secondary structure wherein two nucleotide bases associate with each other through hydrogen bonds between donor and acceptor sites on the bases. The complementary bases, A:U and G:C, form stable base pairs through hydrogen bonds between donor and acceptor sites on the bases; the A:U and G:C base pairs are called Watson-Crick base pairs. A weaker base pair (called Wobble base pair) is formed by the bases G and U (G:U). The base pairs A:U and G:C are called canonical base pairs. Other base pairs like G:U (which occurs fairly often in RNA) and other rare base-pairs (e.g. A:C; U:U) are called non-canonical base pairs.

According to the invention, “nucleotide pairing” refers to two nucleotides that associate with each other so that their bases form a base pair (canonical or non-canonical base pair, preferably canonical base pair, most preferably Watson-Crick base pair).

According to the invention, the terms “stem loop” or “hairpin” or “hairpin loop”, with reference to a nucleic acid molecule, all interchangeably refer to a particular secondary structure of a nucleic acid molecule, typically a single-stranded nucleic acid molecule, such as single-stranded RNA. The particular secondary structure represented by the stem loop consists of a consecutive nucleic acid sequence comprising a stem and a (terminal) loop, also called hairpin loop, wherein the stem is formed by two neighbored entirely or partially complementary sequence elements; which are separated by a short sequence (e.g. 3-10 nucleotides), which forms the loop of the stem-loop structure. The two neighbored entirely or partially complementary sequences may be defined as, e.g., stem loop elements stem 1 and stem 2. The stem loop is formed when these two neighbored entirely or partially reverse complementary sequences, e.g. stem loop elements stem 1 and stem 2, form base-pairs with each other, leading to a double stranded nucleic acid sequence comprising an unpaired loop at its terminal ending formed by the short sequence located between stem loop elements stem 1 and stem 2. Thus, a stem loop comprises two stems (stem 1 and stem 2), which—at the level of secondary structure of the nucleic acid molecule—form base pairs with each other, and which—at the level of the primary structure of the nucleic acid molecule—are separated by a short sequence that is not part of stem 1 or stem 2. For illustration, a two-dimensional representation of the stem loop resembles a lollipop-shaped structure. The formation of a stem-loop structure requires the presence of a sequence that can fold back on itself to form a paired double strand; the paired double strand is formed by stem 1 and stem 2. The stability of paired stem loop elements is typically determined by the length, the number of nucleotides of stem 1 that are capable of forming base pairs (preferably canonical base pairs, more preferably Watson-Crick base pairs) with nucleotides of stem 2, versus the number of nucleotides of stem 1 that are not capable of forming such base pairs with nucleotides of stem 2 (mismatches or bulges). According to the present invention, the optimal loop length is 3-10 nucleotides, more preferably 4 to 7, nucleotides, such as 4 nucleotides, 5 nucleotides, 6 nucleotides or 7 nucleotides. If a given nucleic acid sequence is characterized by a stem loop, the respective complementary nucleic acid sequence is typically also characterized by a stem loop. A stem loop is typically formed by single-stranded RNA molecules. For example, several stem loops are present in the 5′ replication recognition sequence of alphaviral genomic RNA.

According to the invention, “disruption” or “disrupt”, with reference to a specific secondary structure of a nucleic acid molecule (e.g., a stem loop) means that the specific secondary structure is absent or altered. Typically, a secondary structure may be disrupted as a consequence of a change of at least one nucleotide that is part of the secondary structure. For example, a stem loop may be disrupted by change of one or more nucleotides that form the stem, so that nucleotide pairing is not possible.

According to the invention, the term “tertiary structure”, with reference to a nucleic acid molecule, refers to the three-dimensional structure of a nucleic acid molecule, as defined by the atomic coordinates.

According to the invention, a nucleic acid such as RNA, e.g., rRNA, may encode a peptide or protein. Accordingly, a transcribable nucleic acid sequence or a transcript thereof may contain an open reading frame (ORF) encoding a peptide or protein.

According to the invention, the term “nucleic acid encoding a peptide or protein” means that the nucleic acid, if present in the appropriate environment, preferably within a cell, can direct the assembly of amino acids to produce the peptide or protein during the process of translation. Preferably, coding RNA according to the invention is able to interact with the cellular translation machinery allowing translation of the coding RNA to yield a peptide or protein.

According to the invention, the term “peptide” comprises oligo- and polypeptides and refers to substances which comprise two or more, preferably 3 or more, preferably 4 or more, preferably 6 or more, preferably 8 or more, preferably 10 or more, preferably 13 or more, preferably 16 or more, preferably 20 or more, and up to preferably 50, preferably 100 or preferably 150, consecutive amino acids linked to one another via peptide bonds. The term “protein” refers to large peptides, preferably peptides having at least 151 amino acids, but the terms “peptide” and “protein” are used herein usually as synonyms.

The terms “peptide” and “protein” comprise, according to the invention, substances which contain not only amino acid components but also non-amino acid components such as sugars and phosphate structures, and also comprise substances containing bonds such as ester, thioether or disulfide bonds.

According to the invention, the terms “initiation codon” and “start codon” synonymously refer to a codon (base triplet) of an RNA molecule that is potentially the first codon that is translated by a ribosome. Such codon typically encodes the amino acid methionine in eukaryotes and a modified methionine in prokaryotes. The most common initiation codon in eukaryotes and prokaryotes is AUG. Unless specifically stated herein that an initiation codon other than AUG is meant, the terms “initiation codon” and “start codon”, with reference to an RNA molecule, refer to the codon AUG. According to the invention, the terms “initiation codon” and “start codon” are also used to refer to a corresponding base triplet of a deoxyribonucleic acid, namely the base triplet encoding the initiation codon of an RNA. If the initiation codon of messenger RNA is AUG, the base triplet encoding the AUG is ATG. According to the invention, the terms “initiation codon” and “start codon” preferably refer to a functional initiation codon or start codon, i.e., to an initiation codon or start codon that is used or would be used as a codon by a ribosome to start translation. There may be AUG codons in an RNA molecule that are not used as codons by a ribosome to start translation, e.g., due to a short distance of the codons to the cap. These codons are not encompassed by the term functional initiation codon or start codon.

The following provides specific and/or preferred variants of the individual features of the invention. The present invention also contemplates as particularly preferred embodiments those embodiments, which are generated by combining two or more of the specific and/or preferred variants described for two or more of the features of the present invention.

“Isolated” means altered or removed from the natural state. For example, a cell, a nucleic acid or a peptide naturally present in a living animal is not “isolated”, but the same cell, nucleic acid or peptide partially or completely separated from the coexisting materials of its natural state is “isolated”. Preferably an isolated cell, nucleic acid or peptide exists in a purified or substantially purified state. An isolated cell or cell population preferably does exist without cells of a different cell type, e.g., an isolated T cell exists without other blood cells such as dendritic cells. Preferably an isolated cell does exist only with isogeneic cells of the same cell type.

The term “isogeneic” is used to describe a cell that has the same genetic information as another cell or cell population.

The term “autologous” is used to describe anything that is derived from the same subject. For example, “autologous transplant” refers to a transplant of tissue or organs derived from the same subject. Such procedures are advantageous because they overcome the immunological barrier which otherwise results in rejection.

The term “allogeneic” is used to describe anything that is derived from different individuals of the same species. Two or more individuals are said to be allogeneic to one another when the genes at one or more loci are not identical.

The term “syngeneic” is used to describe anything that is derived from individuals or tissues having identical genotypes, i.e., identical twins or animals of the same inbred strain, or their tissues.

The term “heterologous” is used to describe something consisting of multiple different elements. As an example, the transfer of one individual's bone marrow into a different individual constitutes a heterologous transplant. A heterologous gene is a gene derived from a source other than the subject.

The term “recombinant” in the context of the present invention means “made through genetic engineering”. Preferably, a “recombinant object” such as a recombinant cell in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “specifically binds”, as used herein, is meant a molecule such as an antibody or CAR which recognizes a specific antigen, but does not substantially recognize or bind other molecules in a sample or in a subject. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding”, can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “minicircle” refers to vectors which are supercoiled DNA molecules that lack a bacterial origin of replication and an antibiotic resistance gene. They are primarily composed of a eukaryotic expression cassette (see, for instance, F. Jia et al. Nature methods, Vol. 7, no. 3, p. 197-199, March 2010).

### Immune Effector Cells

The cells used in connection with the present invention and into which nucleic acids (DNA and/or RNA) may be introduced are immune effector cells such as cells with lytic potential, in particular lymphoid cells, and are preferably T cells, in particular cytotoxic lymphocytes, preferably selected from cytotoxic T cells, natural killer (NK) cells, and lymphokine-activated killer (LAK) cells. Upon activation, each of these cytotoxic lymphocytes triggers the destruction of target cells. For example, cytotoxic T cells trigger the destruction of target cells by either or both of the following means. First, upon activation T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target cells. The cells used in connection with the present invention will preferably be autologous cells, although heterologous cells or allogenic cells can be used.

The term “immune effector cell” or “immunoreactive cell” in the context of the present invention relates to a cell which exerts effector functions during an immune reaction.

The term “effector functions” in the context of the present invention includes any functions mediated by components of the immune system that result, for example, in the killing of diseased cells such as tumor cells, or in the inhibition of tumor growth and/or inhibition of tumor development, including inhibition of tumor dissemination and metastasis. Preferably, the effector functions in the context of the present invention are T cell mediated effector functions. Such functions comprise in the case of a helper T cell (CD4⁺ T cell) the release of cytokines and/or the activation of CD8⁺ lymphocytes (CTLs) and/or B cells, and in the case of CTL the elimination of cells, i.e., cells characterized by expression of an antigen, for example, via apoptosis or perforin-mediated cell lysis, production of cytokines such as IFN-γ and TNF-α, and specific cytolytic killing of antigen expressing target cells.

An “immune effector cell” in one embodiment is capable of binding an antigen such as an antigen presented in the context of MHC on a cell or expressed on the surface of a cell and mediating an immune response. For example, immune effector cells comprise T cells (cytotoxic T cells, helper T cells, tumor infiltrating T cells), B cells, natural killer cells, neutrophils, macrophages, and dendritic cells. Preferably, in the context of the present invention, “immune effector cells” are T cells, preferably CD4⁺ and/or CD8⁺ T cells. According to the invention, the term “immune effector cell” also includes a cell which can mature into an immune cell (such as T cell, in particular T helper cell, or cytolytic T cell) with suitable stimulation. Immune effector cells comprise CD34⁺ hematopoietic stem cells, immature and mature T cells and immature and mature B cells. The differentiation of T cell precursors into a cytolytic T cell, when exposed to an antigen, is similar to clonal selection of the immune system.

Preferably, an “immune effector cell” recognizes an antigen with some degree of specificity, in particular if presented in the context of MHC or present on the surface of diseased cells such as cancer cells. Preferably, said recognition enables the cell that recognizes an antigen to be responsive or reactive. If the cell is a helper T cell (CD4⁺ T cell) such responsiveness or reactivity may involve the release of cytokines and/or the activation of CD8⁺ lymphocytes (CTLs) and/or B cells. If the cell is a CTL such responsiveness or reactivity may involve the elimination of cells, i.e., cells characterized by expression of an antigen, for example, via apoptosis or perforin-mediated cell lysis. According to the invention, CTL responsiveness may include sustained calcium flux, cell division, production of cytokines such as IFN-γ and TNF-α, up-regulation of activation markers such as CD44 and CD69, and specific cytolytic killing of antigen expressing target cells. CTL responsiveness may also be determined using an artificial reporter that accurately indicates CTL responsiveness. Such CTL that recognizes an antigen and are responsive or reactive are also termed “antigen-responsive CTL” herein.

In one embodiment, the immune effector cells are CAR-expressing immune effector cells. In one embodiment, the immune effector cells are TCR-expressing immune effector cells.

The immune effector cells to be used according to the invention may express an endogenous antigen receptor such as T cell receptor or B cell receptor or may lack expression of an endogenous antigen receptor.

A “lymphoid cell” is a cell which, optionally after suitable modification, e.g., after transfer of an antigen receptor such as a TCR or a CAR, is capable of producing an immune response such as a cellular immune response, or a precursor cell of such cell, and includes lymphocytes, preferably T lymphocytes, lymphoblasts, and plasma cells. A lymphoid cell may be an immune effector cell as described herein. A preferred lymphoid cell is a T cell which can be modified to express an antigen receptor on the cell surface. In one embodiment, the lymphoid cell lacks endogenous expression of a T cell receptor.

The terms “T cell” and “T lymphocyte” are used interchangeably herein and include T helper cells (CD4⁺ T cells) and cytotoxic T cells (CTLs, CD8⁺ T cells) which comprise cytolytic T cells. The term “antigen-specific T cell” or similar terms relate to a T cell which recognizes the antigen to which the T cell is targeted and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured.

T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptors (TCR). The thymus is the principal organ responsible for the maturation of T cells. Several different subsets of T cells have been discovered, each with a distinct function.

T helper cells assist other white blood cells in immunologic processes, including maturation of B cells into plasma cells and activation of cytotoxic T cells and macrophages, among other functions. These cells are also known as CD4⁺ T cells because they express the CD4 glycoprotein on their surface. Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8⁺ T cells since they express the CD8 glycoprotein on their surface. These cells recognize their targets by binding to antigen associated with MHC class I, which is present on the surface of nearly every cell of the body.

“Regulatory T cells” or “Tregs” are a subpopulation of T cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FoxP3, and CD25.

As used herein, the term “naïve T cell” refers to mature T cells that, unlike activated or memory T cells, have not encountered their cognate antigen within the periphery. Naïve T cells are commonly characterized by the surface expression of L-selectin (CD62L), the absence of the activation markers CD25, CD44 or CD69 and the absence of the memory CD45RO isoform.

As used herein, the term “memory T cells” refers to a subgroup or subpopulation of T cells that have previously encountered and responded to their cognate antigen. At a second encounter with the antigen, memory T cells can reproduce to mount a faster and stronger immune response than the first time the immune system responded to the antigen. Memory T cells may be either CD4⁺ or CD8⁺ and usually express CD45RO.

According to the invention, the term “T cell” also includes a cell which can mature into a T cell with suitable stimulation.

A majority of T cells have a T cell receptor (TCR) existing as a complex of several proteins. The actual T cell receptor is composed of two separate peptide chains, which are produced from the independent T cell receptor alpha and beta (TCRα and TCRβ) genes and are called α- and β-TCR chains. γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the αβ T cells.

All T cells originate from hematopoietic stem cells in the bone marrow. Hematopoietic progenitors derived from hematopoietic stem cells populate the thymus and expand by cell division to generate a large population of immature thymocytes. The earliest thymocytes express neither CD4 nor CD8, and are therefore classed as double-negative (CD4⁻CD8⁻) cells. As they progress through their development they become double-positive thymocytes (CD4⁺CD8⁺), and finally mature to single-positive (CD4⁺CD8⁻ or CD4⁻CD8⁺) thymocytes that are then released from the thymus to peripheral tissues.

T cells may generally be prepared in vitro or ex vivo, using standard procedures. For example, T cells may be isolated from bone marrow, peripheral blood or a fraction of bone marrow or peripheral blood of a mammal, such as a patient, using a commercially available cell separation system. Alternatively, T cells may be derived from related or unrelated humans, non-human animals, cell lines or cultures. A sample comprising T cells may, for example, be peripheral blood mononuclear cells (PBMC).

As used herein, the term “NK cell” or “Natural Killer cell” refers to a subset of peripheral blood lymphocytes defined by the expression of CD56 or CD16 and the absence of the T cell receptor. As provided herein, the NK cell can also be differentiated from a stem cell or progenitor cell.

### Nucleic Acids

The term “polynucleotide” or “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA. According to the invention, a polynucleotide is preferably isolated.

Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In one embodiment of all aspects of the invention, nucleic acid such as nucleic acid encoding an antigen receptor or nucleic acid encoding an immune effector cell-activator molecule is expressed in immune effector cells and provide the antigen receptor or activator molecule.

The nucleic acids of the present invention can be introduced into immune effector cells by various means, e.g., by a particle or complex of the present disclosure or any composition comprising one or both of particle and complex, or by electroporation, or by virus-based systems, or by any, in particular lipid or polymer-based, particle which is able to introduce nucleic acids into a cell. In some embodiments, the nucleic acids are introduced into a cell or taken up by a cell, wherein the cell may be present in a subject, e.g., a patient. Thus, according to the present invention, a cell into which nucleic acids described herein are introduced can be present in vitro or in vivo, e.g., the cell can form part of an organ, a tissue and/or an organism of a patient.

In some embodiments, the first nucleic acid molecule is a DNA or RNA molecule introduced into the cell. In other embodiments, the first nucleic acid molecule is a DNA molecule into which the first nucleotide sequence has been integrated and which has been present in the cell before, but without the first nucleotide sequence. In this embodiment the first nucleotide sequence has been introduced into the cell previously with another first nucleic acid molecule, which can be DNA or RNA, from which it is taken and integrated into a DNA molecule already present in the cell, e.g., genomic DNA. The DNA molecule into which the first nucleotide sequence has been integrated becomes the first nucleic acid molecule upon integration.

In some embodiments, the first nucleic acid is an RNA molecule which is to be integrated into the genome of an immune effector cell via a retrotransposon-based system, preferably a viral-based retrotransposon system or a poly-A-based retrotransposon system.

The terms “genome” or “genomic DNA” or “genomic nucleic acid molecule” are meant to refer to any kind of DNA molecule that is propagated and equally distributed from mother to daughter cells. Genomic DNA refers to both chromosomal DNA and extra-chromosomal DNA such as episomes, preferably non-viral episomes.

The term “episome” is to be understood to refer to a DNA molecule that remains as a part of the eukaryotic genome without integration. Episomes manage this by replicating together with the rest of the genome and subsequently being distributed like chromosomes to each daughter cell equally.

In some embodiments, the second nucleic acid molecule is not integrated into a genomic nucleic acid molecule of the immune effector cell, in particular not comprised within an episome present in the immune effector cell. Preferably, the second nucleic acid is an RNA molecule, preferably an mRNA, more preferably a modified RNA or mRNA. The RNA molecule can in some embodiments be degraded or lost upon cell division.

In another embodiment, the second nucleic acid molecule is a DNA molecule, preferably a plasmid, which is preferably degraded in the immune effector cell or epigenetically silenced or not equally propagated during cell division.

In some embodiments, the second nucleic acid is transiently expressed.

The term “transiently expressed” is to be understood to mean that a nucleic acid or transcription product is only expressed for a limited period of time. It preferably means that the nucleic acid encoding a transcriptional product, a non-coding RNA or a protein, is lost from a cell either due to degradation or due to unequal distribution of the nucleic acid during cell division, in particular in that the nucleic acid is not replicated in the cell in order to replenish any lost nucleic acid. A transiently expressed nucleic acid preferably is not part of the genome of a cell. For example, a DNA plasmid encoding a certain transcriptional product without a eukaryotic origin of replication, preferably not a mammalian, preferably human, origin of replication, is only transiently expressed because, even though the encoded transcriptional product is expressed, the plasmid is lost as without an origin of replication it is not replenished after degradation or replicated before cell division. A protein for example is to be understood to be transiently expressed, when it is only expressed for a limited time, e.g., because the nucleic acid encoding the protein is lost and not replicated. Another example for a transiently expressed nucleic acid is an mRNA molecule introduced into the cell, e.g., via electroporation.

In some embodiments, the third nucleic acid molecule is a DNA or RNA. The third nucleic acid molecule provides, in case the first nucleotide sequence is to be integrated into the genome of the immune effector cell, the necessary enzymes, e.g., transposase, reverse transcriptase or integrase, for the integration of a first nucleotide sequence into a genomic nucleic acid molecule. Providing the means for integrating the first nucleotide sequence via a separate nucleic acid has a particular advantage that it helps preventing the loss of the integrated first nucleotide sequence as the means for integration, which can also remove the integrated nucleotide sequence, are only provided for a limited time. The third nucleic acid molecule can be one or more nucleic acid molecules. In case one than one enzyme is necessary for integration, providing separate nucleic acids encoding the required enzymes helps in providing further flexibility, e.g., with respect to amounts of enzymes produced in the cell or in combining different enzymes.

In some embodiments, the third nucleic acid molecule is not integrated into a genomic nucleic acid molecule of the immune effector cell, in particular not comprised within an episome present in the immune effector cell. Preferably, the third nucleic acid is an RNA molecule, preferably an mRNA, more preferably a modified RNA or mRNA. The RNA molecule can in some embodiments be degraded or lost upon cell division.

In another embodiment, the third nucleic acid molecule is a DNA molecule, preferably a plasmid, which is preferably not propagated during cell division or degraded in the immune effector cell or epigenetically silenced.

In another embodiment, the enzymes required for integration can also be provided in other forms in comparison to be encoded on a nucleic acid molecule.

In some embodiments, the immune effector cell or the particle of the present disclosure comprises a fourth and/or fifth nucleic acid molecule. In some embodiments, the fourth and/or fifth nucleic acid molecule is one or more nucleic acid molecules.

In some embodiments, the fourth nucleic acid molecule encodes one or more antigens to which the first cell-surface expressed antigen receptor binds.

In some embodiments, the fifth nucleic acid molecule encodes one or more antigens to which the second cell-surface expressed antigen receptor binds.

In some embodiments, the fourth nucleic acid molecule is not integrated into a genomic nucleic acid molecule of the immune effector cell, in particular not comprised within an episome present in the immune effector cell. Preferably, the fourth nucleic acid is an RNA molecule, preferably an mRNA, more preferably a modified RNA or mRNA. The RNA molecule can in some embodiments be degraded or lost upon cell division.

In another embodiment, the fourth nucleic acid molecule is a DNA molecule, preferably a plasmid, which is preferably not propagated during cell division or degraded in the immune effector cell or epigenetically silenced.

In some embodiments, the fifth nucleic acid molecule is not integrated into a genomic nucleic acid molecule of the immune effector cell, in particular not comprised within an episome present in the immune effector cell. Preferably, the fifth nucleic acid is an RNA molecule, preferably an mRNA, more preferably a modified RNA or mRNA. The RNA molecule can in some embodiments be degraded or lost upon cell division.

In another embodiment, the fifth nucleic acid molecule is a DNA molecule, preferably a plasmid, which is preferably not propagated during cell division or degraded in the immune effector cell or epigenetically silenced.

In some embodiments, at least one or all of the first, second, third, fourth and fifth nucleic acid molecule is a modified RNA molecule.

### Modified RNA

In some embodiments, the RNA or RNA molecules described herein are modified RNA. In some embodiments, the modified RNA contains at least one functional analog of A, C, G and/or U.

In an embodiment, the RNA described herein may have modified nucleotides/nucleosides/backbone modifications. The term “RNA modification” as used herein may refer to chemical modifications comprising backbone modifications as well as sugar modifications or base modifications.

In this context, a modified RNA molecule as defined herein may contain nucleotide analogues/modifications, e.g., backbone modifications, sugar modifications or base modifications. A backbone modification in connection with the present disclosure is a modification, in which phosphates of the backbone of the nucleotides contained in an RNA molecule as defined herein are chemically modified. A sugar modification in connection with the present disclosure is a chemical modification of the sugar of the nucleotides of the RNA molecule as defined herein. Furthermore, a base modification in connection with the present disclosure is a chemical modification of the base moiety of the nucleotides of the RNA molecule. In this context, nucleotide analogues or modifications are preferably selected from nucleotide analogues, which are applicable for transcription and/or translation.

Sugar Modifications: The modified nucleosides and nucleotides, which may be incorporated into a modified RNA molecule as described herein, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH₂CH₂O)nCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy. “Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O. The sugar group can also contain one or more carbons that possess the opposite stereochemical configuration than that of the corresponding carbon in ribose. Thus, a modified RNA molecule can include nucleotides containing, for instance, arabinose as the sugar.

Backbone Modifications: The phosphate backbone may further be modified in the modified nucleosides and nucleotides, which may be incorporated into a modified RNA molecule as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

Base Modifications: The modified nucleosides and nucleotides, which may be incorporated into a modified RNA molecule as described herein can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particular embodiments of the present disclosure, the nucleotide analogues/modifications are selected from base modifications, which are preferably selected from 2-amino-6-chloropurineriboside-5′-triphosphate, 2-aminopurine-riboside-5′-triphosphate; 2-aminoadenosine-5′-triphosphate, 2′-amino-2′-deoxy-cytidine-triphosphate, 2-thiocytidine-5′-triphosphate, 2-thiouridine-5′-triphosphate, 2′-fluorothymidine-5′-triphosphate, 2′-O-methyl inosine-5′-triphosphate 4-thio-uridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-tri-phosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deaza-adenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, N6-methylguanosine-5′-triphosphate, pseudo-uridine-5′-triphosphate, or puromycin-5′-triphosphate, xanthosine-5′-triphosphate. Particular preference may be given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate. In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thiouridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydro-pseudouridine, 2-thio-dihydrouridine, 2-thio-dihydropseudouridine, 2-methoxy-uridine, 2-methoxy-4-thio-uridine, 4-methoxy-pseudouridine, and 4-methoxy-2-thio-pseudouridine. In a preferred embodiment the functional analog replacing uridine is N1-methyl-pseudouridine (m1ψ).

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diamino-purine, 1-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyladenosine, 2-methyl-thio-N6-threonylcarbamoyladenosine, N6,N6-dimethyladenosine, 7-methyladenine, 2-methylthio-adenine, and 2-methoxy-adenine. In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group. In specific embodiments, a modified nucleoside is 5′-O-(1-thiophosphate)-adenosine, 5′-O-(1-thiophosphate)-cytidine, 5′-O-(1-thiophosphate)-guanosine, 5′-O-(1-thiophosphate)-uridine or 5′-O-(1-thiophosphate)-pseudouridine.

In further embodiments, a modified RNA may comprise nucleoside modifications selected from 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, pyrrolo-cytidine, inosine, a-thio-guanosine, 6-methyl-guanosine, 5-methyl-cytidine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, a-thio-adenosine, 8-azido-adenosine, 7-deaza-adenosine.

In certain preferred embodiments, the RNA comprises a modified nucleoside in place of at least one (e.g., every) uridine.

The term “uracil,” as used herein, describes one of the nucleobases that can occur in the nucleic acid of RNA. The structure of uracil is:

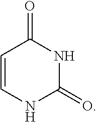

The term “uridine,” as used herein, describes one of the nucleosides that can occur in RNA. The structure of uridine is:

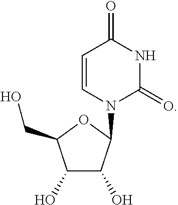 [SMILES: O=c1ccn([C@@H]2O[C@H](CO)[C@@H](O)[C@H]2O)c(=O)[nH]1]

UTP (uridine 5′-triphosphate) has the following structure:

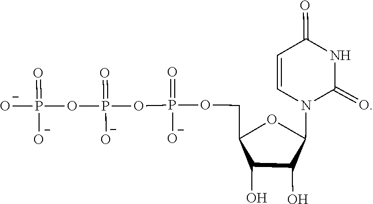

Pseudo-UTP (pseudouridine 5′-triphosphate) has the following structure:

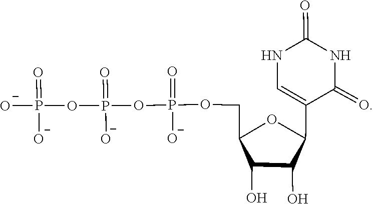

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1ψ), which has the structure:

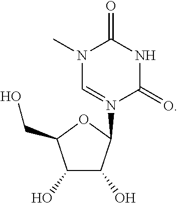

N1-methyl-pseudo-UTP has the following structure:

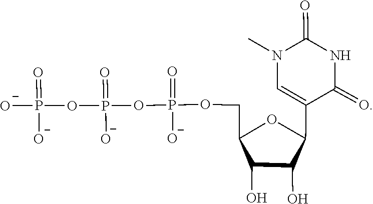

Another exemplary modified nucleoside is 5-methyl-uridine (m5U), which has the structure:

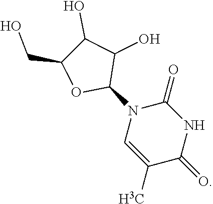 [SMILES: [3CH3]c1cn([C@H]2O[C@@H](CO)C(O)C2O)c(=O)[nH]c1=O]

In certain preferred embodiments, one or more uridine in the RNA described herein is replaced by a modified nucleoside. In some embodiments, the modified nucleoside is a modified uridine.

In certain preferred embodiments, RNA comprises a modified nucleoside in place of at least one uridine. In some embodiments, RNA comprises a modified nucleoside in place of each uridine.

In certain preferred embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (N1) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In certain preferred embodiments, the modified nucleoside replacing one or more, e.g., all, uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Urn), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In an embodiment, the RNA comprises other modified nucleosides or comprises further modified nucleosides, e.g., modified cytidine such as those described above. For example, in one embodiment, in the RNA 5-methylcytidine is substituted partially or completely, preferably completely, for cytidine. In one embodiment, the RNA comprises 5-methylcytidine and one or more selected from pseudouridine (ψ), N1-methyl-pseudouridine (m1ψ), and 5-methyl-uridine (m5U). In an embodiment, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

### Cap

In some embodiments, an RNA or RNA molecule described herein may optionally comprise a 5′cap, 5′ UTR, a coding sequence, a 3′ UTR and/or a poly-(A) tail. In some embodiments the coding sequence or open reading frame may be optimized with respect to the codon usage.

“RNA which comprises a 5′-cap” or “RNA which is provided with a 5′-cap” or “RNA which is modified with a 5′-cap” or “capped RNA” refers to RNA which comprises a 5′-cap. For example, providing an RNA with a 5′-cap may be achieved by in vitro transcription of a DNA template in presence of said 5′-cap, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus. In capped RNA, the 3′ position of the first base of a (capped) RNA molecule is linked to the 5′ position of the subsequent base of the RNA molecule (“second base”) via a phosphodiester bond.

In the present disclosure, a natural occurring cap is typically selected from the group consisting of a non-methylated cap dinucleotide (G(5′)ppp(5′)N; also termed GpppN) and a methylated cap dinucleotide ((m⁷G(5′)ppp(5′)N; also termed m⁷GpppN). m⁷GpppN (wherein N is G) is represented by the following formula:

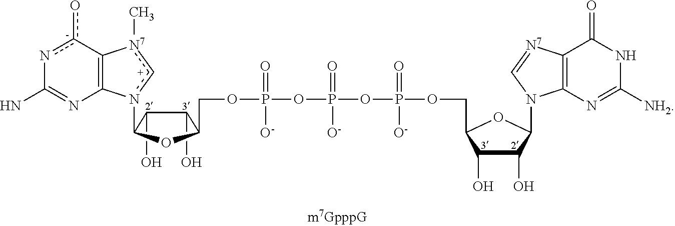

Capped RNA of the present disclosure can be prepared in vitro, and therefore, does not depend on a capping machinery in a host cell. Co-transcriptional capping works by transcribing in vitro a DNA template with either a bacterial or bacteriophage nucleic acid polymerase in the presence of all four ribonucleoside triphosphates or functional analogs thereof and a capping reagent such as m⁷G(5′)ppp(5′)G (also called m⁷GpppG). The nucleic acid polymerase initiates transcription with a nucleophilic attack by the 3′-OH of the guanosine moiety of m⁷GpppG on the α-phosphate of the next templated nucleoside triphosphate (pppN), resulting in the intermediate m⁷GpppGpN (wherein N is the second base of the RNA molecule).

In preferred embodiments of the present disclosure, the RNA molecule comprises a 5′-cap analog. Cap analogs have been initially described to facilitate large scale synthesis of RNA transcripts by means of in vitro transcription.

For messenger RNA, some cap analogs (also called synthetic caps) have been generally described to date, and they can all be used in the context of the present disclosure. Ideally, a cap analog is selected that is associated with higher translation efficiency and/or increased resistance to in vivo degradation and/or increased resistance to in vitro degradation.

Preferably, a cap analog is used that can only be incorporated into an RNA chain in one orientation. Pasquinelli et al., 1995, RNA J. 1:957-967) demonstrated that during in vitro transcription, bacteriophage RNA polymerases use the 7-methylguanosine unit for initiation of transcription, whereby around 40-50% of the transcripts with cap possess the cap dinucleotide in a reverse orientation (i.e., the initial reaction product is Gpppm⁷GpN).

Compared to the RNAs with a correct cap, RNAs with a reverse cap are not functional with respect to translation of a nucleic acid sequence into protein. Thus, it is desirable to incorporate the cap in the correct orientation, i.e., resulting in an RNA with a structure essentially corresponding to m⁷GpppGpN etc. It has been shown that the reverse integration of the cap-dinucleotide is inhibited by the substitution of either the 2′- or the 3′-OH group of the methylated guanosine unit (Stepinski et al., 2001, RNA J. 7:1486-1495; Peng et al., 2002, Org. Lett. 24:161-164). RNAs which are synthesized in presence of such “anti reverse cap analogs” are translated more efficiently than RNAs which are in vitro transcribed in presence of the conventional 5′-cap m⁷GpppG. To that end, one cap analog in which the 3′ OH group of the methylated guanosine unit is replaced by OCH₃ is described, e.g., by Holtkamp et al., 2006, Blood 108:4009-4017 (7-methyl(3′-O-methyl)GpppG; anti-reverse cap analog (ARCA)). ARCA is a suitable cap dinucleotide according to the present disclosure:

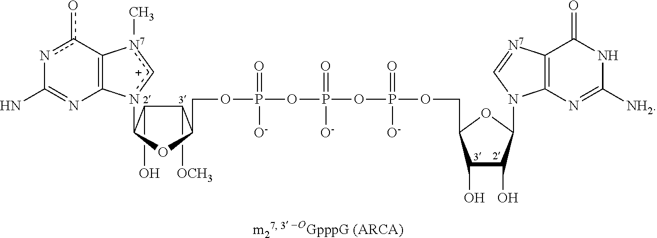

In an embodiment, the cap is having the effect that RNA with such a cap is essentially not susceptible to decapping. This is important because, in general, the amount of protein produced from synthetic mRNAs introduced into cultured mammalian cells is limited by the natural degradation of mRNA. One in vivo pathway for mRNA degradation begins with the removal of the mRNA cap. This removal is catalyzed by a heterodimeric pyrophosphatase, which contains a regulatory subunit (Dcp1) and a catalytic subunit (Dcp2). The catalytic subunit cleaves between the α and β phosphate groups of the triphosphate bridge. In the present disclosure, a cap may be selected that is not susceptible, or less susceptible, to that type of cleavage. A suitable cap analog for this purpose may be selected from a cap dinucleotide according to formula (I):

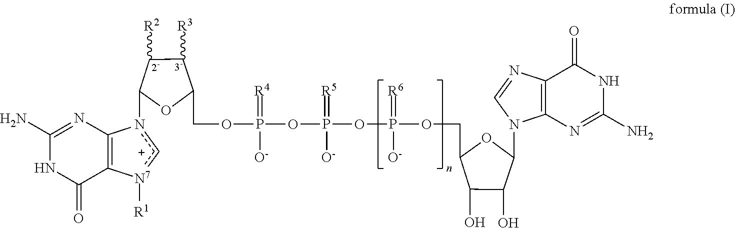

wherein R¹ is selected from the group consisting of optionally substituted alkyl, optionally substituted alkenyl, optionally substituted alkynyl, optionally substituted cycloalkyl, optionally substituted heterocyclyl, optionally substituted aryl, and optionally substituted heteroaryl,
R² and R³ are independently selected from the group consisting of H, halo, OH, and optionally substituted alkoxy, or R² and R³ together form O—X—O, wherein X is selected from the group consisting of optionally substituted CH₂, CH₂CH₂, CH₂CH₂CH₂, CH₂CH(CH₃), and
C(CH₃)₂, or R² is combined with the hydrogen atom at position 4′ of the ring to which R² is attached to form —O—CH₂— or —CH₂—O—,
R⁵ is selected from the group consisting of S, Se, and BH₃,
R⁴ and R⁶ are independently selected from the group consisting of O, S, Se, and BH₃.
n is 1, 2, or 3.

Preferred embodiments for R¹, R², R³, R⁴, R⁵, R⁶ are disclosed in WO 2011/015347 A1 and may be selected accordingly in the present disclosure.

For example, in an embodiment, the RNA molecules of the present disclosure comprise a phosphorothioate-cap-analog. Phosphorothioate-cap-analogs are specific cap analogs in which one of the three non-bridging O atoms in the triphosphate chain is replaced with an S atom, i.e., one of R⁴, R⁵ or R⁶ in Formula (I) is S. Phosphorothioate-cap-analogs have been described by Kowalska et al., 2008, RNA, 14:1119-1131, as a solution to the undesired decapping process, and thus to increase the stability of RNA in vivo. In particular, the substitution of an oxygen atom for a sulphur atom at the beta-phosphate group of the 5′-cap results in stabilization against Dcp2. In that embodiment, which is preferred in the present disclosure, R⁵ in Formula (I) is S; and R⁴ and R⁶ are O.

In a further embodiment, the RNA molecules of the present disclosure comprise a phosphorothioate-cap-analog wherein the phosphorothioate modification of the RNA 5′-cap is combined with an “anti-reverse cap analog” (ARCA) modification. Respective ARCA-phosphorothioate-cap-analogs are described in WO 2008/157688 A2, and they can all be used in the RNA of the present disclosure. In that embodiment, at least one of R² or R³ in Formula (I) is not OH, preferably one among R² and R³ is methoxy (OCH₃), and the other one among R² and R³ is preferably OH. In a preferred embodiment, an oxygen atom is substituted for a sulphur atom at the beta-phosphate group (so that R⁵ in Formula (I) is S; and R⁴ and R⁶ are O). It is believed that the phosphorothioate modification of the ARCA ensures that the α, β, and γ phosphorothioate groups are precisely positioned within the active sites of cap-binding proteins in both the translational and decapping machinery. At least some of these analogs are essentially resistant to pyrophosphatase Dcp1/Dcp2. Phosphorothioate-modified ARCAs were described to have a much higher affinity for eIF4E than the corresponding ARCAs lacking a phosphorothioate group.

A respective cap that is particularly preferred in the present disclosure, i.e., m₂^{7,2-O}GppₛpG, is termed beta-S-ARCA (WO 2008/157688 A2; Kuhn et al., 2010, Gene Ther. 17:961-971). Thus, in one embodiment of the present disclosure, the RNA of the present disclosure is modified with beta-S-ARCA. beta-S-ARCA is represented by the following structure:

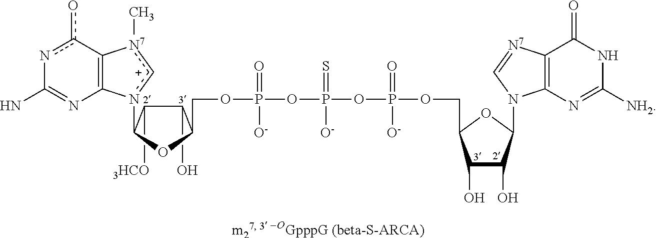

In general, the replacement of an oxygen atom for a sulphur atom at a bridging phosphate results in phosphorothioate diastereomers which are designated D1 and D2, based on their elution pattern in HPLC. Briefly, the D1 diastereomer of beta-S-ARCA” or “beta-S-ARCA(D1)” is the diastereomer of beta-S-ARCA which elutes first on an HPLC column compared to the D2 diastereomer of beta-S-ARCA (beta-S-ARCA(D2)) and thus exhibits a shorter retention time. Determination of the stereochemical configuration by HPLC is described in WO 2011/015347 A1.

In a first particularly preferred embodiment of the present disclosure, RNA of the present disclosure is modified with the beta-S-ARCA(D2) diastereomer. The two diastereomers of beta-S-ARCA differ in sensitivity against nucleases. It has been shown that RNA carrying the D2 diastereomer of beta-S-ARCA is almost fully resistant against Dcp2 cleavage (only 6% cleavage compared to RNA which has been synthesized in presence of the unmodified ARCA 5′-cap), whereas RNA with the beta-S-ARCA(D1) 5′-cap exhibits an intermediary sensitivity to Dcp2 cleavage (71% cleavage). It has further been shown that the increased stability against Dcp2 cleavage correlates with increased protein expression in mammalian cells. In particular, it has been shown that RNAs carrying the beta-S-ARCA(D2) cap are more efficiently translated in mammalian cells than RNAs carrying the beta-S-ARCA(D1) cap. Therefore, in one embodiment of the present disclosure, RNA of the present disclosure is modified with a cap analog according to Formula (I), characterized by a stereochemical configuration at the P atom comprising the substituent R⁵ in Formula (I) that corresponds to that at the P_{β} atom of the D2 diastereomer of beta-S-ARCA. In that embodiment, R⁵ in Formula (I) is S; and R⁴ and R⁶ are O. Additionally, at least one of R² or R³ in Formula (I) is preferably not OH, preferably one among R² and R³ is methoxy (OCH₃), and the other one among R² and R³ is preferably OH.

In a second particularly preferred embodiment, RNA of the present disclosure is modified with the beta-S-ARCA(D1) diastereomer. This embodiment is particularly suitable for transfer of capped RNA into immature antigen presenting cells, such as for vaccination purposes. It has been demonstrated that the beta-S-ARCA(D1) diastereomer, upon transfer of respectively capped RNA into immature antigen presenting cells, is particularly suitable for increasing the stability of the RNA, increasing translation efficiency of the RNA, prolonging translation of the RNA, increasing total protein expression of the RNA, and/or increasing the immune response against an antigen or antigen peptide encoded by said RNA (Kuhn et al., 2010, Gene Ther. 17:961-971). Therefore, in an alternative embodiment of the present disclosure, RNA of the present disclosure is modified with a cap analog according to Formula (I), characterized by a stereochemical configuration at the P atom comprising the substituent R⁵ in Formula (I) that corresponds to that at the P_{β} atom of the D1 diastereomer of beta-S-ARCA. Respective cap analogs and embodiments thereof are described in WO 2011/015347 A1 and Kuhn et al., 2010, Gene Ther. 17:961-971. Any cap analog described in WO 2011/015347 A1, wherein the stereochemical configuration at the P atom comprising the substituent R⁵ corresponds to that at the P_{β} atom of the D1 diastereomer of beta-S-ARCA, may be used in the present disclosure. Preferably, R⁵ in Formula (I) is S; and R⁴ and R⁶ are O. Additionally, at least one of R² or R³ in Formula (I) is preferably not OH, preferably one among R² and R³ is methoxy (OCH₃), and the other one among R² and R³ is preferably OH.

In one embodiment, RNA of the present disclosure is modified with a 5′-cap structure according to Formula (I), wherein any one phosphate group is replaced by a boranophosphate group or a phosphoroselenoate group. Such caps have increased stability both in vitro and in vivo. Optionally, the respective compound has a 2′-O— or 3′-O-alkyl group (wherein alkyl is preferably methyl); respective cap analogs are termed BH₃-ARCAs or Se-ARCAs. Compounds that are particularly suitable for capping of mRNA include the β-BH₃-ARCAs and 3-Se-ARCAs, as described in WO 2009/149253 A2. For these compounds, a stereochemical configuration at the P atom comprising the substituent R⁵ in Formula (I) that corresponds to that at the P_{β} atom of the D1 diastereomer of beta-S-ARCA is preferred.

In some embodiments, the RNA comprises a cap which may be suitable in the context of the present disclosure is a cap0 (methylation of the first nucleobase, e.g. ^{m7}GpppN), cap1 (additional methylation of the ribose of the adjacent nucleotide of ^{m7}GpppN), cap2 (additional methylation of the ribose of the 2nd nucleotide downstream of the ^{m7}GpppN), cap3 (additional methylation of the ribose of the 3rd nucleotide downstream of the ^{m7}GpppN), cap4 (additional methylation of the ribose of the 4th nucleotide downstream of the ^{m7}GpppN), ARCA (anti-reverse cap analogue), modified ARCA (e.g. phosphothioate modified ARCA, e.g., beta-S-ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In some embodiments, the RNA comprises a cap that is a Cap-0 (also referred herein as “Cap0”), a Cap-1 (also referred herein as “Cap1”), or Cap-2 (also referred herein as “Cap2”). See, e.g., FIG. 1 of Ramanathan A et al., and FIG. 1 of Decroly E et al.

In some embodiments, a Cap0 comprises a guanosine nucleoside methylated at the 7-position of guanine (^{m7}G). In some embodiments, a Cap0 is connected to an RNA via a 5′- to 5′-triphosphate linkage and is also referred to herein as ^{m7}Gppp or ^{m7}G(5′)ppp(5′).

In some embodiments, a Cap1 comprises a guanosine nucleoside methylated at the 7-position of guanine (^{m7}G or ^{7m}G) and a 2′O methylated first nucleotide in an RNA (^{2′OMe}N₁ or N₁2′OMe or N₁^{2′OMe}). In some embodiments, a Cap1 is connected to an RNA via a 5′- to 5′-triphosphate linkage; in some embodiments, a Cap1 may be represented as ^{m7}Gppp(N₁^{2′OMe}) or ^{m7}G(5′)ppp(5′)(N₁^{2′OMe}) or ^{7m}G(5′)ppp(5′)N₁^{2′-OMe}). In some embodiments, N₁ is chosen from A, C, G, or U. In some embodiments, N₁ is A. In some embodiments, N₁ is C. In some embodiments, N₁ is G. In some embodiments, N₁ is U.

In some embodiments, a ^{m7}G(5′)ppp(5′)(N₁^{2′OMe}) Cap1 comprises a second nucleotide, N₂ which is a cap proximal A, G, C, or U at position +2. In some embodiments, such Cap1's are represented as (^{m7}G(5′)ppp(5′)(N₁^{2′OMe})pN₂). In some embodiments, N₂ is A. In some embodiments, N₂ is C. In some embodiments, N₂ is G. In some embodiments, N₂ is U.

In some embodiments, a Cap1 is or comprises ^{m7}G(5′)ppp(5′)(A₁^{2′OMe})pG₂ wherein A₁ is a cap proximal A at position +1 and G₂ is a cap proximal G at position +2, and has the following structure:

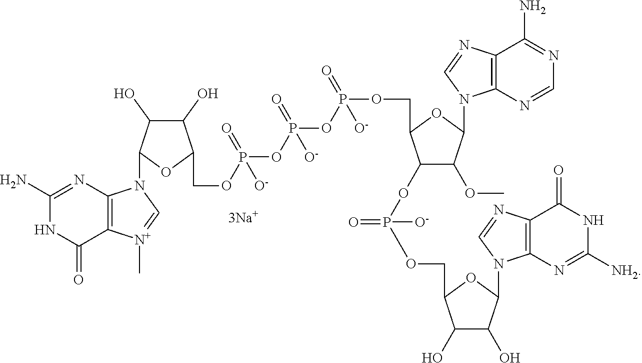 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

In some embodiments, a Cap1 is or comprises ^{m7}G(5′)ppp(5′)(A₁^{2′OMe})pU₂ wherein A₁ is a cap proximal A at position +1 and U₂ is a cap proximal U at position +2, and has the following structure:

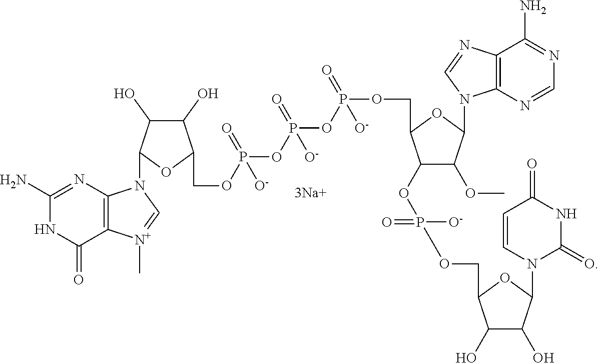 [SMILES: COC1C(OP(=O)([O-])OCC2OC(n3ccc(=O)[nH]c3=O)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

In some embodiments, a Cap1 is or comprises ^{m7}G(5′)ppp(5′)(G₁^{2′OMe})pG₂ wherein G₁ is a cap proximal G at position +1 and G₂ is a cap proximal G at position +2, and has the following structure:

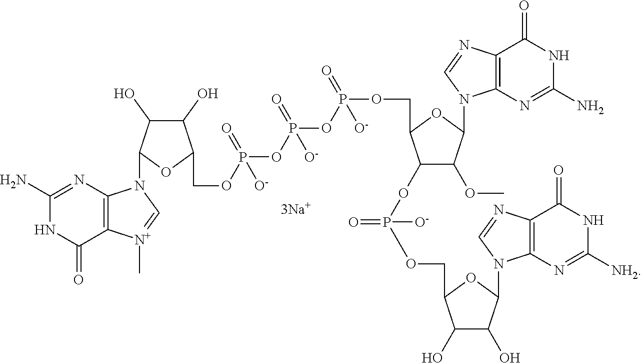 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(=O)[nH]c(N)nc21]

In some embodiments, a Cap1 comprises a guanosine nucleoside methylated at the 7-position of guanine (^{m7}G) and one or more additional modifications, e.g., methylation on a ribose, and a 2′O methylated first nucleotide in an RNA. In some embodiments, a Cap1 comprises a guanosine nucleoside methylated at the 7-position of guanine and a 3′O methylation at a ribose (m7G3′OMe or ^{7m}G^{3′OMe}); and a 2′O methylated first nucleotide in an RNA (N₁^{2′OMe}). In some embodiments, a Cap1 is connected to an RNA via a 5′- to 5′-triphosphate linkage and is also referred to herein as (m7G3′OMe)ppp(2′OMeN₁) or (^{m7}G^{3′OMe})(5′)ppp(5′)(^{2′OMe}N₁). In some embodiments, N₁ is chosen from A, C, G, or U. In some embodiments, N₁ is A. In some embodiments, N₁ is C. In some embodiments, N₁ is G. In some embodiments, N₁ is U.

In some embodiments, a (^{m7}G^{3′OMe})(5′)ppp(5′)(N₁^{2′OMe}) Cap1 comprises a second nucleotide, N₂ which is a cap proximal nucleotide at position 2 and is chosen from A, G, C, or U (^{m7}G^{3′OMe})(5′)ppp(5′)(N₁^{2′OMe})pN₂). In some embodiments, N₂ is A. In some embodiments, N₂ is C. In some embodiments, N₂ is G. In some embodiments, N₂ is U.

In some embodiments, a Cap1 is or comprises (^{m7}G^{3′OMe})(5′)ppp(5′)(A₁^{2′OMe})pG₂ wherein A₁ is a cap proximal A at position +1 and G₂ is a cap proximal G at position +2, and has the following structure:

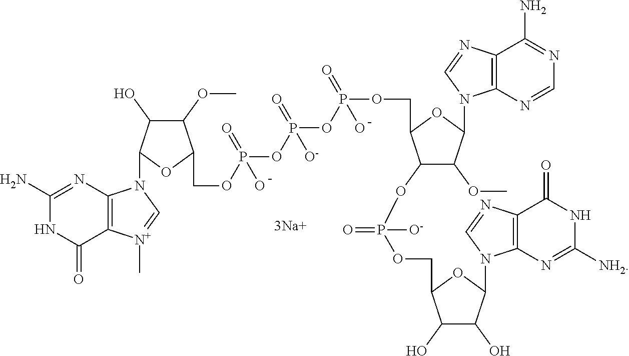 [SMILES: COC1C(COP(=O)(O)OP(=O)([O-])OP(=O)(O)OCC2OC(n3cnc4c(N)ncnc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O]

In some embodiments, a Cap1 is or comprises (^{m7}G^{3′OMe})(5′)ppp(5′)(G₁^{2′OMe})pG₂ wherein G₁ is a cap proximal G at position +1 and G₂ is a cap proximal G at position +2, and has the following structure:

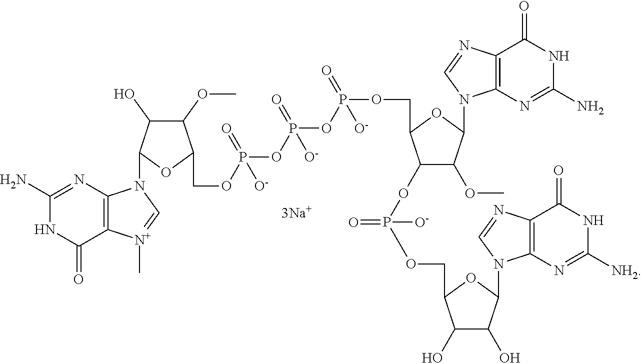 [SMILES: COC1C(COP(=O)([O-])OP(=O)([O-])OP(=O)([O-])OCC2OC(n3cnc4c(=O)[nH]c(N)nc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O]

In some embodiments, a second nucleotide in a Cap1 can comprise one or more modifications, e.g., methylation. In some embodiments, a Cap1 comprising a second nucleotide comprising a 2′O methylation is a Cap2 structure.

In some embodiments, an RNA polynucleotide comprising a Cap1 has increased translation efficiency, increased translation rate and/or increased expression of an encoded payload relative to an appropriate reference comparator. In some embodiments, an RNA polynucleotide comprising a Cap1 having (^{m7}G^{3′OMe})(5′)ppp(5′)(A₁^{2′OMe})pG₂ wherein A₁ is a cap proximal nucleotide at position +1 and G₂ is a cap proximal nucleotide at position +2, has increased translation efficiency relative to an RNA polynucleotide comprising a Cap1 having (^{m7}G^{3′OMe})(5′)ppp(5′)(G₁^{2′OMe})pG₂ wherein G₁ is a cap proximal nucleotide at position 1 and G2 is a cap proximal nucleotide at position 2. In some embodiments, increased translation efficiency is assessed upon administration of an RNA polynucleotide to a cell or an organism.

In some embodiments, a cap analog used in an RNA polynucleotide is ^{m7}G^{3′OMe}Gppp(m1^{2′-OMe})ApG (also sometimes referred to as m2^{7,3′-OMe}G(5′)ppp(5′)m^{2′-OMe}ApG or (^{m7}G^{3′OMe})(5′)ppp(5′)(A^{2′OMe})pG), which has the following structure:

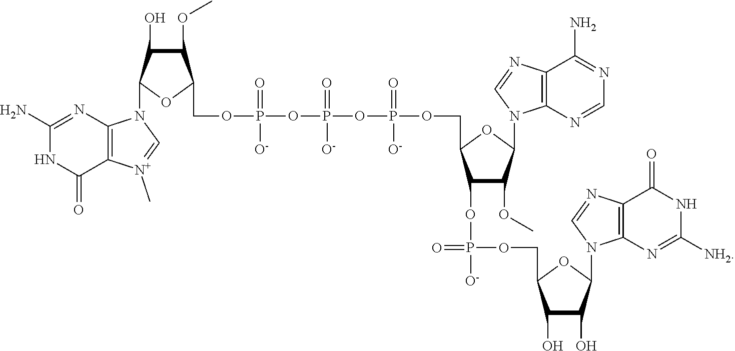

Below is an exemplary Cap1 RNA, which comprises RNA and m2^{7,3′OMe}G(5′)ppp(5′)m^{2′-OMe}APG:

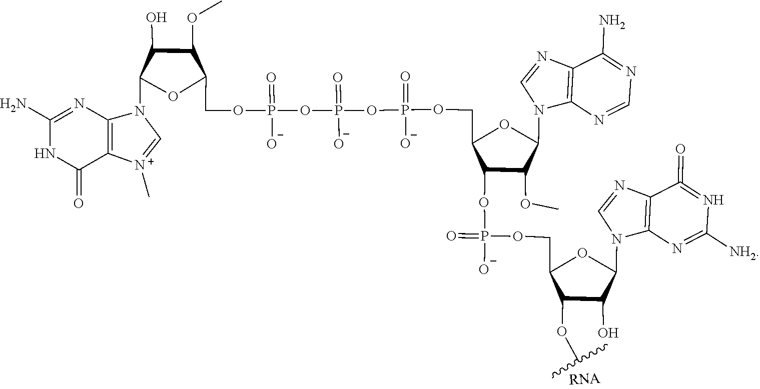

Below is another exemplary Cap1 RNA:

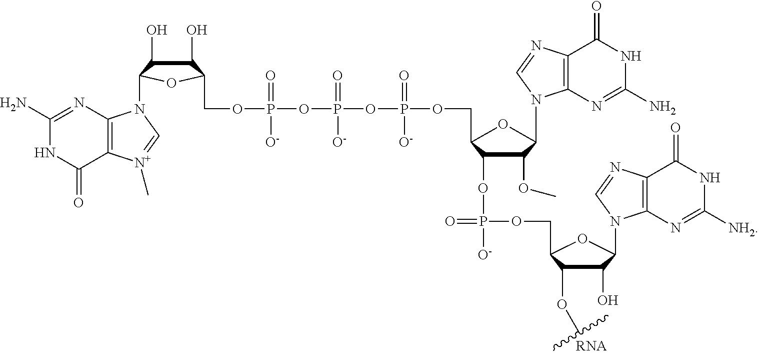

### UTR

The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR).

A 3′-UTR, if present, is located at the 3′ end of a gene, downstream of the termination codon of a protein-encoding region, but the term “3′-UTR” does preferably not include the poly(A) tail. Thus, the 3′-UTR is upstream of the poly(A) tail (if present), e.g. directly adjacent to the poly(A) tail.

A 5′-UTR, if present, is located at the 5′ end of a gene, upstream of the start codon of a protein-encoding region. A 5′-UTR is downstream of the 5′-cap, e.g. directly adjacent to the 5′-cap.

5′- and/or 3′-untranslated regions may, according to the disclosure, be functionally linked to an open reading frame, so as for these regions to be associated with the open reading frame in such a way that the stability and/or translation efficiency of the RNA comprising said open reading frame are increased.

In some embodiments, the RNA molecules according to the present disclosure comprise a 5′-UTR and/or a 3′-UTR.

UTRs are implicated in stability and translation efficiency of RNA. Both can be improved, besides structural modifications concerning the 5′-cap and/or the 3′ poly(A)-tail as described herein, by selecting specific 5′ and/or 3′ untranslated regions (UTRs). Sequence elements within the UTRs are generally understood to influence translational efficiency (mainly 5′-UTR) and RNA stability (mainly 3′-UTR). It is preferable that a 5′-UTR is present that is active in order to increase the translation efficiency and/or stability of the RNA. Independently or additionally, it is preferable that a 3′-UTR is present that is active in order to increase the translation efficiency and/or stability of the RNA molecule.

The terms “active in order to increase the translation efficiency” and/or “active in order to increase the stability”, with reference to a first nucleic acid sequence (e.g. a UTR), means that the first nucleic acid sequence is capable of modifying, in a common transcript with a second nucleic acid sequence, the translation efficiency and/or stability of said second nucleic acid sequence in such a way that said translation efficiency and/or stability is increased in comparison with the translation efficiency and/or stability of said second nucleic acid sequence in the absence of said first nucleic acid sequence.

A 5′-UTR according to the present disclosure can comprise any combination of more than one nucleic acid sequence, optionally separated by a linker. A 3′-UTR according to the present disclosure can comprise any combination of more than one nucleic acid sequence, optionally separated by a linker.

The term “linker” according to the disclosure relates to a nucleic acid sequence added between two nucleic acid sequences to connect said two nucleic acid sequences. There is no particular limitation regarding the linker sequence.

A 3′-UTR typically has a length of 200 to 2000 nucleotides, e.g. 500 to 1500 nucleotides. The 3′-untranslated regions of immunoglobulin mRNAs are relatively short (fewer than about 300 nucleotides), while the 3′-untranslated regions of other genes are relatively long. For example, the 3′-untranslated region of tPA is about 800 nucleotides in length, that of factor VIII is about 1800 nucleotides in length and that of erythropoietin is about 560 nucleotides in length. The 3′-untranslated regions of mammalian mRNA typically have a homology region known as the AAUAAA hexanucleotide sequence. This sequence is presumably the poly(A) attachment signal and is frequently located from 10 to 30 bases upstream of the poly(A) attachment site. 3′-untranslated regions may contain one or more inverted repeats which can fold to give stem-loop structures which act as barriers for exoribonucleases or interact with proteins known to increase RNA stability (e.g. RNA-binding proteins).

The human beta-globin 3′-UTR, particularly two consecutive identical copies of the human beta-globin 3′-UTR, contributes to high transcript stability and translational efficiency (Holtkamp et al., 2006, Blood 108:4009-4017). Thus, in one embodiment, the RNA molecule according to the present disclosure comprises two consecutive identical copies of the human beta-globin 3′-UTR. Thus, it comprises in the 5′ Q 3′ direction: (a) optionally a 5′-UTR; (b) an open reading frame; (c) a 3′-UTR; said 3′-UTR comprising two consecutive identical copies of the human beta-globin 3′-UTR, a fragment thereof, or a variant of the human beta-globin 3′-UTR or fragment thereof.

In an embodiment, the RNA molecules according to the present disclosure comprise a 3′-UTR which is active in order to increase translation efficiency and/or stability, but which is not the human beta-globin 3′-UTR, a fragment thereof, or a variant of the human beta-globin 3′-UTR or fragment thereof.

In an embodiment, the RNA molecules according to the present disclosure comprise a 5′-UTR which is active in order to increase translation efficiency and/or stability.

### Poly(A) Sequence

In some embodiments, the RNA molecules according to the present disclosure comprise a 3′-poly(A) sequence.

According to the disclosure, in one embodiment, a poly(A) sequence comprises or essentially consists of or consists of at least 20, preferably at least 26, preferably at least 40, preferably at least 80, preferably at least 100 and preferably up to 500, preferably up to 400, preferably up to 300, preferably up to 200, and in particular up to 150 A nucleotides, and in particular about 120 A nucleotides. In this context “essentially consists of” means that most nucleotides in the poly(A) sequence, typically at least 50%, and preferably at least 75% by number of nucleotides in the “poly(A) sequence”, are A nucleotides (adenylate), but permits that remaining nucleotides are nucleotides other than A nucleotides, such as U nucleotides (uridylate), G nucleotides (guanylate), C nucleotides (cytidylate). In this context “consists of” means that all nucleotides in the poly(A) sequence, i.e., 100% by number of nucleotides in the poly(A) sequence, are A nucleotides. The term “A nucleotide” or “A” refers to adenylate.

Indeed, it has been demonstrated that a 3′-poly(A) sequence of about 120 A nucleotides has a beneficial influence on the levels of RNA in transfected eukaryotic cells, as well as on the levels of protein that is translated from an open reading frame that is present upstream (5′) of the 3′-poly(A) sequence (Holtkamp et al., 2006, Blood, vol. 108, pp. 4009-4017).

The present disclosure provides for a 3′-poly(A) sequence to be attached during RNA transcription, i.e. during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments of the present disclosure, the 3′-poly(A) cassette present in the coding strand of DNA template molecules essentially consists of dA nucleotides, but is interrupted by a random sequence having an equal distribution of the four nucleotides (dA, dC, dG, dT). Such random sequence may be 5 to 50, preferably 10 to 30, more preferably 10 to 20 nucleotides in length. Such a cassette is disclosed in WO 2016/005004 A1. Any poly(A) cassette disclosed in WO 2016/005004 A1 may be used in the present disclosure. A poly(A) cassette that essentially consists of dA nucleotides, but is interrupted by a random sequence having an equal distribution of the four nucleotides (dA, dC, dG, dT) and having a length of, e.g., 5 to 50 nucleotides shows, on DNA level, constant propagation of plasmid DNA in E. coli and is still associated, on RNA level, with the beneficial properties with respect to supporting RNA stability and translational efficiency.

Consequently, in some embodiments of the present disclosure, the 3′-poly(A) sequence contained in an RNA molecule described herein essentially consists of A nucleotides, but is interrupted by a random sequence having an equal distribution of the four nucleotides (A, C, G, U). Such random sequence may be 5 to 50, preferably 10 to 30, more preferably 10 to 20 nucleotides in length.

### Codon Usage

In general, the degeneracy of the genetic code will allow the substitution of certain codons (base triplets coding for an amino acid) that are present in an RNA sequence by other codons (base triplets), while maintaining the same coding capacity (so that the replacing codon encodes the same amino acid as the replaced codon). In some embodiments of the present disclosure, at least one codon of an open reading frame comprised by an RNA molecule differs from the respective codon in the respective open reading frame in the species from which the open reading frame originates. In that embodiment, the coding sequence of the open reading frame is said to be “adapted” or “modified”. The coding sequence of an open reading frame comprised by the RNA molecule may be adapted.

For example, when the coding sequence of an open reading frame is adapted, frequently used codons may be selected: WO 2009/024567 A1 describes the adaptation of a coding sequence of a nucleic acid molecule, involving the substitution of rare codons by more frequently used codons. Since the frequency of codon usage depends on the host cell or host organism, that type of adaptation is suitable to fit a nucleic acid sequence to expression in a particular host cell or host organism. Generally, speaking, more frequently used codons are typically translated more efficiently in a host cell or host organism, although adaptation of all codons of an open reading frame is not always required.

For example, when the coding sequence of an open reading frame is adapted, the content of G (guanylate) residues and C (cytidylate) residues may be altered by selecting codons with the highest GC-rich content for each amino acid. RNA molecules with GC-rich open reading frames were reported to have the potential to reduce immune activation and to improve translation and half-life of RNA (Thess et al., 2015, Mol. Ther. 23:1457-1465).

### Antibodies

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (abbreviated herein as C_{H} or CH). The heavy chain constant region typically is comprised of three domains, CH1, CH2, and CH3. The hinge region is the region between the CH1 and CH2 domains of the heavy chain and is highly flexible. Disulphide bonds in the hinge region are part of the interactions between two heavy chains in an IgG molecule. Each light chain typically is comprised of a light chain variable region (abbreviated herein as V_{L} or VL) and a light chain constant region (abbreviated herein as C_{L} or CL). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)).

The term “antibody” (Ab) as used herein refers to an immunoglobulin molecule, a fragment of an immunoglobulin molecule, or a derivative of either thereof, which has the ability to bind, preferably specifically bind to an antigen. In some embodiments, binding takes place under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen). The variable regions of the heavy and light chains of the immunoglobulin molecule contain a binding domain that interacts with an antigen. The term “antigen-binding region”, “binding region” or “binding domain”, as used herein, refers to the region or domain which interacts with the antigen and typically comprises both a VH region and a VL region. The term antibody when used herein comprises not only monospecific antibodies, but also multispecific antibodies which comprise multiple, such as two or more, e.g. three or more, different antigen-binding regions. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody as used herein, unless otherwise stated or clearly contradicted by context, includes fragments of an antibody that are antigen-binding fragments, i.e., retain the ability to specifically bind to the antigen, and antibody derivatives, i.e., constructs that are derived from an antibody. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO2007059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al; Trends Biotechnol. 2003 November; 21(11):484-90); (vi) camelid or Nanobody molecules (Revets et al; Expert Opin Biol Ther. 2005 January; 5(1):111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present disclosure, exhibiting different biological properties and utility. These and other useful antibody fragments in the context of the present disclosure, as well as bispecific formats of such fragments, are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques.

The phrase “single chain Fv” or “scFv” refers to an antibody in which the variable domains of the heavy chain and of the light chain (VH and VL) of a traditional two chain antibody have been joined to form one chain. Optionally, a linker (usually a peptide) is inserted between the two chains to allow for proper folding and creation of an active binding site.

A single-domain antibody, also known as a nanobody, is an antibody fragment consisting of a single monomeric variable antibody domain. In some embodiments, a single-domain antibody is a variable domain (V_{H}) of a heavy-chain antibody. These are called VHH fragments. Like a whole antibody, a single-domain antibody is able to bind selectively to a specific antigen. The first single-domain antibodies were engineered from heavy-chain antibodies found in camelids. Cartilaginous fishes also have heavy-chain antibodies (IgNAR, ‘immunoglobulin new antigen receptor’), from which single-domain antibodies called VNAR fragments can be obtained. An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes.

An antibody can possess any isotype. As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody may be a sequence variant of a naturally-occurring IgG1 antibody, including variations in the constant regions.

In various embodiments, an antibody is an IgG1 antibody, more particularly an IgG1, kappa or IgG1, lambda isotype (i.e. IgG1, κ, λ), an IgG2a antibody (e.g. IgG2a, κ, ×), an IgG2b antibody (e.g. IgG2b, κ, λ), an IgG3 antibody (e.g. IgG3, κ, λ) or an IgG4 antibody (e.g. IgG4, κ, λ).

The term “monoclonal antibody” as used herein refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

The term “chimeric antibody” as used herein, refers to an antibody wherein the variable region is derived from a non-human species (e.g. derived from rodents) and the constant region is derived from a different species, such as human. Chimeric monoclonal antibodies for therapeutic applications are developed to reduce antibody immunogenicity. The terms “variable region” or “variable domain” as used in the context of chimeric antibodies, refer to a region which comprises the CDRs and framework regions of both the heavy and light chains of the immunoglobulin. Chimeric antibodies may be generated by using standard DNA techniques as described in Sambrook et al., 1989, Molecular Cloning: A laboratory Manual, New York: Cold Spring Harbor Laboratory Press, Ch. 15. The chimeric antibody may be a genetically or an enzymatically engineered recombinant antibody. It is within the knowledge of the skilled person to generate a chimeric antibody, and thus, generation of the chimeric antibody may be performed by other methods than described herein.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “human antibody” as used herein, refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse or rat, have been grafted onto human framework sequences. Human monoclonal antibodies can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well established procedure. Immunization protocols and techniques for isolation of immunized splenocytes for fusion are known in the art. Fusion partners (e.g., murine myeloma cells) and fusion procedures are also known. Human monoclonal antibodies can thus e.g. be generated using transgenic or transchromosomal mice or rats carrying parts of the human immune system rather than the mouse or rat system. Accordingly, in some embodiments, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogeneous animal antibody machinery, see e.g. Mendez et al. 1997 Nat Genet. 15(2):146-56.

When used herein, unless contradicted by context, the term “Fab-arm”, “binding arm” or “arm” includes one heavy chain-light chain pair and is used interchangeably with “half-molecule” herein.

The term “full-length” when used in the context of an antibody indicates that the antibody is not a fragment, but contains all of the domains of the particular isotype normally found for that isotype in nature, e.g. the VH, CH1, CH2, CH3, hinge, VL and CL domains for an IgG1 antibody.

When used herein, unless contradicted by context, the term “Fc region” refers to an antibody region consisting of the two Fc sequences of the heavy chains of an immunoglobulin, wherein said Fc sequences comprise at least a hinge region, a CH2 domain, and a CH3 domain.

The present disclosure also envisions antibodies comprising functional variants of the VL regions, VH regions, or one or more CDRs of the antibodies described herein. A functional variant of a VL, VH, or CDR used in the context of an antibody still allows the antibody to retain at least a substantial proportion (at least about 50%, 60%, 70%, 80%, 90%, 95% or more) of the affinity and/or the specificity/selectivity of the “reference” or “parent” antibody and in some cases, such an antibody may be associated with greater affinity, selectivity and/or specificity than the parent antibody.

Such functional variants typically retain significant sequence identity to the parent antibody.

Exemplary variants include those which differ from VH and/or VL and/or CDR regions of the parent antibody sequences mainly by conservative substitutions; for instance, up to 10, such as 9, 8, 7, 6, 5, 4, 3, 2 or 1 of the substitutions in the variant are conservative amino acid residue replacements.

Functional variants of antibody sequences described herein such as VL regions, or VH regions, or antibody sequences having a certain degree of homology or identity to antibody sequences described herein such as VL regions, or VH regions preferably comprise modifications or variations in the non-CDR sequences, while the CDR sequences preferably remain unchanged.

The term “specificity” as used herein is intended to have the following meaning unless contradicted by context. Two antibodies have the “same specificity” if they bind to the same antigen and the same epitope.

An antibody or fragment useful herein may compete with a specific antibody or fragment described herein.

The term “competes” and “competition” may refer to the competition between a first antibody and a second antibody to the same antigen. It is well known to a person skilled in the art how to test for competition of antibodies for binding to a target antigen. An example of such a method is a so-called cross-competition assay, which may e.g. be performed as an ELISA or by flow-cytometry. Alternatively, competition may be determined using biolayer interferometry.

Antibodies which compete for binding to a target antigen may bind different epitopes on the antigen, wherein the epitopes are so close to each other that a first antibody binding to one epitope prevents binding of a second antibody to the other epitope. In other situations, however, two different antibodies may bind the same epitope on the antigen and would compete for binding in a competition binding assay. Such antibodies binding to the same epitope are considered to have the same specificity herein. Thus, in some embodiments, antibodies binding to the same epitope are considered to bind to the same amino acids on the target molecule. That antibodies bind to the same epitope on a target antigen may be determined by standard alanine scanning experiments or antibody-antigen crystallization experiments known to a person skilled in the art. Preferably, antibodies or binding domains binding to different epitopes are not competing with each other for binding to their respective epitopes.

Naturally occurring antibodies are generally monospecific, i.e. they bind to a single antigen. Described herein are binding agents, e.g., docking compounds, binding to different epitopes on e.g. a primary target and a connector compound. Such binding agents are at least bispecific or multispecific such as trispecific, tetraspecific and so on. Thus, the binding agent may comprise two or more antibodies as described herein or fragments thereof. In particular, a binding agent described herein may be an artificial protein that is composed of two different antibodies, an antibody and a fragment of a different antibody, and fragments of two different antibodies (said fragments of two different antibodies forming two binding domains).

According to the disclosure, a bispecific binding agent, in particular a bispecific protein, such as a bispecific antibody is a molecule that has two different binding specificities and thus may bind to two epitopes. Particularly, the term “bispecific antibody” as used herein refers to an antibody comprising two antigen-binding sites, a first binding site having affinity for a first epitope and a second binding site having binding affinity for a second epitope distinct from the first.

The term “bispecific” as used herein refers to an agent having two different antigen-binding regions binding to different epitopes.

“Multispecific binding agents” are molecules which have more than two different binding specificities.

Many different formats and uses of bispecific antibodies are known in the art, and were reviewed by Kontermann; Drug Discov Today, 2015 July; 20(7):838-47 and; MAbs, 2012 March-April; 4(2):182-97.

A bispecific binding agent according to the present disclosure is not limited to any particular bispecific format or method of producing it.

Examples of bispecific antibody molecules which may be used herein comprise (i) a single antibody that has two arms comprising different antigen-binding regions; (ii) a single chain antibody that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (iv) a chemically-linked bispecific (Fab′)2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody.

The term “bispecific antibody” includes diabodies. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90: 6444-6448; Poljak, R. J., et al. (1994) Structure 2: 1121-1123). Bispecific antibodies also include bispecific single chain antibodies. The term “bispecific single chain antibody” denotes a single polypeptide chain comprising two binding domains. In particular, the term “bispecific single chain antibody” or “single chain bispecific antibody” or related terms as used herein preferably mean antibody constructs resulting from joining at least two antibody variable regions in a single polypeptide chain devoid of the constant and/or Fc portion(s) present in full immunoglobulins. For example, a bispecific single chain antibody may be a construct with a total of two antibody variable regions, for example two VH regions, each capable of specifically binding to a separate epitope, and connected with one another through a short polypeptide spacer such that the two antibody variable regions with their interposed spacer exist as a single contiguous polypeptide chain. Another example of a bispecific single chain antibody may be a single polypeptide chain with three antibody variable regions. Here, two antibody variable regions, for example one VH and one VL, may make up an scFv, wherein the two antibody variable regions are connected to one another via a synthetic polypeptide linker, the latter often being genetically engineered so as to be minimally immunogenic while remaining maximally resistant to proteolysis. This scFv is capable of specifically binding to a particular epitope, and is connected to a further antibody variable region, for example a VH region, capable of binding to a different epitope than that bound by the scFv. Yet another example of a bispecific single chain antibody may be a single polypeptide chain with four antibody variable regions. Here, the first two antibody variable regions, for example a VH region and a VL region, may form one scFv capable of binding to one epitope, whereas the second VH region and VL region may form a second scFv capable of binding to another epitope. Within a single contiguous polypeptide chain, individual antibody variable regions of one specificity may advantageously be separated by a synthetic polypeptide linker, whereas the respective scFvs may advantageously be separated by a short polypeptide spacer as described above. According to some embodiments, the first binding domain of the bispecific antibody comprises one antibody variable domain, preferably a VHH domain. According to some embodiments, the first binding domain of the bispecific antibody comprises two antibody variable domains, preferably a scFv, i.e. VH-VL or VL-VH. According to some embodiments, the second binding domain of the bispecific antibody comprises one antibody variable domain, preferably a VHH domain. According to some embodiments, the second binding domain of the bispecific antibody comprises two antibody variable domains, preferably a scFv, i.e. VH-VL or VL-VH. In its minimal form, the total number of antibody variable regions in the bispecific antibody is thus only two. For example, such an antibody could comprise two VH or two VHH domains. According to some embodiments, the first binding domain and the second binding domain of the bispecific antibody each comprise one antibody variable domain, preferably a VHH domain. According to some embodiments, the first binding domain and the second binding domain of the bispecific antibody each comprise two antibody variable domains, preferably a scFv, i.e. VH-VL or VL-VH. In this embodiment, the binding agent preferably comprises (i) a heavy chain variable domain (VH) of a first antibody, (ii) a light chain variable domain (VL) of a first antibody, (iii) a heavy chain variable domain (VH) of a second antibody and (iv) a light chain variable domain (VL) of a second antibody.

In some embodiments, the bispecific molecules comprise two Fab regions, each being directed against different epitopes. In some embodiments, the molecule of the disclosure is an antigen binding fragment (Fab)2 complex. The Fab2 complex is composed of two Fab fragments, one Fab fragment comprising a Fv domain, i.e. VH and VL domains, specific for one epitope, and the other Fab fragment comprising a Fv domain specific for another epitope. Each of the Fab fragments may be composed of two single chains, a VL-CL module and a VH-CH module. Alternatively, each of the individual Fab fragments may be arranged in a single chain, preferably, VL-CL-CH-VH, and the individual variable and constant domains may be connected with a peptide linker.

In some embodiments, the binding agent according to the disclosure includes various types of bivalent and trivalent single-chain variable fragments (scFvs), fusion proteins mimicking the variable domains of two antibodies. Divalent (or bivalent) single-chain variable fragments (di-scFvs, bi-scFvs) can be engineered by linking two scFvs. This can be done by producing a single peptide chain with two VH and two VL regions, yielding tandem scFvs. The disclosure also includes multispecific molecules comprising more than two scFvs binding domains.

Another possibility is the creation of scFvs with linker peptides that are too short for the two variable regions to fold together (about five amino acids), forcing scFvs to dimerize. This type is known as diabodies. Still shorter linkers (one or two amino acids) lead to the formation of trimers, so-called triabodies or tribodies. Tetrabodies have also been produced. They exhibit an even higher affinity to their targets than diabodies.

A particularly preferred example of a bispecific antibody fragment is a diabody (Kipriyanov, Int. J. Cancer 77 (1998), 763-772), which is a small bivalent and bispecific antibody fragment. Diabodies comprise a heavy chain variable domain (VH) and a light chain variable domain (VL) on the same polypeptide chain (VH-VL) connected by a peptide linker that is too short to allow pairing between the two domains on the same chain. This forces pairing with the complementary domains of another chain and promotes the assembly of a dimeric molecule with two functional antigen binding sites.

In some embodiments, the bispecific or multispecific molecule according to the disclosure comprises variable (VH, VL) and constant domains (C) of immunoglobulins. In some embodiments the bispecific molecule is a minibody, preferably, a minibody comprising two single VH-VL-C chains that are connected with each other via the constant domains (C) of each chain. According to this aspect, the corresponding variable heavy chain regions (VH), corresponding variable light chain regions (VL) and constant domains (C) are arranged, from N-terminus to C-terminus, in the order VH(Epitope 1)-VL(Epitope 1)—(C) and VH(Epitope 2)-VL(Epitope 2)—C, wherein C is preferably a CH3 domain, Epitope 1 refers to a first epitope and Epitope 2 refers to a second epitope. Pairing of the constant domains results in formation of the minibody.

According to another aspect, the bispecific binding agent of the disclosure is in the format of a bispecific single chain antibody construct, whereby said construct comprises or consists of at least two binding domains. In some embodiments, each binding domain comprises one variable region from an antibody heavy chain (“VH region”), wherein the VH region of the first binding domain specifically binds to Epitope 1, and the VH region of the second binding domain specifically binds to Epitope 2. The two binding domains are optionally linked to one another by a short polypeptide spacer. Each binding domain may additionally comprise one variable region from an antibody light chain (“VL region”), the VH region and VL region within each of the first and second binding domains being linked to one another via a polypeptide linker long enough to allow the VH region and VL region of the first binding domain and the VH region and VL region of the second binding domain to pair with one another.

In some embodiments, the binding agent described herein comprises an antibody, e.g., a full-length antibody, comprising the first binding domain. In some embodiments, the binding agent described herein comprises an antibody fragment such as scFv or VHH comprising the second binding domain which is covalently linked to the antibody comprising the first binding domain. In some embodiments, the binding agent comprises the antibody fragment such as scFv or VHH covalently linked to the N-terminus or C-terminus of the light chain or heavy chain of the antibody.

In some embodiments, a binding moiety described herein, e.g., a binding moiety comprised in a docking compound binding to a primary target, comprises a DARPin. In some embodiments, the binding moiety directs a particle to immune effector cells, in particular T cells such as CD8⁺ T cells.

The term “DARPin” refers to designed ankyrin repeat proteins. DARPins are based on naturally occurring ankyrin repeat proteins, yet contain one or more amino acid mutations that can affect, for example, their binding affinity to a target molecule, their cell surface expression, and the like. DARPins preferably include 2 to 3 ankyrin repeat modules flanked by N- and C-capping repeats. Each ankyrin repeat module includes about 33 amino acid residues.

Ankyrin repeat proteins have been identified in 1987 through sequence comparisons between four such proteins in Saccharomyces cerevisiae, Drosophila melanogaster and Caenorhabditis elegans. Breeden and Nasmyth reported multiple copies of a repeat unit of approximately 33 residues in the sequences of swi6p, cddOp, notch and lin-12 (Breeden et al., Nature 329, 651-654 (1987)). The subsequent discovery of 24 copies of this repeat unit in the ankyrin protein led to the naming of this repeat unit as the ankyrin repeat (Lux et al., Nature 344, 36-42 (1990)). Later, this repeat unit has been identified in several hundreds of proteins of different organisms and viruses (Bork, Proteins 17(4), 363-74 (1993)). These proteins are located in the nucleus, the cytoplasm or the extracellular space. This is consistent with the fact that the ankyrin repeat domain of these proteins is independent of disulfide bridges and thus independent of the oxidation state of the environment. The number of repeat units per protein varies from two to more than twenty. Tertiary structures of ankyrin repeat units share a characteristic fold (Sedgwick and Smerdon, Trends Biochem Sci. 24(8), 311-6 (1999)) composed of a β-hairpin followed by two antiparallel α-helices and ending with a loop connecting the repeat unit with the next one. Domains built of ankyrin repeat units are formed by stacking the repeat units to an extended and curved structure. Proteins containing ankyrin repeat domains often contain additional domains. While the latter domains have variable functions, the function of the ankyrin repeat domain is most often the binding of other proteins. When analysing the repeat units of these proteins, the target interaction residues are mainly found in the β-hairpin and the exposed part of the first α-helix. These target interaction residues are hence forming a large contact surface on the ankyrin repeat domain. This contact surface is exposed on a framework built of stacked units of α-helix 1, α-helix 2 and the loop.

DARPins that bind to specific targets can be identified by screening combinatorial libraries of DARPins and selecting those with desired binding properties for the target. Such screening methods are described in, e.g., Muench et al., Molecular Therapy, 16(4), 686-693, 2011. For example, ribosomal display or phage display methods can be used to select target-specific DARPins from diverse libraries.

The term “repeat protein” refers to a (poly)peptide/protein comprising one or more repeat domains. In one embodiment, a repeat protein comprises up to four repeat domains. In one embodiment, a repeat protein comprises up to three repeat domains. In one embodiment, a repeat protein comprises up to two repeat domains. In the most preferred embodiment, a repeat protein comprises one repeat domain.

The individual domains of a repeat protein may be connected to each other directly or via (poly)peptide linkers. The term “(poly)peptide linker” refers to an amino acid sequence which is able to link two protein domains. Such linkers include, for example, glycine-serine-linkers of variable lengths and are known to the person skilled in the relevant art.

The term “repeat domain” refers to a protein domain comprising two or more consecutive repeat units (modules). In one embodiment, said repeat units are structural units having the same or a similar folding structure, and preferably stack tightly to preferably create a superhelical structure having a joint hydrophobic core.

The term “structural unit” refers to a locally ordered part of a (poly)peptide, formed by three-dimensional interactions between two or more segments of secondary structure that are near one another along the (poly)peptide chain. Such a structural unit comprises a structural motif.

The term “structural motif” refers to a three-dimensional arrangement of secondary structure elements present in at least one structural unit. Structural motifs are well known to the person skilled in the relevant art. Said structural units may alone not be able to acquire a defined three-dimensional arrangement; however, their consecutive arrangement as repeat modules in a repeat domain leads to a mutual stabilization of neighbouring units which may result in a superhelical structure.

The term “repeat modules” refers to the repeated amino acid sequences of the repeat proteins, which are derived from the repeat units of naturally occurring proteins. Each repeat module comprised in a repeat domain is derived from one or more repeat units of a family of naturally occurring repeat proteins, e.g., ankyrin repeat proteins.

The term “set of repeat modules” refers to the total number of repeat modules present in a repeat domain. Such “set of repeat modules” present in a repeat domain comprises two or more consecutive repeat modules, and may comprise just one type of repeat module in two or more copies, or two or more different types of modules, each present in one or more copies. Such set of repeat modules comprising, for example, 3 repeat modules may comprise consecutively, form N- to C-terminus, repeat module 1, repeat module 2, and repeat module 3.

Different repeat domains may have an identical number of repeat modules per repeat domain or may differ in the number of repeat modules per repeat domain.

Preferably, the repeat modules comprised in a set are homologous repeat modules. In the context of the present disclosure, the term “homologous repeat modules” refers to repeat modules, wherein more than 70% of the framework residues of said repeat modules are homologous. Preferably, more than 80% of the framework residues of said repeat modules are homologous. Most preferably, more than 90% of the framework residues of said repeat modules are homologous. Computer programs to determine the percentage of homology between polypeptides, such as Fasta, Blast or Gap, are known to the person skilled in the relevant art.

The term “repeat unit” refers to amino acid sequences comprising sequence motifs of one or more naturally occurring proteins, wherein said “repeat units” are found in multiple copies, and which exhibit a defined folding topology common to all said motifs determining the fold of the protein. Such repeat units comprise framework residues and interaction residues.

One example of such repeat units is an ankyrin repeat unit. Naturally occurring proteins containing two or more such repeat units are referred to as “naturally occurring repeat proteins”. The amino acid sequences of the individual repeat units of a repeat protein may have a significant number of mutations, substitutions, additions and/or deletions when compared to each other, while still substantially retaining the general pattern, or motif, of the repeat units.

The term “repeat sequence motif” or “repeat consensus sequence” refers to an amino acid sequence, which is deduced from one or more repeat units. Such repeat sequence motifs comprise framework residue positions and target interaction residue positions. Said framework residue positions correspond to the positions of framework residues of said repeat units. Said target interaction residue positions correspond to the positions of target interaction residues of said repeat units. Such repeat sequence motifs comprise fixed positions and randomized positions. The term “fixed position” refers to an amino acid position in a repeat sequence motif, wherein said position is set to a particular amino acid. Frequently, such fixed positions correspond to the positions of framework residues.

The term “randomized position” refers to an amino acid position in a repeat sequence motif, wherein two or more amino acids are allowed at said amino acid position. Frequently, such randomized positions correspond to the positions of target target interaction residues. However, some positions of framework residues may also be randomized.

The term “folding topology” refers to the tertiary structure of said repeat units. The folding topology will be determined by stretches of amino acids forming at least parts of α-helices or β-sheets, or amino acid stretches forming linear polypeptides or loops, or any combination of α-helices, β-sheets and/or linear polypeptides/loops.

The term “consecutive” refers to an arrangement, wherein said modules are arranged in tandem.

In repeat proteins, there are at least 2, frequently 6 or more, 10 or more, or 20 or more repeat units, usually about 2 to 6 repeat units. For the most part, the repeat proteins are structural proteins and/or adhesive proteins, being present in prokaryotes and eukaryotes, including vertebrates and non-vertebrates.

In most cases, said repeat units will exhibit a high degree of sequence identity (same amino acid residues at corresponding positions) or sequence similarity (amino acid residues being different, but having similar physicochemical properties), and some of the amino acid residues might be key residues being strongly conserved in the different repeat units found in naturally occurring proteins.

However, a high degree of sequence variability by amino acid insertions and/or deletions, and/or substitutions between the different repeat units found in naturally occurring proteins will be possible as long as the common folding topology is maintained.

The term “framework residues” relates to amino acid residues of the repeat units, or the corresponding amino acid residues of the repeat modules, which contribute to the folding topology, i.e. which contribute to the fold of said repeat unit (or module) or which contribute to the interaction with a neighboring unit (or module). Such contribution might be the interaction with other residues in the repeat unit (module), or the influence on the polypeptide backbone conformation as found in α-helices or β-sheets, or amino acid stretches forming linear polypeptides or loops.

The term “target interaction residues” refers to amino acid residues of the repeat units, or the corresponding amino acid residues of the repeat modules, which contribute to the interaction with target substances. Such contribution might be the direct interaction with the target substances, or the influence on other directly interacting residues, e.g. by stabilising the conformation of the (poly)peptide of said repeat unit (module) to allow or enhance the interaction of said directly interacting residues with said target.

A “target” may be an individual molecule such as a nucleic acid molecule, a (poly)peptide protein, a carbohydrate, or any other naturally occurring molecule, including any part of such individual molecule, or complexes of two or more of such molecules. The target may be, in particular, a molecule on immune effector cells, in particular CD8.

In one embodiment, the repeat modules are directly connected. In the context of the present invention, the term “directly connected” refers to repeat modules, which are arranged as direct repeats in a repeat protein without an intervening amino acid sequence.

In another embodiment, the repeat modules are connected by a (poly)peptide linker. Thus, the repeat modules may be linked indirectly via a (poly)peptide linker as intervening sequence separating the individual modules. An “intervening sequence” may be any amino acid sequence, which allows to connect the individual modules without interfering with the folding topology or the stacking of the modules. Preferentially, said intervening sequences are short (poly)peptide linkers of less than 10, and even more preferably, of less than 5 amino acid residues.

In one embodiment, a repeat protein further comprises an N- and/or a C-terminal capping module having an amino acid sequence different from any one of said repeat modules. The term “capping module” refers to a polypeptide fused to the N- or C-terminal repeat module of a repeat domain, wherein said capping module forms tight tertiary interactions with said repeat module thereby providing a cap that shields the hydrophobic core of said repeat module at the side not in contact with the consecutive repeat module from the solvent.

Said N- and/or C-terminal capping module may be, or may be derived from, a capping unit or other domain found in a naturally occurring repeat protein adjacent to a repeat unit.

The term “capping unit” refers to a naturally occurring folded (poly)peptide, wherein said (poly)peptide defines a particular structural unit which is N- or C-terminally fused to a repeat unit, wherein said (poly)peptide forms tight tertiary interactions with said repeat unit thereby providing a cap that shields the hydrophobic core of said repeat unit at one side from the solvent. Such capping units may have sequence similarities to said repeat sequence motif.

### Antigen Receptor

Immune effector cells according to the disclosure express at least one antigen receptor such as a chimeric antigen receptor (CAR) or a T cell receptor (TCR) binding an antigen or a procession product thereof, in particular when present on or presented by a target cell. Immune effector cells may be modified (e.g., ex vivo/in vitro or in vivo in a subject to be treated) to express an antigen receptor. In one embodiment, modification to express an antigen receptor takes place ex vivo/in vitro. Subsequently, modified cells may be administered to a patient. In one embodiment, modification to express an antigen receptor takes place in vivo. The cells may be endogenous cells of the patient or may have been administered to a patient.

In one embodiment of all aspects of the invention, expression of an antigen receptor is at the cell surface.

The immune effector cells, in particular T cells, have a first cell-surface expressed antigen receptor. In some embodiments, the immune effector cells, in particular T cells, have a first and a second cell-surface expressed antigen receptor. The terms “cell-surface expressed antigen receptor” and “antigen receptor” are used synonymously throughout this application.

In an embodiment, antigen receptor of the present disclosure may also bind to a cell-surface or soluble cytokine. In this embodiment, the antigen receptor is a cytokine receptor.

### T Cell Receptor

The term “T cell receptor” or “TCR” as used herein refers to a protein receptor on T cells that is composed of a heterodimer of an alpha (α) and beta (β) chain, although in some cells the TCR consists of gamma and delta (γδ) chains. In some embodiments, the TCR may be derived from any cell comprising a TCR, including a helper T cell, a cytotoxic T cell, a memory T cell, regulatory T cell, natural killer T cell, and gamma delta T cell, for example. Each α, β, γ, and δ chain is composed of two Ig-like domains: a variable domain (V) that confers antigen recognition through the complementarity determining regions (CDR), followed by a constant domain (C) that is anchored to cell membrane by a connecting peptide and a transmembrane (TM) region. The TM region associates with the invariant subunits of the CD3 signaling apparatus. Each of the V domains has three CDRs. These CDRs interact with a complex between an antigenic peptide bound to a protein encoded by the major histocompatibility complex (MHC).

### Chimeric Antigen Receptors

Adoptive cell transfer therapy with CAR-engineered T cells expressing chimeric antigen receptors is a promising anti-cancer therapeutic as CAR-modified T cells can be engineered to target virtually any tumor antigen. For example, patient's T cells may be genetically engineered (genetically modified) to express CARs specifically directed towards antigens on the patient's tumor cells, then infused back into the patient.

In some embodiments, the first antigen receptor is a CAR. In some embodiments, the first and second antigen receptor are a CAR.

According to the invention, the term “CAR” (or “chimeric antigen receptor”) is synonymous with the terms “chimeric T cell receptor” and “artificial T cell receptor” and relates to an artificial receptor comprising a single molecule or a complex of molecules which recognizes, i.e., binds to, a target structure (e.g., an antigen) on a target cell such as a cancer cell (e.g., by binding of an antigen binding domain to an antigen expressed on the surface of the target cell) and may confer specificity onto an immune effector cell such as a T cell expressing said CAR on the cell surface. Such cells do not necessarily require processing and presentation of an antigen for recognition of the target cell, but rather may recognize preferably with specificity any antigen present on a target cell. Preferably, recognition of the target structure by a CAR results in activation of an immune effector cell expressing said CAR. A CAR may comprise one or more protein units said protein units comprising one or more domains as described herein. The term “CAR” does not include T cell receptors.

A CAR comprises a target-specific binding element otherwise referred to as an antigen binding moiety or antigen binding domain that is generally part of the extracellular domain of the CAR. The antigen binding domain recognizes a ligand that acts as a cell surface marker on target cells associated with a particular disease state. Specifically, a CAR of the present disclosure targets the antigen such as tumor antigen on a diseased cell such as tumor cell.

In one embodiment, the binding domain in the CAR binds specifically to the antigen. In one embodiment, the antigen to which the binding domain in the CAR binds is expressed in a cancer cell (tumor antigen). In one embodiment, the antigen is expressed on the surface of a cancer cell. In one embodiment, the binding domain binds to an extracellular domain or to an epitope in an extracellular domain of the antigen. In one embodiment, the binding domain binds to native epitopes of the antigen present on the surface of living cells.

In one embodiment of the invention, an antigen binding domain comprises a variable region of a heavy chain of an immunoglobulin (VH) with a specificity for the antigen and a variable region of a light chain of an immunoglobulin (VL) with a specificity for the antigen. In one embodiment, an immunoglobulin is an antibody. In one embodiment, said heavy chain variable region (VH) and the corresponding light chain variable region (VL) are connected via a peptide linker. Preferably, the antigen binding moiety portion in the CAR is a scFv.

The CAR is designed to comprise a transmembrane domain that is fused to the extracellular domain of the CAR. In one embodiment, the transmembrane domain is not naturally associated with one of the domains in the CAR. In one embodiment, the transmembrane domain is naturally associated with one of the domains in the CAR. In one embodiment, the transmembrane domain is modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain.

In some instances, the CAR of the present disclosure comprises a hinge domain which forms the linkage between the transmembrane domain and the extracellular domain.

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

In one embodiment, the CAR comprises a primary cytoplasmic signaling sequence derived from CD3-zeta. Further, the cytoplasmic domain of the CAR may comprise the CD3-zeta signaling domain combined with a costimulatory signaling region.

The identity of the co-stimulation domain is limited only in that it has the ability to enhance cellular proliferation and survival upon binding of the targeted moiety by the CAR. Suitable co-stimulation domains include CD28, CD137 (4-1BB), a member of the tumor necrosis factor receptor (TNFR) superfamily, CD134 (OX40), a member of the TNFR-superfamily of receptors, and CD278 (ICOS), a CD28-superfamily co-stimulatory molecule expressed on activated T cells. The skilled person will understand that sequence variants of these noted co-stimulation domains can be used without adversely impacting the invention, where the variants have the same or similar activity as the domain on which they are modeled. Such variants will have at least about 80% sequence identity to the amino acid sequence of the domain from which they are derived. In some embodiments of the invention, the CAR constructs comprise two co-stimulation domains. While the particular combinations include all possible variations of the four noted domains, specific examples include CD28+CD137 (4-1BB) and CD28+CD134 (OX40).

The cytoplasmic signaling sequences within the cytoplasmic signaling portion of the CAR may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the CAR comprises a signal peptide which directs the nascent protein into the endoplasmic reticulum. In one embodiment, the signal peptide precedes the antigen binding domain. In one embodiment, the signal peptide is derived from an immunoglobulin such as IgG.

A CAR may comprise the above domains, together in the form of a fusion protein. Such fusion proteins will generally comprise an antigen binding domain, one or more co-stimulation domains, and a signaling sequence, linked in a N-terminal to C-terminal direction. However, the CARs of the present invention are not limited to this arrangement and other arrangements are acceptable and include a binding domain, a signaling domain, and one or more co-stimulation domains. It will be understood that because the binding domain must be free to bind antigen, the placement of the binding domain in the fusion protein will generally be such that display of the region on the exterior of the cell is achieved. In the same manner, because the co-stimulation and signaling domains serve to induce activity and proliferation of the cytotoxic lymphocytes, the fusion protein will generally display these two domains in the interior of the cell.

In one embodiment, a CAR molecule comprises:
i) a target antigen (e.g., CLDN6 or CLDN18.2) binding domain;
ii) a transmembrane domain; and
iii) an intracellular domain that comprises a 4-1BB costimulatory domain, and a CD3-zeta signaling domain.

In one embodiment, the antigen binding domain comprises an scFv. In one embodiment, the transmembrane domain comprises a transmembrane domain of a protein selected from the group consisting of the alpha, beta or zeta chain of the T cell receptor, CD28, CD3 epsilon, CD45, CD4, CD5, CD8, CD9, CD16, CD22, CD33, CD37, CD64, CD80, CD86, CD134, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. In one embodiment, the transmembrane domain comprises a CD8a transmembrane domain. In one embodiment, the antigen binding domain is connected to the transmembrane domain by a hinge domain. In one embodiment, the hinge domain is a CD8a hinge domain.

In one embodiment, the CAR molecule of the invention comprises:
i) a target antigen binding domain;
ii) a CD8α hinge domain;
iii) a CD8α transmembrane domain; and
iv) an intracellular domain that comprises a 4-1BB costimulatory domain, and a CD3-zeta signaling domain.

According to the disclosure, a CAR which when present on a T cell recognizes an antigen such as on the surface of antigen presenting cells or diseased cells such as cancer cells, such that the T cell is stimulated, and/or expanded or exerts effector functions as described above.

### Immune Effector Cell Activator Molecule

Immune effector cells or particles of the present invention comprise a second nucleic acid encoding for an immune effector activator molecule, also called “activator molecule” throughout this application.

In some embodiments, the activator molecule is transiently expressed.

Different kinds of molecules can be an activator molecule as long as they may allow for the activation, expansion, differentiation and/or proliferation of an immune effector cell, in particular a T cell.

In some embodiments the activator molecule is a cytokine.

Examples of cytokines include interferons, such as interferon-alpha (IFN-α) or interferon-gamma (IFN-γ), interleukins, such as IL-2, IL-7, IL-10, IL-12, IL-15 and IL-23, colony stimulating factors, such as M-CSF and GM-CSF, and tumor necrosis factor. According to another aspect, the immunostimulant includes an adjuvant-type immunostimulatory agent such as APC Toll-like Receptor agonists or costimulatory/cell adhesion membrane proteins. Examples of Toll-like Receptor agonists include costimulatory/adhesion proteins such as CD80, CD86, and ICAM-1.

The term “cytokines” relates to proteins which have a molecular weight of about 5 to 60 kDa and which participate in cell signaling (e.g., paracrine, endocrine, and/or autocrine signaling). In particular, when released, cytokines exert an effect on the behavior of cells around the place of their release. Examples of cytokines include lymphokines, interleukins, chemokines, interferons, and tumor necrosis factors (TNFs). According to the present disclosure, cytokines do not include hormones or growth factors. Cytokines differ from hormones in that (i) they usually act at much more variable concentrations than hormones and (ii) generally are made by a broad range of cells (nearly all nucleated cells can produce cytokines). Interferons are usually characterized by antiviral, antiproliferative and immunomodulatory activities. Interferons are proteins that alter and regulate the transcription of genes within a cell by binding to interferon receptors on the regulated cell's surface, thereby preventing viral replication within the cells. The interferons can be grouped into two types. Particular examples of cytokines include erythropoietin (EPO), colony stimulating factor (CSF), granulocyte colony stimulating factor (G-CSF), granulocyte-macrophage colony stimulating factor (GM-CSF), tumor necrosis factor (TNF), bone morphogenetic protein (BMP), interferon alfa (IFNα), interferon beta (IFNβ), interferon gamma (INFγ), interleukin 2 (IL-2), interleukin 4 (IL-4), interleukin 10 (IL-10), interleukin 11 (IL-11), interleukin 12 (IL-12), interleukin 15 (IL-15), and interleukin 21 (IL-21), as well as variants and derivatives thereof.

According to the disclosure, a cytokine may be a naturally occurring cytokine or a functional fragment or variant thereof. A cytokine may be human cytokine and may be derived from any vertebrate, especially any mammal.

In some embodiments, the activator molecule is a second cell-surface expressed antigen receptor as described herein, preferably a CAR or a TCR.

In some embodiments, the activator molecule is an antigen, in particular an antigen to which the first cell-surface expressed antigen receptor specifically binds. In some embodiments, the antigen is an antigen as described herein.

The term “antigen” may also be referred to as “antigen targeted by the antigen receptor”, “cognate antigen molecule” or simply “antigen molecule”.

In one embodiment, the cognate antigen molecule comprises the antigen expressed by a target cell to which the antigen receptors are targeted or a fragment thereof, or a variant of the antigen or the fragment.

In one embodiment, expression of the cognate antigen molecule is at the cell surface.

Binding of the antigen by the antigen receptor preferably results in stimulation, priming and/or expansion of immune effector cells. Said stimulated, primed and/or expanded immune effector cells are preferably directed against a target antigen, in particular a target antigen expressed by diseased cells, tissues and/or organs, i.e., a disease-associated antigen. Thus, an antigen may comprise the disease-associated antigen, or a fragment or variant thereof. In one embodiment, such fragment or variant is immunologically equivalent to the disease-associated antigen. In the context of the present disclosure, the term “fragment of an antigen” or “variant of an antigen” means an agent which results in stimulation, priming and/or expansion of immune effector cells which stimulated, primed and/or expanded immune effector cells target the antigen, i.e. a disease-associated antigen, in particular when presented by diseased cells, tissues and/or organs. Thus, the antigen may correspond to or may comprise the disease-associated antigen, may correspond to or may comprise a fragment of the disease-associated antigen or may correspond to or may comprise an antigen which is homologous to the disease-associated antigen or a fragment thereof. If the antigen comprises a fragment of the disease-associated antigen or an amino acid sequence which is homologous to a fragment of the disease-associated antigen said fragment or amino acid sequence may comprise an epitope of the disease-associated antigen to which the first and/or second antigen receptor of the immune effector cells is targeted or a sequence which is homologous to an epitope of the disease-associated antigen. Thus, according to the disclosure, an antigen may comprise an immunogenic fragment of a disease-associated antigen or an amino acid sequence being homologous to an immunogenic fragment of a disease-associated antigen. An “immunogenic fragment of an antigen” according to the disclosure preferably relates to a fragment of an antigen which is capable of stimulating, priming and/or expanding immune effector cells carrying an antigen receptor binding to the antigen or cells expressing the antigen. It is preferred that the antigen (similar to the disease-associated antigen) provides the relevant epitope for binding by the antigen binding domain present in the immune effector cells. In one embodiment, the antigen (similar to the disease-associated antigen) is expressed on the surface of the immune effector cell so as to provide the relevant epitope for binding by other immune effector cells. In one embodiment, the antigen (similar to the disease-associated antigen) is expressed by and presented on the surface of an immune effector cell in the context of MHC so as to provide the relevant epitope for binding by other immune effector cells. The antigen may be a recombinant antigen.

In one embodiment of all aspects of the invention, the second nucleic acid encoding the antigen is expressed in immune effector cells to provide the antigen or a procession product thereof for binding by the antigen receptor expressed by other immune effector cells, said binding resulting in stimulation, priming and/or expansion of the other immune effector cells.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization. For example, an amino acid sequence is immunologically equivalent to a reference amino acid sequence if said amino acid sequence when exposed to the immune system of a subject such as T cells binding to the reference amino acid sequence or cells expressing the reference amino acid sequence induces an immune reaction having a specificity of reacting with the reference amino acid sequence. Thus, a molecule which is immunologically equivalent to an antigen exhibits the same or essentially the same properties and/or exerts the same or essentially the same effects regarding the stimulation, priming and/or expansion of T cells as the antigen to which the T cells are targeted.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In the context of the present disclosure, the term is preferably used in the context of an immunological response in which lymphocytes are stimulated by an antigen, proliferate, and the specific lymphocyte recognizing said antigen is amplified. Preferably, clonal expansion leads to differentiation of the lymphocytes.

The term “antigen” relates to an agent comprising an epitope against which an immune response can be generated. The term “antigen” includes, in particular, proteins and peptides. In one embodiment, an antigen is presented or present on the surface of cells of the immune system such as antigen presenting cells like dendritic cells or macrophages. An antigen or a procession product thereof such as a T cell epitope is in one embodiment bound by an antigen receptor. Accordingly, an antigen or a procession product thereof may react specifically with immune effector cells such as T-lymphocytes (T cells). In one embodiment, an antigen is a disease-associated antigen, such as a tumor antigen, a viral antigen, or a bacterial antigen and an epitope is derived from such antigen.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. The disease-associated antigen or an epitope thereof may therefore be used for therapeutic purposes. Disease-associated antigens may be associated with infection by microbes, typically microbial antigens, or associated with cancer, typically tumors.

The term “tumor antigen” or “tumor-associated antigen” refers to a constituent of cancer cells which may be derived from the cytoplasm, the cell surface and the cell nucleus. In particular, it refers to those antigens which are produced intracellularly or as surface antigens on tumor cells. A tumor antigen is typically expressed preferentially by cancer cells (e.g., it is expressed at higher levels in cancer cells than in non-cancer cells) and in some instances it is expressed solely by cancer cells. Examples of tumor antigens include, without limitation, p53, ART-4, BAGE, beta-catenin/m, Bcr-abL CAMEL, CAP-1, CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A 10, MAGE-A 1 1, or MAGE-A12, MAGE-B, MAGE-C, MART-1/Melan-A, MC1R, Myosin/m, MUC1, MUM-1, MUM-2, MUM-3, NA88-A, NF1, NY-ESO-1, NY-BR-1, p190 minor BCR-abL, Pm1/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE, WT, and WT-1. Particularly, preferred tumor antigens are proteins of the claudin family, such as CLAUDIN-6 or CLAUDIN-18.2.

The term “viral antigen” refers to any viral component having antigenic properties, i.e. being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “expressed on the cell surface” or “associated with the cell surface” means that a molecule such as a receptor or antigen is associated with and located at the plasma membrane of a cell, wherein at least a part of the molecule faces the extracellular space of said cell and is accessible from the outside of said cell, e.g., by antibodies located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by e.g. antigen-specific antibodies added to the cells. In one embodiment, an antigen expressed on the surface of cells is an integral membrane protein having an extracellular portion recognized by a CAR.

The term “extracellular portion” or “exodomain” in the context of the present invention refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class 11 molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

In one embodiment, the target antigen of the first antigen receptor is a tumor antigen and the antigen as an activator molecule or a fragment thereof (e.g., an epitope) is derived from the tumor antigen. The tumor antigen may be a “standard” antigen, which is generally known to be expressed in various cancers. The tumor antigen may also be a “neo-antigen”, which is specific to an individual's tumor and has not been previously recognized by the immune system. A neo-antigen or neo-epitope may result from one or more cancer-specific mutations in the genome of cancer cells resulting in amino acid changes. If the tumor antigen is a neo-antigen, the vaccine antigen preferably comprises an epitope or a fragment of said neo-antigen comprising one or more amino acid changes.

Cancer mutations vary with each individual. Thus, cancer mutations that encode novel epitopes (neo-epitopes) represent attractive targets in the development of vaccine compositions and immunotherapies. The efficacy of tumor immunotherapy relies on the selection of cancer-specific antigens and epitopes capable of inducing a potent immune response within a host.

The peptide and protein antigen can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

In one embodiment of all aspects of the invention, an antigen is expressed in a diseased cell such as a cancer cell. In one embodiment, an antigen is expressed on the surface of a diseased cell such as a cancer cell. In one embodiment, an antigen receptor is a CAR which binds to an extracellular domain or to an epitope in an extracellular domain of an antigen. In one embodiment, a CAR binds to native epitopes of an antigen present on the surface of living cells. In one embodiment, binding of a CAR when expressed by T cells and/or present on T cells to an antigen present on diseased cells such as cancer cells results in cytolysis and/or apoptosis of the diseased cells, wherein said T cells preferably release cytotoxic factors, e.g. perforins and granzymes.

### Particle

The particles according to the present disclosure comprise at least a first and second nucleic acid molecule. In some embodiments, the particles may comprise further nucleic acids. In some embodiments, the particles comprise a third nucleic acid, preferably more than one third nucleic acid. In some embodiments, the particles contain a third and a fourth nucleic acid, preferably more than one third and/or fourth nucleic acid. In some embodiments, the particles contain a third and a fifth nucleic acid, preferably more than one third and/or fifth nucleic acid. In some embodiments, the particles contain a third, a fourth and a fifth nucleic acid, preferably more than one third, fourth and/or fifth nucleic acid.

To overcome the barriers to safe and effective nucleic acid delivery, nucleic acids may be administered with one or more delivery vehicles that protect the nucleic acids from degradation, maximize delivery to on-target cells and minimize exposure to off-target cells. Such nucleic acid delivery vehicles may complex or encapsulate nucleic acids and include a range of materials, including polymers and lipids. In some embodiments, such nucleic acid delivery vehicles may form particles with nucleic acids, preferably DNA and/or RNA.

DNA or RNA, in particular mRNA, described herein may be present in particles comprising (i) the DNA and/or RNA, and (ii) at least one cationic or cationically ionizable compound such as a polymer or lipid complexing the DNA and/or RNA. Electrostatic interactions between positively charged molecules such as polymers and lipids and negatively charged DNA and/or RNA are involved in particle formation. This results in complexation and spontaneous formation of nucleic acid, in particular DNA and/or RNA, particles.

Different types of nucleic acid containing particles have been described previously to be suitable for delivery of DNA and/or RNA in particulate form (cf., e.g., Kaczmarek, J. C. et al., 2017, Genome Medicine 9, 60). For non-viral DNA and/or RNA delivery vehicles, nanoparticle encapsulation of nucleic acids physically protects the nucleic acids from degradation and, depending on the specific chemistry, can aid in cellular uptake and endosomal escape.

In the context of the present disclosure, the term “particle” relates to a structured entity formed by molecules or molecule complexes, in particular particle forming compounds. In some embodiments, the particle contains an envelope (e.g., one or more layers or lamellas) made of one or more types of amphiphilic substances (e.g., amphiphilic lipids). In this context, the expression “amphiphilic substance” means that the substance possesses both hydrophilic and lipophilic properties. The envelope may also comprise additional substances (e.g., additional lipids) which do not have to be amphiphilic. Thus, the particle may be a monolamellar or multilamellar structure, wherein the substances constituting the one or more layers or lamellas comprise one or more types of amphiphilic substances (in particular selected from the group consisting of amphiphilic lipids) optionally in combination with additional substances (e.g., additional lipids) which do not have to be amphiphilic. In some embodiments, the term “particle” relates to a micro- or nano-sized structure, such as a micro- or nano-sized compact structure. According to the present disclosure, the term “particle” includes nanoparticles.

A “DNA particle”, “RNA particle” or “DNA and RNA particle” can be used to deliver DNA and/or RNA to a target site of interest (e.g., cell, tissue, organ, and the like). A DNA and/or RNA particle may be formed from lipids comprising at least one cationic or cationically ionizable lipid. Without intending to be bound by any theory, it is believed that the cationic or cationically ionizable lipid combines together with the nucleic acids to form aggregates, and this aggregation results in colloidally stable particles.

DNA and/or RNA particles described herein include lipid nanoparticle (LNP)-based and lipoplex (LPX)-based formulations.

A lipoplex (LPX) described herein is obtainable from mixing two aqueous phases, namely a phase comprising RNA and a phase comprising a dispersion of lipids. In some embodiments, the lipid phase comprises liposomes.

In some embodiments, liposomes are self-closed unilamellar or multilamellar vesicular particles wherein the lamellae comprise lipid bilayers and the encapsulated lumen comprises an aqueous phase. A prerequisite for using liposomes for nanoparticle formation is that the lipids in the mixture as required are able to form lamellar (bilayer) phases in the applied aqueous environment.

In some embodiments, liposomes comprise unilamellar or multilamellar phospholipid bilayers enclosing an aqueous core (also referred to herein as an aqueous lumen). They may be prepared from materials possessing polar head (hydrophilic) groups and nonpolar tail (hydrophobic) groups. In some embodiments, cationic lipids employed in formulating liposomes designed for the delivery of DNA and/or RNA are amphiphilic in nature and consist of a positively charged (cationic) amine head group linked to a hydrocarbon chain or cholesterol derivative via glycerol.

In some embodiments, lipoplexes are multilamellar liposome-based formulations that form upon electrostatic interaction of cationic liposomes with nucleic acids. In some embodiments, formed lipoplexes possess distinct internal arrangements of molecules that arise due to the transformation from liposomal structure into compact DNA and/or RNA-lipoplexes.

In some embodiments, an LPX particle comprises an amphiphilic lipid, in particular cationic or cationically ionizable amphiphilic lipid, and DNA and/or RNA (especially mRNA) as described herein. In some embodiments, electrostatic interactions between positively charged liposomes (made from one or more amphiphilic lipids, in particular cationic or cationically ionizable amphiphilic lipids) and negatively charged RNA (especially mRNA) results in complexation and spontaneous formation of RNA lipoplex particles. Positively charged liposomes may be generally synthesized using a cationic or cationically ionizable amphiphilic lipid, such as DOTMA and/or DODMA, and optionally additional lipids, such as DOPE or DSPC.

In general, a lipid nanoparticle (LNP) is typically obtainable from direct mixing of DNA and/or RNA in an aqueous phase with lipids in a phase comprising an organic solvent, such as ethanol. In that case, lipids or lipid mixtures can be used for particle formation, which do not form lamellar (bilayer) phases in water.

In some embodiments, LNPs comprise or consist of a cationic/cationically ionizable lipid and helper lipids such as phospholipids, cholesterol, and/or polymer-conjugated lipids (e.g., polyethylene glycol (PEG) lipids). In some embodiments, in the DNA and/or RNA LNPs described herein the DNA and/or RNA (in particular, mRNA) is bound by cationically ionizable lipid that occupies the central core of the LNP. In some embodiments, polymer-conjugated lipid forms the surface of the LNP, along with phospholipids. In some embodiments, cholesterol and cationically ionizable lipid in charged and uncharged forms can be distributed throughout the LNP.

In some embodiments, DNA and/or RNA (e.g., mRNA) described herein may be noncovalently associated with a particle as described herein. In embodiments, the DNA and/or RNA (especially mRNA) may be adhered to the outer surface of the particle (surface RNA (especially surface mRNA)) and/or may be contained in the particle (encapsulated DNA and/or RNA (especially encapsulated mRNA)).

In some embodiments, the particles (e.g., LNPs and LPXs) described herein have a size (such as a diameter) in the range of about 10 to about 2000 nm, such as at least about 15 nm (e.g., at least about 20 nm, at least about 25 nm, at least about 30 nm, at least about 35 nm, at least about 40 nm, at least about 45 nm, at least about 50 nm, at least about 55 nm, at least about 60 nm, at least about 65 nm, at least about 70 nm, at least about 75 nm, at least about 80 nm, at least about 85 nm, at least about 90 nm, at least about 95 nm, or at least about 100 nm) and/or at most about 1900 nm (e.g., at most about 1800 nm, at most about 1700 nm, at most about 1600 nm, at most about 1500 nm, at most about 1400 nm, at most about 1300 n, at most about 1200 nm, at most about 1100 nm, at most about 1000 nm, at most about 950 nm, at most about 900 nm, at most about 850 nm, at most about 800 nm, at most about 750 nm, at most about 700 nm, at most about 650 nm, at most about 600 nm, at most about 550 nm, or at most about 500 nm), such as in the range of about 20 to about 1500 nm, such as about 30 to about 1200 nm, about 40 to about 1100 num, about 50 to about 1000 nm, about 60 to about 900 nm, about 70 to about 800 nm, about 80 to about 700 nm, about 90 to about 600 nm, or about 50 to about 500 nm or about 100 to about 500 nm, such as in the range of 10 to 1000 nm, 15 to 500 nm, 20 to 450 nm, 25 to 400 nm, 30 to 350 nm, 40 to 300 nm, 50 to 250 nm, 60 to 200 nm, 70 to 150 nm, or 80 to 150 nm. In some embodiments, the particles (e.g., LNPs and LPXs) described herein have a size (such as a diameter) in the range of from about 40 nm to about 200 nm, such as from about 50 nm to about 180 nm, from about 60 nm to about 160 nm, from about 80 nm to about 150 nm or from about 80 nm to about 120 nm.

In some embodiments, the particles (e.g., LNPs and LPXs) described herein have an average diameter that in some embodiments ranges from about 50 nm to about 1000 nm, from about 50 nm to about 800 nm, from about 50 nm to about 700 nm, from about 50 nm to about 600 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 100 nm to about 1000 nm, from about 100 nm to about 800 nm, from about 100 nm to about 700 nm, from about 100 nm to about 600 nm, from about 100 nm to about 500 nm, from about 100 nm to about 450 nm, from about 100 nm to about 400 nm, from about 100 nm to about 350 nm, from about 100 nm to about 300 nm, from about 100 nm to about 250 nm, from about 100 nm to about 200 nm, from about 150 nm to about 1000 nm, from about 150 nm to about 800 nm, from about 150 nm to about 700 nm, from about 150 nm to about 600 nm, from about 150 nm to about 500 nm, from about 150 nm to about 450 nm, from about 150 nm to about 400 nm, from about 150 nm to about 350 nm, from about 150 nm to about 300 nm, from about 150 nm to about 250 nm, from about 150 nm to about 200 nm, from about 200 nm to about 1000 nm, from about 200 nm to about 800 nm, from about 200 nm to about 700 nm, from about 200 nm to about 600 nm, from about 200 nm to about 500 nm, from about 200 nm to about 450 nm, from about 200 nm to about 400 nm, from about 200 nm to about 350 nm, from about 200 nm to about 300 nm, from about 200 nm to about 250 nm, or from about 80 to about 150 nm. In some embodiments, the particles (e.g., LNPs and LPXs) described herein have an average diameter that in some embodiments ranges from about 40 nm to about 200 nm, such as from about 50 nm to about 180 nm, from about 60 nm to about 160 nm, from about 80 nm to about 150 nm or from about 80 nm to about 120 nm.

DNA and/or RNA particles (especially mRNA particles) described herein may exhibit a polydispersity index (PDI) less than about 0.5, less than about 0.4, less than about 0.3, less than about 0.2, less than about 0.1, or less than about 0.05. By way of example, the DNA and/or RNA particles can exhibit a polydispersity index in a range of about 0.01 to about 0.4 or about 0.1 to about 0.3.

The N/P ratio gives the ratio of the nitrogen groups in the lipid to the number of phosphate groups in the nucleic acid. It is correlated to the charge ratio, as the nitrogen atoms (depending on the pH) are usually positively charged and the phosphate groups are negatively charged. The N/P ratio, where a charge equilibrium exists, depends on the pH. Lipid formulations may be formed at N/P ratios larger than four up to twelve, because positively charged nanoparticles can be favorable for transfection. In that case, DNA and/or RNA is considered to be completely bound to nanoparticles.

The present disclosure describes compositions comprising DNA and/or RNA (especially mRNA) and at least one cationic or cationically ionizable lipid which associates with the DNA and/or RNA to form DNA and/or RNA particles and formulations comprising such particles. The DNA and/or RNA particles may comprise DNA and/or RNA which is complexed in different forms by non-covalent interactions to the particle. The particles described herein are not viral particles, in particular infectious viral particles, i.e., they are not able to virally infect cells.

Suitable cationic or cationically ionizable lipids are those that form DNA and/or RNA particles and are included by the term “particle forming components” or “particle forming agents”. The term “particle forming components” or “particle forming agents” relates to any components which associate with DNA and/or RNA to form DNA and/or RNA particles. Such components include any component which can be part of DNA and/or RNA particles.

In some embodiments, DNA and/or RNA particles (especially mRNA particles) comprise more than one type of DNA and/or RNA molecules, where the molecular parameters of the DNA and/or RNA molecules may be similar or different from each other, like with respect to molar mass or fundamental structural elements such as molecular architecture, capping, coding regions or other features,

In particulate formulation, it is possible that each DNA and/or RNA species is separately formulated as an individual particulate formulation. In that case, each individual particulate formulation will comprise one DNA and/or RNA species. The individual particulate formulations may be present as separate entities, e.g. in separate containers. Such formulations are obtainable by providing each DNA and/or RNA species separately (typically each in the form of an DNA and/or RNA-containing solution) together with a particle-forming agent, thereby allowing the formation of particles. Respective particles will contain exclusively the specific DNA and/or RNA species that is being provided when the particles are formed (individual particulate formulations). In some embodiments, a composition such as a pharmaceutical composition comprises more than one individual particle formulation. Respective pharmaceutical compositions are referred to as mixed particulate formulations. Mixed particulate formulations according to the present disclosure are obtainable by forming, separately, individual particulate formulations, followed by a step of mixing of the individual particulate formulations. By the step of mixing, a formulation comprising a mixed population of DNA and/or RNA-containing particles is obtainable. Individual particulate populations may be together in one container, comprising a mixed population of individual particulate formulations. Alternatively, it is possible that all DNA and/or RNA species of the pharmaceutical composition are formulated together as a combined particulate formulation. Such formulations are obtainable by providing a combined formulation (typically combined solution) of all DNA and/or RNA species together with a particle-forming agent, thereby allowing the formation of particles. As opposed to a mixed particulate formulation, a combined particulate formulation will typically comprise particles which comprise more than one DNA and/or RNA species. In a combined particulate composition different DNA and/or RNA species are typically present together in a single particle.

### Polymers

Given their high degree of chemical flexibility, polymers are commonly used materials for nanoparticle-based delivery. Typically, cationic polymers are used to electrostatically condense the negatively charged DNA and/or RNA into particles, in particular nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly(β-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneimine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In one embodiment, the polyalkyleneimine comprises polyethylenimine and/or polypropylenimine, preferably polyethyleneimine. A preferred polyalkyleneimine is polyethyleneimine (PEI). The average molecular weight of PEI is preferably 0.75-102 to 107 Da, preferably 1000 to 105 Da, more preferably 10000 to 40000 Da, more preferably 15000 to 30000 Da, even more preferably 20000 to 25000 Da.

Preferred according to the disclosure is linear polyalkyleneimine such as linear polyethyleneimine (PEI).

Cationic polymers (including polycationic polymers) contemplated for use herein include any cationic polymers which are able to electrostatically bind nucleic acid. In one embodiment, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

### Lipids

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually insoluble or poorly soluble in water, but soluble in many organic solvents. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “hydrophobic” refers to any a molecule, moiety or group which is substantially immiscible or insoluble in aqueous solution. The term hydrophobic group includes hydrocarbons having at least 6 carbon atoms. The monovalent radical of a hydrocarbon is referred to as hydrocarbyl herein. The hydrophobic group can have functional groups (e.g., ether, ester, halide, etc.) and atoms other than carbon and hydrogen as long as the group satisfies the condition of being substantially immiscible or insoluble in aqueous solution.

The term “hydrocarbon” includes non-cyclic, e.g., linear (straight) or branched, hydrocarbyl groups, such as alkyl, alkenyl, or alkynyl as defined herein. It should be appreciated that one or more of the hydrogen atoms in alkyl, alkenyl, or alkynyl may be substituted with other atoms, e.g., halogen, oxygen or sulfur. Unless stated otherwise, hydrocarbon groups can also include a cyclic (alkyl, alkenyl or alkynyl) group or an aryl group, provided that the overall polarity of the hydrocarbon remains relatively nonpolar.

The term “alkyl” refers to a saturated linear or branched monovalent hydrocarbon moiety which may have one to thirty, typically one to twenty, often six to eighteen carbon atoms. Exemplary nonpolar alkyl groups include, but are not limited to, methyl, ethyl, propyl, isopropyl, hexyl, decyl, dodecyl, tetradecyl, hexadecyl, octadecyl, and the like.

The term “alkenyl” refers to a linear or branched monovalent hydrocarbon moiety having at least one carbon-carbon double bond in which the total carbon atoms may be six to thirty, typically six to twenty often six to eighteen. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 6 (such as 1 to 4), i.e., 1, 2, 3, 4, 5, or 6, carbon-carbon double bonds.

The term “alkynyl” refers to a linear or branched monovalent hydrocarbon moiety having at least one carbon-carbon triple bond in which the total carbon atoms may be six to thirty, typically six to twenty, often six to eighteen. Alkynyl groups can optionally have one or more carbon-carbon double bonds. Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 6 (such as 1 to 4), i.e., 1, 2, 3, 4, 5, or 6, more preferably 1 or 2 carbon-carbon triple bonds.

The term “alkylene” refers to a saturated linear or branched divalent hydrocarbon moiety which may have one to thirty, typically two to twenty, often four to twelve carbon atoms. Exemplary nonpolar alkylene groups include, but are not limited to, methylene, ethylene, trimethylene, hexamethylene, decamethylene, dodecamethylene, tetradecamethylene, hexadecamethylene, octadecmethylene, and the like.

The term “alkenylene” refers to a linear or branched divalent hydrocarbon moiety having at least one carbon-carbon double bond in which the total carbon atoms may be two to thirty, typically two to twenty, often four to twelve. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 6 (such as 1 to 4), i.e., 1, 2, 3, 4, 5, or 6, carbon-carbon double bonds.

The term “cycloalkyl” represents cyclic non-aromatic versions of “alkyl” and “alkenyl” with preferably 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkyl groups include cyclopropyl, cyclopropenyl, cyclobutyl, cyclobutenyl, cyclopentyl, cyclopentenyl, cyclohexyl, cyclohexenyl, cycloheptyl, cycloheptenyl, cyclooctyl, cyclooctenyl, cyclononyl, cyclononenyl, cylcodecyl, cylcodecenyl, and adamantyl. The cycloalkyl group may consist of one ring (monocyclic), two rings (bicyclic), or more than two rings (polycyclic).

The term “aryl” refers to a monoradical of an aromatic cyclic hydrocarbon. Preferably, the aryl group contains 3 to 14 (e.g., 5, 6, 7, 8, 9, or 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. Aryl does not encompass fullerenes.

The term “aromatic” as used in the context of hydrocarbons means that the whole molecule has to be aromatic. For example, if a monocyclic aryl is hydrogenated (either partially or completely) the resulting hydrogenated cyclic structure is classified as cycloalkyl for the purposes of the present disclosure. Likewise, if a bi- or polycyclic aryl (such as naphthyl) is hydrogenated the resulting hydrogenated bi- or polycyclic structure (such as 1,2-dihydronaphthyl) is classified as cycloalkyl for the purposes of the present disclosure (even if one ring, such as in 1,2-dihydronaphthyl, is still aromatic).

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances, in particular amphiphilic substances, that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term includes molecules, which comprise hydrophilic and hydrophobic moieties with different structural organization, which may or may not be similar to that of lipids. Examples of lipid-like compounds capable of spontaneous integration into cell membranes include functional lipid constructs such as synthetic function-spacer-lipid constructs (FSL), synthetic function-spacer-sterol constructs (FSS) as well as artificial amphipathic molecules. Lipids comprising two long alkyl chains and a polar head group are generally cylindrical. The area occupied by the two alkyl chains is similar to the area occupied by the polar head group. Such lipids have low solubility as monomers and tend to aggregate into planar bilayers that are water insoluble. Traditional surfactant monomers comprising only one linear alkyl chain and a hydrophilic head group are generally cone shaped. The hydrophilic head group tends to occupy more molecular space than the linear alkyl chain. In some embodiments, surfactants tend to aggregate into spherical or elliptoid micelles that are water soluble. While lipids also have the same general structure as surfactants—a polar hydrophilic head group and a nonpolar hydrophobic tail—lipids differ from surfactants in the shape of the monomers, in the type of aggregates formed in solution, and in the concentration range required for aggregation. As used herein, the term “lipid” is to be construed to cover both lipids and lipid-like materials unless otherwise indicated herein or clearly contradicted by context.

Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as steroids, i.e., sterol-containing metabolites such as cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more cis double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

### Cationic/Cationically Ionizable Lipids

In some embodiments, the DNA and/or RNA compositions and formulations and nucleic acid particles described herein comprise at least one cationic or cationically ionizable lipid as particle forming agent. Cationic or cationically ionizable lipids contemplated for use herein include any cationic or cationically ionizable lipids (including lipid-like materials) which are able to electrostatically bind nucleic acid. In some embodiments, cationic or cationically ionizable lipids contemplated for use herein can be associated with nucleic acid, e.g. by forming complexes with the nucleic acid or forming vesicles in which the nucleic acid is enclosed or encapsulated.

As used herein, a “cationic lipid” refers to a lipid or lipid-like material having a net positive charge. Cationic lipids bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In some embodiments, a cationic lipid has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

As used herein, a “cationically ionizable lipid” refers to a lipid or lipid-like material which has a net positive charge or is neutral, i.e., which is not permanently cationic. Thus, depending on the pH of the composition in which the cationically ionizable lipid is solved, the cationically ionizable lipid is either positively charged or neutral. For purposes of the present disclosure, cationically ionizable lipids are covered by the term “cationic lipid” unless contradicted by the circumstances.

In some embodiments, the cationic or cationically ionizable lipid comprises a head group which includes at least one nitrogen atom (N) which is positive charged or capable of being protonated, e.g., under physiological conditions.

Examples of cationic or cationically ionizable lipids include, but are not limited to N,N-dimethyl-2,3-dioleyloxypropylamine (DODMA), 1,2-dioleoyl-3-trimethylammonium propane (DOTAP); 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (PAE-DMRIE), N-(4-carboxybenzyl)-N,N-dimethyl-2,3-bis(oleoyloxy)propan-1-aminium (DOBAQ), 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In some embodiments, the cationic or cationically ionizable lipid is DOTMA. In some embodiments, the cationic or cationically ionizable lipid is DODMA.

DOTMA is a cationic lipid with a quaternary amine headgroup. The structure of DOTMA may be represented as follows:

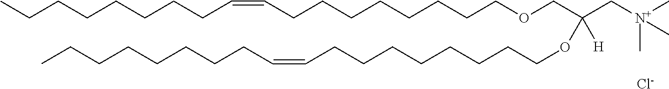 [SMILES: CCCCCCCC/C=C\CCCCCCCCOCC(C[N+](C)(C)C)OCCCCCCCC/C=C\CCCCCCCC ; [Cl-]]

DODMA is an ionizable cationic lipid with a tertiary amine headgroup. The structure of DODMA may be represented as follows:

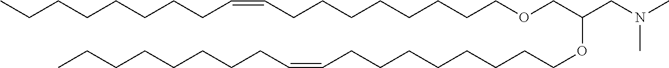 [SMILES: CCCCCCCC/C=C\CCCCCCCCOCC(CN(C)C)OCCCCCCCC/C=C\CCCCCCCC]

In some embodiments, the cationic or cationically ionizable lipid may comprise from about 10 mol % to about 95 mol %, from about 20 mol % to about 95 mol %, from about 20 mol % to about 90 mol %, from about 30 mol % to about 90 mol %, from about 40 mol % to about 90 mol %, or from about 40 mol % to about 80 mol % of the total lipid present in the particle.

### Additional Lipids

The DNA and/or RNA compositions and formulations and DNA and/or RNA particles described herein may also comprise lipids (including lipid-like materials) other than cationic or cationically ionizable lipids (also collectively referred to herein as cationic lipids), i.e., non-cationic lipids (including non-cationic or non-cationically ionizable lipids or lipid-like materials). Collectively, anionic and neutral lipids or lipid-like materials are referred to herein as non-cationic lipids. Optimizing the formulation of DNA and/or RNA particles by addition of other hydrophobic moieties, such as cholesterol and lipids, in addition to a cationic or cationically ionizable lipid may enhance particle stability and efficacy of nucleic acid delivery.

One or more additional lipids may or may not affect the overall charge of the DNA and/or RNA particles. In some embodiments, the or more additional lipids are a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH.

In some embodiments, the DNA and/or RNA compositions and formulations and DNA and/or RNA particles described herein comprise a cationic or cationically ionizable lipid and one or more additional lipids.

Without wishing to be bound by theory, the amount of the cationic or cationically ionizable lipid compared to the amount of the one or more additional lipids may affect important DNA and/or RNA particle characteristics, such as charge, particle size, stability, tissue selectivity, and bioactivity of the DNA and/or RNA. Accordingly, in some embodiments, the molar ratio of the cationic or cationically ionizable lipid to the one or more additional lipids is from about 10:0 to about 1:9, about 4:1 to about 1:2, about 4:1 to about 1:1, about 3:1 to about 1:1, or about 3:1 to about 2:1.

In some embodiments, the one or more additional lipids comprised in the DNA and/or RNA compositions and formulations and DNA and/or RNA particles described herein comprise one or more of the following: neutral lipids, steroids, and combinations thereof.

In some embodiments, the one or more additional lipids comprise a neutral lipid which is a phospholipid. In some embodiments, the phospholipid is selected from the group consisting of phosphatidylcholines, phosphatidylethanolamines, phosphatidylglycerols, phosphatidic acids, phosphatidylserines and sphingomyelins. Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), 1,2-di-(9Z-octadecenoyl)-sn-glycero-3-phosphocholine (DOPG), 1,2-dipalmitoyl-sn-glycero-3-phospho-(1′-rac-glycerol) (DPPG), 1-palmitoyl-2-oleoyl-sn-glycero-3-phosphoethanolamine (POPE), N-palmitoyl-D-erythro-sphingosylphosphorylcholine (SM), and further phosphatidylethanolamine lipids with different hydrophobic chains. In some embodiments, the neutral lipid is selected from the group consisting of DSPC, DOPC, DMPC, DPPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC. In some embodiments, the neutral lipid is DOPE.

In some embodiments, the additional lipid comprises one of the following: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Thus, in some embodiments, the DNA and/or RNA compositions and formulations and DNA and/or RNA particles described herein comprise (1) a cationic or cationically ionizable lipid, and a phospholipid such as DSPC or DOPE or (2) a cationic or cationically ionizable lipid and a phospholipid such as DSPC or DOPE and cholesterol.

In some embodiments, the DNA and/or RNA particles (especially the particles comprising mRNA) described herein comprise (1) DOTMA and DOPE, (2) DOTMA, DOPE and cholesterol, (3) DODMA and DOPE or (4) DODMA, DOPE and cholesterol.

DSPC is a neutral phospholipid. The structure of DSPC may be represented as follows:

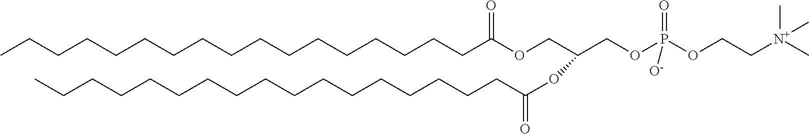 [SMILES: CCCCCCCCCCCCCCCCCC(=O)OC[C@H](COP(=O)([O-])OCC[N+](C)(C)C)OC(=O)CCCCCCCCCCCCCCCCC]

DOPE is a neutral phospholipid. The structure of DOPE may be represented as follows:

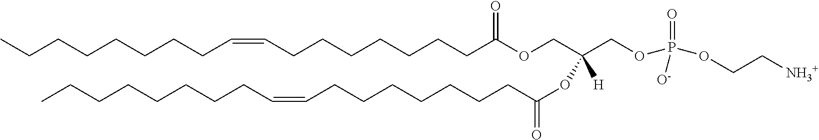 [SMILES: CCCCCCCC/C=C\CCCCCCCC(=O)OC[C@H](COP(=O)([O-])OCC[NH3+])OC(=O)CCCCCCC/C=C\CCCCCCCC]

The structure of cholesterol may be represented as follows:

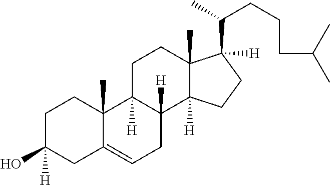

In some embodiments, DNA and/or RNA compositions and formulations and DNA and/or RNA particles described herein do not include a polymer conjugated lipid such as a pegylated lipid. The term “pegylated lipid” refers to a molecule comprising both a lipid portion and a polyethylene glycol portion. Pegylated lipids are known in the art.

In some embodiments, the additional lipid (e.g., one or more phospholipids and/or cholesterol) may comprise from about 0 mol % to about 90 mol %, from about 0 mol % to about 80 mol %, from about 2 mol % to about 80 mol %, from about 5 mol % to about 80 mol %, from about 5 mol % to about 60 mol %, from about 5 mol % to about 50 mol %, from about 7.5 mol % to about 50 mol %, or from about 10 mol % to about 40 mol % of the total lipid present in the particle. In some embodiments, the additional lipid (e.g., one or more phospholipids and/or cholesterol) comprises about 10 mol %, about 15 mol %, or about 20 mol % of the total lipid present in the particle.

In some embodiments, the additional lipid comprises a mixture of: (i) a phospholipid such as DOPE; and (ii) cholesterol or a derivative thereof. In some embodiments, the molar ratio of the phospholipid such as DOPE to the cholesterol or a derivative thereof is from about 9:0 to about 1:10, about 2:1 to about 1:4, about 1:1 to about 1:4, or about 1:1 to about 1:3.

### Polymer-Conjugated Lipids

In some embodiments, DNA and/or RNA compositions and formulations and DNA and/or RNA particles described herein may comprise at least one polymer-conjugated lipid. A polymer-conjugated lipid is typically a molecule comprising a lipid portion and a polymer portion conjugated thereto. In some embodiments, a polymer-conjugated lipid is a PEG-conjugated lipid, also referred to herein as pegylated lipid or PEG-lipid. The term “pegylated lipid” refers to a molecule comprising both a lipid portion and a polyethylene glycol portion. Pegylated lipids are known in the art. In some embodiments, a polymer-conjugated lipid is a polysarcosine-conjugated lipid, also referred to herein as sarcosinylated lipid or pSar-lipid. The term “sarcosinylated lipid” refers to a molecule comprising both a lipid portion and a polysarcosine portion.

In some embodiments, a polymer-conjugated lipid is designed to sterically stabilize a lipid particle by forming a protective hydrophilic layer that shields the hydrophobic lipid layer. In some embodiments, a polymer-conjugated lipid can reduce its association with serum proteins and/or the resulting uptake by the reticuloendothelial system when such lipid particles are administered in vivo.

### Polyethyleneglycol (PEG)-Conjugated Lipids

In some embodiments, DNA and/or RNA compositions/formulations and DNA and/or RNA particles described herein comprise a PEG-conjugated lipid.

In some embodiments, the PEG-conjugated lipid (pegylated lipid) is a lipid having the structure of the following general formula:

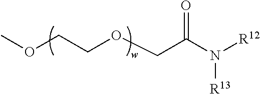

or a pharmaceutically acceptable salt, tautomer or stereoisomer thereof, wherein:
each of R12 and R13 is each independently a straight or branched, alkyl or alkenyl chain containing from 10 to 30 carbon atoms, wherein the alkyl/alkenyl chain is optionally interrupted by one or more ester bonds; and w has a mean value ranging from 30 to 60.

In some embodiments of this formula, each of R12 and R13 is independently a straight alkyl chain containing from 10 to 18 carbon atoms, preferably from 12 to 16 carbon atoms.

In some embodiments of this formula, R12 and R13 are identical. In some embodiments, each of R12 and R13 is a straight alkyl chain containing 12 carbon atoms. In some embodiments, each of R12 and R13 is a straight alkyl chain containing 14 carbon atoms. In some embodiments, each of R12 and R13 is a straight alkyl chain containing 16 carbon atoms.

In some embodiments of this formula, R12 and R13 are different. In some embodiments, one of R12 and R13 is a straight alkyl chain containing 12 carbon atoms and the other of R12 and R13 is a straight alkyl chain containing 14 carbon atoms.

In some embodiments of this formula, w has a mean value ranging from 40 to 50, such as a mean value of 45.

In some embodiments of this formula, w is within a range such that the PEG portion of the pegylated lipid has an average molecular weight of from about 400 to about 6000 g/mol, such as from about 1000 to about 5000 g/mol, from about 1500 to about 4000 g/mol, or from about 2000 to about 3000 g/mol. In some embodiments, each of R12 and R13 is a straight alkyl chain containing 14 carbon atoms and w has a mean value of 45.

Various PEG-conjugated lipids are known in the art and include, but are not limited to pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω methoxy(polyethoxy)ethyl)carbamate, and the like.

In some embodiments, the PEG-conjugated lipid (pegylated lipid) is or comprises 2-[(polyethylene glycol)-2000]-N,N-ditetradecylacetamide. In some embodiments, the pegylated lipid has the following structure:

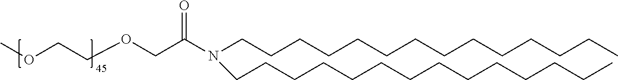

In some embodiments, the PEG-conjugated lipid (pegylated lipid) is DMG-PEG 2000, e.g., having the following structure:

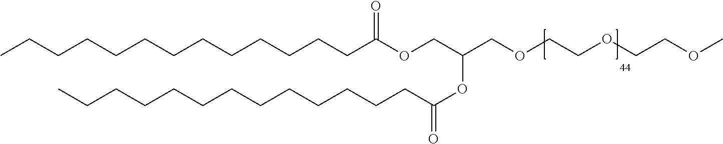

In some embodiments, the PEG-conjugated lipid (pegylated lipid) has the following structure:

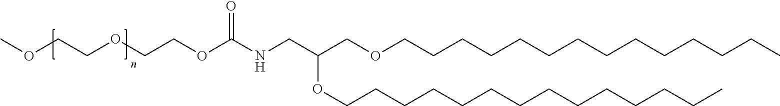

wherein n has a mean value ranging from 30 to 60, such as about 50. In one embodiment, the PEG-conjugated lipid (pegylated lipid) is PEG2000-C-DMA which preferably refers to 3-N-[(ω-methoxy poly(ethylene glycol)2000)carbamoyl]-1,2-dimyristyloxy-propylamine (MPEG-(2 kDa)-C-DMA) or methoxy-polyethylene glycol-2,3-bis(tetradecyloxy)propylcarbamate (2000).

In some embodiments, DNA and/or RNA compositions/formulations described herein may comprise one or more PEG-conjugated lipids or pegylated lipids as described in WO 2017/075531 and WO 2018/081480, the entire contents of each of which are incorporated herein by reference for the purposes described herein.

In some embodiments, the pegylated lipid comprises from about 1 mol % to about 10 mol %, preferably from about 1 mol % to about 5 mol %, more preferably from about 1 mol % to about 2.5 mol % of the total lipid present in the DNA and/or RNA compositions/formulations and DNA and/or RNA particles described herein.

### Embodiments of Lipoplex Particles

In some embodiments of the present disclosure, the DNA and/or RNA described herein may be present in DNA and/or RNA lipoplex particles.

Lipoplexes (LPX) are electrostatic complexes which are generally formed by mixing preformed cationic lipid liposomes with anionic nucleic acids. Formed lipoplexes possess distinct internal arrangements of molecules that arise due to the transformation from liposomal structure into compact DNA and/or RNA-lipoplexes.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

DNA and/or RNA lipoplex particles described herein have an average diameter that in some embodiments ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the DNA and/or RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 run. In some embodiments, the DNA and/or RNA lipoplex particles have an average diameter that ranges from about 250 nm to about 700 n. In some embodiments, the DNA and/or RNA lipoplex particles have an average diameter that ranges from about 300 nm to about 500 nm. In an exemplary embodiment, the DNA and/or RNA lipoplex particles have an average diameter of about 400 nm.

The DNA and/or RNA lipoplex particles and compositions comprising DNA and/or RNA lipoplex particles described herein are useful for delivery of DNA and/or RNA to a target tissue after parenteral administration, in particular after intravenous administration.

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the DNA and/or RNA lipoplex particles, DNA and/or RNA accumulation and/or DNA and/or RNA expression in the spleen occurs. Thus, DNA and/or RNA lipoplex particles of the disclosure may be used for expressing DNA and/or RNA in the spleen. In an embodiment, after administration of the DNA and/or RNA lipoplex particles, no or essentially no DNA and/or RNA accumulation and/or DNA and/or RNA expression in the lung and/or liver occurs. In some embodiments, after administration of the DNA and/or RNA lipoplex particles, DNA and/or RNA accumulation and/or DNA and/or RNA expression in antigen presenting cells, such as professional antigen presenting cells in the spleen occurs. Thus, DNA and/or RNA lipoplex particles of the disclosure may be used for targeting DNA and/or RNA, e.g., DNA and/or RNA encoding an antigen or at least one epitope, to the lymphatic system, in particular secondary lymphoid organs, more specifically spleen. Targeting the lymphatic system, in particular secondary lymphoid organs, more specifically spleen is in particular preferred if the DNA and/or RNA administered is DNA and/or RNA encoding vaccine antigen. In some embodiments, the target cell is a spleen cell. In some embodiments, the target cell is an antigen presenting cell such as a professional antigen presenting cell in the spleen. In some embodiments, the target cell is a dendritic cell in the spleen.

The electric charge of the DNA and/or RNA lipoplex particles of the present disclosure is the sum of the electric charges present in the at least one cationic lipid and the electric charges present in the DNA and/or RNA. The charge ratio is the ratio of the positive charges present in the at least one cationic lipid to the negative charges present in the DNA and/or RNA. The charge ratio of the positive charges present in the at least one cationic lipid to the negative charges present in the DNA and/or RNA is calculated by the following equation: charge ratio=[(cationic lipid concentration (mol))*(the total number of positive charges in the cationic lipid)]/[(DNA and/or RNA concentration (mol))*(the total number of negative charges in DNA and/or RNA)]. The concentration of DNA and/or RNA and the at least one cationic lipid amount can be determined using routine methods by one skilled in the art.

In some embodiments, at physiological pH the charge ratio of positive charges to negative charges in the DNA and/or RNA lipoplex particles is from about 1.6:2 to about 1:2, or about 1.6:2 to about 1.1:2. In specific embodiments, the charge ratio of positive charges to negative charges in the DNA and/or RNA lipoplex particles at physiological pH is about 1.6:2.0, about 1.5:2.0, about 1.4:2.0, about 1.3:2.0, about 1.2:2.0, about 1.1:2.0, or about 1:2.0.

### Embodiments of Lipid Nanoparticles (LNPs)

In some embodiments, DNA and/or RNA described herein is present in the form of lipid nanoparticles (LNPs). LNPs typically comprise four components: cationically ionizable lipid, neutral lipids such as phospholipids, a steroid such as cholesterol, and a polymer-conjugated lipid such as PEG-lipid. LNPs may be prepared by mixing lipids dissolved in ethanol with DNA and/or RNA in an aqueous buffer.

In some embodiments, in the DNA and/or RNA LNPs described herein the DNA and/or RNA is bound by cationically ionizable lipid that occupies the central core of the LNP. Polymer-conjugated lipid forms the surface of the LNP, along with phospholipids. In some embodiments, cholesterol and cationically ionizable lipid can be distributed throughout the LNP.

In some embodiments, the LNP comprises one or more cationically ionizable lipids, and one or more stabilizing lipids. Stabilizing lipids include neutral lipids and polymer-conjugated lipids.

In some embodiments, the LNP comprises a cationically ionizable lipid, a neutral lipid, a steroid, a polymer-conjugated lipid; and the DNA and/or RNA, encapsulated within or associated with the lipid nanoparticle.

In some embodiments, the LNP comprises from 35 to 65 mol percent, 40 to 60 mol percent, 40 to 55 mol percent, from 45 to 55 mol percent, or from 45 to 50 mol percent of the cationically ionizable lipid.

In some embodiments, the neutral lipid is present in a concentration ranging from 5 to 15 mol percent, from 7 to 13 mol percent, or from 9 to 11 mol percent.

In some embodiments, the steroid is present in a concentration ranging from 30 to 50 mol percent, from 30 to 45 mol percent, from 35 to 45 mol percent or from 35 to 43 mol percent.

In some embodiments, the LNP comprises from 1 to 10 mol percent, from 1 to 5 mol percent, or from 1 to 2.5 mol percent of the polymer-conjugated lipid.

In some embodiments, the LNP comprises from 45 to 55 mol percent of a cationically ionizable lipid; from 5 to 15 mol percent of a neutral lipid; from 30 to 45 mol percent of a steroid; from 1 to 5 mol percent of a polymer-conjugated lipid; and the DNA and/or RNA, encapsulated within or associated with the lipid nanoparticle.

In some embodiments, the mol percent is determined based on total mol of lipid present in the lipid nanoparticle. In some embodiments, the mol percent is determined based on total mol of cationically ionizable lipid, neutral lipid, steroid and polymer-conjugated lipid present in the lipid nanoparticle.

In some embodiments, the neutral lipid is selected from the group consisting of DSPC, DPPC, DMPC, DOPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

In some embodiments, the steroid is cholesterol.

In some embodiments, the polymer conjugated lipid is a pegylated lipid, e.g., a pegylated lipid as described above.

In some embodiments, the cationically ionizable lipid component of the LNPs has the structure of Formula (III):

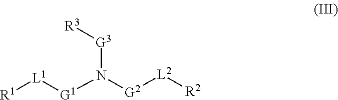

or a pharmaceutically acceptable salt, tautomer, prodrug or stereoisomer thereof, wherein:
one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, NRaC(═O)NRa—, —OC(═O)NRa- or —NRaC(═O)O—, and the other of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, NRaC(═O)NRa—, —OC(═O)NRa- or —NRaC(═O)O— or a direct bond;
G1 and G2 are each independently unsubstituted C1-C12 alkylene or C1-C12 alkenylene;
G3 is C1-C24 alkylene, C1-C24 alkenylene, C3-C8 cycloalkylene, C3-C8 cycloalkenylene;
Ra is H or C1-C12 alkyl;
R1 and R2 are each independently C6-C24 alkyl or C6-C24 alkenyl;
R3 is H, OR5, CN, —C(═O)OR4, —OC(═O)R4 or —NR5C(═O)R4;
R4 is C1-C12 alkyl;
R5 is H or C1-C6 alkyl; and
x is 0, 1 or 2.

In some of the foregoing embodiments of Formula (III), the lipid has one of the following structures (IIIA) or (IIIB):

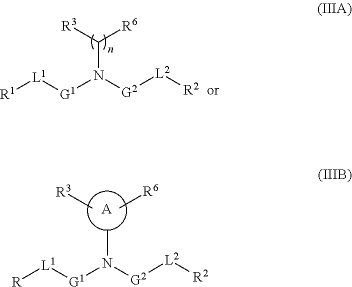

wherein:
A is a 3 to 8-membered cycloalkyl or cycloalkylene ring;
R6 is, at each occurrence, independently H, OH or C1-C24 alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

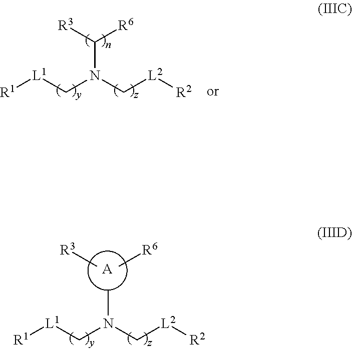

wherein y and z are each independently integers ranging from 1 to 12.

In any of the foregoing embodiments of Formula (III), one of L1 or L2 is —O(C═O)—. For example, in some embodiments each of L1 and L2 are —O(C═O)—. In some different embodiments of any of the foregoing, L1 and L2 are each independently —(C═O)O— or —O(C═O)—. For example, in some embodiments each of L1 and L2 is —(C═O)O—.

In some different embodiments of Formula (III), the lipid has one of the following structures (IIIE) or (IIIF):

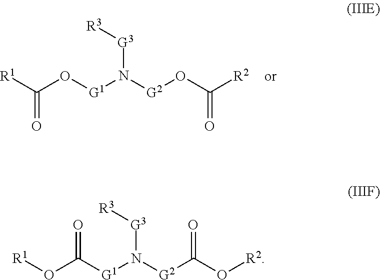

In some of the foregoing embodiments of Formula (III), the lipid has one of the following structures (IIIG), (IIIH), (IIII), or (IIIJ):

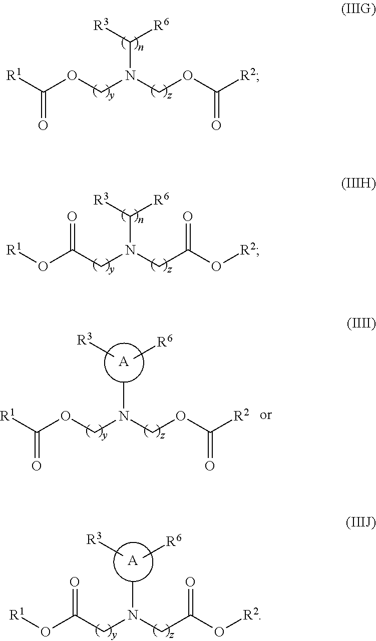

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R6 is H. In other of the foregoing embodiments, R6 is C1-C24 alkyl. In other embodiments, R6 is OH.

In some embodiments of Formula (III), G3 is unsubstituted. In other embodiments, G3 is substituted. In various different embodiments, G3 is linear C1-C24 alkylene or linear C1-C24 alkenylene.

In some other foregoing embodiments of Formula (III), R1 or R2, or both, is C6-C24 alkenyl. For example, in some embodiments, R1 and R2 each, independently have the following structure:

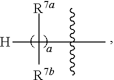

wherein:
R7a and R7b are, at each occurrence, independently H or C1-C12 alkyl; and
a is an integer from 2 to 12,
wherein R7a, R7b and a are each selected such that R1 and R2 each independently comprise from 6 to 20 carbon atoms. For example, in some embodiments a is an integer ranging from 5 to 9 or from 8 to 12.

In some of the foregoing embodiments of Formula (III), at least one occurrence of R7a is H.

For example, in some embodiments, R7a is H at each occurrence. In other different embodiments of the foregoing, at least one occurrence of R7b is C1-C8 alkyl. For example, in some embodiments, C1-C8 alkyl is methyl, ethyl, n-propyl, iso-propyl, n-butyl, iso-butyl, tert-butyl, n-hexyl or n-octyl.

In different embodiments of Formula (III), R1 or R2, or both, has one of the following structures:

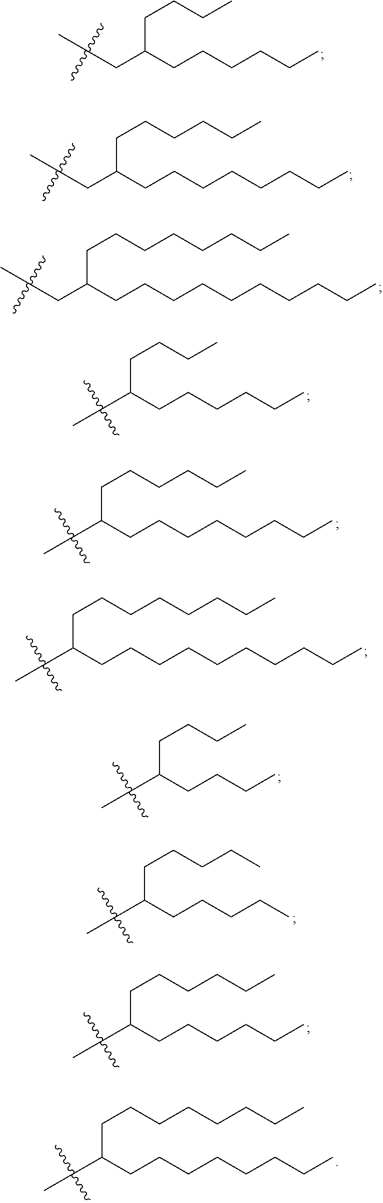

In some of the foregoing embodiments of Formula (III), R3 is OH, CN, —C(═O)OR4, —OC(═O)R4 or —NHC(═O)R4. In some embodiments, R4 is methyl or ethyl.

In various different embodiments, the cationic lipid of Formula (III) has one of the structures set forth in the table below.

Representative Compounds of Formula (III).

| No. | Structure |
|---|---|
| III-1 | 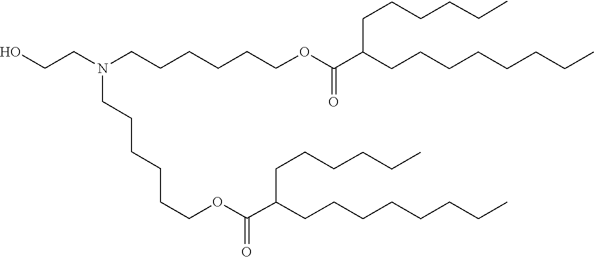 [SMILES: CCCCCCCCC(CCCCCC)C(=O)OCCCCCCN(CCO)CCCCCCOC(=O)C(CCCCCC)CCCCCCCC] |
| III-2 | 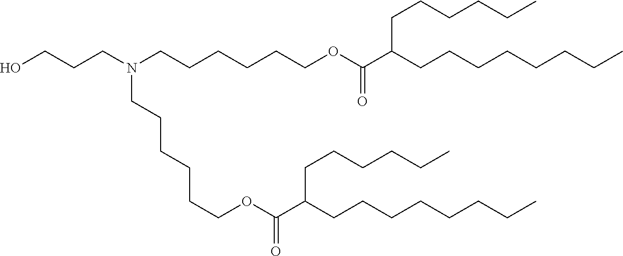 [SMILES: CCCCCCCCC(CCCCCC)C(=O)OCCCCCCN(CCCO)CCCCCCOC(=O)C(CCCCCC)CCCCCCCC] |
| III-3 | 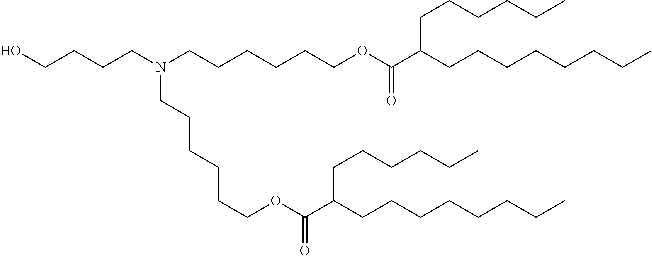 [SMILES: CCCCCCCCC(CCCCCC)C(=O)OCCCCCCN(CCCCO)CCCCCCOC(=O)C(CCCCCC)CCCCCCCC] |
| III-4 | 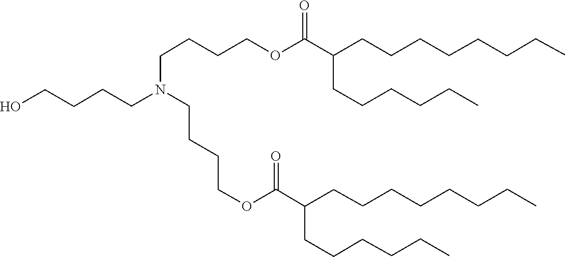 [SMILES: CCCCCCCCC(CCCCCC)C(=O)OCCCCN(CCCCO)CCCCOC(=O)C(CCCCCC)CCCCCCCC] |
| III-5 | 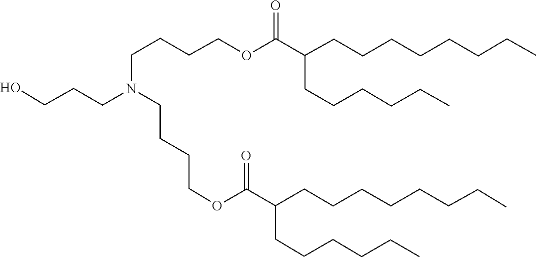 [SMILES: CCCCCCCCC(CCCCCC)C(=O)OCCCCN(CCCO)CCCCOC(=O)C(CCCCCC)CCCCCCCC] |
| III-6 | 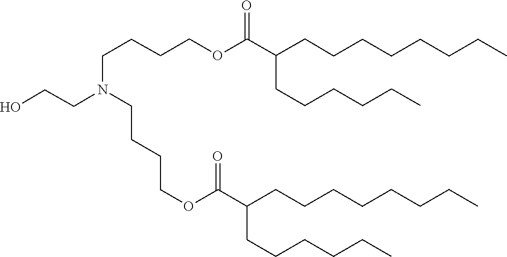 [SMILES: CCCCCCCCC(CCCCCC)C(=O)OCCCCN(CCO)CCCCOC(=O)C(CCCCCC)CCCCCCCC] |
| III-7 | 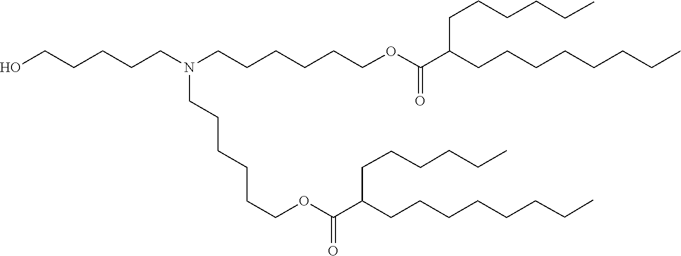 [SMILES: CCCCCCCCC(CCCCCC)C(=O)OCCCCCCN(CCCCCO)CCCCCCOC(=O)C(CCCCCC)CCCCCCCC] |
| III-8 | 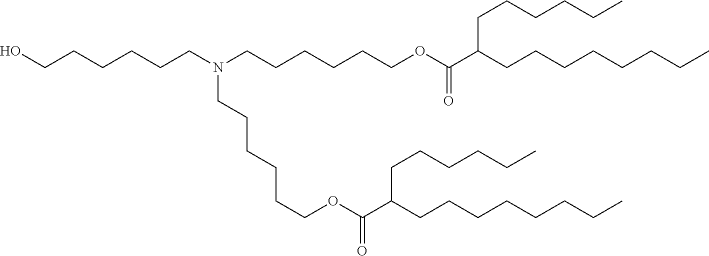 [SMILES: CCCCCCCCC(CCCCCC)C(=O)OCCCCCCN(CCCCCCO)CCCCCCOC(=O)C(CCCCCC)CCCCCCCC] |
| III-9 | 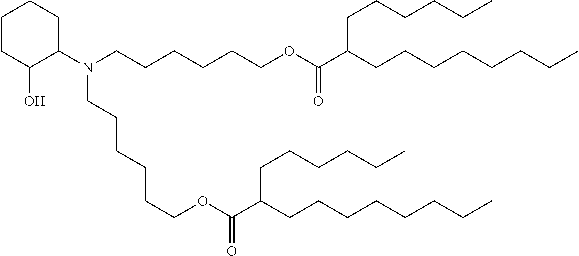 [SMILES: CCCCCCCCC(CCCCCC)C(=O)OCCCCCCN(CCCCCCOC(=O)C(CCCCCC)CCCCCCCC)C1CCCCC1O] |
| III-10 | 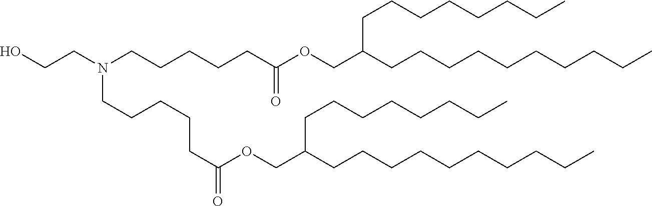 [SMILES: CCCCCCCCCCC(CCCCCCCC)COC(=O)CCCCCN(CCO)CCCCCC(=O)OCC(CCCCCCCC)CCCCCCCCCC] |
| III-11 | 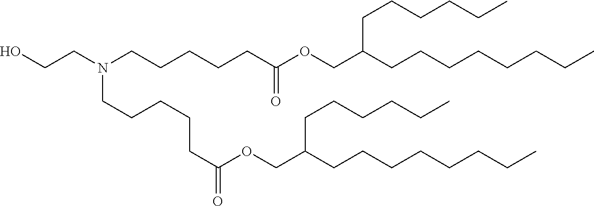 [SMILES: CCCCCCCCC(CCCCCC)COC(=O)CCCCCN(CCO)CCCCCC(=O)OCC(CCCCCC)CCCCCCCC] |
| III-12 | 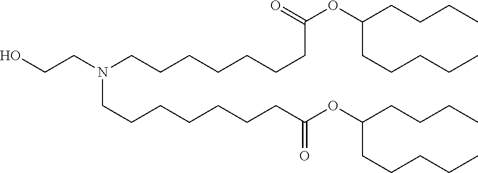 [SMILES: CCCCCC(CCCC)OC(=O)CCCCCCCN(CCO)CCCCCCCC(=O)OC(CCCC)CCCCC] |
| III-13 | 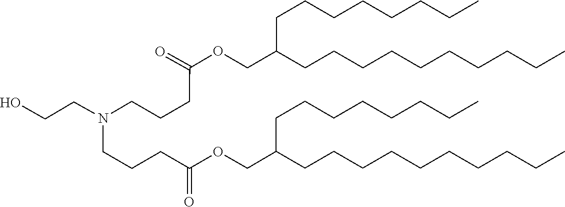 [SMILES: CCCCCCCCCCC(CCCCCCCC)COC(=O)CCCN(CCO)CCCC(=O)OCC(CCCCCCCC)CCCCCCCCCC] |
| III-14 | 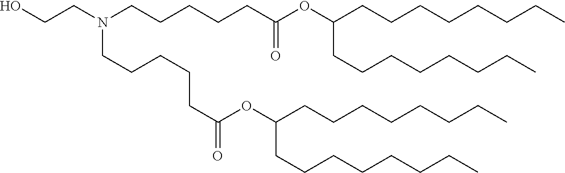 [SMILES: CCCCCCCCC(CCCCCCCC)OC(=O)CCCCCN(CCO)CCCCCC(=O)OC(CCCCCCCC)CCCCCCCC] |
| III-15 | 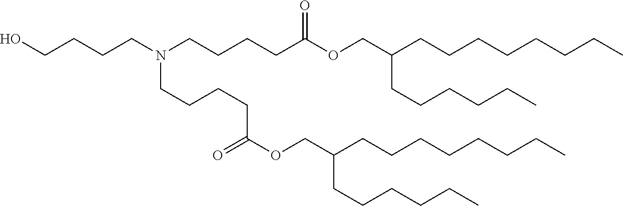 [SMILES: CCCCCCCCC(CCCCCC)COC(=O)CCCCN(CCCCO)CCCCC(=O)OCC(CCCCCC)CCCCCCCC] |
| III-16 | 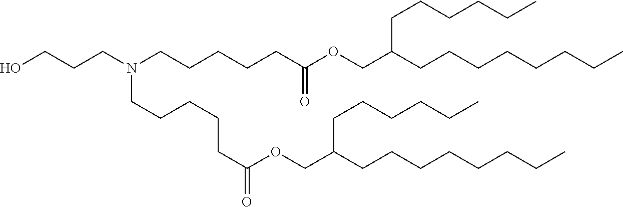 [SMILES: CCCCCCCCC(CCCCCC)COC(=O)CCCCCN(CCCO)CCCCCC(=O)OCC(CCCCCC)CCCCCCCC] |
| III-17 | 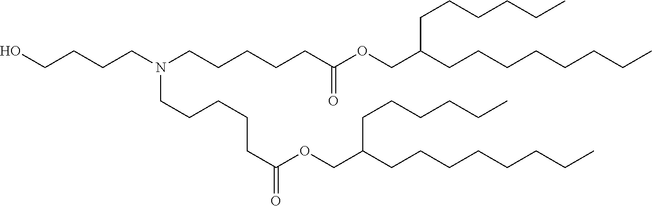 [SMILES: CCCCCCCCC(CCCCCC)COC(=O)CCCCCN(CCCCO)CCCCCC(=O)OCC(CCCCCC)CCCCCCCC] |
| III-18 | 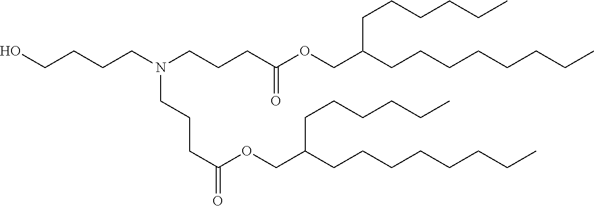 [SMILES: CCCCCCCCC(CCCCCC)COC(=O)CCCN(CCCCO)CCCC(=O)OCC(CCCCCC)CCCCCCCC] |
| III-19 | 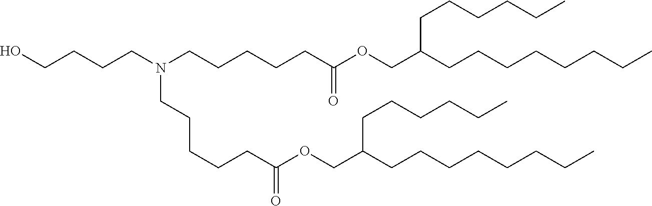 [SMILES: CCCCCCCCC(CCCCCC)COC(=O)CCCCCN(CCCCO)CCCCCC(=O)OCC(CCCCCC)CCCCCCCC] |
| III-20 | 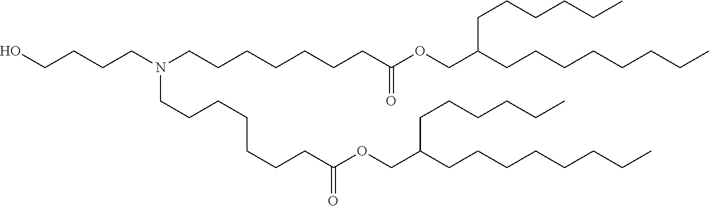 [SMILES: CCCCCCCCC(CCCCCC)COC(=O)CCCCCCCN(CCCCO)CCCCCCCC(=O)OCC(CCCCCC)CCCCCCCC] |
| III-21 | 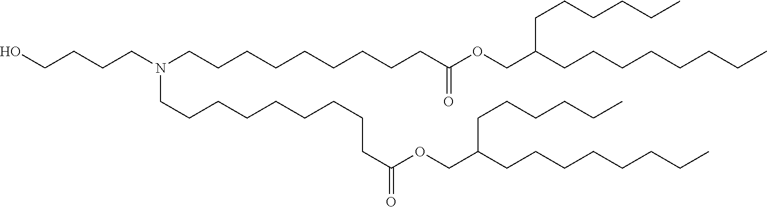 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 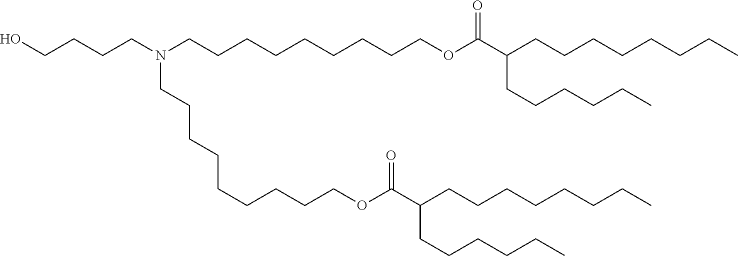 [SMILES: CCCCCCCCC(CCCCCC)C(=O)OCCCCCCCCCN(CCCCO)CCCCCCCCCOC(=O)C(CCCCCC)CCCCCCCC] |
| III-23 | 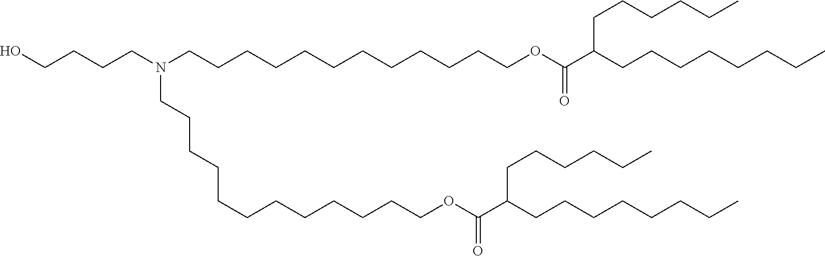 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 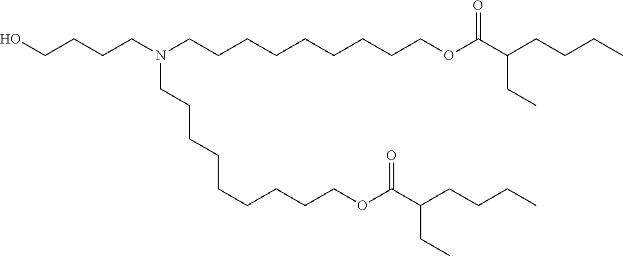 [SMILES: CCCCC(CC)C(=O)OCCCCCCCCCN(CCCCO)CCCCCCCCCOC(=O)C(CC)CCCC] |
| III-25 | 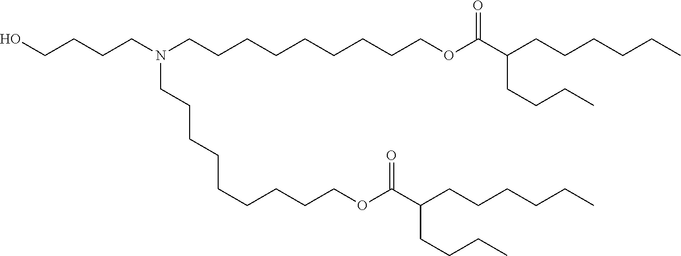 [SMILES: CCCCCCC(CCCC)C(=O)OCCCCCCCCCN(CCCCO)CCCCCCCCCOC(=O)C(CCCC)CCCCCC] |
| III-26 | 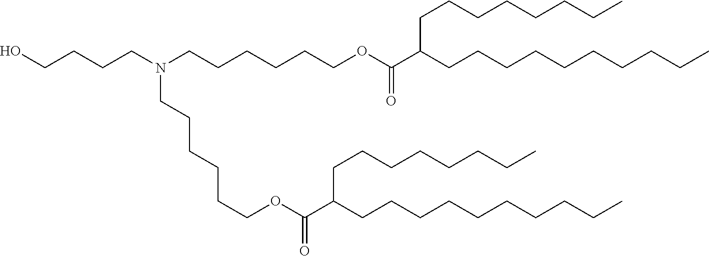 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 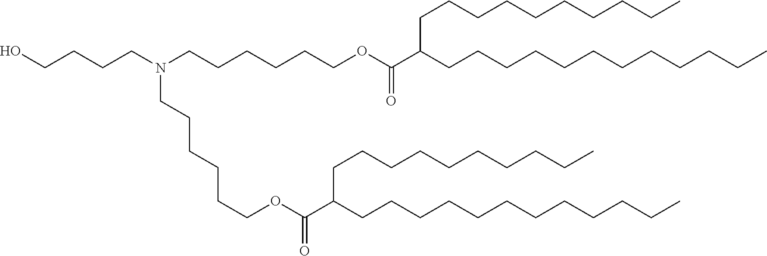 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 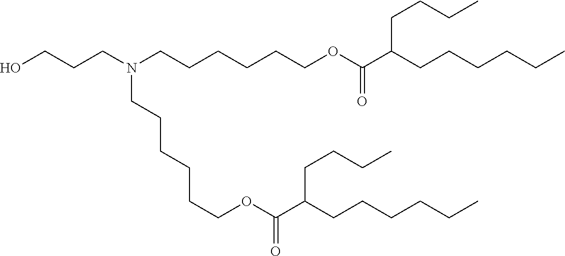 [SMILES: CCCCCCC(CCCC)C(=O)OCCCCCCN(CCCO)CCCCCCOC(=O)C(CCCC)CCCCCC] |
| III-29 | 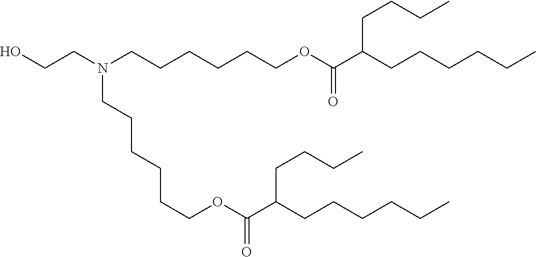 [SMILES: CCCCCCC(CCCC)C(=O)OCCCCCCN(CCO)CCCCCCOC(=O)C(CCCC)CCCCCC] |
| III-30 | 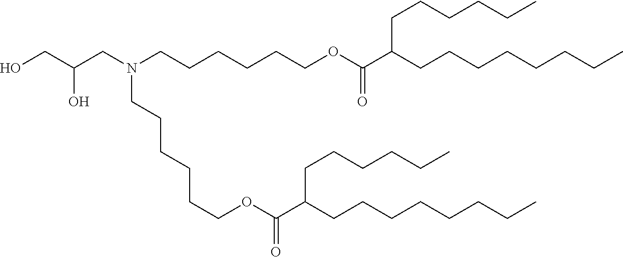 [SMILES: CCCCCCCCC(CCCCCC)C(=O)OCCCCCCN(CCCCCCOC(=O)C(CCCCCC)CCCCCCCC)CC(O)CO] |
| III-31 | 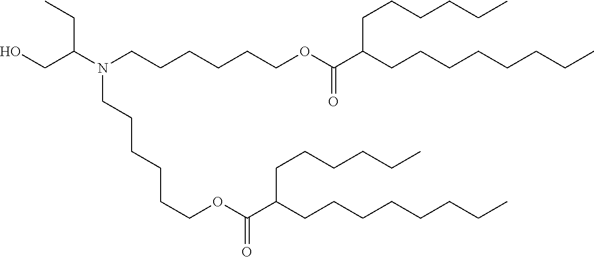 [SMILES: CCCCCCCCC(CCCCCC)C(=O)OCCCCCCN(CCCCCCOC(=O)C(CCCCCC)CCCCCCCC)C(CC)CO] |
| III-32 | 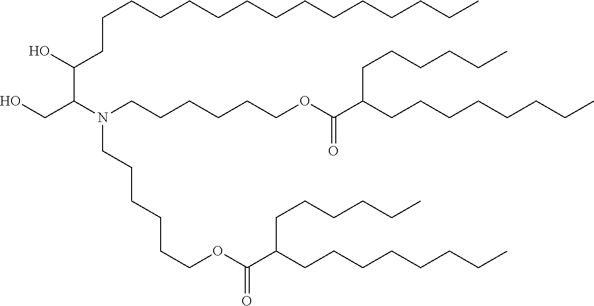 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 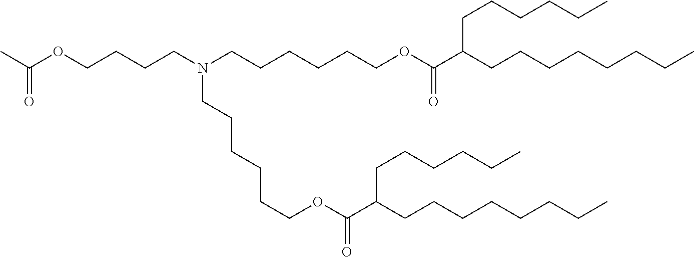 [SMILES: CCCCCCCCC(CCCCCC)C(=O)OCCCCCCN(CCCCCCOC(=O)C(CCCCCC)CCCCCCCC)CCCCOC(C)=O] |
| III-34 | 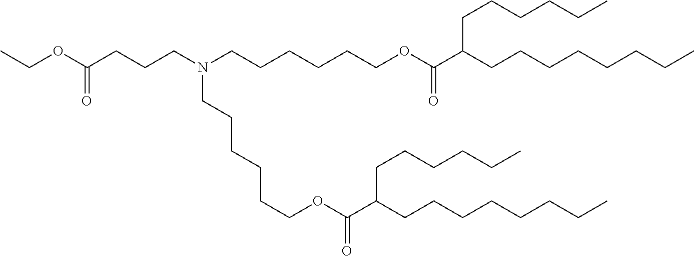 [SMILES: CCCCCCCCC(CCCCCC)C(=O)OCCCCCCN(CCCCCCOC(=O)C(CCCCCC)CCCCCCCC)CCCC(=O)OCC] |
| III-35 | 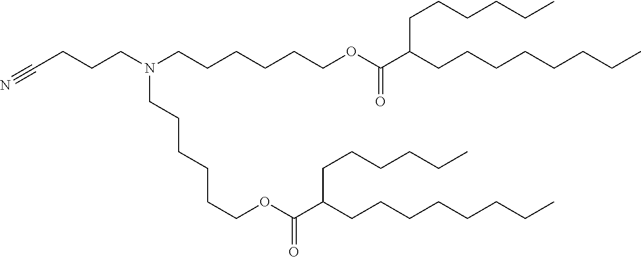 [SMILES: CCCCCCCCC(CCCCCC)C(=O)OCCCCCCN(CCCC#N)CCCCCCOC(=O)C(CCCCCC)CCCCCCCC] |
| III-36 | 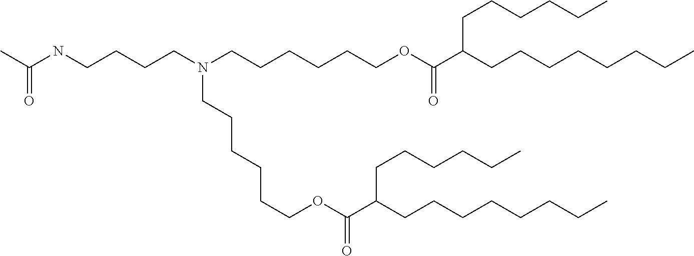 |
| III-37 | 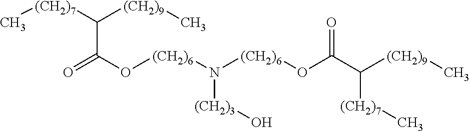 |
| III-38 | 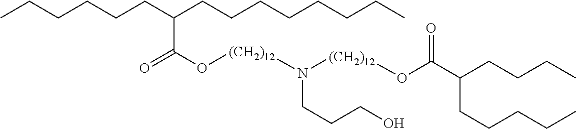 |
| III-39 | 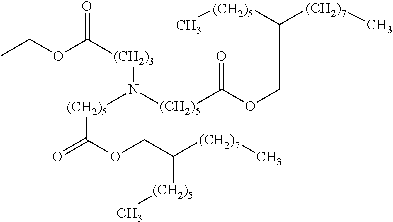 |
| III-40 | 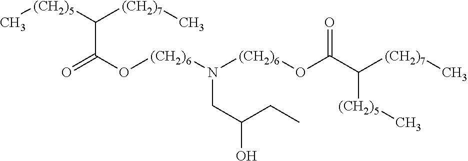 |
| III-41 | 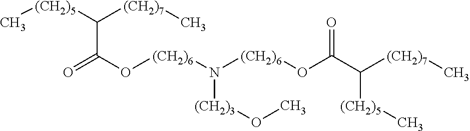 |
| III-42 | 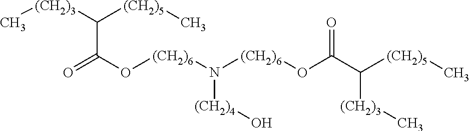 |
| III-43 | 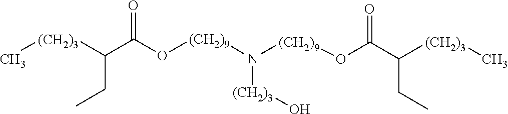 |
| III-44 | 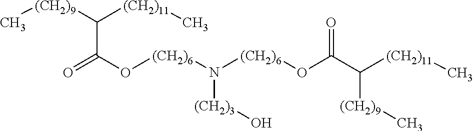 |
| III-45 | 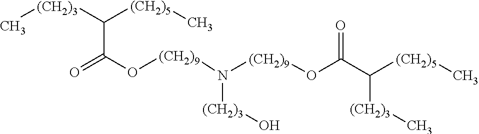 |
| III-46 | 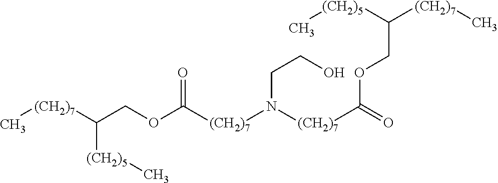 |
| III-47 | 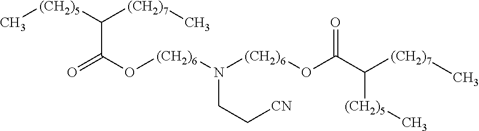 |
| III-48 | 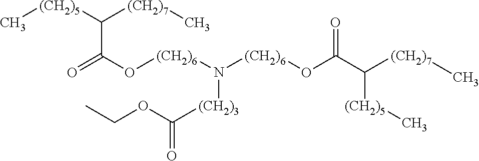 |
| III-49 | 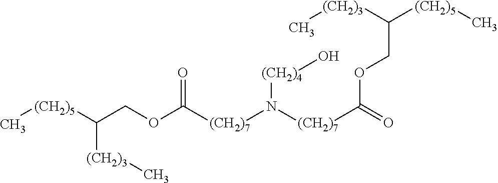 |

Further representative cationically ionizable lipids are as follows:

| No. | Structure |
|---|---|
| A | 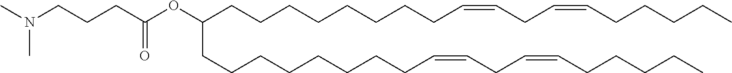 [SMILES: CCCCC/C=C\C/C=C\CCCCCCCCC(CCCCCCCC/C=C\C/C=C\CCCCC)OC(=O)CCCN(C)C] |
| B | 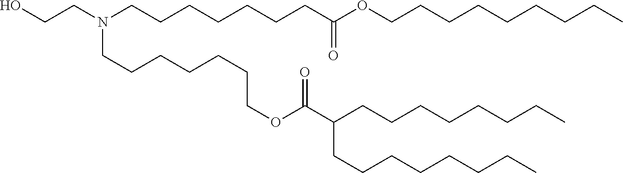 [SMILES: CCCCCCCCCOC(=O)CCCCCCCN(CCO)CCCCCCCOC(=O)C(CCCCCCCC)CCCCCCCC] |
| C | 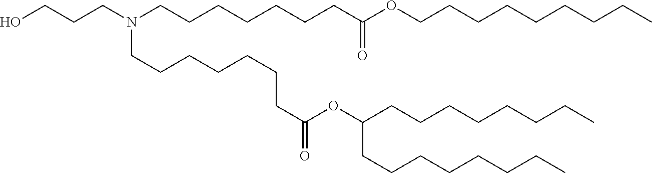 [SMILES: CCCCCCCCCOC(=O)CCCCCCCN(CCCO)CCCCCCCC(=O)OC(CCCCCCCC)CCCCCCCC] |
| D | 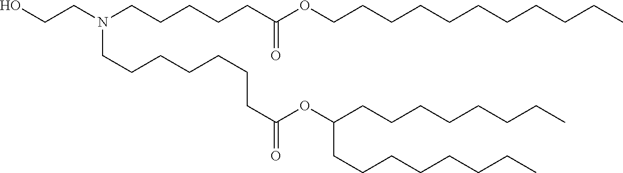 [SMILES: CCCCCCCCCCCOC(=O)CCCCCN(CCO)CCCCCCCC(=O)OC(CCCCCCCC)CCCCCCCC] |
| E | 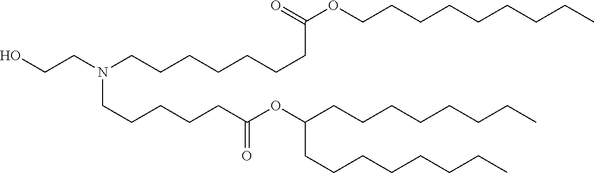 [SMILES: CCCCCCCCCOC(=O)CCCCCCCN(CCO)CCCCCC(=O)OC(CCCCCCCC)CCCCCCCC] |
| F | 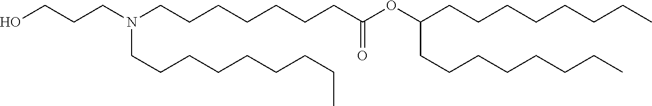 [SMILES: CCCCCCCCCN(CCCO)CCCCCCCC(=O)OC(CCCCCCCC)CCCCCCCC] |

In some embodiments, DNA and/or RNA described herein is formulated in an LNP composition comprising a cationically ionizable lipid, e.g., a cationically ionizable lipid as shown above, a neutral lipid, a steroid, and a polymer conjugated lipid.

In some embodiments, DNA and/or RNA described herein is formulated in an LNP composition comprising a cationically ionizable lipid of Formula III, a neutral lipid, a steroid, and a polymer conjugated lipid.

In some embodiments, DNA and/or RNA described herein is formulated in an LNP composition comprising a cationically ionizable lipid shown in the above tables, a neutral lipid, a steroid, and a polymer conjugated lipid.

In some embodiments, DNA and/or RNA described herein is formulated in an LNP composition comprising 3D-P-DMA, a neutral lipid, a steroid, and a polymer conjugated lipid.

In some embodiments, DNA and/or RNA described herein is formulated in an LNP composition comprising ALC-0366, a neutral lipid, a steroid, and a polymer conjugated lipid.

In some embodiments, DNA and/or RNA described herein is formulated in an LNP composition comprising ALC-0315, a neutral lipid, a steroid, and a polymer conjugated lipid.

In some embodiments, the neutral lipid is DSPC. In some embodiments, the steroid is cholesterol. In some embodiments, the polymer conjugated lipid is a pegylated lipid, e.g., DMG-PEG 2000, PEG2000-C-DMA, or ALC-0159.

In some embodiments, DNA and/or RNA described herein is formulated in an LNP composition comprising a cationically ionizable lipid, e.g., a cationically ionizable lipid as shown above, a neutral lipid, a steroid, and a pegylated lipid.

In some embodiments, DNA and/or RNA described herein is formulated in an LNP composition comprising a cationically ionizable lipid of Formula III, a neutral lipid, a steroid, and a pegylated lipid.

In some embodiments, DNA and/or RNA described herein is formulated in an LNP composition comprising a cationically ionizable lipid shown in the above tables, a neutral lipid, a steroid, and a pegylated lipid.

In some embodiments, DNA and/or RNA described herein is formulated in an LNP composition comprising 3D-P-DMA, a neutral lipid, a steroid, and a pegylated lipid.

In some embodiments, DNA and/or RNA described herein is formulated in an LNP composition comprising ALC-0366, a neutral lipid, a steroid, and a pegylated lipid.

In some embodiments, DNA and/or RNA described herein is formulated in an LNP composition comprising ALC-0315, a neutral lipid, a steroid, and a pegylated lipid.

In some embodiments, the neutral lipid is DSPC. In some embodiments, the steroid is cholesterol. In some embodiments, the pegylated lipid is DMG-PEG 2000, PEG2000-C-DMA, or ALC-0159.

In some embodiments, DNA and/or RNA described herein is formulated in an LNP composition comprising a cationically ionizable lipid, e.g., a cationically ionizable lipid as shown above, DSPC, cholesterol, and a pegylated lipid.

In some embodiments, DNA and/or RNA described herein is formulated in an LNP composition comprising a cationically ionizable lipid of Formula III, DSPC, cholesterol, and a pegylated lipid.

In some embodiments, DNA and/or RNA described herein is formulated in an LNP composition comprising a cationically ionizable lipid shown in the above tables, DSPC, cholesterol, and a pegylated lipid.

In some embodiments, DNA and/or RNA described herein is formulated in an LNP composition comprising 3D-P-DMA, DSPC, cholesterol, and a pegylated lipid.

In some embodiments, DNA and/or RNA described herein is formulated in an LNP composition comprising ALC-0366, DSPC, cholesterol, and a pegylated lipid.

In some embodiments, DNA and/or RNA described herein is formulated in an LNP composition comprising ALC-0315, DSPC, cholesterol, and a pegylated lipid.

In some embodiments, the pegylated lipid is DMG-PEG 2000, PEG2000-C-DMA, or ALC-0159.

In some embodiments, DNA and/or RNA described herein is formulated in an LNP composition comprising a cationically ionizable lipid, e.g., a cationically ionizable lipid as shown above, DSPC, cholesterol, and DMG-PEG 2000.

In some embodiments, DNA and/or RNA described herein is formulated in an LNP composition comprising a cationically ionizable lipid of Formula III, DSPC, cholesterol, and DMG-PEG 2000.

In some embodiments, DNA and/or RNA described herein is formulated in an LNP composition comprising a cationically ionizable lipid shown in the above tables, DSPC, cholesterol, and DMG-PEG 2000.

In some embodiments, DNA and/or RNA described herein is formulated in an LNP composition comprising 3D-P-DMA, DSPC, cholesterol, and DMG-PEG 2000.

In some embodiments, DNA and/or RNA described herein is formulated in an LNP composition comprising ALC-0366, DSPC, cholesterol, and DMG-PEG 2000.

In some embodiments, DNA and/or RNA described herein is formulated in an LNP composition comprising ALC-0315, DSPC, cholesterol, and DMG-PEG 2000.

In some embodiments, DNA and/or RNA described herein is formulated in an LNP composition comprising a cationically ionizable lipid, e.g., a cationically ionizable lipid as shown above, DSPC, cholesterol, and PEG2000-C-DMA.

In some embodiments, DNA and/or RNA described herein is formulated in an LNP composition comprising a cationically ionizable lipid of Formula III, DSPC, cholesterol, and PEG2000-C-DMA.

In some embodiments, DNA and/or RNA described herein is formulated in an LNP composition comprising a cationically ionizable lipid shown in the above tables, DSPC, cholesterol, and PEG2000-C-DMA.

In some embodiments, DNA and/or RNA described herein is formulated in an LNP composition comprising 3D-P-DMA, DSPC, cholesterol, and PEG2000-C-DMA.

In some embodiments, DNA and/or RNA described herein is formulated in an LNP composition comprising ALC-0366, DSPC, cholesterol, and PEG2000-C-DMA.

In some embodiments, DNA and/or RNA described herein is formulated in an LNP composition comprising ALC-0315, DSPC, cholesterol, and PEG2000-C-DMA.

In some embodiments, DNA and/or RNA described herein is formulated in an LNP composition comprising a cationically ionizable lipid, e.g., a cationically ionizable lipid as shown above, DSPC, cholesterol, and ALC-0159.

In some embodiments, DNA and/or RNA described herein is formulated in an LNP composition comprising a cationically ionizable lipid of Formula III, DSPC, cholesterol, and ALC-0159.

In some embodiments, DNA and/or RNA described herein is formulated in an LNP composition comprising a cationically ionizable lipid shown in the above tables, DSPC, cholesterol, and ALC-0159.

In some embodiments, DNA and/or RNA described herein is formulated in an LNP composition comprising 3D-P-DMA, DSPC, cholesterol, and ALC-0159.

In some embodiments, DNA and/or RNA described herein is formulated in an LNP composition comprising ALC-0366, DSPC, cholesterol, and ALC-0159.

In some embodiments, DNA and/or RNA described herein is formulated in an LNP composition comprising ALC-0315, DSPC, cholesterol, and ALC-0159.

3D-P-DMA: (6Z,16Z)-12-((Z)-dec-4-en-1-yl)docosa-6,16-dien-11-yl 5-(dimethylamino)pentanoate

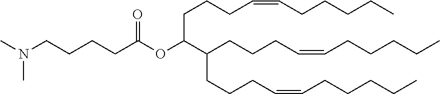 [SMILES: CCCCC/C=C\CCCC(CCC/C=C\CCCCC)C(CCC/C=C\CCCCC)OC(=O)CCCCN(C)C]

ALC-0366: ((3-hydroxypropyl)azanediyl)bis(nonane-9,1-diyl) bis(2-butyloctanoate)

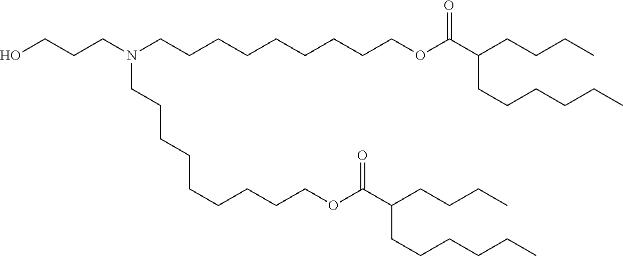 [SMILES: CCCCCCC(CCCC)C(=O)OCCCCCCCCCN(CCCO)CCCCCCCCCOC(=O)C(CCCC)CCCCCC]

ALC-0315: ((4-hydroxybutyl)azanediyl)bis(hexane-6,1-diyl)bis(2-hexyldecanoate)/6-[N-6-(2-hexyldecanoyloxy)hexyl-N-(4-hydroxybutyl)amino]hexyl 2-hexyldecanoate

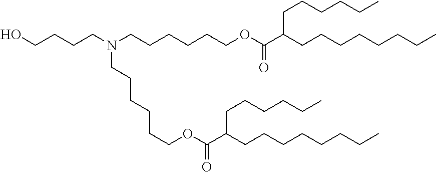 [SMILES: CCCCCCCCC(CCCCCC)C(=O)OCCCCCCN(CCCCO)CCCCCCOC(=O)C(CCCCCC)CCCCCCCC]

DMG-PEG 2000:

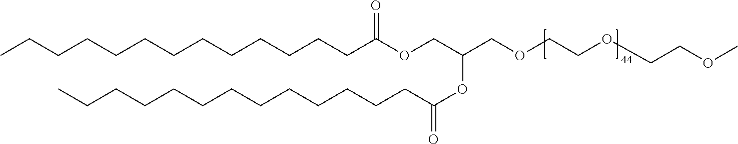

PEG2000-C-DMA: 3-N-[(ω-Methoxy poly(ethylene glycol)2000) carbamoyl]-1,2-dimyristyloxy-propylamine (MPEG-(2 kDa)-C-DMA or Methoxy-polyethylene glycol-2,3-bis(tetradecyloxy)propylcarbamate (2000))

wherein n has a mean value ranging from 30 to 60, such as about 50.

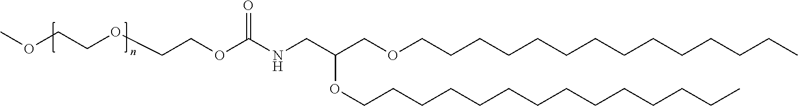

ALC-0159: 2-[(polyethylene glycol)-2000]-N,N-ditetradecylacetamide/2-[2-(ω-methoxy (polyethyleneglycol2000) ethoxy]-N,N-ditetradecylacetamide

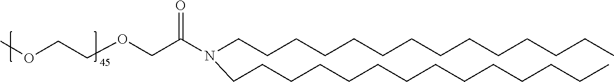

DSPC: 1,2-Distearoyl-sn-glycero-3-phosphocholine

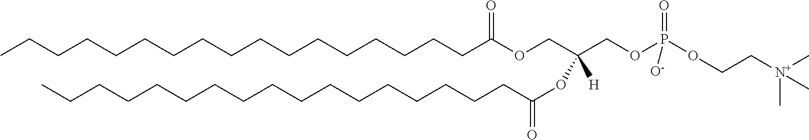 [SMILES: CCCCCCCCCCCCCCCCCC(=O)OC[C@H](COP(=O)([O-])OCC[N+](C)(C)C)OC(=O)CCCCCCCCCCCCCCCCC]

### Cholesterol:

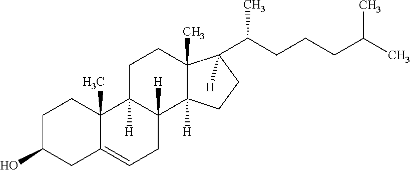

The N/P value is preferably at least about 4. In some embodiments, the N/P value ranges from 4 to 20, 4 to 12, 4 to 10, 4 to 8, or 5 to 7. In some embodiments, the N/P value is about 6.

The particles described herein may comprise a hydrophobic moiety (e.g., lipid) having a binding moiety covalently attached thereto. This hydrophobic moiety having a binding moiety covalently attached thereto is also referred to herein as “connector compound”. The hydrophobic moiety of the connector compound relates to the part of the connector compound that integrates into the particle comprising a payload. The binding moiety of the connector compound relates to the part of the connector compound that forms the binding partner for the docking compound. Generally, the connector compound is non-covalently incorporated into the particle comprising a payload, i.e., it forms an integral part of the particle, and the binding moiety of the connector compound is covalently attached to a hydrophobic moiety in a manner such that it is available for binding to the docking compound.

In some embodiments, the binding moiety of the connector compound comprises a peptide or protein (e.g., an antibody or antibody fragment or a peptide tag).

In some embodiments, the binding moiety of the connector compound comprises a peptide or protein (e.g., an antibody or antibody fragment or a peptide tag) and is chemically linked, e.g., through a linker, to the hydrophobic moiety (e.g., lipid).

The connector compound used herein comprises a hydrophobic component (e.g., lipid component) which allows it to be anchored in the particle. In some embodiments, the hydrophobic component comprises a moiety selected from vitamin E, dialkylamine, e.g., dimyristylamine (DMA), diacylglyceride, e.g., 1,2-dimyristoyl-sn-glycerol (DMG) and ceramide. In some embodiments, the hydrophobic moiety comprises two C8-C24 hydrocarbon chains. In some embodiments, the hydrophobic moiety comprises two C10-C18 hydrocarbon chains.

In some embodiments, the connector compound used herein has as a hydrophobic group (e.g., lipid) a phospholipid, e.g., a biodegradable phospholipid such as phosphatidylethanolamine. In some embodiments, the connector compound used herein has as a hydrophobic group (e.g., lipid) a glycerophospholipid. In some embodiments, the phospholipid is selected from the group consisting of DSPE (distearoylphosphatidylethanolamine), DPPE (dipalmitoylphosphatidylethanolamine), DOPE (dioleoylphosphatidylethanolamine), and POPE (palmitoyloleylphosphatidylethanolamine), and mixtures thereof. In some embodiments, as a phospholipid, DSPE will be used for its qualities of stability in the particles described herein. Moreover, as hydrophobic group (e.g., lipid), a compound having at least one alkyl chain providing hydrophobic anchoring to a particle as described herein may be used.

In some embodiments, the connector compound comprises a polymer. In some embodiments, the hydrophobic moiety (e.g., lipid) of the connector compound and the binding moiety of the connector compound are connected through the polymer.

In some embodiments, the polymer is a hydrophilic polymer and the connector compound comprises an amphiphilic derivative of the polymer. In some embodiments, the amphiphilic derivative of a polymer comprises a hydrophobic component (e.g., lipid component) which allows it to be anchored in the particle and a hydrophilic component of the polymer facing the outside of said particle, conferring hydrophilic properties at the surface thereof. In some embodiments, the amphiphilic derivatives of a polymer is inserted into the particle via its hydrophobic end. Consequently, the polymer component faces the outside of said particle and forms a protective hydrophilic shell surrounding the particle. In some embodiments, the polymer portion of the amphiphilic derivative contributes to conferring stealth properties on the particles. In some embodiments, the plasmatic half-life of the particles described herein is greater than 2 hours, e.g., between 3 and 10 hours. This characteristic advantageously allows the particles to accumulate at the target cells and to liberate therein their contents (payload) within reasonable amounts of time. The effectiveness of the targeted delivery described herein therefore increases as a result.

The term “stealth” is used herein to describe the ability of the particles described herein not to be detected and then sequestered and/or degraded, or to be hardly detected and then sequestered and/or degraded, and/or to be detected and then sequestered and/or degraded late, by the immune system of the host to which they are administered.

Macrophages constitute one of the most important components of the immune system and play a predominant role in eliminating foreign particles, including liposomes and other colloidal particles, from the blood circulation. At the molecular level, the clearance of particles takes place in two steps: opsonization by the depositing of serum proteins (or “opsonins”) at the surface of the particles followed by recognition and capture of the opsonized particles by macrophages.

Modification of the surface of particles with chains of hydrophilic and flexible polymers, e.g., polymers of the poly(ethylene glycol) type, confers them a steric protection by preventing the opsonins reaching the surface of the particles.

In some embodiments, the amphiphilic derivative of a polymer used herein has as a hydrophobic group (e.g., lipid) as specified herein. In some embodiments, the amphiphilic derivative of a polymer used herein has as a hydrophobic group (e.g., lipid) a phospholipid, e.g., a biodegradable phospholipid such as phosphatidylethanolamine. In some embodiments, the phospholipid is selected from the group consisting of DSPE (distearoylphosphatidylethanolamine), DPPE (dipalmitoylphosphatidylethanolamine), DOPE (dioleoylphosphatidylethanolamine), and POPE (palmitoyloleylphosphatidylethanolamine), and mixtures thereof. In some embodiments, as a phospholipid, DSPE will be used for its qualities of stability in the particles described herein. Moreover, as hydrophobic group (e.g., lipid), a compound having at least one alkyl chain providing hydrophobic anchoring to a particle as described herein may be used.

In some embodiments, the polymer for use herein is selected from the group consisting of poly(ethylene glycol) (PEG), polysarcosine (pSar) (poly(N-methylglycine), polyoxazoline (POX), polyoxazine (POZ), and poly-2-(2-(2-aminoethoxy)ethoxy)acetic acid (pAEEA) (including derivatives thereof).

In some embodiments, a polymer is designed to sterically stabilize a particle by forming a protective hydrophilic layer. In some embodiments, a polymer can reduce association of a particle with serum proteins and/or the resulting uptake by the reticuloendothelial system when such particles are administered in vivo.

In some embodiments, the PEG is an optionally substituted linear or branched polymer of ethylene glycol or ethylene oxide. In some embodiments, the PEG is unsubstituted. In some embodiments, the PEG is substituted, e.g., by one or more alkyl, alkoxy, acyl, hydroxy or aryl groups. In some embodiments, the PEG has a molecular weight of from about 130 to about 50,000, in another embodiment about 150 to about 30,000, in another embodiment about 150 to about 20,000, in another embodiment about 150 to about 15,000, in another embodiment about 150 to about 10,000, in another embodiment about 150 to about 6000, in another embodiment about 150 to about 5000, in another embodiment about 150 to about 4000, in another embodiment about 150 to about 3000, in another embodiment about 300 to about 3000, in another embodiment about 1000 to about 3000, and in still another embodiment about 1500 to about 2500.

In some embodiments, the PEG moiety of the amphiphilic derivative of a polymer has a molecular weight of 1000 or more. In some embodiments, the PEG moiety of the amphiphilic derivative of a polymer comprises 10 units or more of formula (O-CH2-CH2)n. In some embodiments, the PEG comprises from 20 to 200 ethylene oxide units, such as about 45 ethylene oxide units.

In some embodiments, the PEG comprises “PEG2k”, also termed “PEG 2000”, which has an average molecular weight of about 2000 Daltons.

In some embodiments, DSPE-PEG2000, DSPE-PEG3000 and DSPE-PEG5000 are used as the amphiphilic derivative of a polymer.

In some embodiments, a pSar comprises between 2 and 200 sarcosine units, such as between 5 and 100 sarcosine units, between 10 and 50 sarcosine units, between 15 and 40 sarcosine units, e.g., about 23 sarcosine units.

In some embodiments, a pSar comprises the structure of the following general formula:

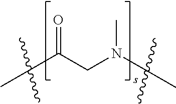

wherein s is the number of sarcosine units.

In some embodiments, the POX and/or POZ polymer comprises between 2 and 200, between 2 and 190, between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70 POX and/or POZ repeating units.

In some embodiments, the POX and/or POZ polymer comprises the following general formula:

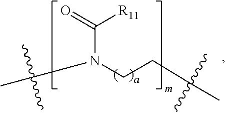

wherein a is an integer between 1 and 2; R11 is alkyl, in particular C1-3 alkyl, such as methyl, ethyl, iso-propyl, or n-propyl, and is independently selected for each repeating unit; and m refers to the number of POX and/or POZ repeating units.

In some embodiments, the POX and/or POZ polymer is a polymer of POX and comprises repeating units of the following general formula:

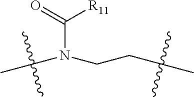

In some embodiments, the POX and/or POZ polymer is a polymer of POZ and comprises repeating units of the following general formula:

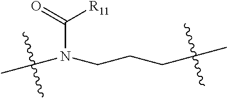

In any of the above embodiments of formulas, m (i.e., the number of repeating units in the polymer) preferably is between 2 and 190, such as between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70. In certain embodiments, m is 2 to 180, such as 4 to 160, 6 to 140, 8 to 120 or 10 to 100, e.g., 20 to 80, 30 to 70, or 40 to 50.

In some embodiments, the POX and/or POZ polymer is a copolymer comprising repeating units of the following general formulas:

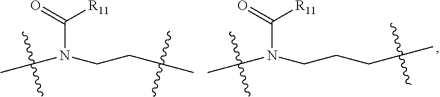

wherein the number of repeating units shown on the left in the copolymer is 1 to 199; the number of repeating units of formula on the right in the copolymer is 1 to 199; and the sum of the number of repeating units of formula on the left and the number of repeating units of formula on the right in the copolymer is 2 to 200.

In some embodiments of the oxazolinylated and/or oxazinylated hydrophobic moiety (e.g., lipid), the number of repeating units of formula on the left in the copolymer is 1 to 179, such as 1 to 159, 1 to 139, 1 to 119 or 1 to 99; the number of repeating units of formula on the right in the copolymer is 1 to 179, such as 1 to 159, 1 to 139, 1 to 119 or 1 to 99; and the sum of the number of repeating units of formula on the left and the number of repeating units of formula on the right in the copolymer is 2 to 180, such as 4 to 160, 6 to 140, 8 to 120 or 10 to 100, e.g., 20 to 80, 30 to 70, or 40 to 50.

In some of the above embodiments, R11 at each occurrence (i.e., in each repeating unit) may be the same alkyl group (e.g., R11 may be methyl in each repeating unit). In some alternative embodiments, R11 in at least one repeating unit differs from R11 in another repeating unit (e.g., for at least one repeating unit R11 is one specific alkyl (such as ethyl), and for at least one different repeating unit R11 is a different specific alkyl (such as methyl)). For example, each R11 may be selected from two different alkyl groups (such as methyl and ethyl) and not all R11 are the same alkyl.

In any of the above embodiments, R11 preferably is methyl or ethyl, more preferably methyl. Thus, in some embodiments, each R11 is methyl or each R11 is ethyl. In some alternative embodiments, R11 is independently selected from methyl and ethyl for each repeating unit, wherein in at least one repeating unit R11 is methyl, and in at least one repeating unit R11 is ethyl.

In some embodiments, the polymer comprises poly-2-(2-(2-aminoethoxy)ethoxy)acetic acid (pAEEA) or poly-2-(2-(2-methylaminoethoxy)ethoxy)acetic acid (pMAEEA), or a derivative thereof.

In some embodiments, the polymer comprises the following general formula:

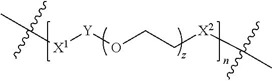

wherein
X2 and X1 taken together are optionally substituted amide, optionally substituted thioamide or ester;
Y is —CH2-, —(CH2)2-, or —(CH2)3-;
z is 2 to 24; and
n is 1 to 100.

In some embodiments,
(i) when X1 is —C(O)— then X2 is —NR1-;
(ii) when X1 is —NR1- then X2 is —C(O)—;
(iii) when X1 is —C(S)— then X2 is —NR1-;
(iv) when X1 is —NR1- then X2 is —C(S)—;
(v) when X1 is —C(O)— then X2 is —O—; or
(vi) when X1 is —O— then X2 is —C(O)—;
wherein R1 is hydrogen or C1-8 alkyl.

In some embodiments, X1 is —C(O)— and X2 is —NR1-, wherein R1 is hydrogen or C1-8 alkyl.

In some embodiments, X1 is —C(O)— and X2 is —NR1-, wherein R1 is hydrogen or methyl. In some embodiments, X1 is —C(O)— and X2 is —NR1-, wherein R1 is hydrogen.

In some embodiments, Y is —CH2- or —(CH2)2-. In some embodiments, Y is —CH2-.

In some embodiments, the polymer comprises the following general formula:

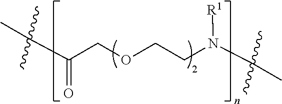

wherein
R1 is hydrogen or C1-8 alkyl;
z is 2 to 24; and
n is 1 to 100.

In some embodiments of the above formulas, z is 2 to 10. In some embodiments, z is 2 to 7. In some embodiments, z is 2 to 5. In some embodiments, z is 2 or 3. In some embodiments, z is 2.

In some embodiments, the polymer comprises the following general formula:

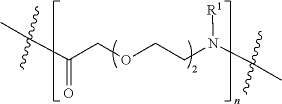

wherein
R1 is hydrogen or C1-8 alkyl; and
n is 1 to 100.

In some embodiments of the above formulas, R1 is hydrogen or methyl. In some embodiments, R1 is hydrogen.

In some embodiments, the polymer comprises the following general formula:

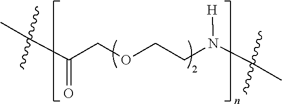

wherein
n is 1 to 100.

In some embodiments of the above formulas, n is 5 to 50. In some embodiments, n is 5 to 25.

In some embodiments, n is 7 to 14. In some embodiments, n is 10 to 25. In some embodiments, n is 14 to 17. In some embodiments, n is 8 or 14.

In some embodiments, the molar proportion of the amphiphilic derivative of a polymer integrated into the particles is between 0.5 and 20 mol % of the lipid molecules making up the particle, preferably between 1 and 10 mol %.

In some embodiments, the connector compound comprises the following general formula:

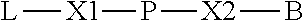

wherein
P comprises a polymer;
L comprises a hydrophobic moiety (e.g., lipid) attached to a first end of the polymer;
B comprises a binding moiety attached to a second end of the polymer;
X1 is absent or a first linking moiety; and
X2 is absent or a second linking moiety.

In some embodiments, X1 comprises a carbonyl group. In some embodiments, L comprises a phosphatidylethanolamine which may be linked to P by an amide group.

In some embodiments, X2 comprises the reaction product of a thiol or cysteine reactive group, e.g., a maleimide group, with a thiol or cysteine group of a compound comprising the binding moiety.

In some embodiments, L comprises a lipid as described above. In some embodiments, L comprises DSPE (distearoylphosphatidylethanolamine), DPPE (dipalmitoylphosphatidylethanolamine), DOPE (dioleoylphosphatidylethanolamine), and POPE (palmitoyloleylphosphatidylethanolamine) which may be linked to P by an amide group.

In some embodiments, P comprises a polymer as described above. In some embodiments, P comprises a polymer which provides stealth property, extends circulation half-life and/or reduces non-specific protein binding or cell adhesion. In some embodiments, P comprises a polymer selected from the group consisting of poly(ethylene glycol) (PEG), polysarcosine (pSar) (poly(N-methylglycine), polyoxazoline (POX), polyoxazine (POZ), and poly-2-(2-(2-aminoethoxy)ethoxy)acetic acid (pAEEA) (including derivatives thereof). In some embodiments, P comprises polyethyleneglycol (PEG); e.g., PEG as described above.

In some embodiments, L-X1-P comprises an amphiphilic derivative of a polymer as described above. In some embodiments, the amphiphilic derivative of a polymer comprises a conjugate of disteroyl-glycero-phosphoethanolamine (DSPE) and a polymer, e.g., a polymer as described above. In some embodiments, the amphiphilic derivative of a polymer comprises a disteroyl-glycero-phosphoethanolamine-polyethyleneglycol-conjugate (DSPE-PEG).

In some embodiments, the connector compound is obtainable by reacting the thiol or cysteine reactive group of a reagent comprising an amphiphilic derivative of a polymer, e.g., a PEG reagent comprising a hydrophobic moiety (e.g., lipid), with a thiol or cysteine group of a compound comprising the binding moiety.

In some embodiments, the thiol or cysteine reactive group comprises a maleimide group.

In some embodiments, the PEG reagent comprises DSPE-PEG-maleimide. In some embodiments, the compound comprising the binding moiety comprises the formula SH(CH2)nC(O)—B, wherein n ranges from 1 to 5 and B comprises the binding moiety. In some embodiments, n is 2.

In some embodiments, the connector compound comprises the reaction product of 1,2-distearoyl-sn-glycero-3-phosphoethanolamine-N-[maleimide(polyethylene glycol)] with a compound comprising the formula SH(CH2)nC(O)—B, wherein n ranges from 1 to 5 and B comprises the binding moiety. In some embodiments, n is 2.

In some embodiments, the connector compound (hydrophobic moiety having a binding moiety covalently attached thereto) comprises the following general formula:

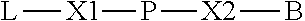

wherein L, X1, P and B are as described above and X2 comprises a thiosuccinimide moiety.

In some embodiments, the connector compound (hydrophobic moiety having a binding moiety covalently attached thereto) comprises the following general formula:

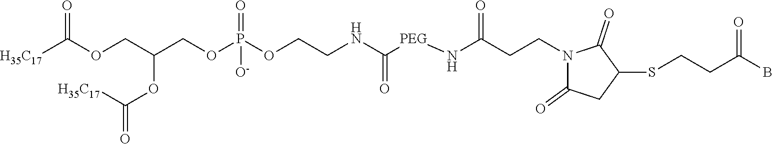

wherein B comprises the binding moiety.

In some embodiments of the above formulas, B comprises a moiety comprising the structure —N-peptide-C(O)—NH2.

In some embodiments, the connector compound (hydrophobic moiety having a binding moiety covalently attached thereto) comprises the following general formula:

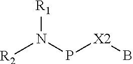

wherein P, X2 and B are as described above and R1 and R2 independently comprise an alkyl moiety. In some embodiments, at least one, e.g., each alkyl moiety is straight or branched, preferably straight. In some embodiments, at least one, e.g., each alkyl moiety has at least 8 carbon atoms, e.g., 8 to 24 such as 10 to 18 carbon atoms. Preferably, at least one, e.g., each alkyl moiety is the alkyl moiety of a fatty acid alcohol, more preferably at least one, e.g., each alkyl moiety is the alkyl moiety of a fatty acid alcohol having at least 8 carbon atoms, e.g., 8 to 24 such as 10 to 18 carbon atoms. Examples of alkyl moieties include —(CH2)17CH3 (stearyl), —(CH2)15CH3 (palmityl), and —(CH2)13CH3 (myristyl). In some embodiments, R1R2N— in the above formula is 1,2-dimyristylamine, wherein both alkyl groups are —(CH2)13CH3 (myristyl).

In some embodiments, the polymer P comprises poly-2-(2-(2-aminoethoxy)ethoxy)acetic acid (pAEEA) or poly-2-(2-(2-methylaminoethoxy)ethoxy)acetic acid (pMAEEA), or a derivative thereof. In some embodiments, the polymer P comprises the following general formula:

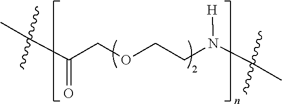

wherein n is 5 to 50, e.g., 5 to 25, e.g., 7 to 14, e.g., 10 to 25, e.g., 14 to 17. In some embodiments, n is 8 or 14. In some embodiments, n is 14. In some embodiments, R1 and R2 in the above formula are —(CH2)13CH3 (myristyl) and the polymer P comprises the following general formula:

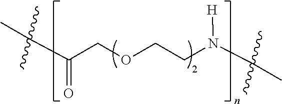

wherein n is 14.

In some embodiments, the connector compound (hydrophobic moiety having a binding moiety covalently attached thereto) comprises the following general formula:

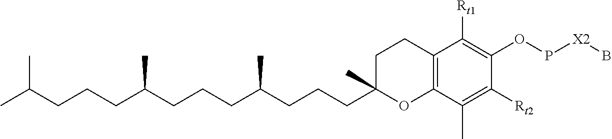

wherein P, X2 and B are as described above and each of Rt1 and Rt2 is independently H or methyl. In some embodiments, Rt1 and Rt2 are both methyl. In some embodiments, Rt1 is methyl, and Rt2 is H. In some embodiments, Rt1 is H, and Rt2 is methyl. In some embodiments, Rt1 and Rt2 are both H.

In some embodiments, the connector compound (hydrophobic moiety having a binding moiety covalently attached thereto) comprises the following general formula:

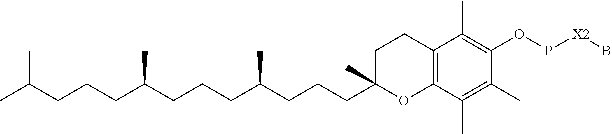

wherein P, X2 and B are as described above.

In some embodiments, the polymer P in the above formulas comprises poly-2-(2-(2-aminoethoxy)ethoxy)acetic acid (pAEEA) or poly-2-(2-(2-methylaminoethoxy)ethoxy)acetic acid (pMAEEA), or a derivative thereof. In some embodiments, the polymer P comprises the following general formula:

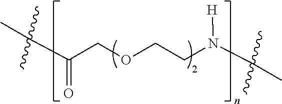

wherein n is 5 to 50, e.g., 5 to 25, e.g., 7 to 14, e.g., 10 to 25, e.g., 14 to 17. In some embodiments, n is 8 or 14. In some embodiments, n is 8. In some embodiments, n is 14.

In some embodiments, the connector compound (hydrophobic moiety having a binding moiety covalently attached thereto) comprises the following general formula:

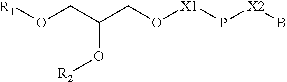

wherein X1, P, X2 and B are as described above and R1 and R2 independently comprise an acyl moiety. In some embodiments, at least one, e.g., each acyl moiety is straight or branched, preferably straight. In some embodiments, at least one, e.g., each acyl moiety has at least 8 carbon atoms, e.g., 8 to 24 such as 10 to 18 carbon atoms. Preferably, at least one, e.g., each acyl moiety is the acyl moiety of a fatty acid, more preferably at least one, e.g., each acyl moiety is the acyl moiety of a fatty acid having at least 8 carbon atoms, e.g., 8 to 24 such as 10 to 18 carbon atoms. Examples of acyl moieties include CH3(CH2)16C(O)— (stearoyl), CH3(CH2)14C(O)— (palmitoyl), and CH3(CH2)12C(O)— (myristoyl). In some embodiments, both acyl groups are CH3(CH2)16C(O)— (stearoyl). In some embodiments, both acyl groups are CH3(CH2)12C(O)— (myristoyl). In some embodiments, X1 is absent or comprises —HPO3-(CH2)n-NH—, wherein n is 1 to 5, e.g., 2.

In some embodiments, the polymer P comprises poly-2-(2-(2-aminoethoxy)ethoxy)acetic acid (pAEEA) or poly-2-(2-(2-methylaminoethoxy)ethoxy)acetic acid (pMAEEA), or a derivative thereof. In some embodiments, the polymer P comprises the following general formula:

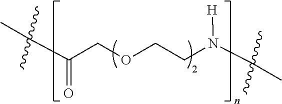

wherein n is 5 to 50, e.g., 5 to 25, e.g., 7 to 14, e.g., 10 to 25, e.g., 14 to 17. In some embodiments, n is 8 or 14. In some embodiments, n is 8. In some embodiments, n is 14.

In some embodiments, the polymer P comprises a pSar. In some embodiments, the polymer P comprises the following general formula:

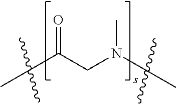

wherein s is 2 to 200, e.g., 5 to 100, e.g., 10 to 50, e.g., 15 to 40. In some embodiments, s is 20 or 23.

In some embodiments, the connector compound (hydrophobic moiety having a binding moiety covalently attached thereto) comprises the following general formula:

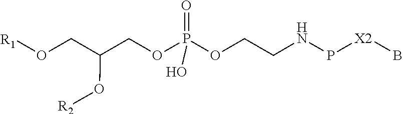

wherein P, X2 and B are as described above and R1 and R2 independently comprise an acyl moiety. In some embodiments, at least one, e.g., each acyl moiety is straight or branched, preferably straight. In some embodiments, at least one, e.g., each acyl moiety has at least 8 carbon atoms, e.g., 8 to 24 such as 10 to 18 carbon atoms. Preferably, at least one, e.g., each acyl moiety is the acyl moiety of a fatty acid, more preferably at least one, e.g., each acyl moiety is the acyl moiety of a fatty acid having at least 8 carbon atoms, e.g., 8 to 24 such as 10 to 18 carbon atoms. Examples of acyl moieties include CH3(CH2)16C(O)— (stearoyl), CH3(CH2)14C(O)— (palmitoyl), and CH3(CH2)12C(O)— (myristoyl). In some embodiments, both acyl groups are CH3(CH2)16C(O)— (stearoyl). In some embodiments, both acyl groups are CH3(CH2)12C(O)— (myristoyl).

In some embodiments, the polymer P comprises poly-2-(2-(2-aminoethoxy)ethoxy)acetic acid (pAEEA) or poly-2-(2-(2-methylaminoethoxy)ethoxy)acetic acid (pMAEEA), or a derivative thereof. In some embodiments, the polymer P comprises the following general formula:

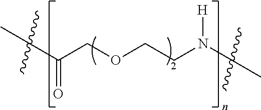

wherein n is 5 to 50, e.g., 5 to 25, e.g., 7 to 14, e.g., 10 to 25, e.g., 14 to 17. In some embodiments, n is 8 or 14. In some embodiments, n is 8. In some embodiments, n is 14.

In some embodiments, n is 8 and R1 and R2 are CH3(CH2)16C(O)— (stearoyl). In some embodiments, n is 14 and R1 and R2 are CH3(CH2)16C(O)— (stearoyl).

In some embodiments, n is 8 and R1 and R2 are CH3(CH2)12C(O)— (myristoyl). In some embodiments, n is 14 and R1 and R2 are CH3(CH2)12C(O)— (myristoyl).

In some embodiments, the polymer P comprises a pSar. In some embodiments, the polymer P comprises the following general formula:

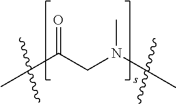

wherein s is 2 to 200, e.g., 5 to 100, e.g., 10 to 50, e.g., 15 to 40. In some embodiments, s is 20 or 23.

In some embodiments, s is 20 and R1 and R2 are CH3(CH2)16C(O)— (stearoyl).

In some embodiments, s is 20 and R1 and R2 are CH3(CH2)12C(O)— (myristoyl).

In some embodiments, X2 in the above formulas comprises the reaction product of a thiol or cysteine reactive group, e.g., a maleimide group, with a compound comprising a thiol or cysteine group. In some embodiments, the compound comprising a thiol or cysteine group comprises the formula SH(CH2)nC(O)—, wherein n ranges from 1 to 5. In some embodiments, n is 2. In some embodiments, X2 comprises a thiosuccinimide moiety.

In some embodiments, X2 comprises the following general formula:

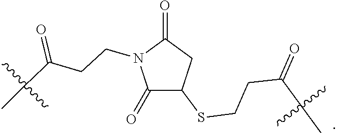

In some embodiments, X2 comprises the following general formula:

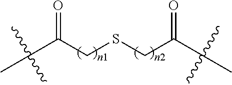

wherein n1 and n2 are independently 1 to 5. In some embodiments, n1 is 1 and n2 is 2. In some embodiments, n1 is 2 and n2 is 1.

The present disclosure provides in one aspect, a connector compound as described herein. In some embodiments of the connector compound, the binding moiety comprises an epitope tag, e.g., an ALFA-tag such as an ALFA-tag described herein.

The present disclosure provides in one aspect, a compound having the following general formula:

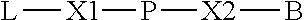

wherein
P comprises a polymer;
L comprises a hydrophobic moiety (e.g., lipid) attached to a first end of the polymer;
B comprises an epitope tag, e.g., an ALFA-tag such as an ALFA-tag described herein, attached to a second end of the polymer;
X1 is absent or a first linking moiety; and
X2 is absent or a second linking moiety.

In some embodiments, X1 comprises a carbonyl group. In some embodiments, L comprises a phosphatidylethanolamine which may be linked to P by an amide group.

In some embodiments, X2 comprises the reaction product of a thiol or cysteine reactive group, e.g., a maleimide group, with a thiol or cysteine group of a compound comprising the epitope tag. In some embodiments, X2 comprises a thiosuccinimide moiety.

In some embodiments, L comprises a lipid as described above. In some embodiments, L comprises DSPE (distearoylphosphatidylethanolamine), DPPE (dipalmitoylphosphatidylethanolamine), DOPE (dioleoylphosphatidylethanolamine), and POPE (palmitoyloleylphosphatidylethanolamine) which may be linked to P by an amide group.

In some embodiments, P comprises a polymer as described above. In some embodiments, P comprises a polymer which provides stealth property, extends circulation half-life and/or reduces non-specific protein binding or cell adhesion. In some embodiments, P comprises a polymer selected from the group consisting of poly(ethylene glycol) (PEG), polysarcosine (pSar) (poly(N-methylglycine), polyoxazoline (POX), polyoxazine (POZ), and poly-2-(2-(2-aminoethoxy)ethoxy)acetic acid (pAEEA) (including derivatives thereof). In some embodiments, P comprises polyethyleneglycol (PEG); e.g., PEG as described above.

In some embodiments, L-X1-P comprises an amphiphilic derivative of a polymer as described above. In some embodiments, the amphiphilic derivative of a polymer comprises a conjugate of disteroyl-glycero-phosphoethanolamine (DSPE) and a polymer, e.g., a polymer as described above. In some embodiments, the amphiphilic derivative of a polymer comprises a disteroyl-glycero-phosphoethanolamine-polyethyleneglycol-conjugate (DSPE-PEG).

In some embodiments, the connector compound is obtainable by reacting the thiol or cysteine reactive group of a reagent comprising an amphiphilic derivative of a polymer, e.g., a PEG reagent comprising a hydrophobic moiety (e.g., lipid), with a thiol or cysteine group of a compound comprising the epitope tag.

In some embodiments, the thiol or cysteine reactive group comprises a maleimide group.

In some embodiments, the PEG reagent comprises DSPE-PEG-maleimide. In some embodiments, the compound comprising the epitope tag comprises the formula SH(CH2)nC(O)—B, wherein n ranges from 1 to 5 and B comprises the epitope tag. In some embodiments, n is 2.

In some embodiments, the connector compound comprises the reaction product of 1,2-distearoyl-sn-glycero-3-phosphoethanolamine-N-[maleimide(polyethylene glycol)] with a compound comprising the formula SH(CH2)nC(O)—B, wherein n ranges from 1 to 5 and B comprises the epitope tag. In some embodiments, n is 2.

In some embodiments, the connector compound (hydrophobic moiety having a binding moiety covalently attached thereto) comprises the following general formula:

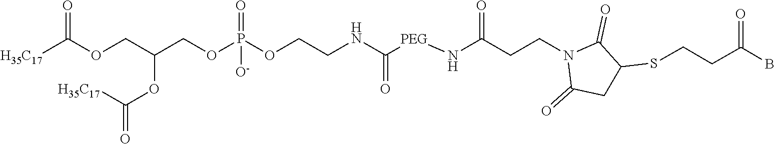

wherein B comprises an epitope tag, e.g., an ALFA-tag such as an ALFA-tag described herein.

In some embodiments, the connector compound (hydrophobic moiety having a binding moiety covalently attached thereto) comprises the following general formula:

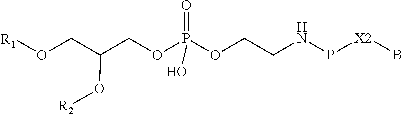

wherein X2 is as described above, R1 and R2 are CH3(CH2)16C(O)— (stearoyl) or CH3(CH2)12C(O)— (myristoyl), polymer P comprises the following general formula:

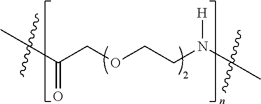

wherein n is 5 to 50, e.g., 5 to 25, e.g., 7 to 14, e.g., 10 to 25, e.g., 14 to 17, e.g., 8 or 14, and B comprises an epitope tag, e.g., an ALFA-tag such as an ALFA-tag described herein.

In some embodiments, n is 8 and R1 and R2 are CH3(CH2)16C(O)— (stearoyl). In some embodiments, n is 14 and R1 and R2 are CH3(CH2)16C(O)— (stearoyl).

In some embodiments, n is 8 and R1 and R2 are CH3(CH2)12C(O)— (myristoyl). In some embodiments, n is 14 and R1 and R2 are CH3(CH2)12C(O)— (myristoyl).

In some embodiments, X2 comprises the following general formula:

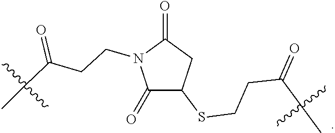

In some embodiments, the connector compound (hydrophobic moiety having a binding moiety covalently attached thereto) comprises the following general formula:

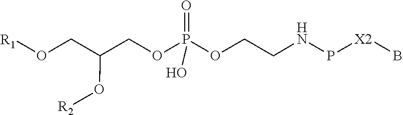

wherein X2 is as described above, R1 and R2 are CH3(CH2)16C(O)— (stearoyl) or CH3(CH2)12C(O)— (myristoyl), polymer P comprises the following general formula:

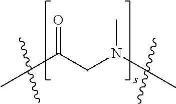

wherein s is 2 to 200, e.g., 5 to 100, e.g., 10 to 50, e.g., 15 to 40, e.g., 20 or 23, and B comprises an epitope tag, e.g., an ALFA-tag such as an ALFA-tag described herein.

In some embodiments, s is 20 and R1 and R2 are CH3(CH2)16C(O)— (stearoyl).

In some embodiments, s is 20 and R1 and R2 are CH3(CH2)12C(O)— (myristoyl).

In some embodiments, X2 comprises the following general formula:

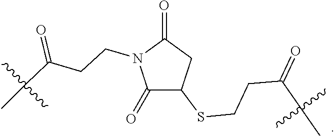

In some embodiments, B comprises a moiety comprising the structure —N-peptide-C(O)—NH2, wherein peptide comprises an epitope tag, e.g., an ALFA-tag such as an ALFA-tag described herein.

The present disclosure provides in one aspect, a connector compound as described above which is integrated in a particle (e.g., a particle as described herein) via a hydrophobic component (e.g., lipid component) of the connector compound.

### Complex

Complexes with particles which are functionalized as described herein (i.e., functionalized with a hydrophobic moiety having a binding moiety covalently attached thereto, also called connector compound), and a compound comprising (i) a moiety binding to the binding moiety covalently attached to the hydrophobic moiety and (ii) a moiety targeting a cell-surface antigen, also called docking compound, may be used ex vivo/in vitro or in vivo for delivering nucleic acids to immune effector cells such as B cells or T cells, in particular CD8+ T cells, thus producing immune effector cells genetically modified to express a first antigen receptor and an activator molecule.

If the immune effector cell to be targeted is a T cell, the cell-surface antigen is a cell surface molecule on T cells, e.g., a T cell marker.

As used herein, the term “T cell marker” refers to surface molecules on T cells which are specific for particular T cells. T cell markers suitable for use herein include, but are not limited to surface CD3, CD4, CD8, CD45RO or any other CD antigen specific for T cells.

If the immune effector cell to be targeted is a B cell, the cell-surface antigen is a cell surface molecule on B cells, e.g., a B cell marker.

As used herein, the term “B cell marker” refers to surface molecules on B cells which are specific for antigen-specific IgG-producing B cells. B cell markers suitable for use herein include, but are not limited to surface IgG, kappa and lambda chains, Ig-alpha (CD79alpha), Ig-beta (CD79beta), CD19, Ia, Fc receptors, B220 (CD45R), CD20, CD21, CD22, CD23, CD81 (TAPA-1) or any other CD antigen specific for B cells.

In some embodiments, the immune effector cell to be targeted is a T cell.

In some embodiments, the moiety targeting a cell-surface antigen of the docking compound is directed against CD8. In some embodiments, the moiety targeting a cell-surface antigen of the docking compound directed against CD8 is selected from the group consisting of an anti-CD8 DARPin, an anti-CD8 VHH and an anti-CD8 scFv. In some embodiments, the moiety binding to a connector compound is a NbALFA-nanobody (NbALFA). Accordingly, in some embodiments, the docking compound may have a structure selected from the group consisting of NbALFA×anti-CD8 DARPin, NbALFA×anti-CD8 VHH and NbALFA×anti-CD8 scFv. In these embodiments, the connector compound may comprise the structure L-X1-P-X2-B described above, wherein B comprises an ALFA-tag.

In some embodiments, the moiety targeting a cell-surface antigen of the docking compound is directed against CD4. In some embodiments, the moiety targeting a cell-surface antigen of the docking compound directed against CD4 is selected from the group consisting of an anti-CD4 DARPin, an anti-CD4 VHH and an anti-CD4 scFv. In some embodiments, the moiety binding to a connector compound is a NbALFA-nanobody (NbALFA). Accordingly, in some embodiments, the docking compound may have a structure selected from the group consisting of NbALFA×anti-CD4 DARPin, NbALFA×anti-CD4 VHH and NbALFA×anti-CD4 scFv. In these embodiments, the connector compound may comprise the structure L-X1-P-X2-B described above, wherein B comprises an ALFA-tag.

In some embodiments, the moiety targeting a cell-surface antigen of the docking compound is directed against CD3. In some embodiments, the moiety targeting a cell-surface antigen of the docking compound directed against CD3 is selected from the group consisting of an anti-CD3 DARPin, an anti-CD3 VHH and an anti-CD3 scFv. In some embodiments, the moiety binding to a connector compound is a NbALFA-nanobody (NbALFA). Accordingly, in some embodiments, the docking compound may have a structure selected from the group consisting of NbALFA×anti-CD3 DARPin, NbALFA×anti-CD3 VHH and NbALFA×anti-CD3 scFv. In these embodiments, the connector compound may comprise the structure L-X1-P-X2-B described above, wherein B comprises an ALFA-tag.

The docking compound may form a connection, such as a non-covalent or covalent connection, to a particle to be delivered to a target cell through a connector compound. The connector compound comprises a binding moiety for binding to the docking compound which is covalently attached to a hydrophobic moiety (e.g., lipid). The hydrophobic moiety (e.g., lipid) forms part of said particle.

In some embodiments, a docking compound comprises a “cell-surface antigen targeting moiety”, e.g., a moiety targeting a cell surface antigen on target cells, that is capable of binding to the cell surface antigen on target cells. A “cell-surface antigen targeting moiety” as used herein relates to the part of the docking compound which binds to a cell-surface antigen. These moieties can be any peptide or protein (e.g. antibodies or antibody fragments) binding to the cell-surface antigen. Particular embodiments of suitable cell-surface antigen targeting moieties for use herein include cell surface antigen binding moieties, such as antibodies, antibody fragments and DARPins.

A cell-surface antigen targeting moiety preferably binds with high specificity and/or high affinity and the bond with the primary target is preferably stable within the body.

In order to allow specific targeting of cell-surface antigens, the cell-surface antigen targeting moiety of the docking compound can comprise compounds including but not limited to antibodies, antibody fragments, e.g. Fab2, Fab, scFV, VHH domains, and other proteins or peptides.

According to some embodiments, the cell-surface antigen is a T cell antigen, e.g., CD3, such as CD3e, CD8 or CD4, and suitable cell-surface antigen targeting moieties include but are not limited to, peptides and polypeptides targeting the cell surface antigen, e.g., antibodies, antibody fragments and DARPins.

According to some embodiments, the cell-surface antigen and cell-surface antigen targeting moiety are selected so as to result in the specific or increased targeting of certain cells. This can be achieved by selecting primary targets with cell-specific expression. For example, T cell antigens, e.g., those described herein, may be expressed in T cells while they are not expressed or expressed in a lower amount in other cells.

The docking compound further comprises a group which serves as a binding partner for a respective binding moiety of a connector compound. The portion of the connector compound comprising the hydrophobic moiety (e.g., lipid) (having a binding moiety for the docking compound covalently attached) integrates into a particle carrying a payload and thus forms a connection between the particle and the docking compound. The moiety of the docking compound binding to the connector compound and the cell-surface antigen targeting moiety are linked to each other, preferably by a covalent linkage.

According to some embodiments, the docking compound comprises a bispecific molecule, such as a bispecific polypeptide, e.g., a bispecific antibody. In some embodiments, the docking compound comprises a binding domain binding to a cell-surface antigen and a binding domain binding to a connector compound. In some embodiments, the docking compound comprises an antibody or antibody fragment binding to a primary target and an antibody or antibody fragment binding to a connector compound. In some embodiments, at least one binding domain comprises a heavy chain variable region (VH) and a light chain variable region (VL) of an antibody. In some embodiments, each binding domain comprises a heavy chain variable region (VH) and a light chain variable region (VL) of an antibody. In some embodiments, at least one binding domain comprises a single-domain antibody such as a VHH. In some embodiments, each binding domain comprises a single-domain antibody such as a VHH. In some embodiments, one binding domain comprises a heavy chain variable region (VH) and a light chain variable region (VL) of an antibody and the other binding domain comprises a single-domain antibody such as a VHH. In some embodiments, the binding domain binding to a cell-surface antigen target comprises a heavy chain variable region (VH) and a light chain variable region (VL) of an antibody. In some embodiments, the binding domain binding to a cell-surface antigen comprises a single-domain antibody such as a VHH. In some embodiments, the binding domain binding to a connector compound comprises a heavy chain variable region (VH) and a light chain variable region (VL) of an antibody. In some embodiments, the binding domain binding to a connector compound comprises a single-domain antibody such as a VHH.

In some embodiments, the docking compound comprises a fusion protein which comprises a binding domain binding to a cell-surface antigen and a binding domain binding to a connector compound.

In some embodiments, the docking compound comprises a single peptide chain. In some embodiments, the single peptide chain comprises a portion, e.g., antibody, antibody fragment or DARPin, binding to a primary target and a portion, e.g., antibody or antibody fragment, binding to a connector compound. In some embodiments, the antibody fragments are VHH, scFv, or a mixture thereof. In different embodiments, the docking compound comprises one of the following structures (from N- to C-terminus):
VHH (α connector compound)-optional linker-VHH (α cell-surface antigen)
VHH (α cell-surface antigen)-optional linker-VHH (α connector compound)
VHH (α connector compound)-optional linker-scFv (α cell-surface antigen)
scFv (α cell-surface antigen)-optional linker-VHH (α connector compound)
VHH (α cell-surface antigen)-optional linker-scFv (α connector compound)
scFv (α connector compound)-optional linker-VHH (α cell-surface antigen)
scFv (α connector compound)-optional linker-scFv (α cell-surface antigen)
scFv (α cell-surface antigen)-optional linker-scFv (α connector compound)

The present disclosure provides in one aspect, a docking compound as described herein. In some embodiments, the docking compound comprises a bispecific molecule, such as a bispecific polypeptide, e.g., a bispecific antibody, wherein one specificity binds to an epitope tag, e.g., an ALFA-tag and the other specificity binds to a cell-surface antigen, e.g., a cell surface antigen on target cells. In some embodiments, the specificity which binds to an epitope tag is an antibody or antibody fragment such as an NbALFA-nanobody (NbALFA). In some embodiments, the specificity which binds to a cell-surface antigen is an antibody, antibody fragment or DARPin. In some embodiments, the moiety targeting a primary target of the docking compound is selected from the group consisting of an anti-cell-surface antigen DARPin, an anti-cell-surface antigen VHH and an anti-cell-surface antigen scFv and/or the moiety binding to a connector compound of the docking compound is an NbALFA-nanobody (NbALFA). In some embodiments, the docking compound has a structure selected from the group consisting of NbALFA×anti-cell-surface antigen DARPin, NbALFA×anti-cell-surface antigen VHH and NbALFA×anti-cell-surface antigen scFv. In some embodiments, the cell-surface antigen is a T cell antigen, e.g., CD3, such CD3e, CD4 or CD8. In some embodiments, the docking compound comprises a bispecific antibody comprising a nanobody which binds to an epitope tag, e.g., an ALFA-tag, and an anti-CD3 VHH. In some embodiments, the docking compound comprises a bispecific antibody comprising a nanobody which binds to an epitope tag, e.g., an ALFA-tag, and an anti-CD3 scFv. In some embodiments, the docking compound comprises a bispecific molecule comprising a nanobody which binds to an epitope tag, e.g., an ALFA-tag, and an anti-CD3 DARPin. In some embodiments, the docking compound comprises a bispecific antibody comprising a nanobody which binds to an epitope tag, e.g., an ALFA-tag, and an anti-CD4 VHH. In some embodiments, the docking compound comprises a bispecific antibody comprising a nanobody which binds to an epitope tag, e.g., an ALFA-tag, and an anti-CD4 scFv. In some embodiments, the docking compound comprises a bispecific molecule comprising a nanobody which binds to an epitope tag, e.g., an ALFA-tag, and an anti-CD4 DARPin. In some embodiments, the docking compound comprises a bispecific antibody comprising a nanobody which binds to an epitope tag, e.g., an ALFA-tag, and an anti-CD8 VHH. In some embodiments, the docking compound comprises a bispecific antibody comprising a nanobody which binds to an epitope tag, e.g., an ALFA-tag, and an anti-CD8 scFv. In some embodiments, the docking compound comprises a bispecific molecule comprising a nanobody which binds to an epitope tag, e.g., an ALFA-tag, and an anti-CD8 DARPin.

In some embodiments, the moiety on the connector compound (binding moiety covalently attached to a hydrophobic moiety) and the moiety on the docking compound (moiety binding to the binding moiety covalently attached to a hydrophobic moiety) interacting which each other non-covalently bind to each other.

In some embodiments, the moieties on the connector compound and on the docking compound interacting which each other bind to each other under physiological conditions.

In some embodiments, the moieties on the connector compound and on the docking compound interacting which each other are antibody/antigen systems.

In some embodiments, the moiety of the connector compound binding to the docking compound comprises a peptide or protein, e.g., a peptide tag, and the moiety of the docking compound binding to the connector compound comprises a binder, e.g., an antibody or antibody fragment, binding to the peptide or protein.

In some embodiments, the moiety of the docking compound binding to the connector compound comprises a peptide or protein, e.g., a peptide tag, and the moiety of the connector compound binding to the docking compound comprises a binder, e.g., an antibody or antibody fragment, binding to the peptide or protein. In some embodiments, the moieties on the connector compound and on the docking compound interacting which each other comprise an epitope tag/binder system.

As used herein, an “epitope tag” refers to a stretch of amino acids to which an antibody or proteinaceous molecule with antibody-like function can bind.

In some embodiments, the epitope tag comprises an ALFA-tag. In some embodiments, the epitope tag/binder system comprises an ALFA-tag and an ALFA-specific single-domain antibody (sdAb), NbALFA-nanobody.

In some embodiments, an ALFA-tag comprises a sequence selected from the group consisting of SRLEEELRRRLTE, PSRLEEELRRRLTE, SRLEEELRRRLTEP, and PSRLEEELRRRLTEP.

In some embodiments, an ALFA-tag comprises the cyclized amino acid sequence
AA0-AA1-AA2-AA3-AA4-AA5-AA6-AA7-AA8-AA9-AA10-AA11-AA12-AA13-AA14-,
   wherein the side-chains of any two of the amino acids of AA0, AA1, AA2, AA3, AA4, AA5, AA6, AA7, AA8, AA9, AA10, AA11, AA12, AA13 and AA14 (X1, X2) are connected covalently; and
   wherein the amino acids of AA0, AA1, AA2, AA3, AA4, AA5, AA6, AA7, AA8, AA9, AA10, AA1 1, AA12, AA13 and AA14 which are not X1 and X2 are:
AA0 is Pro or deleted;
AA1 is Ser, Gly, Thr, or Pro;
AA2 is Arg, Gly, Ala, Glu, or Pro;
AA3 is Leu, Ile, or Val;
AA4 is Glu or Gln;
AA5 is Glu or Gln;
AA6 is Glu or Gln;
AA7 is Leu, Ile, or Val;
AA8 is Arg, Ala, Gln, or Glu;
AA9 is Arg, Ala, Gln, or Glu;
AA10 is Arg;
AA1l is Leu;
AA12 is Thr, Ser, Asp, Glu, Pro, Ala, or deleted;
AA13 is Glu, Lys, Pro, Ser, Ala, Asp, or deleted; and
AA14 is Pro or deleted.

In some embodiments, X1 and X2 are separated by 2 or 3 amino acids.

In some embodiments, AA5 is X1 and AA9 is X2, AA5 is X1 and AA8 is X2, AA9 is X1 and AA13 is X2, AA6 is X1 and AA9 is X2, AA9 is X1 and AA12 is X2, AA10 is X1 and AA13 is X2, AA6 is X1 and AA10 is X2 or AA4 is X1 and AA8 is X2.

In some embodiments, X₁ and X₂ in the peptides disclosed herein are connected covalently via an amide, disulfide, thioether, ether, ester, thioester, thioamide, alkylene, alkenylene, alkynylene, and/or 1,2,3-triazole.

In some embodiments, a cyclized amino acid sequence described herein is generated by linking an amino group of a side-chain of one of X₁ and X₂ to the carboxyl group of a side-chain of the other of X₁ and X₂ via an amide bond. The amino group of the side chain of an amino acid that possesses a pendant amine group, e.g., lysine or a lysine derivative, and the carboxyl group of the side chain of an acidic amino acid, e.g., aspartic acid, glutamic acid or a derivative thereof, can be used to generate a cyclized amino acid sequence via an amide bond.

In some embodiments, a cyclized amino acid sequence described herein is generated by linking a sulfhydryl group of a side-chain of one of X₁ and X₂ to the sulfhydryl group of a side-chain of the other of X₁ and X₂ via a disulfide bond. Sulfhydryl group-containing amino acids include cysteine and other sulfhydryl-containing amino acids as Pen.

In some embodiments, X₁ and X₂ are, independently, selected from the group consisting of Glu, DGlu, Asp, DAsp, Lys, DLys, hLys, DhLys, Orn, DOrn, Dab, DDab, Dap, DDap, Cys, DCys, hCys, DhCys, Pen, and DPen, with the proviso that when X₁ is Glu, DGlu, Asp, or DAsp, X₂ is Lys, DLys, hLys, DhLys, Orn, DOrn, Dab, DDab, Dap, or DDap; when X1 is Lys, DLys, hLys, DhLys, Orn, DOrn, Dab, DDab, Dap, or DDap, X2 is Glu, DGlu, Asp, or DAsp; and when X1 is Cys, DCys, hCys, DhCys, Pen, or DPen, X2 is Cys, DCys, hCys, DhCys, Pen, or DPen.

In some embodiments, the cyclized amino acid sequence is -Ser-Arg-Leu-Glu-cyclo(Glu-Glu-Leu-Arg-Lys)-Arg-Leu-Thr-Glu-. In some other embodiments, the cyclized amino acid sequence is -Ser-Arg-Leu-Glu-cyclo(Asp-Glu-Leu-Arg-Lys)-Arg-Leu-Thr-Glu-. In yet some other embodiments, the cyclized amino acid sequence is -Ser-Arg-Leu-Glu-cyclo(Glu-Glu-Leu-Lys)-Arg-Arg-Leu-Thr-Glu-. In still some other embodiments, the cyclized amino acid sequence is -Ser-Arg-Leu-Glu-Glu-Glu-Leu-Arg-cyclo(Lys-Arg-Leu-Thr-Glu)-.

The cyclic peptides may have different cyclic bridging moieties forming the ring structure. Preferably, chemically stable bridging moieties are included in the ring structure such as, for example, an amide group, a lactone group, an ether group, a thioether group, a disulfide group, an alkylene group, an alkenyl group, or a 1,2,3-triazole. The following are examples illustrating the variability of bridging moieties in a peptide:

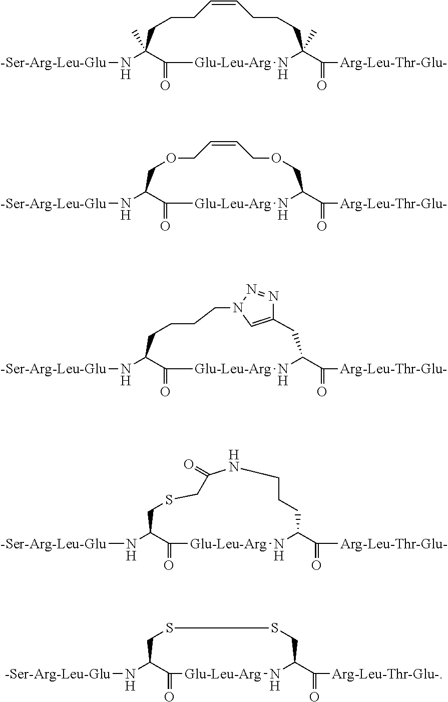

In some embodiments, an ALFA-tag binding moiety comprises an antibody or antibody fragment, e.g., a camelid VHH domain. In some embodiments, an ALFA-tag binding moiety comprises a single-domain antibody (sdAb), NbALFA-nanobody.

In some embodiments, an ALFA-tag binding moiety comprises a single domain antibody, e.g., a camelid VHH domain comprising the CDR1 sequence VTX₁SALNAMAMG, wherein X₁ is I or V, the CDR2 sequence AVSX₂RGNAM, wherein X₂ is E, H, N, D, or S, and the CDR3 sequence LEDRVDSFHDY.

In some embodiments, an ALFA-tag binding moiety comprises a single domain antibody, e.g., a camelid VHH domain comprising the CDR1 sequence GVTX₁SALNAMAMG, wherein X₁ is I or V, the CDR2 sequence AVSX₂RGNAM, wherein X2 is E, H, N, D, or S, and the CDR3 sequence LEDRVDSFHDY.

In some embodiments, an ALFA-tag binding moiety comprises a single domain antibody, e.g., a camelid VHH domain comprising the CDR1 sequence VTISALNAMAMG, the CDR2 sequence AVSERGNAM, and the CDR3 sequence LEDRVDSFHDY.

In some embodiments, an ALFA-tag binding moiety comprises a single domain antibody, e.g., a camelid VHH domain comprising the CDR1 sequence GVTISALNAMAMG, the CDR2 sequence AVSERGNAM, and the CDR3 sequence LEDRVDSFHDY.

In some embodiments, an ALFA-tag binding moiety comprises a single domain antibody, e.g., a camelid VHH domain comprising the amino acid sequence EVQLQESGGGLVQPGGSLRLSCTASGVTISALNAMAMGWYRQAPGERRVMVAAVS ERGNAMYRESVQGRFTVTRDFTNKMVSLQMDNLKPEDTAVYYCHVLEDRVDSFHD YWGQGTQVTVSS, an amino acid sequence having at least 99%, 98%, 97%, 96%, 95%, 90%, 85%, or 80% identity to said amino acid sequence, or a fragment of said amino acid sequence or the amino acid sequence having at least 99%, 98%, 97%, 96%, 95%, 90%, 85%, or 80% identity to said amino acid sequence. In some embodiments, the amino acid sequence comprises CDR1, CDR2 and CDR3 sequences as described above.

In some embodiments, the epitope tag/binder system comprises an epitope tag comprising the sequence PDRVRAVSHWSS (Spot-tag) and the binder comprises a single-domain antibody (sdAb, or nanobody) (Spot-nanobody (14.7 kD)) that specifically binds to the Spot-tag.

In some embodiments, following binding of the moieties on the connector compound and on the docking compound interacting which each other, a covalent connection is formed. In these embodiments, the system used herein may comprise a Tag/Catcher system forming a covalent bond, e.g., SpyTag/SpyCatcher forming an isopeptide bond.

The SpyTag/SpyCatcher system is a technology for irreversible conjugation of recombinant proteins. The peptide SpyTag spontaneously reacts with the protein SpyCatcher to form an intermolecular isopeptide bond between the pair. Using the Tag/Catcher pair, bioconjugation can be achieved between two recombinant proteins.

The present disclosure provides in one aspect, a complex wherein a particle comprising a connector compound (hydrophobic moiety having a binding moiety covalently attached thereto) is bound to a docking compound (compound comprising (i) a moiety binding to the binding moiety covalently attached to a hydrophobic moiety and (ii) a moiety targeting a cell surface antigen). Thus, the connector compound and the docking compound comprise moieties interacting which each other.

Accordingly, the present disclosure provides in one aspect, a complex comprising:
(a) a particle comprising a hydrophobic moiety having a binding moiety covalently attached thereto, and
(b) a compound comprising (i) a moiety binding to the binding moiety covalently attached to the hydrophobic moiety and (ii) a moiety targeting a cell surface antigen.

Different embodiments of the connector compound and the docking compound which are complexed are described herein.

In some embodiments, the connector compound comprises an ALFA-tag. In these embodiments, the moiety binding to a connector compound of the docking compound may be a NbALFA-nanobody (NbALFA). In some embodiments, the docking compound may have a structure selected from the group consisting of NbALFA×anti-cell-surface antigen DARPin, NbALFA×anti-cell-surface antigen VHH and NbALFA×anti-cell-surface antigen scFv.

### Pharmaceutical Compositions

The particles, complexes or immune effector cells described herein may be administered in pharmaceutical compositions or medicaments and may be administered in the form of any suitable pharmaceutical composition. In some embodiments, the pharmaceutical composition is for therapeutic or prophylactic treatments, e.g., for use in treating or preventing a disease involving an antigen such as a cancer disease or an infectious disease.

The term “pharmaceutical composition” relates to a composition comprising a therapeutically effective agent, preferably together with pharmaceutically acceptable carriers, diluents and/or excipients. Said pharmaceutical composition is useful for treating, preventing, or reducing the severity of a disease by administration of said pharmaceutical composition to a subject.

The pharmaceutical compositions of the present disclosure may comprise one or more adjuvants or may be administered with one or more adjuvants. In some embodiments, the pharmaceutical composition does not comprise an adjuvant. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The chemokines may be IL-1, IL-2, IL-3, IL-4, IL-5, IL-6, IL-7, IL-8, IL-9, IL-10, IL-12, INFa, INF-γ, GM-CSF, LT-a. Further known adjuvants are aluminum hydroxide, Freund's adjuvant or oil such as Montanide® ISA51. Other suitable adjuvants for use in the present disclosure include lipopeptides, such as Pam3Cys, as well as lipophilic components, such as saponins, trehalose-6,6-dibehenate (TDB), monophosphoryl lipid-A (MPL), monomycoloyl glycerol (MMG), or glucopyranosyl lipid adjuvant (GLA).

The pharmaceutical compositions of the present disclosure may be in a storable form (e.g., in a frozen or lyophilized/freeze-dried form) or in a “ready-to-use form” (i.e., in a form which can be immediately administered to a subject, e.g., without any processing such as diluting). Thus, prior to administration of a storable form of a pharmaceutical composition, this storable form has to be processed or transferred into a ready-to-use or administrable form. E.g., a frozen pharmaceutical composition has to be thawed, or a freeze-dried pharmaceutical composition has to be reconstituted, e.g. by using a suitable solvent (e.g., deionized water, such as water for injection) or liquid (e.g., an aqueous solution).

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In some embodiments relating to the treatment of a particular disease, the desired reaction may relate to inhibition of the course of the disease. This comprises slowing down the progress of the disease and, in some embodiments, interrupting or reversing the progress of the disease. The desired reaction in a treatment of a disease may also be delay of the onset or a prevention of the onset of said disease or said condition, or symptoms thereof. An effective amount of the pharmaceutical compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the pharmaceutical compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain buffers, preservatives, and optionally other therapeutic agents. In some embodiments, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some embodiments, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

### Methods of Producing Immune Effector Cells

The immune effector cells described herein may be produced by any method known to the skilled person, as long as it produces an immune effector cell having the first and second nucleic acid and being capable of expressing these nucleic acids.

The order of introducing the nucleic acids into the cell is not particularly limited. In some embodiments, the first nucleic acid is introduced into the immune effector before the second nucleic acid. In some embodiments, the first nucleic acid is introduced into the immune effector cell after the second nucleic acid. In some embodiments, the first nucleic acid is introduced into the immune effector cell after the second nucleic acid, but before the fourth and/or fifth nucleic acid.

In some embodiments, the first and third nucleic acid are introduced into the immune effector cell simultaneously and before the second nucleic acid.

In some embodiments, the first and third nucleic acid are introduced into the immune effector cell simultaneously and after the second nucleic acid.

In some embodiments, the first, second, third, fourth and fifth nucleic acid are introduced into the immune effector cell simultaneously, in particular with a particle or complex as described herein.

Various methods can be used to introduce the nucleic acids into the immune effector cell. These include, for example, electroporation, lipid-based transfection, polymer-based transfections, or viral-based transfection.

In some embodiments, in the methods of producing immune effector cells particles or complexes described herein are used.

In some embodiments, the immune effector cells may be produced in vivo, and therefore nearly instantaneously, using particles such as nanoparticles described herein targeted to immune effector cells, in particular T cells. For example, particles may be coupled to a docking compound, forming a complex, comprising a moiety for binding to CD3, e.g., CD3e, on T cells, e.g., anti-CD3 VHH or anti-CD3 F(ab) fragment. Upon binding to immune effector cells, in particular T cells, these particles may be endocytosed. Their contents, for example nucleic acid encoding antigen receptor, e.g., plasmid DNA encoding an anti-tumor antigen CAR, may be directed to the T cell nucleus due to, for example, the inclusion of peptides containing microtubule-associated sequences (MTAS) and nuclear localization signals (NLSs). The inclusion of transposons flanking the nucleic acid encoding antigen receptor, e.g., the CAR gene expression cassette, and a separate nucleic acid, e.g., plasmid, encoding a hyperactive transposase, may allow for the efficient integration of the nucleic acid encoding antigen receptor, e.g., the CAR vector, into chromosomes.

Another possibility is to use the CRISPR/Cas9 method to deliberately place a peptide/polypeptide coding sequence, e.g., an antigen receptor coding sequence such as a CAR coding sequence, at a specific locus. For example, existing T cell receptors (TCR) may be knocked out, while knocking in the CAR and placing it under the dynamic regulatory control of the endogenous promoter that would otherwise moderate TCR expression.

### Methods of Treatment

The agents, compositions and methods described herein can be used to treat a subject with a disease, e.g., a disease characterized by the presence of diseased cells expressing an antigen. The agents, compositions and methods described herein may be used in the therapeutic or prophylactic treatment of various diseases. Particularly preferred diseases are cancer diseases. In some embodiments, the agents, compositions and methods described herein are useful in a prophylactic and/or therapeutic treatment of a disease involving an antigen.

Such antigen may serve as target for immune effector cells genetically modified to express an antigen receptor. For example, if the antigen is derived from a virus, the agents, compositions and methods may be useful in the treatment of a viral disease caused by said virus. If the antigen is a tumor antigen, the agents, compositions and methods may be useful in the treatment of a cancer disease wherein cancer cells express said tumor antigen.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder (e.g., cancer) but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In some embodiments of the disclosure, the aim is to deliver a pharmaceutically active agent, such as immune effector cells, particles or complexes, to diseased cells expressing an antigen such as cancer cells expressing a tumor antigen, and to treat a disease such as a cancer disease involving cells expressing an antigen such as a tumor antigen.

In some embodiments of the disclosure, the aim is to deliver at least two nucleic acids encoding an antigen receptor and an activator molecule, respectively, to immune effector cells to generate immune effector cells genetically modified to express the antigen receptor either in vivo or in vitro. In some embodiments, immune effector cells genetically modified to express an antigen receptor are for targeting diseased cells expressing an antigen such as cancer cells expressing a tumor antigen, and treating a disease such as a cancer disease involving cells expressing an antigen such as a tumor antigen. In some embodiments, immune effector cells expressing an antigen receptor exert one or more immune effector functions on diseased cells, e.g., kill diseased cells by means of a cellular immune response.

The term “disease involving an antigen”, “disease involving cells expressing an antigen” or similar terms refer to any disease which implicates an antigen, e.g., a disease which is characterized by the presence of an antigen. The disease involving an antigen can be an infectious disease, or a cancer disease or simply cancer. As mentioned above, the antigen may be a disease-associated antigen, such as a tumor-associated antigen, a viral antigen, or a bacterial antigen. In some embodiments, a disease involving an antigen is a disease involving cells expressing an antigen, preferably on the cell surface.

The term “infectious disease” refers to any disease which can be transmitted from individual to individual or from organism to organism, and is caused by a microbial agent (e.g. common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer, lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” according to the disclosure also comprises cancer metastases.

The term “solid tumor” or “solid cancer” as used herein refers to the manifestation of a cancerous mass, as is well known in the art for example in Harrison's Principles of Internal Medicine, 14th edition. Preferably, the term refers to a cancer or carcinoma of body tissues other than blood, preferably other than blood, bone marrow, and lymphoid system. For example, but not by way of limitation, solid tumors include cancers of the prostate, lung cancer, colorectal tissue, bladder, oropharyngeal/laryngeal tissue, kidney, breast, endometrium, ovary, cervix, stomach, pancrease, brain, and central nervous system.

The methods and agents described herein are, in particular, useful for the treatment of cancers, e.g., solid cancers, characterized by diseased cells expressing an antigen the first antigen receptor is directed to.

“Cell-mediated immunity”, “cellular immunity”, “cellular immune response”, or similar terms are meant to include a cellular response directed to cells characterized by expression of an antigen, in particular characterized by presentation of an antigen with class I or class II MHC. The cellular response relates to cells called T cells or T lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naive T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

In some embodiments, the immune effector cells, particles, complexes or pharmaceutical compositions described herein may be administered intravenously, intraarterially, subcutaneously, intradermally, dermally, intranodally, intramuscularly, intratumorally, or peritumorally. In some embodiments, the immune effector cells, particles, complexes or pharmaceutical compositions described herein may be administered intramuscularly. In some embodiments, the immune effector cells, particles, complexes or pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In some embodiments, the immune effector cells, particles, complexes or pharmaceutical compositions are formulated for systemic administration. In some embodiments, the systemic administration is by intravenous administration. In some embodiments, the immune effector cells, particles, complexes or pharmaceutical compositions are formulated for intramuscular administration.

Combination strategies in cancer treatment may be desirable due to a resulting synergistic effect, which may be considerably stronger than the impact of a monotherapeutic approach. In one embodiment, immune effector cells, particles, complexes or pharmaceutical compositions described herein may be administered with an immunotherapeutic agent. As used herein “immunotherapeutic agent” relates to any agent that may be involved in activating a specific immune response and/or immune effector function(s). The present disclosure contemplates the use of an antibody as an immunotherapeutic agent. Without wishing to be bound by theory, antibodies are capable of achieving a therapeutic effect against cancer cells through various mechanisms, including inducing apoptosis, block components of signal transduction pathways or inhibiting proliferation of tumor cells. In certain embodiments, the antibody is a monoclonal antibody. A monoclonal antibody may induce cell death via antibody-dependent cell mediated cytotoxicity (ADCC), or bind complement proteins, leading to direct cell toxicity, known as complement dependent cytotoxicity (CDC). Non-limiting examples of anti-cancer antibodies and potential antibody targets (in brackets) which may be used in combination with the present disclosure include: Abagovomab (CA-125), Abciximab (CD41), Adecatumumab (EpCAM), Afutuzumab (CD20), Alacizumab pegol (VEGFR2), Altumomab pentetate (CEA), Amatuximab (MORAb-009), Anatumomab mafenatox (TAG-72), Apolizumab (HLA-DR), Arcitumomab (CEA), Atezolizumab (PD-L1), Bavituximab (phosphatidylserine), Bectumomab (CD22), Belimumab (BAFF), Bevacizumab (VEGF-A), Bivatuzumab mertansine (CD44 v6), Blinatumomab (CD 19), Brentuximab vedotin (CD30 TNFRSF8), Cantuzumab mertansin (mucin CanAg), Cantuzumab ravtansine (MUC1), Capromab pendetide (prostatic carcinoma cells), Carlumab (CNT0888), Catumaxomab (EpCAM, CD3), Cetuximab (EGFR), Citatuzumab bogatox (EpCAM), Cixutumumab (IGF-1 receptor), Claudiximab (Claudin), Clivatuzumab tetraxetan (MUC1), Conatumumab (TRAIL-R2), Dacetuzumab (CD40), Dalotuzumab (insulin-like growth factor I receptor), Denosumab (RANKL), Detumomab (B-lymphoma cell), Drozitumab (DR5), Ecromeximab (GD3 ganglioside), Edrecolomab (EpCAM), Elotuzumab (SLAMF7), Enavatuzumab (PDL192), Ensituximab (NPC-1C), Epratuzumab (CD22), Ertumaxomab (HER2/neu, CD3), Etaracizumab (integrin αvβ3), Farletuzumab (folate receptor 1), FBTA05 (CD20), Ficlatuzumab (SCH 900105), Figitumumab (IGF-1 receptor), Flanvotumab (glycoprotein 75), Fresolimumab (TGF-β), Galiximab (CD80), Ganitumab (IGF-I), Gemtuzumab ozogamicin (CD33), Gevokizumab (ILIP), Girentuximab (carbonic anhydrase 9 (CA-IX)), Glembatumumab vedotin (GPNMB), Ibritumomab tiuxetan (CD20), Icrucumab (VEGFR-1), Igovoma (CA-125), Indatuximab ravtansine (SDC1), Intetumumab (CD51), Inotuzumab ozogamicin (CD22), Ipilimumab (CD 152), Iratumumab (CD30), Labetuzumab (CEA), Lexatumumab (TRAIL-R2), Libivirumab (hepatitis B surface antigen), Lintuzumab (CD33), Lorvotuzumab mertansine (CD56), Lucatumumab (CD40), Lumiliximab (CD23), Mapatumumab (TRAIL-R1), Matuzumab (EGFR), Mepolizumab (IL5), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Namatumab (RON), Necitumumab (EGFR), Nimotuzumab (EGFR), Nivolumab (IgG4), Ofatumumab (CD20), Olaratumab (PDGF-R a), Onartuzumab (human scatter factor receptor kinase), Oportuzumab monatox (EpCAM), Oregovomab (CA-125), Oxelumab (OX-40), Panitumumab (EGFR), Patritumab (HER3), Pemtumoma (MUC1), Pertuzuma (HER2/neu), Pintumomab (adenocarcinoma antigen), Pritumumab (vimentin), Racotumomab (N-glycolylneuraminic acid), Radretumab (fibronectin extra domain-B), Rafivirumab (rabies virus glycoprotein), Ramucirumab (VEGFR2), Rilotumumab (HGF), Rituximab (CD20), Robatumumab (IGF-1 receptor), Samalizumab (CD200), Sibrotuzumab (FAP), Siltuximab (IL6), Tabalumab (BAFF), Tacatuzumab tetraxetan (alpha-fetoprotein), Taplitumomab paptox (CD 19), Tenatumomab (tenascin C), Teprotumumab (CD221), Ticilimumab (CTLA-4), Tigatuzumab (TRAIL-R2), TNX-650 (IL13), Tositumomab (CD20), Trastuzumab (HER2/neu), TRBS07 (GD2), Tremelimumab (CTLA-4), Tucotuzumab celmoleukin (EpCAM), Ublituximab (MS4A1), Urelumab (4-1 BB), Volociximab (integrin α5β1), Votumumab (tumor antigen CTAA 16.88), Zalutumumab (EGFR), and Zanolimumab (CD4).

In one embodiment, immune effector cells, particles, complexes or pharmaceutical compositions described herein may be administered with a cancer vaccine. Preferably, the cancer vaccine is administered after an immune effector cell, particle or complex as described herein has been administered, preferably 2 to 30, 2 to 15, 5, 10, 20, days after administration. In an embodiment, the cancer vaccine is an RNA vaccine. In an embodiment, the RNA of the cancer vaccine encodes an antigen, preferably an antigen to which the first or second cell-surface expressed antigen receptor described herein binds.

The present invention is described in detail and is illustrated by the figures and examples, which are used only for illustration purposes and are not meant to be limiting. Owing to the description and the examples, further embodiments which are likewise included in the invention are accessible to the skilled worker.

### DESCRIPTION OF THE FIGURES

FIG. 1:

FIG. 1 shows four potential approaches using three component (3C) and four component (4C) systems to generate CAR-T cells from unactivated T cells using the Sleeping Beauty transposon system. Top half: components required for each approach. Bottom half: expression of the cargo components—encoded by DNA or RNA molecules supplied to the cells—at different time points post-delivery to the cells and potential interactions between them. 2C=two component system comprising only transposon and transposase. i) 3C system additionally comprising an RNA-encoded receptor that is stimulated by exogenously supplied soluble ligand. ii) 2C system additionally comprising an RNA-encoding a second antigen receptor which is used to stimulate the cells by contact with ligand-expressing cells (also called transCAR system or protocol in some embodiments). iii) 2C system additionally comprising an RNA encoding the target of the DNA-encoded CAR. iv) 4C system: 2C system additionally comprising an RNA encoding a CAR and an RNA encoding the antigen targeted by the CAR.

FIG. 2:

FIG. 2 shows a detailed overview of a transCAR protocol embodiment to generate CAR-T cells in vivo. Nanoparticles (NPs) functionalized with T cell-targeting ligands trigger T cell-specific NP cargo uptake via receptor-mediated endocytosis.

FIG. 3:

FIG. 3 shows data generated during studies of the transCAR protocol embodiment. A) Unactivated primary human CD8+ T cells transfected with CD8-targeting nanoparticles (NP) readily express transfected mRNA. NP-mediated delivery of CAR mRNA (CLDN18.2-CAR-RNA) to CD8+ T cells enables CAR-specific, antigen-mediated T cell-activation and induction of proliferation when transfected T cells are co-cultivated with autologous immature dendritic cells (iDCs) expressing the CAR antigen (CLDN18.2) [black/right bars]. B) When a functional transposon system, consisting of SB100X-encoding RNA and CLDN6 CAR-encoding Minicircle (MC), is co-delivered to the T cells via the NPs together with the CAR mRNA, the proliferating CD8+ T cells acquire increasing amount of CLDN6 CAR expression over time [black bars]. Observed CLDN6 CAR expression results from successful transposition in CD8+ T cells, as indicated by the lack of the expression in the corresponding control group where SB100X-encoding mRNA was replaced by filler RNA (Thy1.1-encoding mRNA). C) Generated CLDN6 CAR-T cells have high antigen-specific anti-tumor functionality demonstrated by efficient eradication of CLDN6-expressing 3D tumor spheroids while CLDN6 negative tumor spheroids were left intact.

FIG. 4:

FIG. 4 shows a scheme for a transposon-based, two day protocol for generation of CAR-T cells by electroporation of a 3-component cocktail of nucleic acids delivered to the cell. A) The starting substrate can be peripheral blood mononuclear cells (PBMCs) or isolated, naïve human T cells. The three components are transposon donor DNA carrying a CAR expression cassette flanked by transposon end sequences, i.e., in a transposable element, transposase mRNA, and mRNA encoding for the cognate antigen for the CAR. By day 2 after electroporation, the modified T cells express CAR that can recognize the antigen expressed on surrounding cells. During the incubation, this specific CAR-antigen interaction results in triggering of signaling pathways that lead to T cell activation and expansion. B) Comparison of CAR expression 2 and 8 days after electroporation of naïve human T cells with either the three components (+DNA/+SB/+Ag), two transposon-system components (+DNA/+SB/−Ag) or two components consisting of transposon DNA carrying CAR and cognate antigen mRNA, but without the transposase (+DNA/−SB/+Ag) as the negative control. Comparable CAR expression between 3-component and 2-component protocols is typically observed on day 2. As the process continues, on day 8 CAR expression levels in the 3-component protocol exceed the expression in 2-component protocol. C) CAR-T cell activation in the presence of the cognate antigen in the 3-component setting (InstaCAR). InstaCAR protocol performed on isolated, naïve human T cells as the starting substrate results in antigen-specific activation of CAR-T cells on day 2 as monitored by CD25 upregulation and consequential co-expression of CAR and CD25 on T cells. This trend is maintained on day 6, as T cells co-expressing CAR and CD25 electroporated with the three components (+DNA/+SB/+Ag) outnumber the cells that received two components only (+DNA/+SB/−Ag), as the stimulation in the 3-component protocol increases the number of modified T cells more. The negative controls have not received transposase RNA (+DNA/−SB/+Ag) and do not express CAR. D) Activation of T cells is specifically observed in the 3-component protocol. In the same experiment as in FIG. 4C, CD25 expression on T cells is quantified by the mean fluorescence intensity (MFI). High CD25 expression level is observed only with the 3-component protocol (+DNA/+SB/+RNA). It remains low with the 2-component protocol and remains at basal levels (mock sample: −DNA/−SB/−Ag) in the absence of transposase (+DNA/−SB/+Ag) as well as in case of transposition of irrelevant DNA (irrDNA, transposon encoding for Venus fluorescent protein).

FIG. 5:

FIG. 5 sets out an InstaCAR protocol leading to detectable CLDN6 CAR expression on day 2 post-electroporation and subsequent expansion of CLDN6 CAR-T cells. Freshly isolated CD3+ T cells (5×10⁶) were electroporated in 100 μL electroporation reaction. Cells were electroporated with either the three components, 2.5 μg of nanoplasmid encoding CLDN6 CAR, 12.5 μg of mRNA-encoded SB100×transposase and 2.5 μg mRNA-encoded CLDN6-antigen (referred to as +DNA/+SB/+Ag, or also InstaCAR), two components only (referred to as +DNA/+SB/−Ag, reference protocol) or without the SB100X transposase (referred to as +DNA/−SB/+Ag). Mock control cells were electroporated without nucleic acids (referred to as −DNA/−SB/−Ag). A) Efficiency of CLDN6 CAR transposition assessed by flow cytometry, showing CLDN6 CAR expression in live CD3+ T cells from six donors at multiple timepoints post-electroporation. B) Proliferation capacity of CLDN6 CAR-T cells produced with InstaCAR protocol (+DNA/+SB/+Ag) in comparison with those produced by the reference protocol (+DNA/+SB/−Ag). Engineered T cells were counted using a Cellaca counter device and propidium iodide live/dead staining at multiple timepoints post-electroporation. Number of CAR+ T cells is shown. C) CAR copy number determination in InstaCAR protocol T cells by droplet digital PCR assay analysis of genomic DNA on day 13 post-electroporation. Data show mean±SD from different donors. CAR, chimeric antigen receptor; CLDN6, claudin 6; SB, Sleeping Beauty; Ag, antigen.

FIG. 6:

FIG. 6 shows that CLDN6 InstaCAR-T cells upregulate CAR expression after dilution with autologous PBMCs and cryopreservation. Freshly isolated CD3+, naïve T cells (5×10⁶) were electroporated in 100 μL electroporation reaction with either the three components, 2.5 μg of nanoplasmid encoding CLDN6 CAR, 12.5 μg of mRNA-encoded SB100X transposase and 2.5 μg mRNA-encoded CLDN6-antigen (referred to as +DNA/+SB/+Ag, also called InstaCAR) or without the SB100X transposase (referred to as +DNA/−SB/+Ag). Mock control cells were electroporated without nucleic acids (referred to as −DNA/−SB/−Ag). A) CAR expression post dilution with CFSE-labelled autologous PBMCs: On day 2 post-electroporation, InstaCAR-T cells were cocultured with autologous PBMCs at a 1:10 ratio of InstaCAR cells: PBMCs (to resemble the dilution of InstaCAR-T cells in blood post adoptive cell transfer, where approximately 85-100×10⁶ T cells would be transferred to the patient). The effect of cell dilution on CAR expression was assessed by flow cytometry at different timepoints, with non-engineered autologous PBMCs. PBMCs used as diluent in co-culture were identified with CFSE and excluded from the analysis. B) CAR expression following freeze/thaw cycle. On day 2 post-electroporation, InstaCAR-T cells were frozen and after thawing were cultured for 11 further days in X-VIVO 15 medium (from Lonza) supplemented with 5% human serum (HS), IL-7 (500 U/mL) and IL-15 (5,000 U/mL). CAR expression was measured at the indicated timepoints by flow cytometry. Data show mean±SD from two representative donors. Ag, antigen; CAR, chimeric antigen receptor; CFSE, carboxyfluorescein succinimidyl ester; CLDN6, claudin 6; DOT, day of thawing; HS, human serum; PBMC, peripheral blood mononuclear cells; SB, Sleeping Beauty.

FIG. 7:

FIG. 7 shows that CLDN6 InstaCAR-T cells retain their viability and proliferation capacity after freeze/thaw cycle. Freshly isolated CD3+, naïve T cells (5×10⁶) from two donors were electroporated in 100 μL electroporation reaction with either the three components, 2.5 μg of nanoplasmid encoding CLDN6 CAR, 12.5 μg of mRNA-encoded SB100X transposase and 2.5 μg mRNA-encoded CLDN6-antigen (referred to as +DNA/+SB/+Ag, InstaCAR) or without the SB100X transposase (referred to as +DNA/−SB/+Ag). Mock control cells were electroporated without nucleic acids (referred to as −DNA/−SB/−Ag). A-B) Percentage of live cells determined by flow cytometry with a flexible live/dead marker 48 h post-electroporation and at multiple timepoints following freeze/thaw. The viable cell percentage was determined including all events (A) or using the gating strategy of clinically relevant product (B). In the latter case, the cell debris that does not trigger clotting or immune response are excluded in contrast to the gating strategy used in (A). Data show mean±SD from two representative donors. DOT, day of thawing.

FIG. 8:

FIG. 8 shows that CLDN6 InstaCAR-T retain their proliferation capacity after freeze/thaw cycle. Freshly isolated CD3+, naïve T cells (5×10⁶) from two donors were electroporated in 100 μL electroporation reaction with either the three components, 2.5 μg of nanoplasmid encoding CLDN6 CAR, 12.5 μg of mRNA-encoded SB100X transposase and 2.5 μg mRNA-encoded CLDN6-antigen (referred to as +DNA/+SB/+Ag, also called InstaCAR), two components only (referred to as +DNA/+SB/−Ag, reference protocol) or without the SB100X transposase (referred to as +DNA/−SB/+Ag). A) Kinetics of CLDN6 InstaCAR-T cell expansion after 1.5×10⁶ cells were frozen on day 2 post-electroporation, and then thawed and cultured for further 16 days in X-VIVO 15 medium supplemented with 5% human serum, IL-7 (500 U/mL) and IL-15 (5,000 U/mL). T cells were counted at the indicated timepoints using Cellaca and propidium iodide live/dead staining. B) Proliferation capacity of frozen CDLN6 InstaCAR-T cells after co-culture with target tumor cells on the day of thawing at 1:1 effector-to-target ratio, without exogenous cytokines. Absolute numbers of InstaCAR-T cells were calculated by flow cytometry using counting beads. Data show mean±SD from two representative donors. DOF, day of freezing; DOT, day of thawing; CAR, chimeric antigen receptor; CLDN6, claudin 6; Ag, antigen.

FIG. 9:

FIG. 9 shows that CLDN6 InstaCAR-T cells show antigen specific, sequential tumor killing activity in vitro after thawing. Freshly isolated CD3+, naïve T cells (5×10⁶) from two donors were electroporated in 100 μL electroporation reaction with either the three components, 2.5 μg of nanoplasmid encoding CLDN6 CAR, 12.5 μg of mRNA-encoded SB100X transposase and 2.5 μg mRNA-encoded CLDN6-antigen (referred to as +DNA/+SB/+Ag, also called InstaCAR), two components only (referred to as +DNA/+SB/−Ag, reference protocol) or without the SB100X transposase (referred to as +DNA/−SB/+Ag). The cells were frozen on day 2 post-electroporation. On the day of thawing, the cells were co-cultured with PA-1/CLDN6+ target tumor cells stably expressing firefly luciferase at different effector:target (E:T) ratios. T cells were used without normalization to CAR expression level. The cells were challenged for the first time with tumor cells on the day of thawing (A), on day 7 post first challenge, the cells were re-challenged with fresh tumor cells for the second time (B) and after 120 h post second challenge the cell were again challenged with fresh tumor cells for the third time (C). The killing potency at first (A), second (B) and third (C) challenge at the indicated timepoints is shown. Cytotoxicity was evaluated with a bioluminescence-based assay. Data points represent mean of % killing of tumor cells using CLDN6 CAR-T cells from two donors. CLDN6, claudin 6; SB, Sleeping Beauty; Ag, antigen.

### EXAMPLES

### Example 1

### Three and Four Compound Systems for Effector Cell Engineering

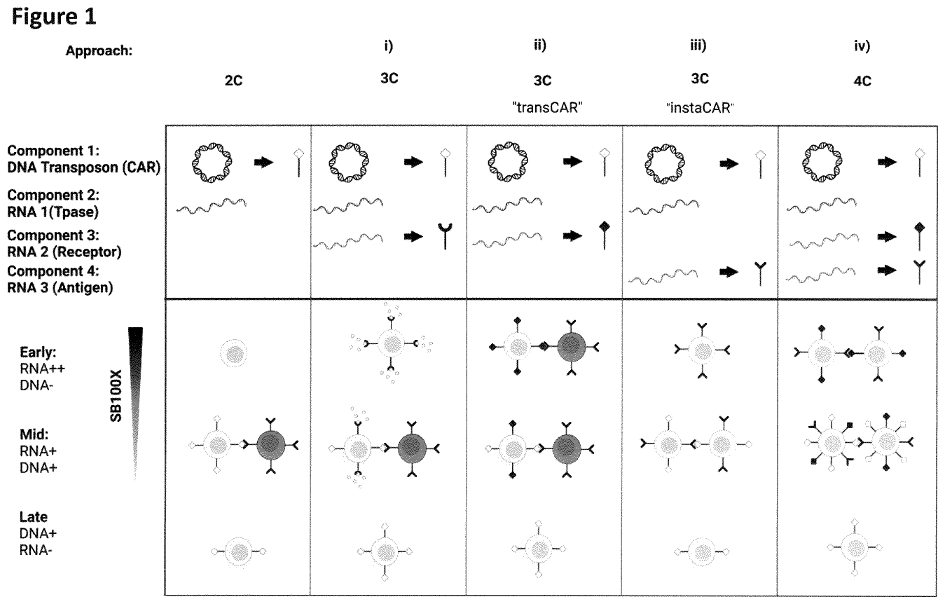

An immune effector cell according to the disclosure may for example comprise (i) a DNA molecule comprising a transposable element, which element comprises a first nucleotide sequence encoding a first chimeric antigen receptor, which binds to a tumor-associated antigen; (ii) a third mRNA molecule encoding for a transposase, and (iii) as second nucleic acid a) an mRNA molecule encoding a second cell-surface expressed receptor, which binds to a target different from the tumor-associated antigen; or (b) an mRNA molecule encoding the binding target of the first chimeric antigen receptor. FIG. 1 shows an overview of various examples.

### Example 2

### TransCAR Protocol

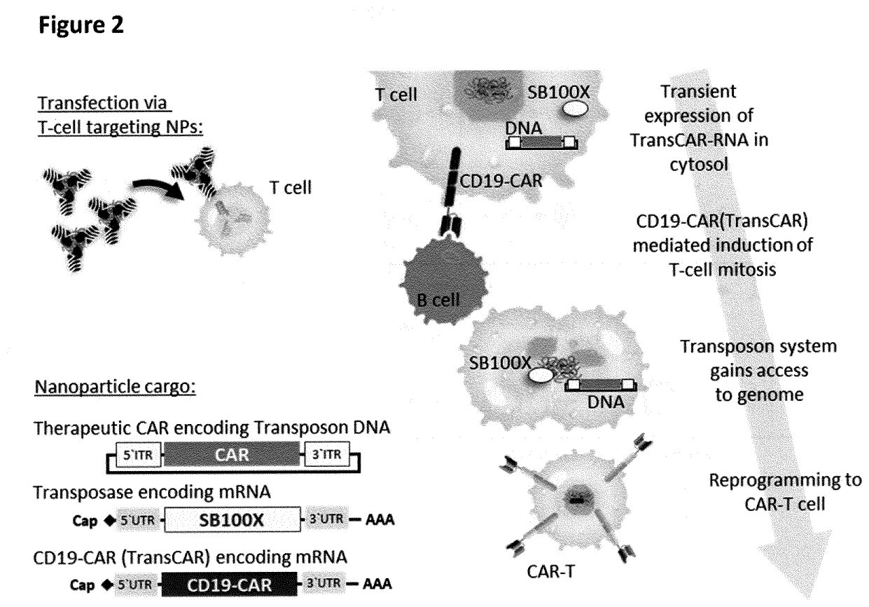

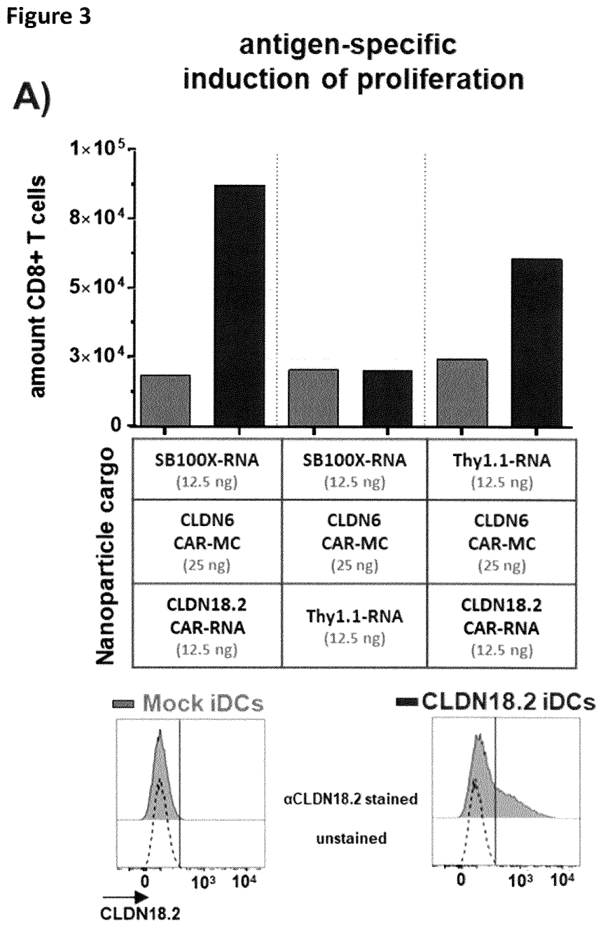

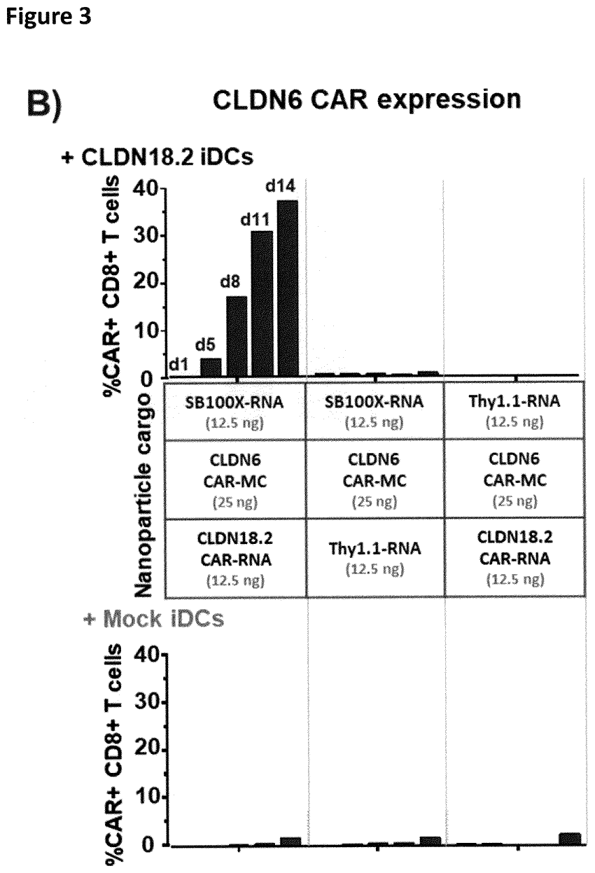

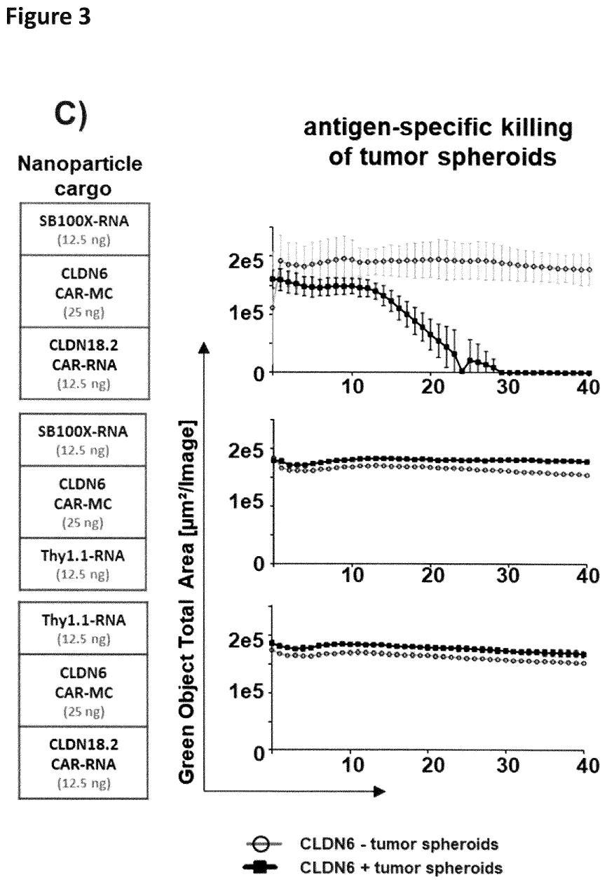

An embodiment of a three-component approach with a first nucleic acid encoding stably expressed, therapeutic CAR, a third nucleic acid RNA molecule encoding a transposase, and a second nucleic acid RNA molecule encoding a transiently expressed CAR for in vivo engineering of T cells is shown in FIG. 2. This embodiment is also called TransCAR protocol in the following. For in vitro validation of the TransCAR protocol, unstimulated primary human T cells, isolated from healthy donor PBMCs, have been transfected with CD8-targeting nanoparticles (CD8-DARPinE20-decorated PLXs; WO 2021/130225) delivering a three-component cargo composition. In short, 50 ng total nucleic acid complexed in CD8-targeting nanoparticles was pre-diluted in 50 μl X-VIVO 15 medium (from Lonza) before addition of 1×10⁶ T cells in 50 μl of the same medium. After 30 min of incubation (37° C., 5% CO₂) nanoparticles were removed via centrifugation. In all three nanoparticle cargo groups tested a RNA:DNA:RNA weight ratio of 1:2:1 was applied (see cargo boxes in FIGS. 3A, B and C). Autologous immature dendritic cells (iDCs), generated in vitro from CD14+ monocytes via continuous IL-4 and GM-CSF stimulation, showed TransCAR antigen (CLDN18.2) surface expression after electroporation with the TransCAR antigen (CLDN18.2)-encoding RNA (see FIG. 3, black bars) and were co-cultivated with transfected T cells in a 1:10 (iDCs:T cell) ratio. Mock electroporated iDCs were used as control (see FIG. 3, grey bars). In the following the time dependent CLDN6 CAR surface expression (FIG. 3B) was analyzed via flow cytometry. T cells were harvested on day 14 post nanoparticle treatment and transferred in an 1:10 effector to target cells ratio to CLDN6 positive and CLDN6 negative tumor cell spheroids which have been previously generated via cultivation of PA-1 ovarian carcinoma cell for 48 h in ultra-low adhesion plates. The GFP reporter expression in both tumor cell lines was hourly monitored in an Incucyte S3 Live cell analysis system (FIG. 3C). The data shown in FIG. 3 shows that the use of a 3C system induces proliferation, increases the T cell reprogramming efficiency post nanoparticle transfection and results in the generation of a highly effective and specific CLDN6 CAR-T cell population.

### Example 3

### InstaCAR Protocol

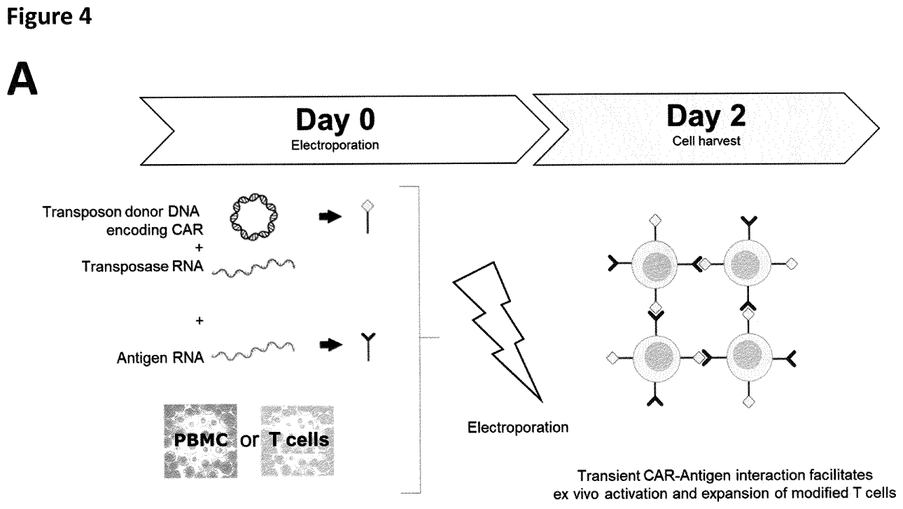

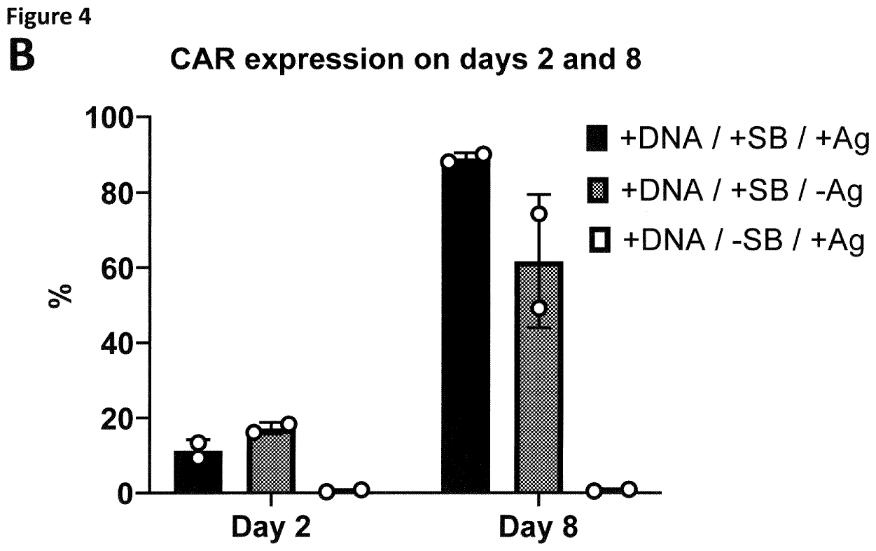

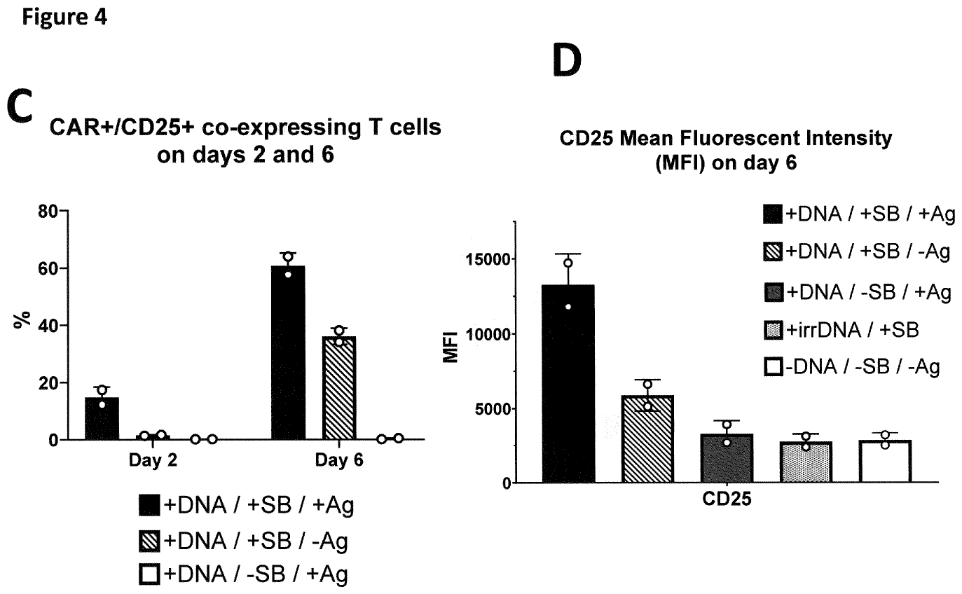

In the following an example of a transposon-based protocol that enables the manufacturing of potent CAR-T cells in 2 days (InstaCAR) is shown (FIG. 4). The following protocol involves the simultaneous electroporation of three active reagents into resting T cells or PBMCs: a transposon vector sequence carried on minimal backbone donor DNA molecule (first nucleic acid) and containing the CAR expression cassette; the transposase encoded in mRNA format (third nucleic acid); and the cognate antigen of the CAR also encoded in mRNA format (second nucleic acid). The transposase mRNA is translated into active transposase, which mediates integration of the transposon vector into the genome, which then becomes the first nucleic acid. Simultaneously, the antigen mRNA is also translated and provides an immediate activation signal for the nascent CAR, which consequently drives T cell activation, proliferation, and further results in increase of the CAR expression and enrichment of CAR-positive T cells.

The InstaCAR protocol enables multiple improvements over established CAR-T cell engineering methods. With the antigen being supplemented as mRNA, the enrichment and expansion of CAR+ T cells is facilitated, potentially improving trafficking and persistence of the engineered T cells after infusion in patients. The use of non-viral delivery of the Sleeping Beauty transposase enables single-step engineering via electroporation of the three components in one reaction, avoiding any biosafety concerns related to potential contamination with remaining infectious viral particles. In addition, as only the cells where the transposase is provided undergo activation and expansion, InstaCAR protocol results in an enrichment of successfully engineered cells only, reducing thereby the competition for space and resources with unmodified cells. Furthermore, as the protocol does not require a strong pre-activation step, gains in time and cost can be achieved, and the resulting T-cell product has improved immunophenotype. Finally, because the in this experiment used embodiment of the InstaCAR protocol involves simplified delivery of nucleic acids, its adaptation to automated processes is straightforward, facilitating throughput and reproducibility of the cell products.

### Data Summary and Observations:

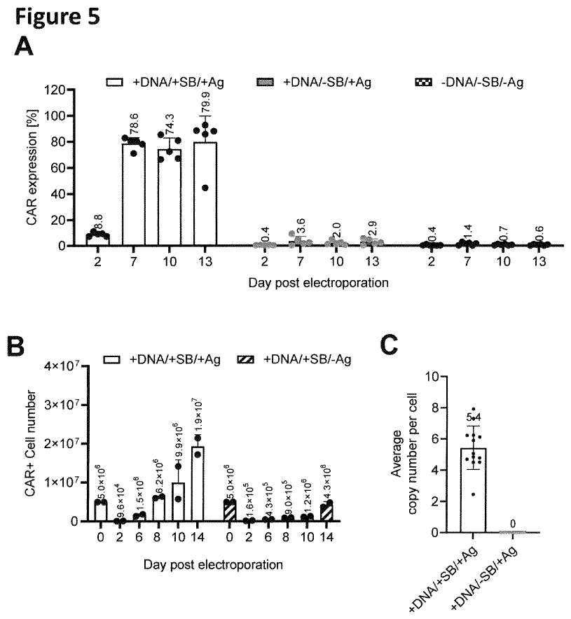

Using the InstaCAR protocol expression of CLDN6 CAR (previously developed at BioNTech SE) was successfully achieved in human CD3+ T cells within 2 days, and CAR+ T cells were further expanded in culture without supplementation of additional factors to the standard culturing medium (X-VIVO 15 medium (from Lonza) supplemented with 5% HS, IL-7 (500 U/mL) and IL-15 (5,000 U/mL)) (FIG. 5). When compared to a reference protocol, where only transposon carrying CAR and transposase mRNA are provided to cells, the present InstaCAR protocol led to stronger proliferation of CAR+ T cells over multiple timepoints (2, 8, 10, and 14 days post-electroporation; FIG. 5B). The average copy number per cell was determined to be 5.4 (FIG. 5C).

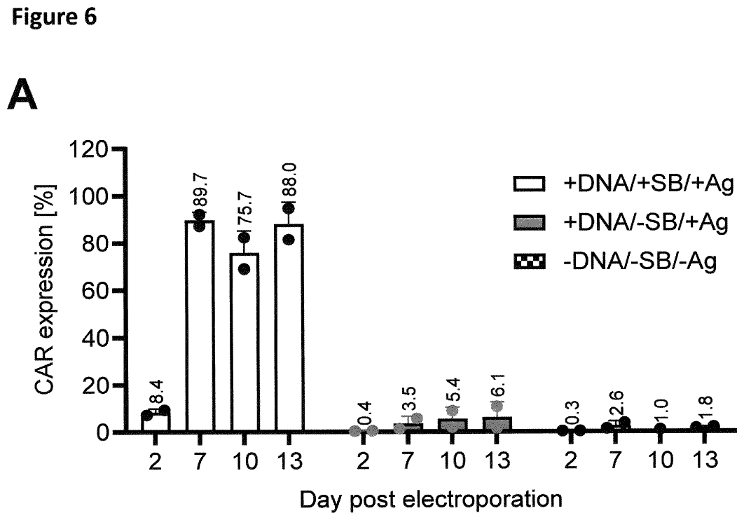

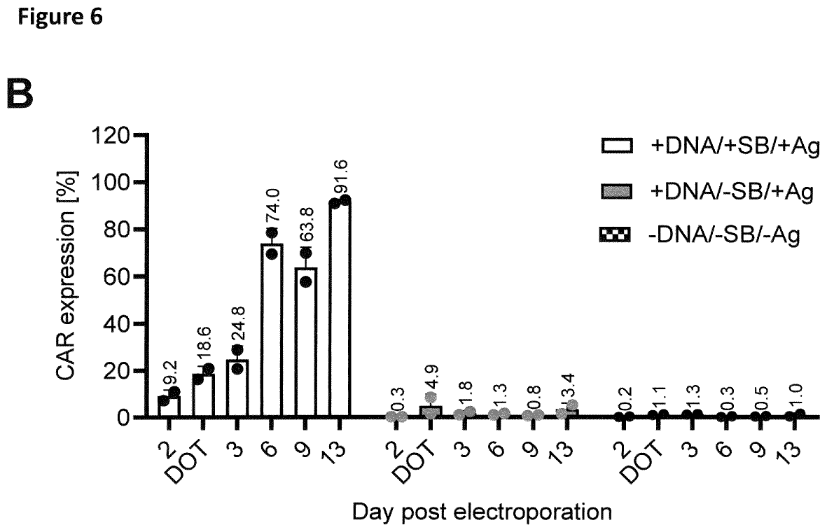

To evaluate CAR expression following dilution with autologous PBMCs aimed to resemble the loss of cell-cell interaction that engineered cells will experience after adoptive T cell transfer to the patients and under cryopreservation, CLDN6 CAR-T cells produced via an InstaCAR protocol were studied at multiple timepoints (FIG. 6). As shown previously in FIG. 5A, CLDN6 CAR expression is detected 2 days after electroporation, and is then increased >10-fold after dilution with PBMCs (FIG. 6A), suggesting that the loss of direct contact with cells expressing the antigen after first 48 hours is not detrimental for the efficiency of the InstaCAR protocol. When assessing the effects of cryopreservation, frequency of CAR-T cells after thawing increased progressively, leading to >85% CAR-T cells after the cells were cultured further for 11 days (FIG. 6B).

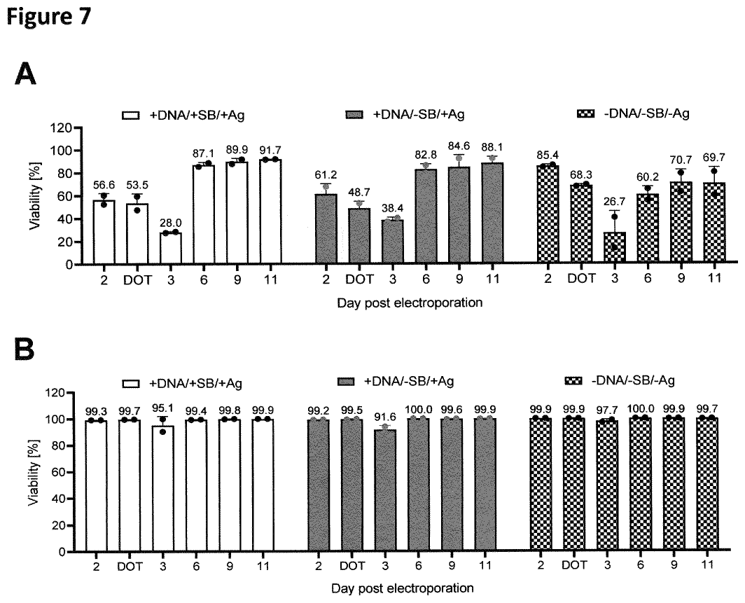

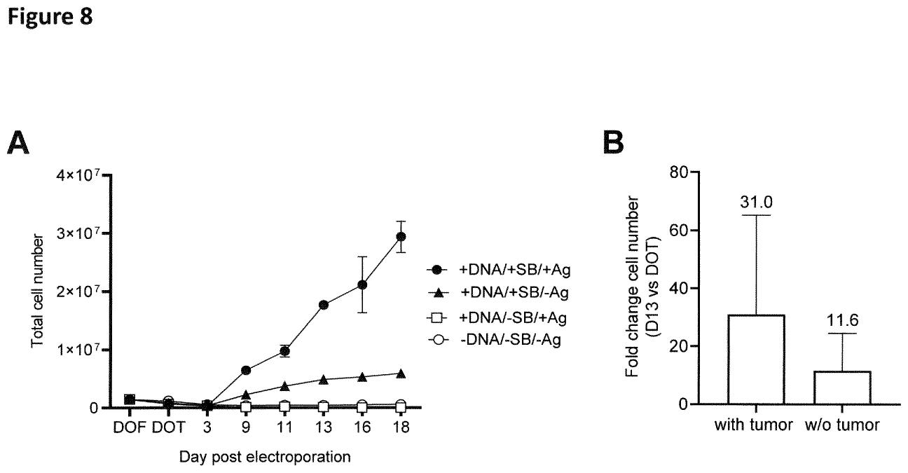

To evaluate the viability and proliferation potential of CLDN6 InstaCAR-T cells in response to antigen following freeze/thaw cycle, cell viability and numbers were followed at multiple timepoints (FIG. 7). Whereas viability of cells produced was reduced with freeze/thaw when gating on all events, it reached >90% when the cells were cultured for 9 further days (FIG. 7A). When gating on clinically relevant events, viability remained >99% post freeze/thaw at all timepoints (FIG. 7B). The cells expanded vigorously in culture after thawing (FIG. 8A), and this expansion could be further boosted when tumor cells were added to the culture (FIG. 8B).

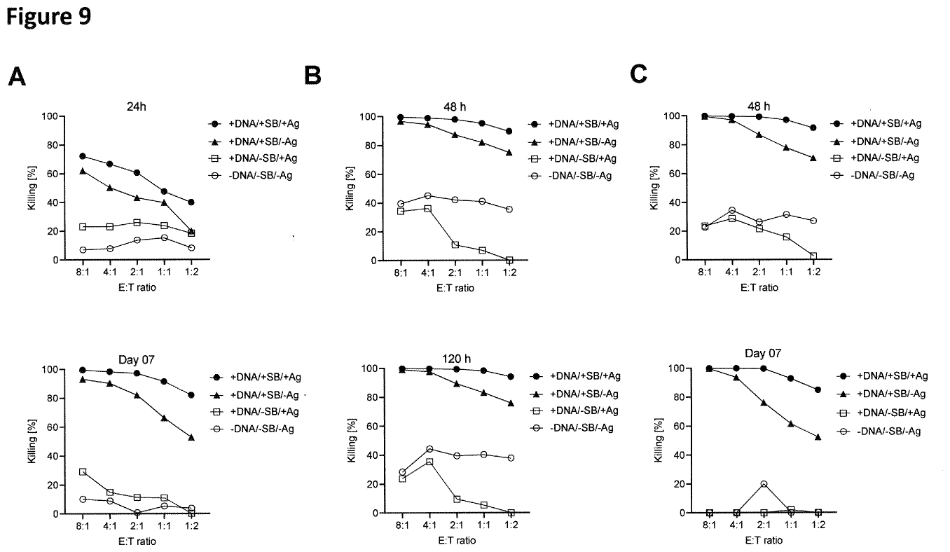

To assess the effector function of CLDN6 InstaCAR-T cells immediately after thawing, the cells were frozen on day 2 post-electroporation and were used in sequential tumor killing assays initiating on the day of thawing (FIG. 9A). 7 days after first tumor challenge, the cells were re-challenged with fresh tumor cells for the second time (FIG. 9B), and were again challenged 5 days after the second challenge for the third time (FIG. 9C). The InstaCAR-T cells show potent killing of tumor cells on the day of thawing, and strong killing on the second and third challenges. Compared with CAR-T cells produced with 2-component reference protocol, InstaCAR-T cells show more potent killing as seen by persistent high frequency killing after progressive addition of target tumor cells.

### Conclusion and Impact of the Protocol

The InstaCAR protocol generates CAR-T cells with detectable CAR expression on day 2 (FIG. 5), with the generated CAR-T cells showing autonomous upregulation of the CAR following dilution and cryopreservation due to expression of the cognate antigen (FIG. 6). The InstaCAR-T cells remain viable and retain a high proliferative capacity following a freeze/thaw cycle often used for clinical cell manufacturing processes (FIGS. 7 and 8), and are endowed with a strong tumor killing effector function that is retained for >2 weeks after thawing (FIG. 9).

The inclusion of mRNA encoding the CAR cognate antigen (CLDN6 in this embodiment) as one of the three components in an InstaCAR protocol enables early and progressive activation and expansion of the InstaCAR-T cells after electroporation. This capacity for autonomous expansion endows InstaCAR-T cells with improved function, trafficking to tumors and persistence following infusion in patients. Furthermore, InstaCAR protocol does not require an activation step before electroporation, therefore reducing manufacturing time, cost, and the need for extensive quality control tests, while simultaneously generating a CAR-T cell product with a preferable immunophenotype that limits the potential for early exhaustion.

Transposon-based non-viral concomitant delivery for CAR and antigen in a nucleic acid package enables single-step engineering of T cells via electroporation of the three components in one step, resulting in single step, non-viral transposon-based cell engineering strategy where CAR and antigen are concomitantly delivered to the T cells as a three component nucleic acids system. The protocol further facilitates efficient CAR-T cell generation without biosafety concerns inherent to cell engineering using viral particles. Furthermore, as InstaCAR protocol relies on delivery of a nucleic acid cocktail, and expands only successfully engineered cells, the method is especially suitable to automation, facilitating scaling up of manufacturing throughput, which is one of the main bottlenecks in CAR-T cell production.

We claim:

1. An immune effector cell comprising a first nucleic acid molecule comprising a first nucleotide sequence encoding a first cell-surface expressed antigen receptor and a second nucleic acid molecule comprising a second nucleotide sequence encoding an immune effector cell-activator molecule, wherein (i) the second nucleotide sequence is not integrated into a genomic nucleic acid molecule of the immune effector cell and/or (ii) the activator molecule is transiently expressed.

2. The immune effector cell according to claim 1, wherein the immune effector cell is isolated.

3. The immune effector cell according to claim 1 or claim 2, wherein the first nucleic acid molecule is DNA or RNA.

4. The immune effector cell according to any one of claims 1 to 3, wherein the first nucleotide sequence is integrated into a genomic nucleic acid molecule of the immune effector cell.

5. The immune effector cell according to any one of claims 1 to 4, wherein the genomic nucleic acid molecule is a chromosome, an episome, or a non-viral episome.

6. The immune effector cell according to claim 4 or claim 5, wherein the first nucleotide sequence is integrated into the genomic nucleic acid molecule via a DNA-based transposon system, a viral-based retrotransposon system, or a poly-A-based retrotransposon system.

7. The immune effector cell according to any one of claims 1 to 6, wherein the first nucleotide sequence is comprised within a transposable element.

8. The immune effector cell according to any one of claims 1 to 7, wherein the immune effector cell further comprises a third nucleic acid molecule comprising a third nucleotide sequence encoding a molecule having transposase activity.

9. The immune effector cell according to claim 8, wherein the third nucleic acid molecule is DNA or RNA.

10. The immune effector cell according to claim 8 or claim 9, wherein the third nucleic acid molecule is mRNA.

11. The immune effector cell according to any one of claims 8 to 10, wherein the molecule having transposase activity is Sleeping Beauty, PiggyBac, Frog, Prince, Himarl, Passport, Minos, hAT, Tol1, Tol2, AciDs, PIF, Harbinger, Harbinger3-DR, Hsmar1, or a functionally equivalent variant thereof having transposase activity.

12. The immune effector cell according to any one of claims 8 to 12, wherein the molecule having transposase activity is Sleeping Beauty transposase SB100X.

13. The immune effector cell according to any one of claims 8 to 12, wherein (i) the third nucleic acid molecule is not integrated into a genomic nucleic acid molecule of the immune effector cell and/or (ii) the encoded molecule having transposase activity is transiently expressed.

14. The immune effector cell according to any one of claims 1 to 13, wherein first cell-surface expressed antigen receptor is stably expressed.

15. The immune effector cell according to any one of claims 1 to 14, wherein the activator molecule allows for the activation, expansion, differentiation and/or proliferation of the immune effector cell.

16. The immune effector cell according to any one of claims 1 to 15, wherein the activator molecule is a non-coding RNA or protein.

17. The immune effector cell according to any one of claims 1 to 16, wherein the activator molecule binds to the extracellular portion of the first cell-surfaced expressed antigen receptor.

18. The immune effector cell according to any one of claims 1 to 16, wherein the activator molecule is a cytokine.

19. The immune effector cell according to any one of claims 1 to 16, wherein the activator molecule is a second cell-surface expressed antigen receptor, wherein the extracellular portions of the first and second cell-surfaced expressed antigen receptors do not bind to the same binding target.

20. The immune effector cell according to claim 19, wherein the immune effector cell further comprises a fourth nucleic acid molecule comprising a fourth nucleotide sequence encoding the binding target of the first cell-surface expressed antigen receptor.

21. The immune effector cell according to claim 19 or claim 20, wherein the immune effector cell further comprises a fifth nucleic acid molecule comprising a fifth nucleotide sequence encoding the binding target of the second cell-surface expressed antigen receptor.

22. The immune effector cell according to claim 20 or claim 21, wherein the fourth nucleic acid molecule or the fifth nucleic acid molecule comprise both the fourth nucleotide sequence encoding the binding target of the first cell-surface expressed antigen receptor and the fifth nucleotide sequence encoding the binding target of the second cell-surface expressed antigen receptor.

23. The immune effector cell according to any one of claims 20 to 22, wherein (i) the fourth and/or fifth nucleic acid molecule is not integrated into a genomic nucleic acid molecule of the immune effector cell and/or (ii) the encoded binding target of the fourth and/or fifth nucleic acid molecule is transiently expressed.

24. The immune effector cell according to any one of claims 20 to 23, wherein the fourth and/or fifth nucleic acid molecule is DNA or RNA.

25. The immune effector cell according to any one of claims 20 to 24, wherein the fourth and/or fifth nucleic acid molecule is mRNA.

26. The immune effector cell according to any one of claims 1 to 25, wherein the first cell-surface expressed antigen receptor is a chimeric antigen receptor (CAR) or a T cell receptor (TCR).

27. The immune effector cell according to any one of claims 19 to 26, wherein the second cell-surface expressed antigen receptor is a chimeric antigen receptor (CAR) or a T cell receptor (TCR).

28. The immune effector cell according to any one of claims 19 to 27, wherein the binding target of the second cell-surface expressed antigen receptor is expressed on or from cells different from cells expressing the binding target of the first cell-surface expressed antigen receptor.

29. The immune effector cell according to any one of claims 1 to 28, wherein the binding target of the first cell-surface expressed antigen receptor is a tumor-associated antigen or an antigen of an infectious agent, or an epitope thereof.

30. The immune effector cell according to any one of claims 19 to 30, wherein the binding target of the second cell-surface expressed antigen receptor is a cell-surface-expressed protein or a soluble protein, or an epitope thereof.

31. The immune effector cell according to claim 30, wherein the cell-surface expressed protein is a glycoprotein or a cell-surface expressed cytokine.

32. The immune effector cell according to claim 30 or claim 31, wherein the cell-surface expressed protein is a cluster of differentiation (CD) protein.

33. The immune effector cell according to claim 30, wherein the soluble protein is a soluble cytokine.

34. The immune effector cell according to any one of claims 19 to 34, wherein the binding target of the second cell-surface expressed antigen receptor is a cell-surface protein expressed on a blood cell, which blood cell is preferably another immune effector cell.

35. The immune effector cell according to claim 34, wherein the blood cell is a T cell, a NK cell, a dendritic cell, a macrophage, or a B cell.

36. The immune effector cell according to claim 34 or claim 35, wherein the cell-surface protein is CD19.

37. The immune effector cell according to claim 30, wherein the cell-surface protein is CLDN18.2.

38. The immune effector cell according to any one of claims 1 to 37, wherein the second nucleic acid molecule is DNA or RNA.

39. The immune effector cell according to any one of claims 1 to 38, wherein the second nucleic acid molecule is mRNA

40. The immune effector cell according to claim 3, 9, 10, 24, 25, 38 or 39, wherein the RNA or mRNA comprises a ribonucleobase other than A, C, G and U.

41. The immune effector cell according to claim 40, wherein the ribonucleobase is pseudouridine, preferably 1-methyl-pseudouridine.

42. The immune effector cell according to claim 3, 9, 10, 24, 25, 38 or 39, wherein the RNA comprises a 5′ cap structure.

43. The immune effector cell according to claim 42, wherein the 5′ cap structure is a natural occurring cap.

44. The immune effector cell according to claim 42, wherein the 5′ cap structure is a cap analog.

45. The immune effector cell according to claim 43 or claim 44, wherein the 5′ cap structure is one of the following: cap0, cap1, cap2, cap3, cap4, ARCA (Anti-Reverse Cap Analogs), modified ARCA, inosine, N1-methyl-guanosine, 2′-fluoro-guanosine, 7-deaza-guanosine, 8-oxo-guanosine, 2-amino-guanosine, LNA-guanosine and 2-azido-guanosine.

46. The immune effector cell according to claim 45, wherein the 5′ cap structure is cap0, which is ^{m7}G(5′)ppp(5′).

47. The immune effector cell according to claim 45, wherein the 5′ cap structure is cap1, which is ^{m7}G(5′)ppp(5′)(N₁^{2′-OMe}).

48. The immune effector cell according to claim 47, wherein the N₁ is chosen from A, C, G or U.

49. The immune effector cell according to claim 47 or 48, wherein the cap1 further comprises a second nucleotide N₂, which is a cap proximal A, G, C or U at position +2 and is represented as ^{m7}G(5′)ppp(5′)(N₁^{2′-OMe})pN₂.

50. The immune effector cell according to any one of claims 1 to 49, wherein the immune effector cell is a T cell, a B cell, a dendritic cell, or a NK cell.

51. The immune effector cell according to any one of claims 1 to 50, wherein the immune effector cell is a CD8+ and/or CD4+ T cell.

52. The immune effector cell according to any one of claims 1 to 51, wherein the immune effector cell is a cytotoxic T cell.

53. The immune effector cell according to any one of claims 1 to 52, wherein the second nucleic acid is not inherited by the progeny cells of the immune effector cell in the same manner as chromosomes are inherited, and/or wherein the second nucleic acid is diluted out compared to the total number of cells in each generation of progeny cells of the immune effector cell, and/or wherein after one round of cell division, the amount of the second nucleic acid molecule is less in each daughter cell compared to the amount in the parental cell.

54. An immune effector cell comprising (i) a DNA molecule comprising a transposable element, which element comprises a nucleotide sequence encoding a first T cell receptor or chimeric antigen receptor which binds to a tumor-associated antigen; (ii) an mRNA molecule encoding a second T cell receptor or chimeric antigen receptor which binds to a target different from the tumor-associated antigen; and (iii) an mRNA molecule encoding for a transposase.

55. An immune effector cell comprising (i) a nucleotide sequence encoding a first T cell receptor or a chimeric antigen receptor which binds to a tumor-associated antigen, which nucleotide sequence is integrated into the genome of the immune effector cell or is comprised within an episome present in the immune effector cell; and (ii) a nucleic acid molecule encoding a second T cell receptor or a chimeric antigen receptor which binds to a target different from the tumor-associated antigen, which nucleic acid molecule is not integrated into a genomic nucleic acid molecule of the immune effector cell.

56. An immune effector cell comprising (i) a DNA sequence encoding a first chimeric antigen receptor which binds to a tumor-associated antigen, which DNA sequence is integrated into the genome of the immune effector cell; and (ii) an mRNA molecule encoding a second chimeric antigen receptor which binds to a target different from the tumor-associated antigen.

57. The immune effector cell according to any one of claims 54 to 56, wherein the immune effector cell is a CD8+ cytotoxic T cell.

58. The immune effector cell according to any one of claims 54 to 57, wherein the immune effector cell is activated by the binding of the second T cell receptor or chimeric antigen receptor binding to its target.

59. The immune effector cell according to any one of claims 1 to 58, wherein the immune effector cell does not comprise a DNA nucleotide sequence encoding for the activator molecule.

60. The immune effector cell according to any one of claims 1 to 59, wherein the immune effector cell has reduced cell-surface expression of the endogenous T cell receptor to a level that prevents graft-versus-host activity of the immune effector cell when administered to a subject different from the subject from whom the immune effector cell was derived.

61. The immune effector cell according to any one of claims 1 to 60, wherein the immune effector cell does not express its endogenous T cell receptor on its cell-surface.

62. The immune effector cell according to any one of claims 1 to 61, wherein the immune effector cell has reduced cell-surface expression of the endogenous HLA complex to a level that prevents host-versus-graft activity in a subject to whom the immune effector cell has been administered, wherein the subject administered the immune effector cell is different from the subject from whom the immune effector cell was derived.

63. The immune effector cell according to any one of claims 1 to 62, wherein the immune effector cell does not express its endogenous HLA complex on its cell-surface.

64. A cellular composition comprising the immune effector cell according to any one of claims 1 to 63.

65. The cellular composition according to claim 64, further comprising a cryopreservation agent.

66. A pharmaceutical composition comprising the immune effector cell according to any one of claims 1 to 63 or the cellular composition according to claim 64 or claim 65, and a pharmaceutically acceptable carrier.

67. The immune effector cell according to any one of claims 1 to 63, the cellular composition according to claim 64 or claim 65, or the pharmaceutical composition according to claim 66 for use in a method of treating a subject having a disease, disorder or condition associated with expression or elevated expression of the binding target of the first cell-surface expressed antigen receptor, wherein the method comprises administering the immune effector cell, the cellular composition or the pharmaceutical composition to the subject.

68. The immune effector cell, the cellular composition or the pharmaceutical composition for use according to claim 67, wherein the disease, disorder or condition is cancer.

69. The immune effector cell, the cellular composition or the pharmaceutical composition for use according to claim 68, wherein the cancer is a solid cancer.

70. The immune effector cell, the cellular composition or the pharmaceutical composition for use according to claim 67, wherein the disease, disorder or condition is an infection.

71. The immune effector cell, the cellular composition or the pharmaceutical composition for use according to claim 70, wherein the infection is a viral infection.

72. The immune effector cell, the cellular composition or the pharmaceutical composition for use according to any one of claims 67 to 71, wherein the immune effector cell is autologous or heterologous to the subject being administered the immune effector cell, the cellular composition or the pharmaceutical composition.

73. A particle comprising (i) a first nucleic acid molecule comprising a first nucleotide sequence encoding a first cell-surface expressed antigen receptor, which first nucleotide sequence is comprised within a transposable element; and (ii) a second nucleic acid molecule comprising a second nucleotide sequence encoding an immune effector cell-activator molecule, wherein the second nucleotide sequence not comprised within a transposable element.

74. The particle according to claim 73, wherein the particle further comprises a third nucleic acid molecule comprising a third nucleotide sequence encoding a molecule having transposase activity, wherein the third nucleotide sequence is not comprised within a transposable element.

75. A particle comprising (i) a DNA episome comprising a first nucleotide sequence encoding a first cell-surface expressed antigen receptor, preferably a non-viral episome; and (ii) a second nucleic acid molecule comprising a second nucleotide sequence encoding an immune effector cell-activator molecule, wherein the second nucleic acid molecule, when present in a cell provides for transient expression of the activator molecule.

76. The particle according to claim 73 or claim 74, wherein the first nucleic acid molecule is DNA or RNA.

77. The particle according to any one of claims 73 to 76, wherein the first nucleic acid molecule or episome is a DNA minicircle or a linear DNA molecule.

78. The particle according to any one of claims 73 to 77, wherein the transposable element in the first nucleic acid is derived from a DNA-based transposon system, a viral-based transposon system, or a poly-A-based retrotransposon system.

79. The particle according to claim 74, wherein the third nucleic acid molecule is DNA or RNA.

80. The particle according to claim 78, wherein the third nucleic acid molecule is mRNA.

81. The particle according to any one of claims 74 or 76 to 80, wherein the molecule having transposase activity is Sleeping Beauty, PiggyBac, Frog, Prince, Himarl, Passport, Minos, hAT, Tol1, Tol2, AciDs, PIF, Harbinger, Harbinger3-DR, Hsmar1, or a functionally equivalent variant thereof having transposase/transposition activity.

82. The particle according to any one of claims 74 or 76 to 81, wherein the molecule having transposase activity is Sleeping Beauty transposase SB100X.

83. The particle according to any one of claims 73 to 82, wherein the second nucleic acid molecule is RNA.

84. The particle according to any one of claims 73 to 83, wherein the second nucleic acid molecule is mRNA.

85. The particle according to any one of claims 73 to 84, wherein the first cell-surface expressed antigen receptor binds to a tumor-associated antigen or an antigen of an infectious agent, or epitope thereof.

86. The particle according to any one of claims 73 to 85, wherein the first cell-surface expressed antigen receptor is a chimeric antigen receptor (CAR) or T cell receptor (TCR).

87. The particle according to any one of claims 73 to 86, wherein the activator molecule allows for the activation, expansion, differentiation and/or proliferation of the immune effector cell.

88. The particle according to any one of claims 73 to 87, wherein the activator molecule is a non-coding RNA or protein.

89. The particle according to any one of claims 73 to 88, wherein the activator molecule binds to the extracellular portion of the first cell-surfaced expressed antigen receptor.

90. The particle according to any one of claims 73 to 88, wherein the activator molecule is a cytokine.

91. The particle according to any one of claims 73 to 88, wherein the activator molecule is a second cell-surface expressed antigen receptor, wherein the extracellular portions of the first and second cell-surfaced expressed antigen receptors do not bind to the same binding target.

92. The particle according to claim 91, wherein the particle further comprises a fourth nucleic acid molecule comprising a fourth nucleotide sequence encoding the binding target of the first cell-surface expressed antigen receptor, wherein the fourth nucleotide sequence is not comprised within a transposable element.

93. The particle according to claim 91 or claim 92, wherein the particle further comprises a fifth nucleic acid molecule comprising a fifth nucleotide sequence encoding the binding target of the second cell-surface expressed antigen receptor, wherein the fifth nucleotide sequence is not comprised within a transposable element.

94. The particle according to claim 92 or claim 93, wherein the fourth nucleic acid molecule or the fifth nucleic acid molecule comprise both the fourth nucleotide sequence encoding the binding target of the first cell-surface expressed antigen receptor and the fifth nucleotide sequence encoding the binding target of the second cell-surface expressed antigen receptor.

95. The particle according to any one of claims 92 to 94, wherein the fourth and/or fifth nucleic acid molecule is DNA or RNA.

96. The particle according to any one of claims 92 to 95, wherein the fourth and/or fifth nucleic acid molecule is mRNA.

97. The particle according to any one of claims 91 to 96, wherein the second cell-surface expressed antigen receptor is a chimeric antigen receptor (CAR) or a T cell receptor (TCR).

98. A particle comprising the first, second and third nucleic acid molecule specified in any one of claims 1 to 72.

99. The particle according to claim 98, which particle further comprises the fourth and/or fifth nucleic acid molecule specified in any one of claims 20 to 72.

100. A particle comprising the first, second, third and fourth nucleic acid molecule specified in any one of claims 19 to 72.

101. The particle according to claim 100, which particle further comprises the fifth nucleic acid molecule specified in any one of claims 19 to 72.

102. The particle according to any one of claims 73 to 101, wherein the particle comprises a polyalkyleneimine or a lipid.

103. The particle according to any one of claims 73 to 102, wherein the particle comprises a lipid, preferably comprising a lipid with a cationic headgroup.

104. The particle according to any one of claims 73 to 103, wherein the particle comprises a pH responsive lipid.

105. The particle according to any one of claims 73 to 104, wherein the particle comprises a PEGylated-lipid.

106. The particle according to any one of claims 73 to 105, wherein the particle is a lipid particle, polymer particle, or a mixture thereof.

107. The particle according to any one of claims 73 to 106, wherein the particle is a nanoparticle.

108. The particle according to any one of claims 73 to 107, wherein the particle is a lipid nanoparticle (LNP), a lipoplex, (LPX), a polyplex (PLX), or a lipopolyplex (LPLX) particle.

109. The particle according to any one of claims 73 to 108, wherein the particle further comprises at least one phosphatidylserine.

110. The particle according to any one of claims 73 to 110, wherein the particles are nanoparticles, in which:
(i) the number of positive charges in the nanoparticles does not exceed the number of negative charges in the nanoparticles and/or
(ii) the nanoparticles have a neutral or net negative charge and/or
(iii) the zeta potential of the nanoparticles is 0 or less.

111. The particle according to any one of claims 73 to 102, wherein the particle comprises polyalkyleneimine.

112. The particle according to claim 111, wherein (a) the molar ratio of the number of nitrogen atoms (N) in the polyalkyleneimine to the number of phosphor atoms (P) in the first, second, and optionally third nucleic acid molecules (N:P ratio) is 2.0 to 15.0, preferably 6.0 to 12.0; or (b) the molar ratio of the number of the number of nitrogen atoms (N) in the polyalkyleneimine to the number of phosphor atoms (P) in the first, second, and optionally third nucleic acid molecules (N:P ratio) is at least about 48, optionally about 48 to 300, about 60 to 200, or about 80 to 150.

113. The particle according to claim 111, wherein the ionic strength of the composition is 50 mM or less, preferably wherein the concentration of monovalent cationic ions is 25 mM or less and the concentration of divalent cationic ions is 20 μM or less.

114. The particle according to any one of claims 111 to 113, wherein the particle is a polyplex particle.

115. The particle according to any one of claims 73 to 114, wherein the particle comprises a hydrophobic moiety having a binding moiety covalently attached thereto.

116. The particle according to claim 115, wherein the hydrophobic moiety having a binding moiety covalently attached thereto and the particle are non-covalently associated with each other.

117. The particle according to claim 115 or claim 116, wherein the hydrophobic moiety having a binding moiety covalently attached thereto is an integral part of the particle.

118. The particle according to anyone of claims 115 to 117, wherein the hydrophobic moiety having a binding moiety covalently attached thereto comprises a polymer.

119. The particle according to any one of claims 115 to 118, wherein the hydrophobic moiety having a binding moiety covalently attached thereto comprises a compound of 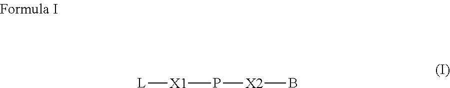
wherein
P comprises a polymer;
L comprises a hydrophobic moiety attached to a first end of the polymer;
B comprises a binding moiety attached to a second end of the polymer;
X1 is absent or a first linking moiety; and
X2 is absent or a second linking moiety.

120. The particle according to claim 119, wherein X1 comprises a carbonyl group.

121. The particle according to claim 119 or claim 120, wherein X2 comprises the reaction product of a maleimide group with a thiol or cysteine group of a compound comprising the binding moiety.

122. The particle according to any one of claims 115 to 121, wherein the hydrophobic moiety is or is comprised in a lipid.

123. The particle according to any one of claims 118 to 122, wherein the polymer provides stealth property, extends circulation half-life and/or reduces non-specific protein binding or cell adhesion.

124. The particle according to any one of claims 118 to 123, wherein the polymer comprises polyethyleneglycol (PEG).

125. The particle according to any one of claims 115 to 124, wherein the hydrophobic moiety having a binding moiety covalently attached thereto comprises a compound of 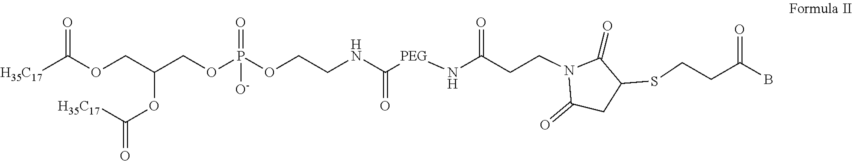
wherein B comprises the binding moiety.

126. The particle according to claim 125, wherein B comprises a moiety comprising the structure —N-peptide-C(O)—NH₂.

127. The particle according to any one of claims 115 to 126, wherein the binding moiety covalently attached to the hydrophobic moiety comprises an antibody or an antibody derivative.

128. The particle according to any one of claims 73 to 127, wherein the particle is complexed with the nucleic acid molecules and/or encapsulates the nucleic acid molecules.

129. A pharmaceutical composition comprising the particle according to any one of claims 73 to 128, and a pharmaceutically acceptable carrier.

130. The particle according to claim 73 to 128 or the pharmaceutical composition according to claim 129 for use in a method of treating a subject having a disease, disorder or condition associated with expression or elevated expression of the binding target of the first cell-surface expressed antigen receptor, wherein the method comprises administering the particle or the pharmaceutical composition to the subject.

131. The particle or the pharmaceutical composition for use according to claim 130, wherein the disease, disorder or condition is cancer.

132. The particle for use according to claim 131, wherein the cancer is a solid cancer.

133. The particle or the pharmaceutical composition for use according to claim 130, wherein the disease, disorder or condition is an infection.

134. The particle for use according to claim 133, wherein the infection is a viral infection.

135. A complex comprising
(a) the particle according to any one of claims 73 to 128, wherein the particle comprises a hydrophobic moiety having a binding moiety covalently attached thereto, and (b) a compound comprising (i) a moiety binding to the binding moiety covalently attached to the hydrophobic moiety and (ii) a moiety targeting a cell-surface antigen.

136. The complex according to claim 135, wherein the moiety binding to the binding moiety covalently attached to the hydrophobic moiety comprises an antibody or an antibody derivative.

137. The complex according to claim 136, wherein the binding moiety covalently attached to the hydrophobic moiety comprises a peptide comprising an ALFA-tag; and the moiety binding to the binding moiety covalently attached to the hydrophobic moiety comprises an antibody or an antibody derivative comprising a VHH domain comprising the CDR1 sequence VTISALNAMAMG, the CDR2 sequence AVSERGNAM, and the CDR3 sequence LEDRVDSFHDY.

138. The complex according to any one of claims 135 to 137, wherein (i) the moiety binding to the binding moiety covalently attached to the hydrophobic moiety and (ii) the moiety targeting a cell-surface antigen are linked to each other.

139. The complex according to any one of claims 135 to 138, wherein the compound under (b) comprises a peptide or polypeptide.

140. The complex according to any one of claims 135 to 139, wherein the moiety targeting a cell-surface antigen comprises an antibody or an antibody derivative.

141. The complex according to any one of claims 135 to 140, wherein the cell-surface antigen is characteristic for an immune effector cell.

142. The complex according to any one of claims 135 to 141, wherein the cell-surface antigen comprises CD4 and/or CD8.

143. The complex according to any one of claims 135 to 142, wherein the cell-surface antigen comprises CD3.

144. The complex according to any one of claims 135 to 143 for use in the treatment of a subject having a disease, disorder or condition associated with expression or elevated expression of the binding target of the first cell-surface expressed antigen receptor.

145. A method of producing an immune effector cell expressing a first antigen receptor on the cell-surface, the method comprising contacting an immune effector cell with (i) a first nucleic acid molecule comprising a first nucleotide sequence encoding a first cell-surface expressed antigen receptor, and (ii) a second nucleic acid molecule comprising a second nucleotide sequence encoding an immune effector cell activator molecule, wherein the second nucleotide sequence is not comprised within a transposable element, and wherein the first cell-surface expressed antigen receptor is stably expressed in the cell and the activator molecule is transiently expressed in the cell.

146. The method according to claim 145, wherein the first nucleic acid molecule is DNA or RNA.

147. The method according to claim 145 or claim 146, wherein the method further comprises integrating the first nucleotide sequence into a genomic nucleic acid molecule of the immune effector cell.

148. The method according to any one of claims 145 to 147, wherein the first nucleotide sequence is comprised within a transposable element.

149. The method according to any one of claims 145 to 148, wherein the method further comprises contacting the immune effector cell with a third nucleic acid molecule comprising a third nucleotide sequence encoding a molecule having transposase activity, wherein the third nucleotide sequence is not comprised within a transposable element.

150. The method according to claim 149, wherein the third nucleic acid molecule is DNA or RNA.

151. The method according to claim 149 or claim 150, wherein the third nucleic acid molecule is mRNA.

152. The method according to any one of claims 149 to 151, wherein the molecule having transposase activity is Sleeping Beauty, PiggyBac, Frog, Prince, Himarl, Passport, Minos, hAT, Tol1, Tol2, AciDs, PIF, Harbinger, Harbinger3-DR, Hsmar1, or a functionally equivalent variant thereof having transposase/transposition activity.

153. The method according to any one of claims 149 to 151, wherein the molecule having transposase activity is Sleeping Beauty transposase SB100X.

154. The method according to claim 145 or claim 146, wherein the first nucleic acid is an episome.

155. The method according to claim 154, wherein the episome is a non-viral episome.

156. The method according to any one of claims 145 to 155, wherein the activator molecule allows for the activation, expansion, differentiation and/or proliferation of the immune effector cell.

157. The method according to any one of claims 145 to 156, wherein the activator molecule is a non-coding RNA or protein.

158. The method according to any one of claims 145 to 157, wherein the activator molecule binds to the extracellular portion of the first cell-surfaced expressed antigen receptor.

159. The method according to any one of claims 145 to 158, wherein the activator molecule is a cytokine.

160. The method according to any one of claims 145 to 159, wherein the activator molecule is a second cell-surface expressed antigen receptor, wherein the extracellular portions of the first and second cell-surfaced expressed antigen receptors do not bind to the same binding target.

161. The method according to claim 160, wherein the method further comprises contacting the immune effector cell with a fourth nucleic acid molecule comprising a fourth nucleotide sequence encoding the binding target of the first cell-surfaced expressed antigen receptor.

162. The method according to claim 160 or claim 161, wherein the method further comprises contacting the immune effector cell with a fifth nucleic acid molecule comprising a fifth nucleotide sequence encoding the binding target of the second cell-surfaced expressed antigen receptor.

163. The method according to claim 161 or claim 162, wherein the fourth nucleic acid molecule or the fifth nucleic acid molecule comprise both the fourth nucleotide sequence encoding the binding target of the first cell-surfaced expressed antigen receptor and the fifth nucleotide sequence encoding the binding target of the second cell-surfaced expressed antigen receptor.

164. The method according to any one of claims 161 to 163, wherein the fourth and/or fifth nucleic acid molecule is DNA or RNA.

165. The method according to any one of claims 161 to 164, wherein the fourth and/or fifth nucleic acid molecule is mRNA.

166. The method according to any one of claims 145 to 165, wherein the first cell-surfaced expressed antigen receptor is a chimeric antigen receptor (CAR) or a T cell receptor (TCR).

167. The method according to any one of claims 160 to 166, wherein the first cell-surfaced expressed antigen receptor and/or the second cell-surfaced expressed antigen receptor is a chimeric antigen receptor (CAR) or a T cell receptor (TCR).

168. The method according to any one of claims 145 to 167, wherein the binding target of the second cell-surfaced expressed antigen receptor is expressed on or from cells different from cells expressing the binding target of the first cell-surfaced expressed antigen receptor.

169. The method according to any one of claims 145 to 168, wherein the binding target of the first cell-surfaced expressed antigen receptor is a tumor-associated antigen or an antigen of an infectious agent, or an epitope thereof.

170. The method according to any one of claims 145 to 169, wherein the second nucleic acid molecule is DNA or RNA.

171. The method according to any one of claims 145 to 170, wherein the second nucleic acid molecule is mRNA.

172. The method according to claim 146, 150, 151, 164, 165, 170 or 171, wherein the RNA or mRNA comprises a ribonucleobase other than A, C, G and U.

173. The method according to claim 172, wherein the ribonucleobase is pseudouridine, preferably 1-methyl-pseudouridine.

174. The method according to claim 146, 150, 151, 164, 165, 170 or 171, wherein the RNA comprises a 5′ cap structure.

175. The method according to claim 174, wherein the 5′ cap structure is a natural occurring cap.

176. The method according to claim 174, wherein the 5′ cap structure is a cap analog.

177. The method according to claim 175 or claim 176, wherein the 5′ cap structure is one of the following: cap0, cap1, cap2, cap3, cap4, ARCA (Anti-Reverse Cap Analogs), modified ARCA, inosine, N1-methyl-guanosine, 2′-fluoro-guanosine, 7-deaza-guanosine, 8-oxo-guanosine, 2-amino-guanosine, LNA-guanosine and 2-azido-guanosine.

178. The method according to any one of claims 145 to 177, wherein the immune effector cell is a T cell, a B cell, a dendritic cell, or a NK cell.

179. The method according to any one of claims 145 to 178, wherein the immune effector cell is a CD8+ and/or CD4+ T cell.

180. The method according to any one of claims 145 to 179, wherein the immune effector cell is a cytotoxic T cell.

181. The method according to any one of claims 145 to 180, wherein the second nucleic acid is not inherited by the progeny cells of the immune effector cell in the same manner as chromosomes are inherited, and/or wherein the second nucleic acid is diluted out in each generation of progeny cells of the immune effector cell, and/or wherein after one round of cell division, the amount of the second nucleic acid molecule is less in each daughter cell compared to the amount in the parental cell.

182. A method of producing an immune effector cell expressing a first antigen receptor on the cell-surface, the method comprising contacting an immune effector cell with a particle according to any one of claims 73 to 134 or a complex according to any one of claims 135 to 144.

183. The method according to any one of claims 145 or 182, wherein the contacting occurs in vitro.

184. The method according to any one of claims 160 to 183, wherein the method further comprises, after contacting the nucleic acid molecules to the immune effector cell, a step of contacting the immune effector cell with the binding target of the second cell-surface expressed antigen receptor or a cell expressing the binding target.

185. A method of producing an immune effector cell expressing two antigen receptors on the cell-surface, the method comprising contacting, in vitro or ex vivo, an immune effector cell with (i) a DNA molecule comprising a first nucleotide sequence encoding a first cell-surface expressed antigen receptor, which first nucleotide sequence is comprised with a transposable element; (ii) an RNA molecule comprising a second nucleotide sequence encoding a second cell-surface expressed antigen receptor, which second nucleotide sequence is not comprised within a transposable element; and (iii) an RNA molecule comprising a third nucleotide sequence encoding a transposase, which third nucleotide sequence is not comprises within a transposable element, wherein the extracellular domains of the first and second cell-surface expressed antigen receptors bind to different targets, preferably wherein the binding target of the first cell-surface expressed antigen receptor is a tumor or tumor-associated antigen and the binding target of the second cell-surface expressed antigen receptor is expressed on the surface of a blood cell.

186. The method according to claim 185, further comprising contacting the immune effector cell with the binding target of the second cell-surface expressed antigen receptor or with a cell expressing the binding target of the second cell-surface expressed antigen receptor.

187. A method of producing an immune effector cell expressing two antigen receptors on the cell-surface, the method comprising contacting an immune effector cell with a particle, which particle comprises (i) a DNA molecule comprising a first nucleotide sequence encoding a first cell-surface expressed antigen receptor, which first nucleotide sequence is comprised with a transposable element; (ii) an mRNA molecule comprising a second nucleotide sequence encoding a second cell-surface expressed antigen receptor; and (iii) an mRNA molecule comprising a third nucleotide sequence encoding a transposase; wherein the extracellular domains of the first and second cell-surface expressed antigen receptors bind to different targets, preferably wherein the binding target of the first cell-surface expressed antigen receptor is a tumor or tumor-associated antigen and the binding target of the second cell-surface expressed antigen receptor is expressed on the surface of a blood cell.

188. The method according to claim 187, wherein the contacting is in vivo.

189. A method of treating a subject having a disease, disorder or condition associated with expression or elevated expression of an antigen, the method comprising administering to the subject a first nucleic acid molecule comprising a first nucleotide sequence encoding a first cell-surface expressed antigen receptor and a second nucleic acid molecule comprising a second nucleotide sequence encoding an immune effector cell activator molecule, wherein the binding target of the first cell-surface expressed antigen receptor is the antigen that is associated with the disease, disorder or condition, wherein (i) the second nucleotide sequence does not integrate into a genomic nucleic acid molecule of the cells of the subject or is comprised within an episome present in the cells of the subject and/or (ii) the activator molecule is transiently expressed in the subject.

190. The method according to claim 189, wherein the nucleic acid molecules are in particle comprising a lipid.

191. A method of treating a subject having a disease, disorder or condition associated with expression or elevated expression of an antigen, the method comprising administering to the subject a particle, which particle comprises (i) a DNA molecule comprising a first nucleotide sequence encoding a first cell-surface expressed antigen receptor, which first nucleotide sequence is comprised with a transposable element and wherein the binding target of the first cell-surface expressed antigen receptor is the antigen that is associated with the disease, disorder or condition; (ii) an mRNA molecule comprising a second nucleotide sequence encoding a second cell-surface expressed antigen receptor; and (iii) an mRNA molecule comprising a third nucleotide sequence encoding a transposase; wherein the extracellular domains of the first and second cell-surface expressed antigen receptors bind to different targets.

192. A method of treating a subject having a disease, disorder or condition associated with expression or elevated expression of an antigen, the method comprising administering to the subject the immune effector cell according to any one of claims 1 to 63, the cellular composition according to claim 64 or claim 65, or the pharmaceutical composition according to claim 66, wherein the binding target of the first cell-surface expressed antigen receptor is the antigen that is associated with the disease, disorder or condition.

193. A method of treating a subject having a disease, disorder or condition associated with expression or elevated expression of an antigen, the method comprising administering to the subject a particle according to any one of claims 73 to 128 or the pharmaceutical composition according to claim 129, wherein the binding target of the first cell-surface expressed antigen receptor is the antigen that is associated with the disease, disorder or condition.

194. A method of treating a subject having a disease, disorder or condition associated with expression or elevated expression of an antigen, the method comprising administering to the subject a complex according to any one of claims 135 to 144, wherein the binding target of the first cell-surface expressed antigen receptor is the antigen that is associated with the disease, disorder or condition.

195. The method according to any one of claims 189 to 194, wherein the antigen associated with a disease, disorder or condition is a tumor-associated antigen.

196. The method according to any one of claims 189 to 195, which is a method for treating or preventing cancer in a subject.

197. The method according to any one of claims 189 to 194, wherein the antigen associated with a disease, disorder or condition comprises an antigen of an infectious agent.

198. The method according to claim 197, wherein the infectious agent is a virus.

199. The method according to any one of claims 189 to 194, 197 or 198, which is a method for treating or preventing an infection in a subject.


SEQUENCE LISTING (26 sequences)

SEQ ID NO: 1 - protein, 12 aa, synthetic construct
  source 1..12: note = CDR1 VHH
VTISALNAMAMG

SEQ ID NO: 2 - protein, 9 aa, synthetic construct
  source 1..9: note = CDR2 VHH
AVSERGNAM

SEQ ID NO: 3 - protein, 11 aa, synthetic construct
  source 1..11: note = CDR3 VHH
LEDRVDSFHDY

SEQ ID NO: 4 - protein, 13 aa, synthetic construct
  source 1..13: note = ALFA-tag
SRLEEELRRRLTE

SEQ ID NO: 5 - protein, 14 aa, synthetic construct
  source 1..14: note = ALFA-tag
PSRLEEELRRRLTE

SEQ ID NO: 6 - protein, 14 aa, synthetic construct
  source 1..14: note = ALFA-tag
SRLEEELRRRLTEP

SEQ ID NO: 7 - protein, 15 aa, synthetic construct
  source 1..15: note = ALFA-tag
PSRLEEELRRRLTEP

SEQ ID NO: 8 - protein, 13 aa, synthetic construct
  source 1..13: note = ALFA-tag
  SITE 5..9: note = glutamic acid lysine amide
SRLEEELRKRLTE

SEQ ID NO: 9 - protein, 13 aa, synthetic construct
  source 1..13: note = ALFA-tag
  SITE 5..9: note = aspartatic acid lysine amide
SRLEDELRKRLTE

SEQ ID NO: 10 - protein, 13 aa, synthetic construct
  source 1..13: note = ALFA-tag
  SITE 5: note = glutamic acid lysine amide
SRLEEELKRRLTE

SEQ ID NO: 11 - protein, 13 aa, synthetic construct
  source 1..13: note = ALFA-tag
  SITE 9..13: note = lysine glutamic acid amide
SRLEEELRKRLTE

SEQ ID NO: 12 - protein, 12 aa, synthetic construct
  source 1..12: note = CDR1 VHH
  VARIANT 3: note = I or V
VTXSALNAMAMG

SEQ ID NO: 13 - protein, 9 aa, synthetic construct
  source 1..9: note = CDR2 VHH
  VARIANT 4: note = E, H, N, D or S
AVSXRGNAM

SEQ ID NO: 14 - protein, 11 aa, synthetic construct
  source 1..11: note = CDR3 VHH
LEDRVDSFHDY

SEQ ID NO: 15 - protein, 13 aa, synthetic construct
  source 1..13: note = CDR1 VHH
  VARIANT 4: note = I or V
GVTXSALNAMAMG

SEQ ID NO: 16 - protein, 9 aa, synthetic construct
  source 1..9: note = CDR2 VHH
  VARIANT 4: note = E, H, N, D or S
AVSXRGNAM

SEQ ID NO: 17 - protein, 12 aa, synthetic construct
  source 1..12: note = CDR1 VHH
VTISALNAMAMG

SEQ ID NO: 18 - protein, 9 aa, synthetic construct
  source 1..9: note = CDR2 VHH
AVSERGNAM

SEQ ID NO: 19 - protein, 13 aa, synthetic construct
  source 1..13: note = CDR1 VHH
GVTISALNAMAMG

SEQ ID NO: 20 - protein, 122 aa, synthetic construct
  source 1..122: note = VHH domain
EVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS

SEQ ID NO: 21 - protein, 12 aa, synthetic construct
  source 1..12: note = spot-tag
PDRVRAVSHWSS

SEQ ID NO: 22 - protein, 13 aa, synthetic construct
  source 1..13: note = ALFA-tag
  SITE 5..9: note = Oct-4-en cyclisation
SRLEAELRARLTE

SEQ ID NO: 23 - protein, 13 aa, synthetic construct
  source 1..13: note = ALFA-tag
  SITE 5..9: note = But-2-en cyclisation
SRLESELRSRLTE

SEQ ID NO: 24 - protein, 13 aa, synthetic construct
  source 1..13: note = ALFA-tag
  SITE 5..9: note = 1-propan-1,2,3 triazol cyclisation
  SITE 9: note = D-alanin
SRLEAELRARLTE

SEQ ID NO: 25 - protein, 13 aa, synthetic construct
  source 1..13: note = ALFA-tag
  SITE 5..9: note = N-methylpropanamide cyclisation
  SITE 9: note = D-alanin
SRLECGLRARLTE

SEQ ID NO: 26 - protein, 13 aa, synthetic construct
  source 1..13: note = ALFA-tag
  SITE 5..9: note = disulfid bridge cyclisation
SRLECELRCRLTE